SEQUENCE LISTING (157 sequences)

SEQ ID NO: 1 - protein, 20 aa, Artificial Sequence
  synthetic peptide from PGP2 sub-library
KEDNPGYSSEQDYNKLDGSC

SEQ ID NO: 2 - protein, 20 aa, Artificial Sequence
  synthetic peptide TRF20
GQTQFAMHRFQQWYKIKGSC

SEQ ID NO: 3 - protein, 20 aa, Artificial Sequence
  synthetic peptide TRF21
QYHHFMNLKRQGRAQAYGSC

SEQ ID NO: 4 - protein, 20 aa, Artificial Sequence
  synthetic peptide TRF22
HAYKGPGDMRRFNHSGMGSC

SEQ ID NO: 5 - protein, 20 aa, Artificial Sequence
  synthetic peptide TRF23
FRGWAHIFFGPHVIYRGGSC

SEQ ID NO: 6 - protein, 20 aa, Artificial Sequence
  synthetic peptide TRF24
SVKPWRPLLTGNRWLNSGSC

SEQ ID NO: 7 - protein, 20 aa, Artificial Sequence
  synthetic peptide TRF25
APYAPQQIHYWSTLGFKGSC

SEQ ID NO: 8 - protein, 20 aa, Artificial Sequence
  synthetic peptide TRF26
AHKVVPQRQIRHAYNRYGSC

SEQ ID NO: 9 - protein, 20 aa, Artificial Sequence
  synthetic peptide TRF27
LDPLFNTSIMVNWHRWMGSC

SEQ ID NO: 10 - protein, 20 aa, Artificial Sequence
  synthetic peptide TRF27
LDPLFNTSIMVNWHRWMGSC

SEQ ID NO: 11 - protein, 20 aa, Artificial Sequence
  synthetic peptide TRF28
RFQLTQHYAQFWGHYTWGSC

SEQ ID NO: 12 - protein, 15 aa, Artificial Sequence
  synthetic peptide BP1
GTEKGTSGWLKTGSC

SEQ ID NO: 13 - protein, 15 aa, Artificial Sequence
  synthetic peptide BP2
EGEWTEGKLSLRGSC

SEQ ID NO: 14 - DNA, 45 bp, Artificial Sequence
  synthetic oligonucleotide
cccgaaacaaccgcgagaggcacgcgcgtagccgtcaccggctat

SEQ ID NO: 15 - DNA, 32 bp, Artificial Sequence
  synthetic oligonucleotide
gctacgcgcgtgcctctcgcggttgtttcggg

SEQ ID NO: 16 - DNA, 29 bp, Artificial Sequence
  synthetic oligonucleotide
tagccggtgtgaagtttctgctagtaatg

SEQ ID NO: 17 - protein, 20 aa, Artificial Sequence
  polypeptide binding elements of SYN2
QYHHFMNLKRQGRAQAYGSG

SEQ ID NO: 18 - protein, 20 aa, Artificial Sequence
  Polypeptide binding elements of SYN22
HAYKGPGDMRRFNHSGMGSG

SEQ ID NO: 19 - protein, 20 aa, Artificial Sequence
  Peptide affinity element TRF23/synthetic polypeptide SYN23-26
FRGWAHIFFGPHVIYRGGSG

SEQ ID NO: 20 - protein, 20 aa, Artificial Sequence
  Peptide affinity element TRF26/synthetic polypeptide SYN23-26
AHKVVPQRQIRHAYNRYGSG

SEQ ID NO: 21 - DNA, 29 bp, Artificial Sequence
  Synthetic oligonucleotide Apt1
agtccgtggtagggcaggttggggtgact

SEQ ID NO: 22 - DNA, 15 bp, Artificial Sequence
  synthetic oligonucleotide Apt2
ggttggtgtggttgg

SEQ ID NO: 23 - protein, 20 aa, Artificial Sequence
  Peptide affinity element TRF24
SVKPWRPLITGNRWLNSGSG

SEQ ID NO: 24 - protein, 10 aa, Artificial Sequence
  synthetic polypeptide
KPGKKKPGKA

SEQ ID NO: 25 - protein, 27 aa, Artificial Sequence
  synthetic polypeptide
  MOD_RES (2)..(3): Hydroxyproline
  MOD_RES (5)..(6): Hydroxyproline
  MOD_RES (8)..(9): Hydroxyproline
  MOD_RES (11)..(12): Hydroxyproline
  MOD_RES (14)..(15): Hydroxyproline
  MOD_RES (17)..(18): Hydroxyproline
  MOD_RES (20)..(21): Hydroxyproline
  MOD_RES (23)..(24): Hydroxyproline
  MOD_RES (26)..(27): Hydroxyproline
GXXGXXGXXGXXGXXGXXGXXGXXGXX

SEQ ID NO: 26 - protein, 4 aa, Artificial Sequence
  polypeptide linker
GSGS

SEQ ID NO: 27 - protein, 20 aa, Artificial Sequence
  synthetic peptide linker
GSGSGSGSGSGSGSGSGSGS

SEQ ID NO: 28 - protein, 30 aa, Artificial Sequence
  synthetic peptide that may encompass 1-10 ′Gly-Hyp-Hyp′ repeating units
  MOD_RES (2)..(3): Hydroxyproline
  MOD_RES (5)..(6): Hydroxyproline
  MOD_RES (8)..(9): Hydroxyproline
  MOD_RES (11)..(12): Hydroxyproline
  MOD_RES (14)..(15): Hydroxyproline
  MOD_RES (17)..(18): Hydroxyproline
  MOD_RES (20)..(21): Hydroxyproline
  MOD_RES (23)..(24): Hydroxyproline
  MOD_RES (26)..(27): Hydroxyproline
  MOD_RES (29)..(30): Hydroxyproline
GXXGXXGXXGXXGXXGXXGXXGXXGXXGXX

SEQ ID NO: 29 - protein, 32 aa, Artificial Sequence
  synthetic peptide that may encompass 1-10 ′G1y-Hyp-Hyp′ repeating units
  MOD_RES (2)..(3): Hydroxyproline
  MOD_RES (5)..(6): Hydroxyproline
  MOD_RES (8)..(9): Hydroxyproline
  MOD_RES (11)..(12): Hydroxyproline
  MOD_RES (14)..(15): Hydroxyproline
  MOD_RES (17)..(18): Hydroxyproline
  MOD_RES (20)..(21): Hydroxyproline
  MOD_RES (23)..(24): Hydroxyproline
  MOD_RES (26)..(27): Hydroxyproline
  MOD_RES (29)..(30): Hydroxyproline
GXXGXXGXXGXXGXXGXXGXXGXXGXXGXXKG

SEQ ID NO: 30 - protein, 10 aa, Artificial Sequence
  Synthetic peptide
KPGKEKPGKA

SEQ ID NO: 31 - protein, 10 aa, Artificial Sequence
  synthetic peptide; Peptide may encompass 1-3 ′Pro-Gly-Pro′ repeating units
PGPPGPPGPK

SEQ ID NO: 32 - protein, 32 aa, Artificial Sequence
  peptide affinity element TRF 26 with (PGP)4 linker
AHKVVPQRQIRHAYNRYGSGPGPPGPPGPPGP

SEQ ID NO: 33 - protein, 12 aa, Artificial Sequence
  synthetic peptide linker
PGPPGPPGPPGP

SEQ ID NO: 34 - protein, 20 aa, Artificial Sequence
  synthetic peptide
FRGWAHIFFGPHVIYRGKSG

SEQ ID NO: 35 - DNA, 32 bp, Artificial Sequence
  synthetic oligonucleotide, template strand
  modified_base (20)..(20): N = amine-modified cytosine
  misc_feature (20)..(20): n is a, c, g, or t
gctacgcgcgtgcctctcgnggttgtttcggg

SEQ ID NO: 36 - DNA, 45 bp, Artificial Sequence
  synthetic oligonucleotide, variant strand
  modified_base (1)..(1): N = amine-modified cytosine
  misc_feature (1)..(1): n is a, c, g, or t
nccgaaacaaccgcgagaggcacgcgcgtagccgtcaccggctat

SEQ ID NO: 37 - DNA, 29 bp, Artificial Sequence
  synthetic oligonucleotide
  modified_base (1)..(1): N = Thymine linked to psoralen
  misc_feature (1)..(1): n is a, c, g, or t
  modified_base (29)..(29): N = Guanosine linked to thiol modifier C3
  misc_feature (29)..(29): n is a, c, g, or t
nagccggtgtgaagtttctgctagtaatn

SEQ ID NO: 38 - protein, 10 aa, Artificial Sequence
  synthetic peptide
  MOD_RES (1)..(1): Xaa = Lys modified with Fmoc
  MOD_RES (4)..(6): Xaa = at position 4 Lys modified with pNZ, at position 5 is Lys modified with Boc and at position 6, Lys modified with Dde
  MOD_RES (9)..(9): Xaa = Lys modified with Aloc protecting group
XPGXXXPGXA

SEQ ID NO: 39 - protein, 20 aa, Artificial Sequence
  peptide affinity element TRF19
KEDNPGYSSEQDYNKLDGSG

SEQ ID NO: 40 - protein, 20 aa, Artificial Sequence
  peptide affinity element TRF20
GQTQFAMHRFQQWYKIKGSG

SEQ ID NO: 41 - protein, 20 aa, Artificial Sequence
  peptide affinity element TRF25
APYAPQQIHYWSTLGFKGSG

SEQ ID NO: 42 - protein, 20 aa, Artificial Sequence
  peptide affinity element TRF27
LDPLFNTSIMVNWHRWMGSG

SEQ ID NO: 43 - protein, 15 aa, Artificial Sequence
  peptide affinity element BP1
GTEKGTSGWLKTGSG

SEQ ID NO: 44 - protein, 15 aa, Artificial Sequence
  peptide affinity element BP2
EGEWTEGKLSLRGSG

SEQ ID NO: 45 - protein, 20 aa, Artificial Sequence
  peptide affinity element TNF-alpha-1
MKSIIPMSVAQHQGPIKGSG

SEQ ID NO: 46 - protein, 20 aa, Artificial Sequence
  peptide affinity element TNF-alpha-2
RTTEMPFVFALGSVHPGGSG

SEQ ID NO: 47 - protein, 20 aa, Artificial Sequence
  peptide affinity element TNF-alpha-3
SMKMVQPGHLLISYGHQGSG

SEQ ID NO: 48 - protein, 20 aa, Artificial Sequence
  peptide affinity element TNF-alpha-4
FMNYPIKVPILVVPIGRGSG

SEQ ID NO: 49 - protein, 20 aa, Artificial Sequence
  peptide affinity element TNF-alpha-5
VMLYNWHIMQHRNNKPVGSG

SEQ ID NO: 50 - protein, 20 aa, Artificial Sequence
  peptide affinity element Bx3
AKGMFKAPYYKTPDRNRGSG

SEQ ID NO: 51 - protein, 20 aa, Artificial Sequence
  peptide affinity element Bx7
LSIMQSERLPHSWKGYRGSG

SEQ ID NO: 52 - protein, 20 aa, Artificial Sequence
  peptide affinity element Bx9
GTQPMVAWKDVYGIVVYGSG

SEQ ID NO: 53 - protein, 20 aa, Artificial Sequence
  peptide affinity element 6′SL
AQYSFVVGVKGFIHAQYGSG

SEQ ID NO: 54 - protein, 4 aa, Artificial Sequence
  synthetic peptide
GSKG

SEQ ID NO: 55 - protein, 8 aa, Artificial Sequence
  peptide linker template PGP1
  MOD_RES (2)..(2): Xaa = modified propargylglycine (Pra) residues
  MOD_RES (5)..(5): Xaa = Lys, Ser, Asp, Asn, Gly and Trp
  MOD_RES (8)..(8): Xaa = modified propargylglycine (Pra) residues
KXPPXPPX

SEQ ID NO: 56 - protein, 11 aa, Artificial Sequence
  peptide linker template PGP2
  MOD_RES (2)..(2): Xaa = modified propargylglycine (Pra) residues
  MOD_RES (5)..(5): Xaa = Arg, Thr, Glu, Gln, Gly and Phe
  MOD_RES (8)..(8): Xaa = Lys, Ser, Asp, Asn, Gly and Trp
  MOD_RES (11)..(11): Xaa = modified propargylglycine (Pra) residues
KXPPXPPXPPX

SEQ ID NO: 57 - protein, 14 aa, Artificial Sequence
  peptide linker template PGP3
  MOD_RES (2)..(2): Xaa = modified propargylglycine (Pra) residues
  MOD_RES (5)..(5): Xaa = His, Tyr, Ala, Met, Gly and Leu
  MOD_RES (8)..(8): Xaa = Arg, Thr, Glu, Gln, Gly and Phe
  MOD_RES (11)..(11): Xaa = Lys, Ser, Asp, Asn, Gly and Trp
  MOD_RES (14)..(14): Xaa = modified propargylglycine (Pra) residues
KXPPXPPXPPXPPX

SEQ ID NO: 58 - protein, 17 aa, Artificial Sequence
  peptide linker template PGP4
  MOD_RES (2)..(2): Xaa = modified propargylglycine (Pra) residues
  MOD_RES (5)..(5): Xaa = Lys, Ser, Asp, Asn, Gly and Trp
  MOD_RES (8)..(8): Xaa = His, Tyr, Ala, Met, Gly and Leu
  MOD_RES (11)..(11): Xaa = Arg, Thr, Glu, Gln, Gly and Phe
  MOD_RES (14)..(14): Xaa = Lys, Ser, Asp, Asn, Gly and Trp
  MOD_RES (17)..(17): Xaa = modified propargylglycine (Pra) residues
KXPPXPPXPPXPPXPPX

SEQ ID NO: 59 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPGPPGPPX

SEQ ID NO: 60 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPGPPSPPX

SEQ ID NO: 61 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPTPPGPPX

SEQ ID NO: 62 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPGPPNPPX

SEQ ID NO: 63 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPGPPDPPX

SEQ ID NO: 64 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPQPPGPPX

SEQ ID NO: 65 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPGPPKPPX

SEQ ID NO: 66 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPEPPGPPX

SEQ ID NO: 67 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPTPPSPPX

SEQ ID NO: 68 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPFPPGPPX

SEQ ID NO: 69 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPRPPGPPX

SEQ ID NO: 70 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPTPPNPPX

SEQ ID NO: 71 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPQPPSPPX

SEQ ID NO: 72 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPTPPDPPX

SEQ ID NO: 73 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPEPPSPPX

SEQ ID NO: 74 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPTPPKPPX

SEQ ID NO: 75 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPFPPSPPX

SEQ ID NO: 76 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPQPPNPPX

SEQ ID NO: 77 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPEPPNPPX

SEQ ID NO: 78 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPQPPDPPX

SEQ ID NO: 79 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPGPPWPPX

SEQ ID NO: 80 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPRPPSPPX

SEQ ID NO: 81 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPEPPDPPX

SEQ ID NO: 82 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPQPPKPPX

SEQ ID NO: 83 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPEPPKPPX

SEQ ID NO: 84 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPFPPNPPX

SEQ ID NO: 85 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPFPPDPPX

SEQ ID NO: 86 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPRPPNPPX

SEQ ID NO: 87 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPRPPDPPX

SEQ ID NO: 88 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPFPPKPPX

SEQ ID NO: 89 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPRPPKPPX

SEQ ID NO: 90 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPTPPWPPX

SEQ ID NO: 91 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPQPPWPPX

SEQ ID NO: 92 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPEPPWPPX

SEQ ID NO: 93 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPFPPWPPX

SEQ ID NO: 94 - protein, 11 aa, Artificial Sequence
  synthetic peptide from PGP2 sub-library
  MOD_RES (2)..(2): Xaa = modified propargylglycine (Pra) residues
  MOD_RES (11)..(11): Xaa = modified propargylglycine (Pra) residues
KXPPRPPWPPX

SEQ ID NO: 95 - protein, 32 aa, Artificial Sequence
  peptide affinity element TRF 26 synthesized with PGP linker and Cys residue
  MOD_RES (32)..(32): Xaa = C-terminus Pro of (PGP)4 linker with attached Ser residue
AHKVVPQRQIRHAYNRYGSGPGPPGPPGPPGX

SEQ ID NO: 96 - protein, 20 aa, Artificial Sequence
  peptide affinity element TRF 23 synthesized with a Ser near C-terminus
  MOD_RES (18)..(18): Xaa = Lys modified with an attached Ser residue
FRGWAHIFFGPHVIYRGXSG

SEQ ID NO: 97 - protein, 23 aa, Artificial Sequence
  peptide TRF-23 synthesized with alkyne functionalization near C-terminus
  MOD_RES (21)..(21): Xaa = Lys modified with an attached alkyne functionalization per Fig. 46
FRGWAHIFFGPHVIYRGGSGXSG

SEQ ID NO: 98 - protein, 21 aa, Artificial Sequence
  synthetic peptide TRF26 modified
  MOD_RES (20)..(20): Xaa = Lys azido-modified at the epsilon amine
AHKVFPQRQIRHAYNRYGSXG

SEQ ID NO: 99 - protein, 21 aa, Artificial Sequence
  synthetic peptide TRF23 modified
  MOD_RES (20)..(20): Xaa = Lys azido-modified at the epsilon amine
FRGWAHIFFGPHVIYRGGSXG

SEQ ID NO: 100 - protein, 20 aa, Artificial Sequence
  synthetic transferrin binding peptide TRF101
ARDLLIQKNSGQDVDHRGSC

SEQ ID NO: 101 - protein, 20 aa, Artificial Sequence
  synthetic transferrin binding peptide
NIRMLLRFTVFPAQKLIGSC

SEQ ID NO: 102 - protein, 20 aa, Artificial Sequence
  synthetic transferrin binding peptide
WMDDIDAPQDEWWVFHHGSC

SEQ ID NO: 103 - protein, 20 aa, Artificial Sequence
  synthetic transferrin binding peptide
DFLWSKSGILSHASWNHGSC

SEQ ID NO: 104 - protein, 20 aa, Artificial Sequence
  synthetic transferrin binding peptide TRF 102
NQYVPIFSQPEDPVQQEGSC

SEQ ID NO: 105 - protein, 20 aa, Artificial Sequence
  synthetic transferrin binding peptide
KMRTITYYHLQAILKQRGSC

SEQ ID NO: 106 - protein, 20 aa, Artificial Sequence
  synthetic transferrin binding peptide
DNSRRSAKQRIFMHVDLGSC

SEQ ID NO: 107 - protein, 20 aa, Artificial Sequence
  synthetic transferrin binding peptide
AMMRFDMAGLNKIVFHQGSC

SEQ ID NO: 108 - protein, 20 aa, Artificial Sequence
  synthetic transferrin binding peptide
DRDTPWETTNKTEEGIEGSC

SEQ ID NO: 109 - protein, 20 aa, Artificial Sequence
  synthetic transferrin binding peptide
QENDQQSFGLGGMMGQAGSC

SEQ ID NO: 110 - protein, 20 aa, Artificial Sequence
  synthetic transferrin binding peptide
TEDNDYMVVSMVVTMEPGSC

SEQ ID NO: 111 - protein, 20 aa, Artificial Sequence
  synthetic peptide TNF1
FERDPLMMPWSFLQSRQGSC

SEQ ID NO: 112 - protein, 15 aa, Artificial Sequence
  synthetic peptide BP2 variant
EGEDTEGKKSKEGSC

SEQ ID NO: 113 - protein, 15 aa, Artificial Sequence
  synthetic peptide BP2 variant
EGEDTEGKKSKGGSC

SEQ ID NO: 114 - protein, 15 aa, Artificial Sequence
  synthetic peptide BP2 variant
EGEDTEGKKSKIGSC

SEQ ID NO: 115 - protein, 15 aa, Artificial Sequence
  synthetic peptide BP2 variant
EGEDTEGKKSKLGSC

SEQ ID NO: 116 - protein, 15 aa, Artificial Sequence
  synthetic peptide BP2 variant
EGEDTEGKKSKRGSC

SEQ ID NO: 117 - protein, 15 aa, Artificial Sequence
  synthetic peptide BP2 variant
EGEDTEGKKSKTGSC

SEQ ID NO: 118 - protein, 15 aa, Artificial Sequence
  synthetic peptide BP2 variant
EGEITEGKGSKKGSC

SEQ ID NO: 119 - protein, 15 aa, Artificial Sequence
  synthetic peptide BP2 variant
EGEITEGKKSKEGSC

SEQ ID NO: 120 - protein, 15 aa, Artificial Sequence
  synthetic peptide BP2 variant
EGEITEGKKSKGGSC

SEQ ID NO: 121 - protein, 15 aa, Artificial Sequence
  synthetic peptide BP2 variant
EGEITEGKKSKIGSC

SEQ ID NO: 122 - protein, 15 aa, Artificial Sequence
  synthetic peptide BP2 variant
EGEITEGKKSKLGSC

SEQ ID NO: 123 - protein, 15 aa, Artificial Sequence
  synthetic peptide BP2 variant
EGEITEGKKSKRGSC

SEQ ID NO: 124 - protein, 15 aa, Artificial Sequence
  synthetic peptide BP2 variant
EGEITEGKKSKSGSC

SEQ ID NO: 125 - protein, 15 aa, Artificial Sequence
  synthetic peptide BP2 variant
EGEITEGKKSKTGSC

SEQ ID NO: 126 - protein, 15 aa, Artificial Sequence
  synthetic peptide BP2 variant
EGEITEGKLSKKGSC

SEQ ID NO: 127 - protein, 15 aa, Artificial Sequence
  synthetic peptide BP2 variant
EGEKTEGKKSKEGSC

SEQ ID NO: 128 - protein, 15 aa, Artificial Sequence
  synthetic peptide BP2 variant
EGEKTEGKKSKGGSC

SEQ ID NO: 129 - protein, 15 aa, Artificial Sequence
  synthetic peptide BP2 variant
EGEKTEGKKSKLGSC

SEQ ID NO: 130 - protein, 15 aa, Artificial Sequence
  synthetic peptide BP2 variant
EGEKTEGKKSKTGSC

SEQ ID NO: 131 - protein, 15 aa, Artificial Sequence
  synthetic peptide BP2 variant
EGEWTEGKKSKEGSC

SEQ ID NO: 132 - protein, 15 aa, Artificial Sequence
  synthetic peptide BP2 variant
EGEWTEGKKSKGGSC

SEQ ID NO: 133 - protein, 15 aa, Artificial Sequence
  synthetic peptide BP2 variant
EGEWTEGKKSKIGSC

SEQ ID NO: 134 - protein, 15 aa, Artificial Sequence
  synthetic peptide BP2 variant
EGEWTEGKKSKLGSC

SEQ ID NO: 135 - protein, 15 aa, Artificial Sequence
  synthetic peptide BP2 variant
EGEWTEGKKSKRGSC

SEQ ID NO: 136 - protein, 15 aa, Artificial Sequence
  synthetic peptide BP2 variant
EGEWTEGKKSKTGSC

SEQ ID NO: 137 - DNA, 116 bp, Artificial Sequence
  synthetic oligonucleotide library sequence
  misc_feature (49)..(50): N = G, C, T or A
  misc_feature (51)..(51): N = G or C
  misc_feature (52)..(53): N = G, C, T or A
  misc_feature (54)..(54): N = G or C
  misc_feature (55)..(56): N = G, C, T or A
  misc_feature (57)..(57): N = G or C
  misc_feature (58)..(59): N = G, C, T or A
  misc_feature (60)..(60): N = G or C
  misc_feature (61)..(62): N = G, C, T or A
  misc_feature (63)..(63): N = G or C
  misc_feature (64)..(65): N = G, C, T or A
  misc_feature (66)..(66): N = G or C
  misc_feature (67)..(68): N = G, C, T or A
  misc_feature (69)..(69): N = G or C
  misc_feature (70)..(71): N = G, C, T or A
  misc_feature (72)..(72): N = G or C
  misc_feature (73)..(74): N = G, C, T or A
  misc_feature (75)..(75): N = G or C
  misc_feature (76)..(77): N = G, C, T or A
  misc_feature (78)..(78): N = G or C
  misc_feature (79)..(80): N = G, C, T or A
  misc_feature (81)..(81): N = G or C
  misc_feature (82)..(83): N = G, C, T or A
  misc_feature (84)..(84): N = G or C
  misc_feature (85)..(86): N = G, C, T or A
  misc_feature (87)..(87): N = G or C
  misc_feature (88)..(89): N = G, C, T or A
  misc_feature (90)..(90): N = G or C
  misc_feature (91)..(92): N = G, C, T or A
  misc_feature (93)..(93): N = G or C
  misc_feature (94)..(95): N = G, C, T or A
  misc_feature (96)..(96): N = G or C
  misc_feature (97)..(98): N = G, C, T or A
  misc_feature (99)..(99): N = G or C
ttctaatacgactcactatagggacaattactatttacaattacaatgnnnnnnnnnnnnnnnnnnnnnnnnnnnnnnnnnnnnnnnnnnnnnnnnnnnatgggaatgtctggatc

SEQ ID NO: 138 - DNA, 48 bp, Artificial Sequence
  synthetic oligonucleotide
atagccggtgctaccgctcagggcctgataagatccagacattcccat

SEQ ID NO: 139 - DNA, 27 bp, Artificial Sequence
  synthetic oligonucleotide
atagccggtgctaccgctcagggcctg

SEQ ID NO: 140 - DNA, 45 bp, Artificial Sequence
  synthetic oligonucleotide
ttctaatacgactcactatagggacaattactatttacaattaca

SEQ ID NO: 141 - protein, 20 aa, Artificial Sequence
  synthetic peptide clone TBPM023
AHKVVPQRQMRHAYSRYGSC

SEQ ID NO: 142 - protein, 20 aa, Artificial Sequence
  synthetic peptide clone TBPM021
ATRWCPSARPATPTTATGSC

SEQ ID NO: 143 - protein, 20 aa, Artificial Sequence
  synthetic peptide clone TBPLOO5
GHKVVPQRQIRHAYNRYGSC

SEQ ID NO: 144 - protein, 20 aa, Artificial Sequence
  synthetic peptide clone TBPL025
GHKVVPQRQMRHAYNRNGSC

SEQ ID NO: 145 - protein, 20 aa, Artificial Sequence
  synthetic peptide clone TBPM003
PTGWCPAPDPPRLHPLHGSC

SEQ ID NO: 146 - protein, 20 aa, Artificial Sequence
  synthetic peptide
  MOD_RES (20)..(20): Xaa = Gly terminally modified by linker (PGP)4 linked to Lys azido-modifed at epsilon amine linked to Fmoc-Cys(Trt)-OH/Fmoc-Cys(tButhio)-Oh/Fmoc-Lys (biotin)-OH/Fmoc-Glu(biotinyl-PEG-OH
HAYKGPGDMRRFNHSGMGSX

SEQ ID NO: 147 - protein, 20 aa, Artificial Sequence
  synthetic peptide
  MOD_RES (18)..(18): Xaa = Fmoc-Lys(ivDde)-OH/Fmoc-propargyiglycine
KEDNPGYSSEQDYNKLDXSG

SEQ ID NO: 148 - protein, 50 aa, Artificial Sequence
  synthetic peptide synbody A-(PGP)1-B
AHKVVGQRGIRHAYNRYGSGPGPKGKGFRGWAHIFFGPHVIYRGGSGKSG

SEQ ID NO: 149 - protein, 63 aa, Artificial Sequence
  synthetic peptide synbody A-(PPP)6-G
AHKVVPQRQIRHAYNRYGSGPPPPPPPPPPPPPPPPPPKCFRGWAHIFFGPHVIYRGGSGKSG

SEQ ID NO: 150 - protein, 42 aa, Artificial Sequence
  synthetic peptide synbody A-B
AHKVVPQRQIRHAYNRYGSGKCFRGWAHIFFDPHVIYRGGSG

SEQ ID NO: 151 - protein, 51 aa, Artificial Sequence
  synthetic peptide synbody A-(PGP)1-G
AHKVVPQRQIRHAYNRYGSGPGPKGKGHAYKGPGDMRRFNHSGMSGSGKSG

SEQ ID NO: 152 - protein, 42 aa, Artificial Sequence
  synthetic peptide synbody mutA-B
AHKVVYQRQIRFAYNRYGSGKCFRGWAHIFFGPHVIYRGGSG

SEQ ID NO: 153 - protein, 32 aa, Artificial Sequence
  synthetic peptide synbody C-D
EGEWTEGKLSLRGSGKCGTEKGTSGWLKTGSG

SEQ ID NO: 154 - protein, 32 aa, Artificial Sequence
  synthetic peptide synbody mutC-D
EGWWTEGKLSLRGSGKCGTEKGTSGWLKTGSG

SEQ ID NO: 155 - protein, 41 aa, Artificial Sequence
  synthetic peptide synbody E-F
FERSYLKMPWKFLQSRQSGKCWGPSYKFKITRFHQQSSGSG

SEQ ID NO: 156 - protein, 42 aa, Artificial Sequence
  synthetic peptide synbody B-A
FRGWAHIFFGPHVIYRGGSGKCAHKVVPQRQIRHAYNRYGSG

SEQ ID NO: 157 - protein, 42 aa, Artificial Sequence
  synthetic peptide synbody B-B
FRGWAHIFFGPHVIYRGGSGKCFRGWAHIFFGPHVIYRGGSG

(19) United States
(12) Patent Application Publication
(10) Pub. No.: US 2019/0194358 A1
(43) Pub. Date: Jun. 27, 2019

(54) Synthetic Antibodies

(71) Applicants: Stephen A Johnston, Tempe, AZ (US); Neal Woodbury, Tempe, AZ (US); Christopher W. Diehnelt, Chandler, AZ (US); Paul Belcher, Boston, MA (US); Nidhi Gupta, Phoenix, AZ (US); Zhan-Gong Zhoa, Tucson, AZ (US); Matthew Greving, Phoenix, AZ (US); Jack Emery, Tempe, AZ (US)

(72) Inventors: Stephen A Johnston, Tempe, AZ (US); Neal Woodbury, Tempe, AZ (US); Christopher W. Diehnelt, Chandler, AZ (US); Paul Belcher, Boston, MA (US); Nidhi Gupta, Phoenix, AZ (US); Zhan-Gong Zhoa, Tucson, AZ (US); Matthew Greving, Phoenix, AZ (US); Jack Emery, Tempe, AZ (US)

(73) Assignee: Arizona Board of Regents on behalf of Arizona State University, Scottsdale, AZ (US)

(21) Appl. No.: 16/198,288

(22) Filed: Nov. 21, 2018

Related U.S. Application Data
(62) Division of application No. 14/072,152, filed on Nov. 5, 2013, now abandoned.
Application No. 14/072,152 is a continuation-in-part of application No. 12/989,156, filed on Feb. 10, 2011, now abandoned.
(60) Provisional application No. 61/163,034, filed on Mar. 24, 2009.
(60) Provisional application No. 61/047,422, filed on Apr. 23, 2008.

(51) Int. Cl. C07K 17/10; G01N 33/68; C07K 17/06

(52) U.S. Cl. CPC C07K 17/10; C07K 17/06; G01N 33/6845

(57) ABSTRACT

The present invention provides methods for synthetic antibodies, methods for making synthetic antibodies, methods for identifying ligands, and related methods and reagents.

### CROSS REFERENCE

This application is a continuation-in-part of and claims priority from U.S. application Ser. No. 12/989,156 which was national stage application of PCT/US09/41570, filed Apr. 23, 2009 to Johnston et al. entitled “Synthetic Antibodies,” the disclosure of which is incorporated by reference; and further claims the benefit of 61/047,422 filed Apr. 23, 2008 and 61/163,034 filed Mar. 24, 2009, both incorporated by reference in their entirety for all purposes.

### STATEMENT OF GOVERNMENT INTEREST

The invention was made in part funded by U.S. government NIAID grant number 5 U54 A1057156 and NCI grant number 5 U54 CA112952, and thus the U.S. government has certain rights in the invention.

### BACKGROUND OF THE INVENTION

The basic use of antibodies or ligands is that they can distinguish one component from others in a complex mixture. The level of distinction required varies by use. The fundamental problem in antibody (ligand) development is to find some entity that can structurally complement a region or regions on the surface of the target, and that that complementation is higher to a necessary degree above that of other components in the mixture.

Traditional antibodies are produced by injection of a protein or genes encoding proteins into an animal, usually multiple times over 1-4 months. Polyclonal antibodies are directly used from the serum. They can be affinity purified if a sufficient amount of the target protein is available. Using hybridoma technology, individual clones producing one element of the polyclonal population can be identified and the antibody propagated indefinitely. This procedure is generally erratic in the quality of the product, slow, low through put, suffers from contaminants and is expensive. It also requires killing animals. The most advanced form of this approach uses genetic immunization¹. For each antibody the gene corresponding to the protein sequence is chemically synthesized and injected into the animal's skin with a gene gun. In parallel a small amount of protein is in vitro transcribed/translated using the same gene fragment. This protein is attached to beads for a direct assessment of reactivity. This system avoids the necessity of protein production for immunization, contaminants and is relatively high through-put. The quality of the antibodies is generally higher. However, this system still requires labor intensive animal handling². To produce replenishable antibody, this system must be coupled to traditional monoclonal production³.

Alternatives to direct production of antibodies in animals generally involve recurrent selection processes which are expensive, but more importantly not adaptable to high throughput methods. Antibodies used clinically have affinities (Kd) for their targets of 10⁻¹² to 5×10⁻⁸ M/l. This affinity is generated biologically by selecting mutations in the variable region of the antibody. The variable region is basically a flexible peptide held at the N and C-termini. By selecting from the ^{˜}10⁷ variants in any individual and mutationally improving the sequence, antibody maturation can produce a good binder to almost any target. The common approach to replicating this process is to create a very large library (10⁹-10¹⁴ members) of molecules with variable nucleic acids or polypeptides and panning against the target to find the one or few best binders. A selection process is applied where strong binders out compete weaker binders.

This basic approach of panning large libraries is the most commonly used to find antibody-like elements. However, such panning has severe limitations. First, since one is looking for a very good match in interaction using a relatively short peptide or nucleic acid one has to generate and search large libraries. This is both time consuming and does not lend it self to high through put. In most cases, recurrent selection (panning) must be used to find the perfect match so only the best binding area on a target is found. It is difficult to find binders to multiple areas on the target. Other approaches have utilized meticulous application of chemistry and structural determinations to produce a molecule in which two small organic molecules were bound by a short rigid linker. However, this approach demands exquisite chemistry and structural biology, and the small molecules must be perfectly positioned for binding, thus putting severe restrictions on the nature of the linker. Furthermore, the nature of the binding elements, small organic molecules, is inherently limiting. It has proven very difficult to find a second site on a given protein that will sufficiently bind a small organic molecule. On reflection this makes perfect sense. Since the protein concentration in a cell is 60-100 mg/ml most exposed surfaces of a protein must be non-binding or all proteins would agglomerate. Therefore, small molecules will generally only bind in deep pockets on the protein.

Thus, new methods for ligand discovery and resulting ligands for use in constructing, for example, synthetic antibodies are needed in the art.

This application is also related to WO/2008/048970 filed Oct. 15, 2007, and Provisional Patent Application Ser. Nos. 60/852,040 filed Oct. 16, 2006, and 60/975,442 filed Sep. 26, 2007, each incorporated by reference herein in its entirety for all purposes.

### SUMMARY OF THE INVENTION

The invention provides a multimeric peptide. The multimeric peptide comprises a first affinity element conjugated to a second affinity element, wherein the first affinity element comprises a first peptide conjugated to a first DNA strand, the second affinity element comprises a second peptide conjugated to a second DNA strand, the first peptide and second peptide comprise a random combination of amino acids selected from the group of G, T, Q, K, S, W, L, and R; and the first affinity element is conjugated to the second affinity element by hybridization of the first DNA strand and the second DNA strand. Optionally, the first peptide and the second peptide each comprise 8 to 35 amino acids, more preferably 8 to 20 amino acids. Optionally, the first DNA strand and the second DNA strand are synthetic DNA. Optionally, the total distance between the first peptide and the second peptide is between 0.5 nm and 30 nm, preferably between 0.5 nm and 10 nm or 0.5 nm and 4.3 nm or 0.5 nm and 2 nm.

In some embodiments, the multimeric peptide further comprises a first template DNA strand and a second template DNA strand wherein the at least one template DNA strand conjugates the first peptide and the second peptide with the first DNA strand and the second DNA strand, respectively. Optionally, the first template DNA strand and second template DNA strand are conjugated to the first peptide and the second peptides, respectively, at the peptides' C-terminus. Optionally, the first template DNA strand and the second template DNA strand is conjugated to the first peptide and the second peptides, respectively, using standard amine coupling chemistry. Optionally, the first DNA strand and the second DNA strand is conjugated to the first template DNA strand and the second template DNA strand, respectively, by UV cross-linking.

The invention further provides a method of constructing a multimeric peptide comprising hybridizing the DNA strands of two affinity elements, wherein the method of synthesizing the affinity element comprises: conjugating a template DNA strand with a peptide; and conjugating the template DNA strand with a second DNA strand. Optionally, the template DNA strand is conjugated to the peptide at the C-terminus of the peptide. Optionally, the template DNA strand is conjugated to the peptide using standard amine coupling chemistry. Optionally, the template DNA strand is conjugated with the second DNA strand using UV cross-linking. Optionally, the total distance between the peptides in the two affinity elements is between 0.5 nm and 30 nm, preferably between 0.5 nm and 10 nm or 0.5 nm and 4.3 nm or 0.5 nm and 2 nm.

In some embodiments, the methods of constructing a multimeric peptide further comprise conjugating the second DNA strand with a label. Optionally, the label is fluorescent.

The invention further provides a method of screening a multimeric peptide that binds a target comprising: generating a pool of peptides comprising random combinations of amino acids selected from the group of G, T, Q, K, S, W, L, and R; contacting the pool of peptides with a target; determining the peptides in the pool of peptides that binds to a target; mapping the locations on the target that the peptides in the pool of peptides bind; conjugating two peptides in the pool of peptides that binds to different locations on the target with DNA strands to produce multivalent binding agents; contacting the multivalent binding agents with the target; and identifying the multivalent binding agents that binding to the target. Optionally, the random combinations of amino acids comprise tryptophan. Optionally, the random combinations of amino acids comprise 8 to 35 amino acids, more preferably 8 to 20 amino acid. Optionally, the pool of peptides comprises 1000 to 25000 peptides, more preferably 4000 to 25000 peptides.

In some embodiments, conjugating the two peptides in the pool of peptides that binds to different locations on the target with DNA strands comprises standard amine coupling chemistry and UV cross-linking. Optionally, the locations on the target that the peptides in the pool of peptides bind are determined by protein-protein interface mapping.

In some embodiments, a method of screening a multimeric peptide that binds a target further comprises identifying the optimal distance between the two peptides in the multivalent binding agents for the highest binding affinity to the target. Optionally, the binding affinity of the peptides in the pool of peptides to the target is detected using surface plasmon resonance. Optionally, the binding affinity of the peptides in the pool of peptides to the target is detected using ELISA. Optionally, the distance between the two peptides in the multivalent binding agents are less than 10 nm.

### DESCRIPTION OF THE FIGURES

FIG. 1. Legend for conceptual drawings of synbody variations shown FIGS. 2-8.

FIG. 2. Schematic of simple synbody.

FIGS. 3A and B. Schematic of synbodies specific for (A) homodimers and (B) heterodimers.

FIGS. 4A and B. Schematic of synbodies that act as chemical OR gates or switches.

FIG. 5. Schematic of synbodies that bind multiple A molecules cooperatively (a≠1, either positive or negative cooperativity)

FIG. 6. Schematic of synbodies that bind multiple different molecules cooperatively (a≠1, either positive or negative cooperativity)

FIG. 7 Schematic of synbodies that act as signaling molecular sensors; two elements interact to form signal (upper); two elements are displaced to form signal (lower).

FIG. 8. Schematic of synbodies acting as actuators of enzyme activity (homo or heteromultimer)

FIGS. 9A-C. (A) Representation of synthetic antibody. (B) Construction of mini-library of synbodies with different interpeptide distances. (C) One embodiment of a molecular slide rule composition

FIGS. 10A, B (A) Structure of maleimide sulfo-SMCC (sulfosuccinimidyl 4[N-maleimidomethyl]cyclohexane-1-carboxylate) (B) Conjugation of polypeptides to polylysine surface coating by thiol attachment of a C-terminal cysteine of the polypeptide to ε amine of a lysine monomer of the poly-lysine surface coating using sulfo-SMCC.

FIGS. 11A, B. (A) Signal expected during attachment of protein target to SPR chip surface. (B) Steps in attachment of protein target to SPR chip surface.

FIGS. 12A-D. Expected SPR signal upon (A) interaction of a first ligand alone with an immobilized target; (B) interaction of a second ligand alone with an immobilized target; (C) interaction of a first and second ligand with an immobilized target where the ligands do not compete or interfere; (D) binding of two ligands that do not bind distinct sites on the target, but instead compete for the same binding site.

FIG. 13. Results of evaluation for binding to distinct target sites, of a number of pairs of the polypeptides that were identified as described in Example 2 (see Table 1).

FIG. 14. 5′-Dimethoxytrityl-N-dimethylformamidine-5-[N-(trifluoroacetylaminohexyl)-3-acrylimido]-2′-deoxyCytidine, 3′-[(2-cyanoethyl)-(N,N-diisopropyl)]-phosphorarnidite, used to provide amine-modified cytosines in oligonucleotides.

FIG. 15. Schematic representation of a synbody specific for gal80, comprising two polypeptide affinity elements identified as described in Example 3 joined by a DNA linker.

FIG. 16. A synbody comprising polypeptide affinity elements.

FIG. 17. Flow chart of the synthesis of a synbody comprising polypeptide affinity elements.

FIG. 18. Relative SPR responses of BP1 and BP2-containing synbodies with respect to gal80.

FIG. 19. Affinities (Kd) with respect to gal80 of affinity elements BP1 and BP2 alone, BP1-BP2 containing synbody, and BP1 and BP2 alone conjugated to DNA linker.

FIG. 20. Data derived from ELISA-type analyses confirming the binding affinities of BP1 and BP2 alone for gal80 compared to the BP1-BP2 containing synbody.

FIG. 21. Schematic of synbodies constructed by linking the C-terminal glycines of two 20-mer polypeptides to the α and ε amine moieties of a lysine molecule, thereby providing a spacing of about 1 nm.

FIG. 22. Graph showing the 18 proteins to which 1C10 bound with highest intensity, and relative intensities observed.

FIG. 23. Graph showing the 18 proteins to which SYN23-26 bound with highest intensity, and relative intensities observed.

FIG. 24. Graph showing the 18 proteins to which SYN21-22 bound with highest intensity, and relative intensities observed.

FIG. 25. Graph showing the 15 proteins to which the gal80 synbody bound with highest intensity, and relative intensities observed.

FIG. 26. (a) Schematic of the 4-helix DNA tile linker constructed from DNA oligonucleotides. (b) Location of aptamers specific for thrombin incorporated into the single-stranded DNA loops, providing a structure in which the aptamers extend from the tile as shown schematically. (c) Structure having only a single aptamer containing loop. (d) Another structure having only a single aptamer containing loop.

FIG. 27. Graph showing results of thrombin-binding assays on the DNA tile synbodies.

FIG. 28. Pairs of chemical moieties suitable for conjugation by click-type chemistry.

FIG. 29. Four pairs of chemical moieties suitable for conjugation by click-type chemistry that, when conjugations are performed in the order indicated, provide four orthogonal conjugations.

FIG. 30. Diagram of synthesis of a synbody comprising a poly-(Gly-Ser) linker.

FIG. 31. Diagram showing conjugation of a maleimide functionalized polypeptide with a thiol functionalized oligonucleotide.

FIG. 32. Diagram of synthesis of a synbody comprising a poly-(Gly-Hyp-Hyp) linker.

FIG. 33. Diagram of synthesis of a synbody comprising a poly-(Gly-Hyp-Hyp) linker wherein both affinity elements are attached by click-type chemistry conjugation.

FIG. 34. Schematic illustration of a concept underlying a method for identification of optimized affinity elements and/or linkers by allowing a synbody to self-assemble in association with a target.

FIG. 35. Diagram showing three potentially reversible conjugation chemistries.

FIG. 36. Diagram showing synthesis of a tetrapeptide scaffold suitable for use as a synbody linker.

FIG. 37. Diagram illustrating orthogonal conjugation of up to three affinity elements to tetrapeptide scaffold linker.

FIG. 38. Diagram showing synthesis of decapeptide scaffold suitable for use as a synbody linker.

FIG. 39. Diagram illustrating orthogonal conjugation of affinity elements to decapeptide scaffold linker.

FIG. 40 shows azide-alkyne conjugation to link peptides to form a synbody.

FIG. 41 shows synthesis of a poly-(Pro-Gly-Pro) linked synbody.

FIG. 42 shows synthesis of a synbody having two peptide affinity elements, linked by conjugating them to the α and ε amine moieties of a lysine monomer.

FIG. 43 shows synthesis of a synbody.

FIGS. 44A and 44B show MALDI-TOF analysis of synbodies.

FIG. 45 shows synthesis of a peptide affinity element conjugated to a poly-proline or poly-[proline-glycine-proline] linker, with the distal portion of the linker azido-modified to facilitate conjugation of a second peptide affinity element thereto via azide-alkyne “click” conjugation.

FIG. 46 shows alkyne modification of a peptide.

FIG. 47 shows production of a bivalent synbody by azide-alkyne conjugation of an alkyne modified peptide with an azido-modified linker preconjugated to another peptide.

FIG. 48 shows azide-alkyne click conjugation.

FIGS. 49A and B shows an example of the HPLC separation and MALDI-TOF mass spectrographic verification of a synbody.

FIG. 50 shows assembly of a synbody having two peptide affinity elements conjugated to opposite ends of a poly-proline linker.

FIG. 51 depicts a PGP having a single variable position 203.

FIGS. 52-54 show MALDI-mass spectra of the gas phase cleaved sample of a PGP2 sub-library at increasing levels of detail.

FIGS. 55 and 56 show MALDI mass spectra acquired for the solution phase cleavage sample of the PGP2 linker sub-library.

FIG. 57 shows a scheme for synthesis of bivalent synbodies.

FIGS. 58A, B, C shows the MS analysis before addition of catalyst (Cu and vitamin C) (C), immediately after the addition of catalyst (B), and 4 hours after the addition of catalyst and reaction at 45° C. (A).

FIGS. 59A (full spectrum) and 59B (expanded view of 3500-9800 MW range) show a MALDI-MS analysis after synthesis of synbodies.

FIGS. 60A-L show sensorgrams for the binding of 12 selected peptides to transferrin.

FIG. 61 shows kinetic properties of a variant peptide (TRF101).

FIG. 62 compares the binding responses in SPR assay of 768 peptides as against transferrin target vs the same peptides as against ubiquitin target.

FIG. 63 shows MALDI spectra of synbodies screened against various targets.

FIG. 64 shows relatively strong binding kinetics for synbody TNF1-TNF10-KC-stBu and no binding for synbody TNF1-TNF4-KC-stBu.

FIG. 65 shows the affinity profile of peptide variants.

FIG. 66 compares the affinity of variant peptides to a lead peptide.

FIG. 67 shows a plot of the intensities corresponding to spotted peptides under different conditions.

FIG. 68 compares fluorescence intensity of peptides in a peptide-down versus target-down format.

FIG. 69 shows a density plot comparing the end to end length of peptides complexed to proteins in PDB structures.

FIG. 70: Heat matrix of effect of variations at different positions in the peptide TNF-1. Fold-change heat map from the initial SPR screen of TNF1 point-mutants.

FIG. 71: Fold-change in TNF-α affinity across four generations (single/double/triple/quadruple mutants) of TNF1 mutant sequences. Fold-change is calculated from the association constant (Ka=1/Kd) of a mutant divided by the Ka of the TNF1 lead peptide.

FIG. 72: Observed double, triple and quadruple mutant binding free energy versus the predicted binding free energy assuming mutational additivity. Observed binding free energies were calculated from the dissociation constants measured across several replicate experiments, predicted binding free energies were calculated as the sum of component binding free energies from the corresponding point mutants. The 95% confidence interval for the best-fit line (solid line) is shaded. The observed slope (0.97±0.01) of the best-fit line is close to the slope predicted from mutational additivity (predicted=1).

FIG. 73: Molecular dynamics (MD) conformational analysis of the TNF1 (top) and TNF1-opt (bottom) peptides. For each peptide, 2600 conformations were sampled from a total of 1 μs of MD trajectories. These conformations were clustered by backbone structural alignment within 1 Å pair-wise RMSD. The fraction of the total number of conformations for the ten largest clusters is shown in the bar graph on the left. Representative backbone conformations for the mutated region of the peptide (residues 4-11) from each of the top ten clusters are shown on the right, with the N-terminal end at the top and the structures ordered from cluster 1-10, left-to-right.

FIGS. 74 A, B, and C show nine synbodies (A), heat maps of binding to an array of 8000 proteins in which different colors represent different binding strengths (B), and the top five proteins bound by each synbody (C).

### DETAILED DESCRIPTION OF THE INVENTION

The invention provides methods of identifying a multimeric compound that binds to a target of interest. Such a multimeric compound is also known as a synthetic antibody or synbody. Such synthetic antibodies are useful as therapeutics as well as in imaging and diagnostics. The compounds forming the multimer or synthetic antibody are preferably peptides as broadly defined below. For ease of reference, the following description often refers to peptides, although other compounds can be used in place of peptides unless the context requires otherwise. The methods typically begin with a library of monomeric peptides. The size of the library is a balance between two factors. A larger the library is in principle relatively more likely to include members having affinity for any target of interest. However, a larger library also increases the amount of time and effort required to screen individual members for binding to a target. Initial libraries typically contain at least 100 members. A library size between 1000 and 25000 provides a good compromise between likelihood of obtaining members with detectable binding to any target of interest and ease of screening. Libraries of size from 100 to 50,000 members, for example can also be used. Such libraries typically represent only a very small proportion of total sequence space, for example less than 10⁻⁶, 10⁻¹⁰, or 10⁻¹⁵. Sequence space means the total number of permutations of sequence of a given set of monomers. For example, for the set of 20 natural amino acids there are 20ⁿ permutations, where n is the length of a peptide.

The lengths of peptides in an initial library represent a compromise between binding affinity and ease of synthesis. There is some relationship between peptide length and binding affinity with increasing length increasing affinity. However, as peptide length increases the likelihood of binding a binding site on a target that interacts with the full peptide length decreases. Cost of synthesis also increases with increasing length as does the likelihood of insolubility. The methods are typically practiced with initial libraries having peptides having 8-35 residues, with 15-25 being preferred.

The initial libraries are usually made by chemical synthesis. Such a process can increase the diversity of natural peptides in that unnatural amino acids or unnatural linkages between amino acids can easily be included. The diversity of chemically synthesized libraries is also greater than that of genetically encoded libraries because genetic expression selects against some peptide sequences. Although library members can be linked to tags encoding the identity of each member, such is usually unnecessary. Chemical synthesis typically produces peptides in an impure state (e.g., unreacted precursors may be present). A high degree of purity is not necessary in the methods that follow. For example, peptides can be used that are 50-80% or 60-90% pure w/w.

The peptides present in an initial library are typically chosen without regard to the identity of a particular target or natural ligand(s) to the target. In other words, the composition of an initial library is typically not chosen because of a priori knowledge that particular peptides bind to a particular target or have significant sequence identity either with the target or known ligands thereto. A sequence identity between a peptide and a natural sequence (e.g., a target or ligand) is considered significant if at least 30% of the residues in the peptide are identical to corresponding residues in the natural sequence when maximally aligned as measured using a BLAST or BLAST 2.0 sequence comparison algorithm with default parameters described below, or by manual alignment and visual inspection (see, e.g., NCBI web site ncbi.nlm.nih.gov/BLAST or the like).

Often the initial library is randomly selected from total sequence space or a portion thereof (e.g., in which certain amino acids are absent or under-represented). Random selection can be completely random in which case any peptide has an equal chance of being selected from sequence space or partially random in which case the selection involves random choices but is biased toward or against certain amino acids. Random selection of peptides can be made for example by a random computer algorithm. The randomization process can be designed such that different amino acids are equally represented in the resulting peptides, or occur in proportions representing those in nature, or in any desired proportions. Often cysteine residues are omitted from library members with the possible exception of a terminal amino acid, which provides a point of attachment to a support. In some libraries, certain amino acids are held constant in all peptides. For example, in some libraries, the three C-terminal amino acids are glycine, serine and cysteine with cysteine being the final amino acid at the C-terminus.

Other factors that can be taken into account in determining members of the initial library include theta temperatures and charge distributions of peptides. A theta temperature refers to the temperature at which a particular peptide is in a theta state under solvent conditions of interest. In a theta state, the theoretical conformation for a peptide is random flight with a theoretical end-to-end length equal to the distance between monomers times the square root of the number of monomers. The theta state of peptides can be taken into account by estimating the theta temperature for each peptide under the solvent conditions of interest; rejecting or reducing the selection probability of peptides whose estimated theta temperature is equal to or less than the temperature corresponding to the intended temperature of use of a multimer incorporating the peptide, and, optionally, rejecting or reducing the selection probability of peptides when the difference between the temperature corresponding to intended use and the estimated theta temperature of the peptide is sufficiently great that at the temperature corresponding to the intended use, the peptide is expected to adopt an extended conformation that would impose an unduly large entropic penalty on binding of the peptide to the protein target. The theta temperature of a peptide under the conditions of interest can be determined by well known methods (such as, the Flory-Huggins model), or by dynamic light scattering (see, e.g. Adam, Journal De Physique Lettres, 1984. 45(6): p. L279-L282 and Azevedo Journal of Molecular Structure-Theochem, 1999. 464(1-3): p. 95-105).

The selection of peptides in the initial library can also be biased toward peptides with a favored charge distribution. Binding affinity of a peptide to a target is usually conferred mainly by only a few residues, often charged residues, and these residues are usually spaced apart rather than clustered. Thus, in some methods, the initial selection of peptides is biased to result in an increased representation of charged residues (as further defined below) occurring at a spacing of at least three intervening amino acids and sometimes to increase representation of charged amino acids at a spacing of 3-7 intervening amino acids. The same considerations apply in spacing of charged residues in linkers described below.

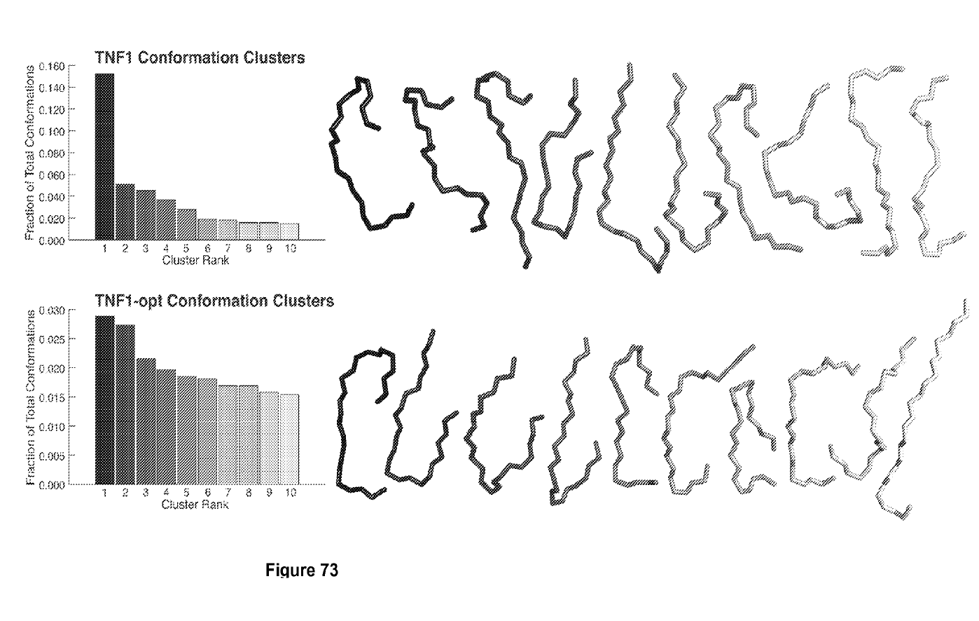

Libraries having members having no more than a single cysteine residue lack intra-chain disulfide bonds. Typically, there is no common secondary structure present in all, most or any members of the initial library. This can be determined in several ways including for example, by circular dichroism analysis that indicates less than 50% alpha helix or beta sheet structure. Often library peptides have a transient existence in many different conformations, such as the fluid hairpin conformations shown in FIG. 73. Because initial libraries are typically not designed with a particular target in mind, the same initial library can be used to identify members with affinities for different targets of interest. After an initial library has been screened to identify members binding to several different targets, certain members of the library are sometimes found to have little if any binding to any target. Such members can optionally be omitted from the initial library in subsequent screenings against different targets. Conversely, members from an initial library binding to one target may also bind to other targets. Thus, an otherwise randomly selected library can be modified by retaining some peptides known to bind to at least one target, and discarding peptides not known to have binding to at least one target. Thus, some initial libraries, can have for example, at least 10, 25 or 50% of members with affinity for at least one target, and can be screened against a different target.

An initial library is screened by a method that provides information about the relative binding of the library members to a target. Screening is, in general, a two-step process in which one first determines a measure of relative binding of peptides to a target and then decides which peptides to take forward and which to reject based on the relative binding data. That is, the process of determining binding affinity does not by itself, separate peptide binders and non-binders. The process does, however, usually allow ranking of all or most peptides (i.e., greater than 50% or 90%) tested by relative binding to the target. For example, when screening a library of 1000-25,000 peptides, a suitable peptide allows ranking of all or at least most of these peptides (i.e., greater than 50% or 90% of the number screened) by relative binding. A screening process also allows comparison of the relative binding of peptides to different targets. By contrast, selection is a process that results in physical separation of two classes of peptides that can be designated as binders and nonbinders depending on whether they bind to the target with sufficient affinity to withstand the selection process (e.g., washing of the target). Selection does not usually provide a measure of relative binding of binding peptides except sometimes inferentially from the relative representations of different peptides in a pool of binders. Selection does not provide any information about relative binding (if any) of peptides classified as non-binders.

The relative binding information can be a measure of dissociation constant, on-rate, off-rate or a composite measure of binding or “stickiness” (i.e., binding strength) to a target. For example, the strength of a signal from a labeled receptor bound to immobilized peptides can provide a value for general stickiness. Lower dissociation constants, slower off-rates and higher on-rates are generally preferred. Association constants are the reciprocal of dissociation constants; thus higher association constants are preferred. Relative binding of peptides revealed by the present screening methods is distinguished from a selection process that reveals the identities of peptides that have survived selection but not their relative binding compared with one another or other peptides that did not survive the selection process. Control compounds known to bind or not to bind a particular target (as more full described below) can serve as either positive or negative controls of binding and can also be included in binding assays together with library compounds being tested for binding.

A subset of peptides is determined based on the relative binding of the different peptides with a higher relative binding (whether measured in terms of a low dissociation constant, high association constant, high on-rate or low off-rate, or some composite measure of binding). That is, the subset of peptides have a higher relative binding to the target than the average binding of members of the initial library. In some methods, a subset of peptides having the strongest relative binding of the initial library is determined. In some methods, a threshold relative binding is defined and the subset of peptides have a relative binding exceeding the threshold. The threshold can optionally be set at a level that distinguishes between specific binding between peptides and a particular target and nonspecific binding between peptides and any target. Specificity of binding can be determined by contacting peptides with two or more different targets (e.g., simultaneously with the targets bearing different labels) and comparing binding of individual peptides to the different targets. Binding that is the same within experimental error to at least 2, and preferably, 3 or 5 different targets (e.g., randomly selected targets) can be classified as non-specific and binding that varies at least beyond experimental error and preferably by a factor of at least 5 or 10 between at least two targets can be classified as specific binding.

Nonspecific binding or background binding is usually the result of van der Waals forces, whereas specific binding is the result of bonds between specific groups, such as hydrogen bonding. However, unless otherwise apparent from the context, specific binding does not necessarily mean unique binding to one and only one target. A threshold can also be set at a level that defines a minimum binding affinity (e.g., dissociation constant less than 1 mM. A threshold can also be set at a level that identifies a certain percentage of peptides as having a binding affinity exceeding the threshold (e.g., 0.1-15% or 1-10%). A subset of peptides can also be identified by comparing values of binding of the peptides to the target with a theoretical maximum value. Peptides having values of binding within 90-110% of the theoretical maximum are of most interest to be taken forward to the next step. Values for binding over 110% of the theoretical maximum are probably due to artifacts, such as aggregation, effects, and thus peptides having these values are not usually taken forward at least without further investigation for artifacts.

The stringency at which an initial library is screened with a target can be controlled to improve distinction between peptides having a relative binding indicative of a target specific interaction and peptides having a relative binding indicative of a background or nonspecific binding not specific to the target. The stringency can be adjusted by varying the salts, ionic strength, organic solvent content and temperature at which library members are contacted with the target. An organic wash is useful in removing peptides noncovalently bound to other peptides rather than directly to the array.

Preferred stringencies typically allow identification of about 0.01 to 15% or 1-10% of peptides being screened as having a relative binding to a particular target in excess of background binding levels not specific to the target. The conditions of screening (e.g., presence or absence of organic solvent, temperature) can also be adjusted to reflect the conditions of intended use. For example, therapeutic applications usually occur at physiological temperature and conditions, in vitro diagnostics are often performed on ice (e.g., about 4° C.), but can also be performed at room temperature, and industrial processes may occur under conditions of high temperature or presence of organic solvents.

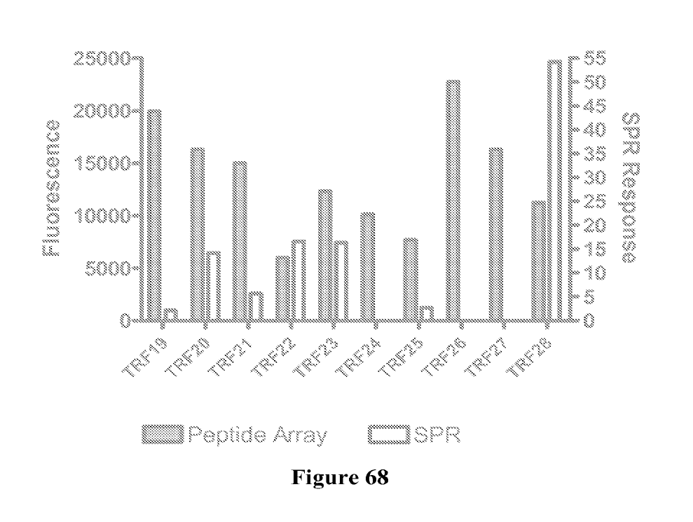

The screening can be performed with the library members immobilized in an array format and a target in solution. Alternatively, one or more targets can be immobilized, e.g., to a column or an array support and contacted with library members in solution. In a further variation particularly useful for peptide optimization as discussed below, library members are contacted with a target with both in solution. The relative binding of the peptides to a target depend in part on the format of the screening assay. FIG. 68 compares the binding of peptides to a target measured in two formats, one in which the peptides are immobilized, the other in which the target is immobilized. Some peptides show stronger relative binding in one format than the other. Thus, the subset of peptides identified sometimes differs depending on the format. A peptide-down array format offers advantages in screening large numbers of peptides, and target-down format has advantages in providing relative binding more representative of solution use of peptides. Solution binding may be more representative of peptide in therapeutic applications.

The accuracy may be improved in the target-down format as a result of avoiding cooperative binding of multiple different peptides in an array, binding of the same immobilized peptide to different sites on a target and or surface effects of an array including aggregation, surface binding and charge effects of the surface. The accuracy of a peptide-down array form can be improved by using spaced arrays; that is, arrays on surfaces coated with nano-structures that result in more uniform spacing between peptides in an array. For example, NSB Postech amine slides coated with trillions of NanoCone apexes functionalized with primary amino groups spaced at 3-4 nm for a density of 0.05-0.06 per nm² can be used. Surface effects can also be reduced by washing arrays with an organic solvent before determining binding. The organic solvent removes peptides that are not directly bound to the support but are noncovalently bound to other peptides that are bound to the support.

On organic wash can also be useful in a target down format, particularly when several different targets are bound to the same support.

In some methods, a peptide-down format is used in an initial screen and a target-down format in a subsequent screen. For example, a peptide-down format can be used on an initial set of 1000-50,000 peptides, and a target-down format on about 1-10% of this population as identified by the peptide-down screen. A target-down format can also be performed with pooled peptides in an initial screen to identify which of different pools of peptides containing one or more members with relatively high binding to a target. The members of such a pool are then retested individually to determine which peptide(s) was/were responsible for the relatively high binding of the pool.

Irrespective of the screening format, a subset of peptides is obtained from the initial library for further development. The subset typically constitutes about 0.01-15% or 1-10% of the initial library. Members of the subset typically have affinity of 1-1000 and sometimes 10-100 micromolar.

As well as binding strength (composite or any of the specific measures discussed above) to a target of interest, other criteria that can be used to select the subset of peptides include relative purity of peptides (higher purity being preferred) and binding specificity (as assessed by relative lack of binding to unrelated targets), greater specificity for a target of interest usually being preferred.

For assays with immobilized peptides, and target in solution, the target can be labeled and bound target detected from the label. The relative labeling of different peptides provides a composite relative measure of binding or stickiness of peptides to the array. Surface plasmon resonance (SPR) provides a suitable technique for measuring relative binding when either target or peptides is immobilized on a support. No label is required. SPR can provide a measure of dissociation constants, and if peptides are tested at different concentrations, dissociation rates. The A-100 Biocore/GE instrument, for example, is suitable for this type of analysis. FLEXchips can be used to analyze up to 400 binding reactions on the same support.

Before or after proceeding to form multimers from a subset of peptides selected based on their relatively high affinity for a target, individual peptides can be optimized to improve binding to the target. The optimization can be performed by making a population of variants of a peptide, and screening or selecting the variants for binding to the target. In some methods, known as linear optimization, a single position in each peptide is varied at a time. That is, each variant tested differs from an initial peptide at a single position, although the position may vary in different peptides, such that most or all positions in an initial peptide are varied. Each position can, for example, be varied with each of the 20 natural amino acids, or a representative subset thereof. The number of positions varied in a peptide can be e.g., at least 10, at least 15 positions or at least 17 positions. In some methods, all or most (over 50%) of position in a peptide are varied. For a 20 amino acid peptide, each position can be varied with each amino acid with a total of 400 peptides. The number of peptides can be reduced by using representative examples of classes of amino acids, rather than all 20 natural amino acids (e.g., hydrophobic, hydrophilic, acid, basic and aromatic). A representative subset of amino acids can include one amino acid from each such class. For example the amino acids I, D, W, L, E, G, T, S, K, R, Q and N provide a representative set of the different natural classes of amino acids. In some methods, a peptide is randomized with a set of up to 10 amino acids including (a) at least one amino acid selected from Y, A, D and S, (b) lysine and (c) at least one amino acid selected from N, V and W. In some methods, a peptide is randomized with a set of amino acids consisting of Y, A, D, S, K, N, V and W.

Screening of such a population of variants indicates which positions in an initial peptide most affect binding to a target, and provides an indication of what type of amino acid at such positions improves binding. A further population of variants can be designed including variation at combinations of positions shown to most affect binding in the previous analysis. The varied positions can be occupied by a more limited subset of amino acids reflecting the amino acids occupying these positions associated with highest binding to a target. Of course, although not necessary any other variant peptides of interest can be synthesized as well as the types of peptides used in the linear optimization strategy.

For example, the linear search may result in 5 positions in which substantial improvement can be made. At 3 of those positions, two amino acids improve binding substantially and at the other 2 positions, only one amino acid improves binding substantially. One then has a total of 3×3×3×2×2=108 possible combinations of amino acids in the different positions (assuming the changes and the original amino acid are included at each position). All of these possible combinations of changes that were found to result in linear improvement can easily be tested allowing only those combination of mutations that do not interfere with one another to be taken forward.

In some methods, differences in binding energies (Gibbs free energy or AG) are associated with variations. Binding energy of a peptide can be calculated from its dissociation constant, measured by e.g., SPR. The binding energy attributable to a particular variation can be obtained by subtracting from the binding energy of a variant peptide the binding energy of the peptide being randomized. Improved binding is indicated by a negative change in free energy. It has been found that combining the changes in free energy binding of single amino acid variations at different positions in a peptide being randomized provides a useful prediction of the free change of a variant peptide having a combination of the variations. The respective binding energy changes can be combined by simple addition. Comparison of the predicted changes in free energy binding of different combinations of variations can be used as a basis for which further variant peptides to synthesis and screen in a further cycle of peptide variation. The higher the combined negative free energy of binding of two or more variations, the stronger the binding strength. Optionally, synthesis and testing of variant peptides can be performed on an iterative basis with changes in free energy associated with variants in one cycle being combined, and the combined changes in free energy being used as a basis to select peptides for synthesis and testing in a subsequent cycle. Usually combinations of variations with the strongest or near highest combined negative free energies of binding are selected. Although combination of binding energies of individual variations may provide the most accurate predictor of the effect on target binding of combining variations, similar predictions can be made based on other measures of binding strength, such as association constants, on-rates or off-rates.

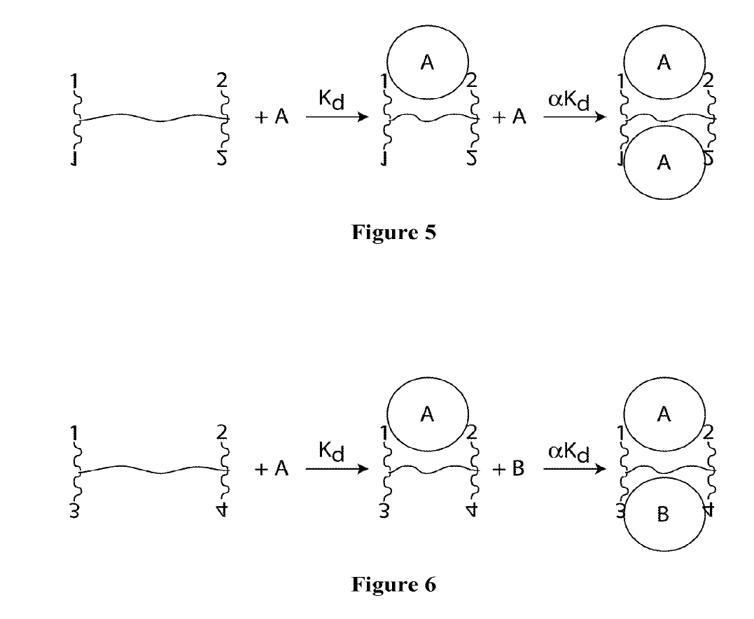

Linear optimization can be automated with a system including a computer and automated apparatus, for testing and synthesizing peptides. A typically computer (see U.S. Pat. No. 6,785,613 FIGS. 4 and 5) includes a bus which interconnects major subsystems such as a central processor, a system memory, an input/output controller, an external device such as a printer via a parallel port, a display screen via a display adapter, a serial port, a keyboard, a fixed disk drive and a floppy disk drive operative to receive a floppy disk. Many other devices can be connected such as a scanner via I/O controller, a mouse connected to serial port or a network interface. The computer contains computer readable media holding codes to allow the computer to perform a variety of functions. These functions include controlling the automated apparatus, receiving input of a peptide sequence to be optimized and output of an optimized sequence, and performing various operations as described above. For example, the operation include design of variant peptide sequences, both in an initial cycle and further variants in subsequent cycle(s), calculation of binding energies, combination of binding energies of different variations. The automated apparatus can include a robotic arm for delivering reagents for peptide synthesis and testing, as well as small vessels, e.g., microtiter wells for performing the synthesis and testing of peptides.

The predictability of determining binding energies attributable to combinations of variations from binding energies attributable to individual variations by simple addition means that it is often possible to converge on an improved peptide (e.g., having a binding strength (Kd, on-rate, off rate, or composite measure) greater by factor of at least 10 or 100 greater than a lead peptide) with only two or three cycles of synthesizing and testing variant peptides and their combination. Some methods involve no more than 2, 3, 4 or 5 cycles of synthesizing and testing variant peptides and their combinations. Linear optimization provides a rapid means to sort through the large gaps in sequence space between the peptides of the initial library arising from the small size of the library relative to total sequence space. Although linear optimization is particularly suitable for peptides screened from the relatively small libraries of the present methods, it can also be used for any lead peptide, such as lead peptides resulting selection from display libraries.

Alanine-scanning mutagenesis is also useful for optimization. In this method, variants of an initial peptide are produced each differing from a selected peptide in one position, occupied by alanine residue. Different variants differ from the initial peptide at different positions. The different variants are compared for binding to the target to determine which alanine substitutions most reduce binding affinity. Positions flanking these positions are identified as candidates for variation. A second set of variants is then produced at which amino acids flanking the positions at which alanine caused the greatest loss of affinity are varied with all of the 20 natural amino acids or a representative sample thereof. The second set of variants can include variation at multiple positions identified by the initial alanine scan. The second set of variants are tested for relative binding to the target. If one or more variants are identified having higher affinity than the peptide originally selected, the one or more variants can be used to make multimers in subsequent steps.

Individual peptides can also be optimized for length. Such a process compares an initial peptide with truncation variants of the peptide in which amino acids are deleted from either or both ends. Optionally, internal amino acids can also be deleted. Such analysis sometimes identifies certain amino acids as not contributing to binding of a peptide. Such amino acids can be deleted in subsequent steps.

During the optimization process, peptide variants can be screened by the same processes as described for the initial library, e.g., SPR. Optionally, peptides are assayed at concentration at least a factor of 2 or 3 or lower than the dissociation constant of the lead peptide (K_{d}^{˜}160 μM) to improve the high-end dynamic range of responses.

Selection methods are also possible, including phage display (see, e.g., Dower, W O 91/19818; Devlin, WO 91/18989) and other display methods and can be used to analyze larger numbers of variants (e.g., 10¹² peptides). In ribosome display, polypeptides are screened as components of display package comprising a polypeptide being screened, and mRNA encoding the polypeptide, and a ribosome holding together the mRNA and polypeptide (see Hanes & Pluckthun, PNAS 94, 4937 4942 (1997); Hanes et al., PNAS 95, 14130 14135 (1998); Hanes et al, FEBS Let. 450, 105 110 (1999); U.S. Pat. No. 5,922,545). mRNA of selected complexes is amplified by reverse transcription and PCR and in vitro transcription, and subject to further screening linked to a ribosome and protein translated from the mRNA. In another method, RNA is fused to a polypeptide encoded by the RNA for screening (Roberts & Szostak, PNAS 94, 12297 12302 (1997), Nemoto et al., FEBS Letters 414, 405 408 (1997). RNA from complexes surviving screening is amplified by reverse transcription PCR and in vitro transcription.

Members of the selected subset of library members having relatively high binding to a target of interest (with or without optimization) can be tested for competition with one another for binding to the target. A competition assay indicates whether two members bind to the same or sufficiently similar epitopes on the target to compete with one another for binding to the target. In general, it is preferable to identify two members that do not compete with one another because such members can bind to the target simultaneously. However, members competing with one another (or two copies of the same members) can also be usefully linked if two binding sites are present on the same target (for example if the target is a homodimeric protein). Competition can be tested by an assay in which two peptides are contacted with a target separately and together. If the combined binding of the peptides together is about the aggregate of that of the peptides separately, then the peptides do not compete. If the combined binding of the peptides together is between that of the individual peptides, then the peptides compete with one another. Competition assays are preferably performed at peptide concentrations above Kd and more preferably close to saturating peptide concentrations. In another embodiment, protein-protein interface mapping may be used to verify that two members of the selected subset of library members having relatively high binding to a target of interest do not bind to the same or sufficient similar epitopes. Protein-protein interface mapping can determine the regions on the target that the members bind. The details of protein-protein interface mapping are apparent to persons having ordinary skill in the art. Briefly, protein-protein interface mapping involves mapping of protein interfaces using chemical cross-linking of protein complexes. To perform mapping, the members are separately incubated with the target of interest in a mixture and a cross-linking reagent is added to the mixture for further incubation. For example, a cross-linking reagent may be BS²G-d₀, BS²G-d₄, or Sulfo-SBED. After unreacted cross-linkers and peptides are removed from the mixture, the cross-linked samples are digested with trypsin. Undigested protein and digested peptides are separated and analyzed by MALDI-TOF mass spectrometry. Identification of cross-linked fragments provide information on where the members bind on the target of interest.

Following selection and optionally optimization and competition assays, members of the subset of members of the initial library having relatively high binding to a target of interest are linked to one another to form multimers. The different members of the subset can be linked to one another en masse, such that any member of the subset can pair with any other. Alternatively, pairs of members (usually pairs not competing with one another) are separately linked. The linkage is usually performed by chemical linkage (i.e., with non-peptidic bonds). A pair of peptides can be joined to one another with one linker in four orientations (N-terminus to N-terminus, C-terminus to C-terminus, N-terminus to C-terminus and C-terminus to N-terminus). The orientation of linkage can be controlled by the reactive groups at the termini of the peptides and the linker. One, some or all of the possible orientations can be synthesized. In some methods, a pair of peptides are joined to one another by two linkers forming a cyclic structure. Again multiple orientations of the same peptides can be joined in a cyclic structure. For example, two peptides can be joined N-terminus to N-terminus and C-terminus to C-terminus, or N-terminus to C-terminus and C-terminus to N-terminus or vice versa. In the more general case of joining n-peptides to one another, the peptides can be joined in 2ⁿ orientations. Usually several different linkers are tested for any given pair of peptides. For example, at least 5, 10, or 20 linkers can be tested. In some methods, 5-100 different linkers are tested. The linkers can be peptides or nonpeptidic (e.g., DNA or PEG). The linker can also be an amino acid flanked by PEG on both sides. Optionally, a library of linkers can be synthesized on beads by a split-pool approach (see, e.g., Burbaum et al., Proc Natl Acad Sci USA. 92(13):6027-31 (1995)). The linkers typically vary in length, flexibility, charge, or charge distribution. The length can be controlled by the number of amino acids or other monomers in a polymeric linker. The length can vary from about 0.1 nm (in the case of direct bonding of one peptide to another by a non-peptidic bond) to about 30 nm. The flexibility can be controlled by the number of proline residues (the more proline residues, the more rigid the linker). Proline and glycines are relative inert with respect to potential interactions with a target. The charge can be controlled by the number and distribution of charged residues. Positively charged residues include arginine, lysine and sometimes histidine. Negatively charged amino acids include glutamate and aspartate. The linkers can also have a branched structure (e.g., multi-antigenic MAP linkers) to form multimers with more than two peptides. A simple example of a MAP linker is a lysine residue in which peptides are attached to alpha and epsilon moieties of the lysine.

One example of a linker is a polyproline or poly (proline glycine proline) in which one or both distal portions of the linker are azido-modified to facilitate conjugation to one or more peptides by azide-alkyne conjugation. Alternatively, such linkers can be alkyne-modified on one or both terminal residues and conjugated to azido-modified peptides. Another example of a linker has the formula (pro pro X pro pro) n, wherein X is an amino acid that varies between linkers and n is between 1 and 10. Other linkers have a propargyl lysine residues as the C- or N-terminal residue or residue adjacent to the C- or N-terminal residue.

The linker plays a role of holding the two peptides together in such a manner that both peptides can interact with their respective binding sites on a target. The length of linker depends on the relative spacing of binding sites on the target. Typically, a minimum length of linker is needed for both binding peptides to bind simultaneously. Thus, if the length of linker is increased for a given peptides, the binding typically shows a steep increase as the minimum length of linker is reached, plateaus and then gradually decreases as the linker length is increased. A more flexible linker typically increases the on-rate and off-rate of a multimer. Because a high on-rate and a low-off rate is usually desired, there is usually an optimum flexibility of a linker for a particular peptide pair. As well as holding two peptides together, a linker can also contribute to binding to the target, particularly via the inclusion of charged amino acids in the linker.

Multimers formed by linking peptides to one another are screened for binding to the target. The same or different types of screen can be used as for the initial library. One type of screen particularly useful for comparing different linkers of different molecular weights is to contact a population of multimers containing such different linkers with an immobilized or immobilizable target. An immobilizable target is typically a target linked to a tag such as biotin or hexa histidine that permits immobilization of the target to a binding moiety of the tag. Multimers having relatively strong affinity to the target bind to the target, whereas multimers with relatively weak affinity remain in solution and can be discarded. The multimers binding to the target are then washed off the target and analyzed by mass spectrometry. The mass spectrometry distinguishes the different molecular weights of the linkers and thus indicates which linkers were most suitable to confer relatively high binding for a given pair of peptides. Mass spectrometry can also be used to distinguish multimers of different molecular weight in which the difference in molecular weight residues in the peptide moieties as well as or instead of in the linkers. MALDI-chips provide a suitable format for mass spectrometry.

The multimer or multimers having highest binding to a target are usually of most interest. Such multimers are characterized by first and second peptides, each having 8-35 amino acids. The peptides typically lack significant sequence identity (i.e., less than 30% sequence identity when maximally aligned) either with each other, with the target or with a known ligand of the target. The peptides typically lack intra or inter chain disulfide bonds and a common secondary structure with each other. Each peptide typically has detectable binding to the target (e.g., 1-1000 or 10-100 micromolar) by one or more of the assays described above. The peptides are typically joined to one another by one or more linkers. The linkages between peptides and such linkers are usually by non-peptide bonds. Such linkers often contain a charged residue that forms a noncovalent bond with the target. The binding affinity of such multimers for a target is usually at least 5-, 10-, 20- or 100-fold greater than that of either of its component peptides. Preferably the binding affinity of such a multimer is at least 10⁷ M⁻¹. Some such multimers have affinities within a range of 10⁷M⁻¹ to 10¹⁰ M⁻¹ or 10⁸M⁻¹ to 10¹⁰ M⁻¹.

Analysis of some multimers bound to targets indicate a tendency for peptide components of the multimers to have end-to-end lengths greater than the theoretical random flight length (equal to the inter-residue distance times the square root of the number of residues) and less than three quarters of the fully stretched out length (that is, three quarters of the product of the number of residues times the inter-residue distance). (For amino acids connected by a peptide bond, the inter-residue distance is approximately 3.8 Angstroms.)

Having identified a multimer with affinity for a target, the multimer can undergo further optimization by substitution, addition or deletion of amino acids chemical modifications of amino acids or replacement of amino acids with unnatural amino acids or other chemical mimetics. Derivatives should have a stabilized electronic configuration and molecular conformation that allows key functional groups to be presented to the target binding sites in substantially the same way as the lead multimer. Identification of derivatives can be performed through use of techniques known in the area of drug design. Such techniques include self-consistent field (SCF) analysis, configuration interaction (CI) analysis, and normal mode dynamics analysis. Computer programs for implementing these techniques are readily available. See Rein et al., Computer-Assisted Modeling of Receptor-Ligand Interactions (Alan Liss, N.Y., 1989). Derivatives may have higher binding affinity, smaller size, and/or improved stability relative to a lead multimer. Modifications can include N terminus modification, C terminus modification, peptide bond modification, including, CH₂—NH, CH₂—S, CH₂—S, O═C—NH, CH₂—O, CH₂—CH₂, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference.

With or without such further optimization, a desired multimer can usually be manufactured by conventional chemical synthesis and provided in purified form appropriate to the intended use (e.g., at least 99% w/w pure for pharmaceutical use). The multimer can then undergo further processing or packaging appropriate for the intended use. For example, for therapeutic uses, a multimer can be combined with a pharmaceutically acceptable carrier to form a pharmaceutical composition. For diagnostic application, a multimer can be linked to a label or attached to a support or incorporated into a diagnostic kit.

The data provided in the examples show that although synbodies show specific binding for a target in the sense that a synbody can preferentially bind to a target in a mixture of unrelated molecules, synbodies do not necessarily show such specificity for one and only one target molecule. In other words, a synbody screened against a large collection of different targets shows a gradation of different binding strengths to different targets. The binding strength to most targets is usually at or near background levels, but the synbody may show usable binding strength to not just one, but several different targets (e.g., 2-10 or 3-5), not necessarily showing any relationship to each other. The target most strongly bound by a synbody is not necessarily the target against which the synbody or its component peptides was originally screened. Accordingly, peptides identified from an initial set as showing relatively high binding to one target can also be screened for binding to one or more different targets. Likewise a multimeric peptide or synbody identified as showing specific binding to one target can be screened for binding to one or more different targets. Simple variants of a multimeric peptide found to bind one target (e.g., peptides attachment sites to linker reversed, orientation of one or both peptides reversed, or different linker) can also be screened for binding to different targets. Such screens with either peptides or multimers can be performed in an array format with at least 100 or 1000 immobilized targets. The targets in such methods are usually proteins.

Although synbodies do not necessarily bind to one and only one target, the same is the case for antibodies and has not prevented their use in diagnostics or therapeutics. In diagnostics, additional specificity can be obtained, if desired, by using two synbodies in a sandwich format, the synbodies having specificity for different epitopes on a target and having different off-target binding specificities. A synbody can also be combined with an antibody having a different epitope specificity to the same target in a sandwich format. In therapeutics, off-target binding does not necessarily cause side effects because off-targets may not be present or accessible in a given disease state in a given organism following administration by a particular route, or off-target binding may have only benign effects.

Various aspects of the invention are now disclosed in further detail.

In a first aspect, the present invention provides methods for identifying affinity elements to a target of interest, comprising

(a) contacting a substrate surface comprising an array of between 10² and 10⁷ different test compounds of known composition with a target of interest under conditions suitable for moderate affinity binding of the target to target affinity elements if present on the substrate, optionally wherein the target is not an Fv portion of an antibody, and wherein the different test compounds are not derived from the target; and

(b) identifying test compounds that bind to the target with at least moderate affinity, wherein such compounds comprise target affinity elements.

The inventors have discovered that screening for affinity elements to a target of interest using an array of different test compounds of known composition permits a large amount of chemical/structural space to be adequately sampled using only a small fraction of the space. The resulting methods provide a rapid and high throughput method for identifying affinity elements to targets of interest.

While not being bound by any specific hypothesis, the inventors propose that the tremendously large number of possible arrangements for a target of a given size actually form a very limited number of structural forms or combinations of patches of smaller sequences, providing the ability to identify affinity elements to a target of interest by screening a target of interest against a much smaller array of test compounds (ie: potential affinity elements) than previously considered possible. In contrast to the “lock and key” metaphor by which highly specific interactions such as small molecule docking or antibody binding are typically described, moderate affinity binding of peptides and peptide-like polymers to proteins can be viewed as a “magnetic bead” model, in which a peptide is represented as a somewhat flexible string of beads, a few of which are magnetic, and the protein surface is represented as a mostly inert surface with a few scattered magnetic spots. In this, each bead represents a single residue, with a few beads distributed along the string being capable of forming relatively strong interactions, and the remaining beads contributing relatively little to binding affinity. Binding then entails the string of beads finding an alignment on the surface of the target protein such that the peptide residues capable of strong interaction are able to align themselves with corresponding protein surface loci in such a way as to form hydrogen bonds, salt bridges, strong hydrophobic interactions, or other interactions that contribute disproportionately to binding energy. Consistent with this model moderate affinity binding (corresponding, for example, to a dissociation constant of 100 μM) requires a AG of only on the order of −5.5 kcal/mole, an amount of energy that can be supplied by a relatively few interactions.

Since the composition of each test compound on the substrate surface is known, the method is a screen for affinity elements, not a selection. Screenable libraries as used in the methods of the present invention are much smaller (˜10² to 10⁷) than selectable libraries (10⁹-10¹⁴). Thus, the process of affinity element discovery is limited only by the rate at which individual targets can be screened on test compound-containing substrate surfaces. In this sense it is distinct from current selection techniques, in which recurrent selections using unknown sequences are required. Exemplary substrate surfaces are described below.

In one embodiment, the substrate surface comprises an addressable test compound array. “Addressable” means that test compounds on the substrate surface are present at a specific location on the substrate, and thus detection of binding events serves to identify which test compound has bound target.

The “different test compounds of known composition” are of known structure and/or composition. Thus, for example, if the test compounds comprise or consist of nucleic acids or polypeptides, their nucleic acid or amino acid sequence is known, while further structural information may also be known (although this is not required).

Furthermore, the test compounds are not all related based on minor variations of a core sequence or structure. Thus, when the test compounds comprise nucleic acids or polypeptides, the nucleic acid or polypeptide sequences are known, but the test compounds are not simply a series of mutants/fragments of a known sequence, nor a series of epitopes/possible epitopes from a given antigen. The different test compounds may include variants of a given test compound (such as polypeptide isoforms), but at least 10% of the test compounds on the array are structurally and/or compositionally unrelated. In various embodiments, 20%, 30%, 40%, 50%, 60%, 70%, 80%, 90%, 95%, 98%, or more of the test compounds on the array are structurally and/or compositionally unrelated.

The different test compounds can comprise or consist of any class of compounds capable of binding to a target of interest, but the different test compounds are not derived from the target. As used herein, “not derived from” means that the test compounds are not fragments of the target to be screened. In this embodiment, for example, if the target is a nucleic acid, the different test compounds do not consist of a polynucleotide found within the target (on its sense or antisense strand). Similarly, if the target is a protein, the test compounds do not individually consist of a polypeptide found within the target, or an “antisense” version thereof (ie: polypeptides which are encoded on the opposite strands of the DNA encoding the protein target in a given reading frame, which can have an affinity to bind each other based on hydropathic complementary of the polypeptides).

The arrays may further comprise control compounds, and that such control compounds may be of any type suitable to serve as appropriate controls for target binding, including but not limited to antibodies, Fv regions of antibodies, variable regions of an antibody, or antigen binding regions of an antibody, and control compounds derived from the target. In various embodiments, up to 25% of the compounds on the substrate surface may be control compounds; in various further embodiments, 20%, 15%, 10%, 5%, 4%, 3%, 2%, 1%, 0.5%, 0.1% or less of the compounds on the substrate surface are control compounds.

In another embodiment, the different test compounds on the array are not antibodies, Fv regions of antibodies, variable regions of an antibody, or antigen binding regions of an antibody.

Classes of test compounds suitable for use in the present invention include, but are not limited to, nucleic acids, polypeptides, peptoids, polysaccharides, organic compounds, inorganic compounds, polymers, lipids, and combinations thereof. The test compounds can be natural or synthetic. The test compounds can comprise or consist of linear or branched heteropolymeric compounds based on any of a number of linkages or combinations of linkages (e.g., amide, ester, ether, thiol, radical additions, metal coordination, etc.), dendritic structures, circular structures, cavity structures or other structures with multiple nearby sites of attachment that serve as scaffolds upon which specific additions are made. In various preferred embodiments, all or a plurality of the test compounds are non-naturally occurring. In other embodiments, the test compounds are selected from the group consisting of nucleic acids and polypeptides. In one specific embodiment, if the different test compounds consist of nucleic acids, then the target is not a nucleic acid. In another embodiment, the different test compounds are not nucleic acids. In a further embodiment, the test target is not a nucleic acid.

In a further embodiment, the different test compounds on the substrate are of the same class of compounds (ie: all polypeptides; all nucleic acids, all polysaccharides, etc.) In other embodiments, the test compounds comprise different classes of compounds in any ratio desired. These test compounds can be spotted on the substrate or synthesized in situ, using standard methods in the art. The test compounds can be spotted or synthesized in situ in combinations in order to detect useful interactions, such as cooperative binding.

The substrates may further comprise control compounds or elements as discussed above, as well as identifying features (RFID tags, etc.) as suitable for any given purpose.

In one embodiment, the different test compounds are chosen at random using any technique for making random selections. In a further embodiment, an algorithmic approach for selecting different test compounds is used.

In a further embodiment, all or a plurality of the test compounds on the array do not naturally occur in an organism from which the target is derived, where the target is a biological molecule. In another embodiment, where the test compounds comprise polypeptides, all or a plurality of the polypeptide test compounds are not found in the SWISSPROT database (web site ebi.ac.uk/swissprot/), either as a full length polypeptide or as a fragment of a polypeptide found in the SWISSPROT database. In other words, the test compounds are not derived from naturally occurring proteins. In another embodiment, where the test compounds comprise nucleic acids, all or a plurality of the nucleic acid test compounds are not found in the GENBANK database (web site ncbi.nlm.nih.gov/Genbank/), either as a full length nucleic acid or as a fragment of a nucleic acid found in the GENBANK database. There are at least two reasons to use such “non-naturally occurring” test compounds. First, there is little known about what potential binding space would be occupied by a particular collection of elements. Arguments could be made for or against many alternatives. Second, life space (ie: naturally occurring compounds) has been selected to meet many requirements beyond simply binding, and the binding is in very specific conditions in life. Thus, naturally occurring compounds suffer from constraints over many degrees of freedom and these constraints would handicap a search for affinity elements to a large number or targets. An unanticipated benefit of using non-naturally occurring different test compounds (as discussed below) is that, overall, at least in the case of polypeptides, the resulting test compounds tend to be more soluble and well behaved in solution than a similarly sized set of compounds derived from life space compounds, which provides advantages in binding assays, such as in the array-based formats disclosed herein. In various further embodiments, at least 10%, 20%, 30%, 40%, 50%, 60%, 70%, 80%, 90%, 95%, 98% or more of the test compounds on the array do not naturally occur in an organism from which the target is derived, where the target is a biological molecule. Similar various further embodiments are contemplated for the specific nucleic acid and polypeptide embodiments disclosed above.

In a further embodiment, the test compounds have a molecular weight of between about (ie: +/−5%) 1000 Daltons (D) and 10,000 D. As discussed below, test compounds within this molecular weight class are of particular utility in preparing synthetic antibodies (also referred to herein as “synbodies”) according to the present invention. In one embodiment, polypeptide test compounds for use in the methods of this aspect of the invention are between about 1000 Daltons and 4000 Daltons (up to approximately 30 amino acid residues); in various further embodiments between 1100 D-4000 D; 1200 D-4000 D; 1300 D-4000 D; 1400 D-4000 D; 15000-4000 D; 1000 D-3500 D; 1100 D-3500 D; 12000-3500 D; 1300 D-3500 D; 1400 D-3500 D; 15000-3500 D; 1000 D-2000 D; 1100 D-3000 D; 1200 D-30000; 13000-3000 D; 1400 D-3000 D; and 1500 D-3000 D. In another embodiment, nucleic acid aptamers of up to 10,000 Daltons are used (ie: approximately 30 bases).

As used herein, “at least moderate affinity binding” of the target to target affinity elements generally means a binding affinity of at least about (ie: +/−5%) 500 μM. In various further embodiments, “at least moderate binding affinity” for the target means at least about 250 μM, 150 μM; 100 μM, 50 or 1 μM. In various further embodiments, the target affinity elements possess binding affinity for the target of between about (ie: +/−5%) 1 μM and 500 μM. In various further embodiments, moderate affinity binding of the target to target affinity elements generally means a binding affinity of between about 1 μM-250 μM; 1 μM-150 μM; 10 μM-500 μM; 25 μM-500 μM; 50 μM-500 μM; 100 μM-500 μM; 10 μM-250 μM; 50 μM-250 μM; and 100 μM-250 μM.

As used herein, “binding” of test compounds to a target refers to selective binding in a complex mixture (ie: above background), and does not require that the binding be specific for a given target (and only to that target), as traditional antibodies often cross-react. The extent of acceptable target cross-reactivity for a given affinity element depends on how it is to be used and can be determined based on the teachings herein. For example, methods to modify the affinity and selectivity of the synthetic antibodies produced using the binders identified in the methods of the invention are described below. Such binding can be of any type, including but not limited to covalent binding, hydrophobic interactions, van der Waals interactions, the combined effect of weak non-covalent interactions, etc.

Specific conditions suitable for moderate affinity binding of the target to the test compounds will depend on the type of target and test compounds (ie: polypeptide, nucleic acid, etc.), as well as the specific structure of each (ie: length, sequence, etc.).

Determination of suitable conditions for moderate affinity binding of a specific target to a specific collection of test compounds is well within the level of skill in the art based on the teachings herein. In various non-limiting embodiments, conditions such as those described in the examples that follow can be used.

For example, the screen can be done under non-biological conditions, such as non-aqueous conditions. This is in contrast to prior methods of selection mentioned above that use a living system in some phase. Most antibodies do not function when applied to the surface of arrays. In contrast, the binding agents developed here are screened to function on surfaces.

The binding can be detected by many other methods, including but not limited to direct labeling of the target, secondary antibody labeling of the target or directly determined by SPR electrochemical detection, micromechanical detection (e.g., frequency shifts in resonant oscillators), electronic detection (changes in conductance or capacitance), mass spectrometry or other methods. The target can also be pre-incubated with another control compound (ie, protein, drug or antibody, etc.) to block the binding of particular classes of affinity targets in order to focus the search. The binding can be done in the presence of competitive inhibitors (including but not limited to E. coli extract or serum) to accentuate specificity.

In another embodiment, the methods comprise identifying affinity elements for more than one target at a time. The methods of the invention are easily amenable to multiplexing. In one embodiment, each target is labeled with a different signaling label, including but not limited to fluorophores, quantum dots, and radioactive labels. Such multiplexing can be accomplished up to the resolution capability of the labels. Targets that bound two or more affinity elements would produce summed signals. Other techniques for multiplexing of the assays can be used based on the teachings herein.

In various embodiments, the substrate surface comprises an array of between 100 and 100,000,000 different test compounds. Such arrays may further comprise control compounds or elements as discussed above. In various other embodiments, the substrate surface comprises between 100-10,000,000; 100-2,000,000; 100-5,000,000; 100-1,000,000; 100-500,000; 100-100,000,100-75,000; 100-50,000; 100-25,000; 100-10,000; 100-5,000,100-4,000,250-1,000,000,250-500,000,250-100,000,250-75,000; 250-50,000; 250-25,000; 250-10,000; 250-5,000,250-4,000; 500-1,000,000; 500-500,000,500-100,000,500-75,000; 500-50,000; 500-25,000; 500-10,000; 500-5,000,500-4,000; 1,000-1,000,000; 1,000-500,000; 1,000-100,000, 1,000-75,000; 1,000-50,000; 1,000-25,000; 1,000-10,000; 1,000-8,000, 1,000-5,000 and 1,000-5,000 different test compounds.

As used herein “nucleic acids” are any and all forms of alternative nucleic acid containing modified bases, sugars, and backbones. These include, but are not limited to DNA, RNA, aptamers, peptide nucleic acids (“PNA”), 2′-5′ DNA (a synthetic material with a shortened backbone that has a base-spacing that matches the A conformation of DNA; 2′-5′ DNA will not normally hybridize with DNA in the B form, but it will hybridize readily with RNA), locked nucleic acids (“LNA”), Nucleic acid analogues include known analogues of natural nucleotides which have similar or improved binding properties. “Analogous” forms of purines and pyrimidines are well known in the art, and include, but are not limited to aziridinylcytosine, 4-acetyl cytosine, 5-fluorouracil, 5-bromouracil, 5-carboxymethylaminomethyl-2-thiouracil, 5-carboxymethylaminomethyluracil, inosine, N6-isopentenyladenine, 1-methyladenine, 1-methylpseudouracil, 1-methylguanine, 1-methylinosine, 2,2-dimethylguanine, 2-methyl adenine, 2-methylguanine, 3-methylcytosine, 5-methylcytosine, N6-methyl adenine, 7-methylguanine, 5-methylaminomethyluracil, 5-methoxyaminomethyl-2-thiouracil, beta-D-mannosylqueosine, 5-methoxyuracil, 2-methylthio-N-6-isopentenyladenine, uracil-5-oxyacetic acid methylester, pseudouracil, queosine, 2-thiocytosine, 5-methyl-2-thiouracil, 2-thiouracil, 4-thiouracil, 5-methyluracil, uracil-5-oxyacetic acid, and 2,6-diaminopurine. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs), methylphosphonate linkages or alternating methylphosphonate and phosphodiester linkages (Strauss-Soukup (1997) Biochemistry 36:8692-8698), and benzylphosphonate linkages, as discussed in U.S. Pat. No. 6,664,057; see also Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS1992); Milligan (1993) J. Med. Chem. 36:1923-1937; Antisense Research and Applications (1993, CRC Press).

The term “polypeptide” is used interchangeably with “peptide” and in its broadest sense to refer to a sequence of subunit amino acids, amino acid analogs, or peptidomimetics. Thus, peptides include polymers of amino acids having the formula H₂NCHRCOOH and/or analog amino acids having the formula HRNCH₂COOH. The subunits are linked by peptide bonds (i.e., amide bonds), except as noted. Usually most and often all subunits are connected by peptide bonds. The polypeptides may be naturally occurring, processed forms of naturally occurring polypeptides (such as by enzymatic digestion), chemically synthesized or recombinantly expressed. Preferably, the polypeptides for use in the methods of the present invention are chemically synthesized using standard techniques. The polypeptides may comprise D-amino acids (which are resistant to L-amino acid-specific proteases), a combination of D- and L-amino acids, β amino acids, and various other “designer” amino acids (e.g., β-methyl amino acids, Cα-methyl amino acids, and Nα-methyl amino acids, etc.) to convey special properties. Synthetic amino acids include omithine for lysine, and norleucine for leucine or isoleucine. Hundreds of different amino acid analogs are commercially available from e.g., PepTech Corp., MA. In general, unnatural amino acids have the same basic chemical structure as a naturally occurring amino acid, i.e., an a carbon that is bound to a hydrogen, a carboxyl group, an amino group, and an R group.

In addition, polypeptides can have peptidomimetic bonds, such as N-methylated bonds (—N(CH₃)—CO—), ester bonds (—C(R)H—C—O—O—C(R)—N—), ketomethylen bonds (—CO—CH₂—), aza bonds (—NH—N(R)—CO—), wherein R is any alkyl, e.g., methyl, carba bonds (—CH₂—NH—), hydroxyethylene bonds (—CH(OH)—CH₂—), thioamide bonds (—CS—NH—), olefinic double bonds (—CH═CH—), retro amide bonds (—NH—CO—), peptide derivatives (—N(R)—CH₂—CO—), wherein R is the “normal” side chain, naturally presented on the carbon atom. These modifications can occur at any of the bonds along the peptide chain and even at several (2-3) at the same time. For example, a peptide can include an ester bond. A polypeptide can also incorporate a reduced peptide bond, i.e., R—CH₂—NH—R₂, where R₁ and R₂ are amino acid residues or sequences. A reduced peptide bond may be introduced as a dipeptide subunit. Such a polypeptide would be resistant to protease activity, and would possess an extended half-live in vivo. The affinity elements can also be peptoids (N-substituted glycines), in which the sidechains are appended to nitrogen atoms along the molecule's backbone, rather than to the α-carbons, as in amino acids.

The term “polysaccharide” means any polymer (homopolymer or heteropolymer) made of subunit monosaccharides, oligimers or modified monosaccharides. The linkages between sugars can include but are not limited to acetal linkages (glycosidic bonds), ester linkages (including phophodiester linkages), amide linkages, ether linkages, etc. The lipids can be any nonpolar-comprising hydrocarbon-based molecule, including amphipathic, amphiphilic, aliphatic, straight chain, branched, aromatic, saturated, or unstaturated lipids. Specific lipid types that can be used as affinity elements here include, but are not limited to phospholipids, fatty acids, glycerides (mono-, di-, tri-, etc.), sphingolipids, and waxes. Similarly, any other suitable organic compounds, inorganic compounds, therapeutic agents, and polymers can be used as affinity elements according to the present invention.

The target can be any structure capable of binding an affinity element including but not limited to nucleic acids, proteins (with or without glycosylation), polypeptides including proteins (with or without glycosylation), peptoids, polysaccharides, organic compounds, inorganic compounds, metabolites, sugar oligomers, sugar polymers, other synthetic polymers (plastics, fibers, etc.), polypeptide complexes, polypeptide aggregates, polypeptide/nucleic acid complexes, lipids, glycoproteins, lipoproteins, polypeptide/carbohydrate structures (such as peptdidogycans), chromatin structures, membrane fragments, cells, tissues, organs, organelles, inorganic surfaces, electrodes, semiconductor substrates including but not limited to silicon-based substrates, dyes, nanoparticles, nanotubes, nanowires, quantum dots, and medical devices. The target can be a single such structure, or a multimer of the same or different such structure (ie: homodimers, heterodimer, etc.), as discussed in more detail below. As is also discussed in more detail below, when additional affinity elements are used, the target(s) for the further affinity elements can be the same as the target for the first and/or second affinity elements, or different. In one embodiment, the target is not an antibody, an antibody bearing cell, or an antibody-binding cell surface receptor (or portion thereof suitable for antibody binding). In another embodiment, the target does not comprise a nucleic acid. In a further embodiment, the target comprises a polypeptide.

Targets of interest include antibodies, including anti-idiotypic antibodies and autoantibodies present in autoimmune diseases, such as diabetes, multiple sclerosis and rheumatoid arthritis. Other targets of interest are growth factor receptors (e.g., FGFR, PDGFR, EFG, NGFR, and VEGF) and their ligands. Other targets are G-protein receptors and include substance K receptor, the angiotensin receptor, the .alpha.- and .beta.-adrenergic receptors, the serotonin receptors, and PAF receptor. See, e.g., Gilman, Ann. Rev. Biochem. 56:625 649 (1987). Other targets include ion channels (e.g., calcium, sodium, potassium channels), muscarinic receptors, acetylcholine receptors, GABA receptors, glutamate receptors, and dopamine receptors (see Harpold, U.S. Pat. Nos. 5,401,629 and 5,436,128). Other targets are adhesion proteins such as integrins, selecting, and immunoglobulin superfamily members (see Springer, Nature 346:425 433 (1990). Osborn, Cell 62:3 (1990); Hynes, Cell 69:11 (1992)). Other targets are cytokines, such as interleukins IL-1 through IL-13, tumor necrosis factors α & β, interferons α, β and γ, tumor growth factor Beta (TGF-β), colony stimulating factor (CSF) and granulocyte monocyte colony stimulating factor (GM-CSF). See Human Cytokines: Handbook for Basic & Clinical Research (Aggrawal et al. eds., Blackwell Scientific, Boston, Mass. 1991). Other targets are hormones, enzymes, and intracellular and intercellular messengers, such as, adenyl cyclase, guanyl cyclase, and phospholipase C. Optionally, the target is a molecule other than an Fv portion of an antibody (ie: the antigen binding portion of an antibody). Drugs are also targets of interest. Target molecules can be human, mammalian or bacterial. Other targets are antigens, such as proteins, glycoproteins and carbohydrates from microbial pathogens, both viral and bacterial, and tumors. Still other targets are described in U.S. Pat. No. 4,366,241. Some agents screened by the target merely bind to a target. Other agents agonize or antagonize the target (e.g., in the case of an enzyme enhance or inhibit its activity).

Any suitable substrate surface can be used in the methods of the invention, including but not limited to surfaces provided by microarrays, beads, columns, optical fibers, wipes, nitrocellulose, nylon, glass, quartz, mica, diazotized membranes (paper or nylon), silicones, polyformaldehyde, cellulose, cellulose acetate, paper, ceramics, metals, metalloids, semiconductive materials, quantum dots, coated beads, other chromatographic materials, magnetic particles; plastics and other organic polymers such as polyethylene, polypropylene, and polystyrene; conducting polymers such as polypyrole and polyindole; micro or nanostructured surfaces such as nucleic acid tiling arrays, nanotube, nanowire, or nanoparticulate decorated surfaces; or porous surfaces or gels such as methacrylates, acrylamides, sugar polymers, cellulose, silicates, and other fibrous or stranded polymers. In one exemplary embodiment, the substrate comprises a substrate suitable for use in a “dipstick” device, such as one or more of the substrates disclosed above.

In one non-limiting embodiment of the methods of this first aspect of the invention, the target is detectably labeled (as discussed above) such as, in the case of peptides or proteins, a tag that can be bound by a labeled antibody. This target is then applied to a spotted array on a slide containing between 5,000 and 1,000,000 test polypeptides of 20 amino acids long. In this example, the polypeptides can be attached to the surface through the C-terminus. The sequence of the polypeptides was generated randomly from 19 amino acids, excluding cysteine. When running this type of experiment, typically 0.1% to 10% of polypeptides show some binding to the target. The binding reaction can include, for example, an excess of E. coli proteins (such as a 100 fold excess) as non-specific competitor labeled with another dye so that the specificity ratio for each polypeptide binding target can be determined. The polypeptides with the highest specificity and binding can be picked. The identity of the polypeptide on each spot is known, and thus they can be readily identified for further use, either through use of stocks of the selected polypeptides or resynthesis of the polypeptides.

Thus, in another embodiment, the methods further comprise contacting the same substrate surface or a separate substrate surface with competitor, and determining a ratio of test compound binding to target versus test compound binding to competitor. This enables identification of test compounds that not only have high affinity for the target but also relatively low affinity for competitor. In one embodiment, the target is a polypeptide and the competitor comprises a cell lysate or protein extract, including but not limited to a bacterial cell lysate or protein extract. In another embodiment, the competitor is differentially labeled from the target for ease of detection and binding ratio determination. In further embodiments, the target/competitor screen is conducted on two or more separate substrate surfaces (for example, E colilysate as the competitor on one, salmon sperm on another, abundant serum proteins on another), and binding ratios compared across the different competitors (such as in a matrix format) to identify probes that are reasonably specific. An exemplary embodiment (E coli lysate competition) is described in detail below.

In one embodiment, the methods further comprise (c) identifying test compounds that do not bind to the target with at least moderate affinity. Since the composition of each test compound on the substrate is known, the methods of this first aspect provide information on the binding affinity of the arrayed test compounds for each target tested. These data can be used for a variety of purposes, including but not limited to creating a database of test compounds and their binding affinity (or lack thereof) to different targets. Thus, in a further embodiment, the methods of any aspect or embodiment of the invention further comprise storing in a database the data obtained using the methods of the invention. Such data includes, but is not limited to, affinity element binding affinity (including quantitative measurements of dissociation constants, binding free energy changes, binding enthalpy changes and binding entropy changes), specificity, and structure/sequence, and non-affinity element (ie: non-binder) structure/sequence. Data from these analyses can be used to create a database that allows predicting which affinity elements bind different structures. Polypeptides in different groups tend to bind different surfaces of the same protein. This information can also be used to design better affinity elements for lead target analysis.

In another embodiment, the methods of the invention further comprise identifying combinations of affinity elements that bind to different sites on the same target. The affinity elements selected using the methods of the invention typically have relatively moderate affinity for the target (^{˜}μM). By linking two affinity elements that bind the same target non-competitively, the affinity and selectivity can be increased (see data below). Thus, combinations of affinity elements that bind to different target sites are first identified. Natural antibodies do this by selection of light and heavy chain variants that bind to sites on the protein with synergy. The space between light and heavy chains is largely fixed so the optimal binding site/spacing combination is selected among millions of antibody variants. The methods disclosed herein have an advantage over the natural process of antibody production by allowing essentially any spacing between sites. If the target is a dimer or a multimer, one affinity element can bind multiple sites on the target complex simultaneously (ie: affinity element binding to each of the monomers). For example, it is estimated that approximately 60% of soluble proteins are dimers or other multimers. Therefore, in many cases joining two (or more) copies of a single affinity element may provide increased affinity and/or selectivity, though affinity and/specificity may be enhanced by using two (or more) different affinity elements when the target comprises a multimer.

Any suitable technique for identifying affinity elements that bind to different sites on the same target can be used, and many such techniques are known. In some cases, particularly for homodimeric proteins, the same affinity element can be used twice to create the synthetic antibody (ie: the binding is still for different sites, one to each member of the homodimeric pair). In one non-limiting example, affinity elements that bind to different sites on the same target are identified by pre-incubating the target with a first affinity element, under conditions to promote binding of the first affinity element to the target, and then contacting the target with one or more further affinity elements, to see which further affinity elements bind to the target in the presence of first affinity element bound to the target. For example, one method to discover polypeptides binding to different sites on the same protein is to pre-incubate the protein target with one polypeptide affinity element and observe which polypeptides on the array still bind. By doing this in an iterative fashion one can classify all the binding polypeptides as to target sites on a protein. Another method is to combine all protein specific polypeptide affinity elements in a pairwise manner and then spot them on the array to assess binding to the original target. Two polypeptide affinity elements that bind to two different areas of the protein should have more than additive affinity. Even though the polypeptide affinity elements are not spaced at a single distance, there is a random distribution of polypeptide spacing. If the average spacing is around the optimal distance, then enhanced binding can occur. This can also be affected by the length and flexibility of the linker arm to the surface. In this way the pairs of polypeptide affinity elements that bind different sites on the target can be discovered in a high through put fashion. Data supporting both approaches to finding pairs is discussed below. The pairs of polypeptide affinity elements can be affixed to a surface as a mixture to take advantage of the cooperative binding. However, only a subset of the polypeptides would be in the optimal spacing. An alternative is to affix the pairs of polypeptides on a surface that has been derivatized with orthogonal chemistries so that the polypeptides can be distributed in a chosen spacing. Another embodiment involves binding the target to a surface plasmon chip and each polypeptide is flowed over to determine its binding to the target. Then the same is done for each pair of polypeptide affinity elements. For polypeptide affinity elements that occupy the same or overlapping sites on the target, the response will be the average of the individual polypeptide affinity elements. For those occupying different sites the response will be the sum. As predicted by our analysis of the effectiveness of screening versus selection, using this technique we readily obtain several polypeptide affinity elements binding two or more sites on the target.

The methods of the invention further comprise connecting two or more affinity elements (for example, as described in any of the synthetic antibody embodiments below) for a given target via a linker to create a synthetic antibody, wherein an affinity and/or specificity of the synthetic antibody for the target is increased relative to an affinity and/or specificity of either affinity element alone for the target, as discussed in more detail below.

The methods of the invention do not try to make one high affinity, perfect match synthetic antibody, but instead takes advantage of it being easier to find two weak binders and link them to produce a higher affinity binder. While not being bound by any specific hypothesis, the inventors believe that since most of the surfaces of proteins are not deeply pocketed, it will be beneficial to use larger molecules to sufficiently bind (near micromolar) the surface. This is difficult to do by selection in a library. Therefore we have developed efficient methods to screen for binding elements. However, screenable libraries are necessarily much smaller than selectable libraries (10⁹-10¹⁴). These two demands seem contradictory. We want to limit the library size but search larger molecule space. For example, the sequence space of 20 amino acid polypeptides using all possible 20 amino acids is ˜10²⁶. Our surprising discovery was that these two demands can be reconciled because the structural space represented on the surface of proteins is covered by a small number of 20 amino acid polypeptides. This allows using a small number of compounds to cover enough space to give at least micromolar Kds on two or more sites per target. In addition, since this system allows arriving at the lead ligands by screening, it has the important implication that these synbodies could be produced in a high through put fashion.

In another embodiment, the method further comprises linking two affinity elements at an appropriate distance to obtain an increase in specificity and affinity. The linker can be any molecule or structure that can connect the first and second affinity elements, including but not limited to nucleic acid linkers, amino acid linkers, any polymeric linker (heteropolymers or homopolymer), PEG linkers, nucleic acid tiles, etc. In some embodiments, the linker is a polymer comprising one or more proline-glycine-proline subunits. In some embodiments the linker is a polymer comprising one or more hydroxyproline subunits. A variety of polymers comprising proline and/or hydroxyproline are capable of forming helical structures having useful and potentially optimizable rigidity and elasticity properties. Such linkers can be naturally occurring compounds/structures or may be non-natural, including but not limited to nucleic acid analogues, amino acid analogues, etc. Connection between an affinity element and a linker can be of any type, including but not limited to covalent binding, hydrogen bonding, ionic bonding, base pairing, electrostatic interaction, and metal coordination depending on the type of linker and the types of affinity elements. Selection of an appropriate linker for use in the synthetic antibodies of the invention is well within the level of skill in the art based on the teachings herein. The linker can be rigid or flexible, depending on the desired characteristics of the linker, as described in more detail below.

Ideal linking can produce an affinity the product of the two individual binding constants of the affinity elements. One approach to this is to make a collection of each pair of affinity elements, such as polypeptides, that bind different sites bound at different distances on one or more linkers and then measure the affinity of each linked pair of affinity elements to the target (this is discussed in more detail below). Those binding cooperatively will have much higher affinity for the target. One could also mix the different constructions, incubate them with the target and then remove and wash the target (for example on nickel beads if the target were histidine tagged). The synthetic antibodies binding from the mixture would be the ones with the optimal spacing of the individual affinity elements. The identity of the high affinity binding synthetic antibody could be determined directly by mass spectrometry or indirectly by including an identifying tag on each construct.

In the process of carrying out this procedure we have noted an unexpected phenomenon. Combinations of some affinity elements will create a synthetic antibody that has an increase in affinity and specificity of about 10 fold. However, this increase is not distance sensitive, although polypeptide affinity elements do not show the increase if they are less than 1 nm apart from each other in the synthetic antibody. We interpret this type of response as a “caging” of the target as opposed to true cooperative binding. The increase in affinity is due we think basically to creating a high local concentration of binding sites that the target bounces between.

In one embodiment, an optimal linker distance provides a spacing of between about (+/−5%) 0.5 nm and about 30 nm between a first affinity element and a second affinity element. In various further embodiments, the spacing is between about 0.5 nm-25 nm, 0.5 nm-20 nm, 0.5 nm-15 nm, 0.5 nm-10 nm, 1 nm-30 nm, 1 nm-25 nm, 1 nm-20 nm, 1 nm-15 nm, and 1 nm-10 nm.

In another embodiment, a net charge of the resulting synthetic antibody at a pH 7 is between +2 and −2, particularly when the affinity elements comprise or consist of polypeptides. The inventors have discovered that synthetic antibodies with this characteristic tend to work better than those without this characteristic.

In another embodiment, the synthetic antibody binds to the target non-specifically. The inventors have surprisingly discovered that some synthetic antibodies developed through binding to a given target show high affinity binding (ie: nM) to other targets as well (see examples below). In this embodiment, the synthetic antibody can be used to selectively target multiple targets, or target specificity can be modified by techniques known to those of skill in the art. For some applications it may be desirable to create synbodies with even higher or otherwise altered affinity or selectivity. Thus, in a further and completely optional embodiment of the different aspects of the invention, the methods further comprise optimizing binding affinity of one or both of the first affinity element and the second affinity element for the target. Such optimization may be desired to produce even higher affinity binding or specificity synbodies or synbodies with specific affinities or selectivities in any range tailored for a particular application (e.g., reversible binding to a chromatographic material). In one embodiment, the optimization is carried out on a substrate, which is not possible with standard antibodies. Any techniques for optimizing the affinity of the synthetic antibody for the target can be used.

In one non-limiting example of a polypeptide-based synbody, one or both of the polypeptides in the synbody is subjected to array alanine scanning. An array is synthesized such that each amino acid in the starting sequence is changed to alanine (or any other amino acid as suitable) one by one. The original target protein is then bound to the array. If the particular amino acid is important for binding, it will bind to the target less well when substituted with alanine (assuming it was not alanine to begin with). This procedure will identify the critical amino acids. The amino acids that need to be optimized may or may not be the ones most strongly affected by the alanine substitutions. Often the alanine substitutions in combination with structural analysis suggest other amino acids or regions of the polypeptide that could be optimized. Once the critical amino acids are identified by this method, a new set of polypeptides with substitutions of the 20 different amino acids at the alanine critical or non-critical sites can be synthesized. These sets of polypeptides can be assayed against the target to find new ones with the improved characteristics. When using larger arrays (30,000 or more) it is actually possible to use a more sophisticated initial scan if desired. For example, all possible pairs of amino acids within the 17 variable positions in the polypeptide can be replaced with all combinations of 10 amino acids (there are 27,200 such polypeptides). This allows one to recognize amino acids that are in themselves important, and also to find pairwise or compensatory interactions as well that can enhance the binding. In many cases, this pairwise approach may alleviate the need for subsequent optimization (by providing substantial local optimization in itself). In other cases, it will simply determine which amino acids should be included in the subsequent optimization rounds as described below. It will be apparent to those skilled in the art based on the teachings herein that there are many variations of this approach possible for an initial screen to locate important structure/function elements of the polypeptides. This may include varying a different number of the amino acid positions at a time (more than 2), changing the number of amino acids tested per position, including non-natural amino acids or amide linked monomers into the polypeptide, creating truncations and deletions instead of substitutions, etc.

The optimization methods may further comprise constructing an array that has a wide variety of amino acids (natural or unnatural) substituted at each critical site. For example, if there were 3 critical amino acids indicated by the alanine scanning, and 20 amino acids variants were used at each of these sites, an array would consist of 8,000 polypeptides. The target protein is then applied to this array. Binding relative to the original polypeptide is compared. The selection on these arrays can be geared towards improved affinity and or specificity. Once selected, the improved polypeptides can be reinserted into the synbody to produce higher or otherwise modified affinity, selectivity, and/or kinetics of binding. For example, it may be desirable to set the affinity at a specific value. This is particularly true for applications associated with chromatography, staining of cells and sensor systems where dynamic binding is useful, and it would thus be desirable to generate synbodies that reversibly bind a target. In fact, the key issue may be to adjust the on and off times rather than the affinity. This can be done by kinetic studies of binding and release. Such studies can be done on the arrays with the proper equipment.

Those of skill in the art will recognize, based on the teachings herein, alternative methods to optimize the synbody. For example, a phage, mRNA display or yeast/bacterial display system could be used to detect the better binders. As an example for mRNA display, a chip with 4000 oligos can be purchased that would have 16 different amino acid encoded substitutions at 3 sensitive positions. These would be primed with a T7 containing primer to make fragments that can be in vitro transcribed/translated to make the polypeptide attached to its encoding mRNA. This library can be panned against the target protein to select the improved binders.

In various embodiments, the methods further comprise connecting to the synthetic antibody further affinity elements (third affinity element, fourth affinity element, etc.) that bind to the first target or other targets. In embodiments where one or more further affinity elements bind to the same target as the first and second affinity elements, the one or more further affinity elements may be connected to the first and/or second affinity element by the linker, or may be connected to the first and/or second affinity element by a one or more further linkers (second linker, third linker, etc.), which may be a further linker or may comprise or consist of a different class of compound. Where multiple linkers are used, the spatial arrangement between affinity elements connected by different linkers can be the same or different. In various further embodiments where the further affinity elements bind to the same target as the first and second affinity elements, the linker or further linker(s) provides a spatial arrangement of the further affinity element(s) to the first and the second affinity element that increases a binding affinity and/or specificity of the synthetic antibody for the target relative to a binding affinity and/or specificity of the further affinity elements for the target.

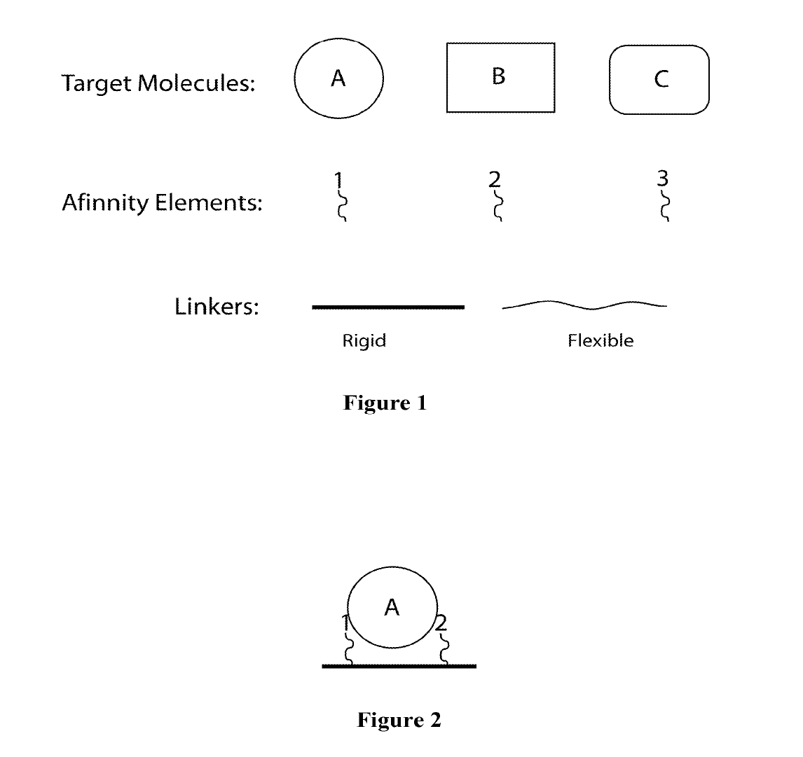

Thus, the methods for making synbodies as disclosed herein can be used to make, for example, any of the synbody embodiments disclosed herein, including but not limited to those disclosed in FIGS. 1-8, and which are discussed in detail below).

In another embodiment, the invention provides synthetic antibodies made by the methods of this first aspect of the invention.

As discussed herein, the structural complexity of the proteome surface space can be covered by ˜1000-10,000 or so affinity elements (such as polypeptides or other polymers) that can bind at ^{˜}micromolar affinity, and linking them together leads to high affinity and specificity synthetic antibodies, one could make a stock of 1000 or so binders (ie: affinity elements) that could be combined in pairs and linked to quickly make a ligand to anything. Thus, the invention further comprises a pool of affinity elements isolated according to the methods of the invention. The stocks could be pre-made in at large quantities so production could be immediately initiated. Recall that an antibody diversity of ˜10⁷ per person is capable of binding to almost anything. 1000 binders would represent 10⁶ pairs and if they can be linked in 10 different ways this stock would represent 10⁷ ligands. The equivalent of antibody diversity could be stored on the shelf for rapid, inexpensive production.

In a second aspect, the present invention provides synthetic antibodies, comprising:

(a) a first affinity element that can bind a first target;

(b) a second affinity element that can bind the first target, and which can bind to the first target in the presence of the first affinity element bound to the first target; and

(c) a linker connecting the first affinity element and the second affinity element, wherein one or both of the first affinity element and the second affinity element have a molecular weight of at least 1000 Daltons;

wherein at least one of the first affinity element and the second affinity element are not derived from the first target;

wherein the synthetic antibody has an increased binding affinity and/or specificity for the first target relative to a binding affinity and/or specificity of the first affinity element for the first target and relative to a binding affinity and/or specificity of the second affinity element for the target; and optionally wherein the first target is not an Fv region of an antibody.

Synthetic antibodies according to this aspect of the invention can be obtained against any target or targets of interest, and can generally bind to the target(s) both in solution and on surfaces, thus increasing the range of applications for their use. The spatial arrangement (ie, specific spacing and/or orientation) of the affinity elements in the synbodies improves affinity for a target relative to the affinity of the individual affinity elements for the target, and thus the synthetic antibodies are suitable for a wide variety of uses, including but not limited to ex-vivo diagnostics, for example in standard ELISA-like formats or in multiplex arrays; in vivo as imaging agents or as therapeutics for specific indications; as binding agents for affinity separation techniques and reagents, including but not limited to affinity columns and affinity beads; as detectors for environmental or biological agents; and as catalysts for chemical reactions. As therapeutics, the synthetic antibodies can be used to bind a target or for mediating binding and uptake in specific cells or as “smart drugs” for drug delivery.

As used herein, an “increased binding affinity and/or specificity of the synthetic antibody” means any increase relative to the binding affinity and/or specificity of the first affinity element for the first target and relative to a binding affinity and/or specificity of the second affinity element for the target. In various embodiments, the increase is 10-fold, 100-fold, 1000-fold, or more over either individual element.

In a further embodiment, one or both of the first and second affinity elements have a molecular weight of between about 1000 Daltons and 10,000 Daltons. In one embodiment, polypeptide compounds for use in the methods of this aspect of the invention are between about 1000 Daltons and 4000 Daltons (up to approximately 30 amino acid residues). In another embodiment, nucleic acid aptamers of up to 10,000 Daltons are used (ie: approximately 30 bases).

Synbodies according to the present invention can be of any suitable size, based on the sizes of the affinity elements and linkers used.

Affinity elements (ie: compounds identified as being affinity elements for a target of interest), targets, linkers, and other terms used in this second aspect have the same meaning as described above in the first aspect of the invention. Furthermore, all embodiments disclosed in the first aspect of the invention can be used in this second aspect of the invention.

In one embodiment, at least one of the first affinity element and the second affinity element are not the Fv portion of antibodies or antigen-binding portions thereof; in a further embodiment, neither the first nor the second affinity elements are the Fv of antibodies or antigen-binding portions thereof. Optionally, the first target is not the Fv of an antibody. In further embodiments, the first target is not an antibody, an antibody bearing cell, or an antibody-binding cell surface receptor (or portion thereof suitable for antibody binding)

Within a given synthetic antibody, the first and second affinity elements can be the same class of compound (ie: nucleic acids, polypeptides, etc.), or they can be different types of compounds. For example, the first affinity element can comprise or consist of a nucleic acid and the second affinity element can comprise or consist of a polypeptide. In one embodiment, one or both of the first and second affinity elements comprise or consist of polypeptides. Those of skill in the art will recognize a wide variety of affinity element combinations according to the present invention. In one embodiment, one or both of the first and second affinity elements comprises or consists of a non-naturally occurring compound, as discussed in the first aspect of the invention. In further embodiments, one or both of the first and second affinity elements does not comprise or consist of a nucleic acid.

In one embodiment, one or both of the first and second affinity elements, prior to inclusion in the synthetic antibodies of this aspect have dissociation constant for binding to the first target of between about 1 μM and 500 μM. Linkage of the first and second affinity elements provides a synthetic antibody with an increased affinity and/or specificity for the first target relative to a binding affinity and/or specificity of the first affinity element for the first target and relative to a binding affinity and/or specificity of the second affinity element for the target. Thus, the synthetic antibodies of the present invention combine two weaker binders by linking them; as discussed above, one surprising discovery herein is that the structural space represented on the surface of proteins is covered by a small number of 20 amino acid polypeptides. This allows using a small number of affinity elements to cover enough space to give micromolar Kds on two or more sites per target. An added advantage is that using these relatively larger molecules makes it less likely that the linker attachment will disrupt the binding of the resulting synbody to the first target.

In various embodiments, the first affinity element and the second affinity element prior to inclusion in the synthetic antibody have dissociation constant for binding to the first target of between about 1 μM-500 μM; 1 μM-150 μM; 10 μM-500 μM; 25 μM-500 μM; 50 μM-500 μM; 100 μM-500 μM; 10 μM-250 μM; 50 μM-250 μM; and 100 μM-250 μM.

In one embodiment, an optimal linker distance provides a spacing of between about 0.5 nm and about 30 nm between a first affinity element and a second affinity element. In various further embodiments, the spacing is between about 0.5 nm-25 nm, 0.5 nm-20 nm, 0.5 nm-15 nm, 0.5 nm-10 nm, 1 nm-30 nm, 1 nm-25 nm, 1 nm-20 nm, 1 nm-15 nm, and 1 nm-10 nm. Those of skill in the art can design linkers for appropriate spacing based on the teachings herein.

In another embodiment, a net charge of the synthetic antibody at a pH 7 is between +2 and −2, particularly when the affinity elements comprise or consist of polypeptides. The inventors have discovered that synthetic antibodies with this characteristic tend to work better than those without this characteristic.

While the synthetic antibodies of the invention comprise first and second affinity elements, they can comprise further such affinity elements (ie, third affinity element, fourth affinity element, etc.), as discussed in more detail below.

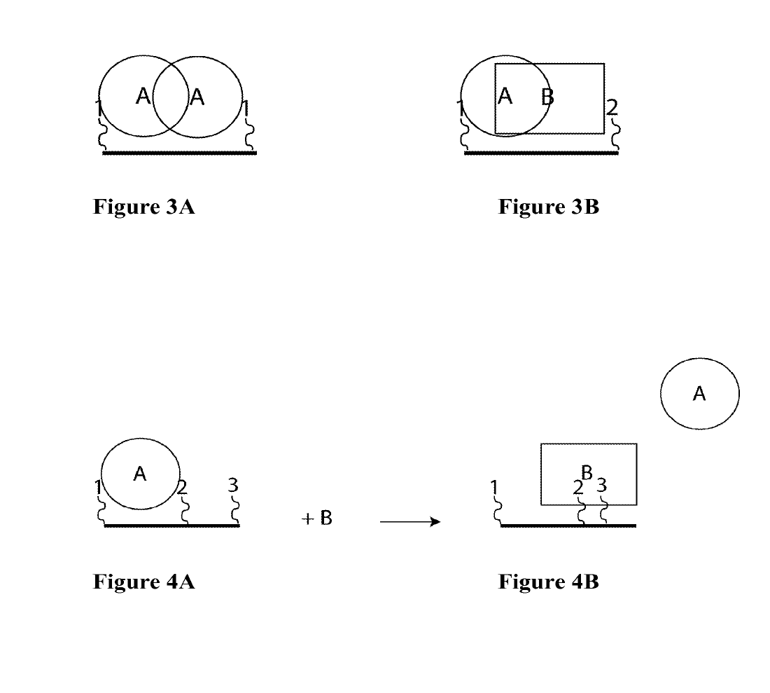

As discussed above, the synthetic antibody has an increased affinity and/or specificity for the first target relative to a binding affinity and/or specificity of the first affinity element for the first target and relative to a binding affinity and/or specificity of the second affinity element for the target. For example, the arrangement of the first and second affinity elements may increase affinity of the resulting synthetic antibody for a monomeric target (See, for example, FIG. 2). Alternatively, the arrangement of the first and second affinity elements may increase affinity and specificity of the synthetic antibody for a homodimeric or heterodimeric target, where the individual affinity elements would otherwise only be able to bind to a monomer (See, for example, FIG. 3).

The first and second affinity element bind to the first target, and their binding to the target is not exclusive, generally by virtue of the first and second affinity elements binding to different regions on the target. For example, where the target is a single structure, the first and second affinity elements may bind to different sites on the target (See, for example, FIG. 2). Alternatively, where the target is a homodimer, the first and second affinity elements may be identical and bind to the same location but one to each monomer in the homodimer (See, for example, FIG. 3A). In a further example, where the target is a heterodimer AB, the first affinity element can bind to A and the second affinity element can bind to B (See, for example, FIG. 3B). Those of skill in the art will recognize many variations based on the present disclosure. The targets for the affinity elements can be at distances not attainable by conventional antibodies. This distance can be to two different targets, as noted.

As used herein, “binding” of affinity elements to a target refers to selective binding in a complex mixture (ie: above background), and does not require that the binding be specific for a given target as traditional antibodies often cross-react. The extent of acceptable target cross-reactivity for a given synthetic antibody depends on how it is to be used and can be determined by those of skill in the art based on the teachings herein. For example, methods to modify the affinity and selectivity of the synthetic antibodies are described herein.

In various embodiments, the synthetic antibodies of the invention can comprise further affinity elements (third affinity element, fourth affinity element, etc.) that bind to the first target or other targets. The one or more further affinity elements may be connected to the first and/or second affinity element by the linker, or may be connected to the first and/or second affinity element by a one or more further linkers (second linker, third linker, etc.), which may comprise or consist of a different class of linker compound. Where multiple linkers are used, the spatial arrangement between affinity elements connected by different linkers can be the same or different. In various further embodiments the binding affinity and/or specificity of the resulting synthetic antibody for any further is increased relative to a binding affinity and/or specificity of the further affinity elements for the target.

Various further embodiments of synthetic antibodies according to this second aspect of the invention include, but are not limited to those provided in the Figures as follows:

FIGS. 4A and B: In this example, the synthetic antibody comprises affinity element 1 that binds to target A, affinity element 2 that binds to targets A and B, and affinity element 3 that binds to target B. The spatial arrangement of the 3 affinity elements by the linker provides that only one of targets A and B can be bound by the synthetic antibody. In one non-limiting embodiment, the K_{d} of binding of target A is decreased by the K_{d} of binding of B. In this particular example, the binding is competitive and a rigid linker, such as a nucleic acid linker, can be used. This synbody acts a chemical OR gate, or to control the binding of one target by the presence of another. This can be generalized to 3 or more targets, for example, by using additional affinity elements.

FIG. 5: In this example, the synthetic antibody comprises affinity elements 1 and 2 that bind to target A. Further affinity elements 3 and 4 are spatially arranged by the linker to affinity elements 1 and 2 to provide cooperative binding of a second target molecule A. For example, the dissociation constant for binding of the second target molecule A is less than or greater than that of the dissociation constant for binding of the first target molecule A-thus, positive or negative cooperativity is possible though only positive cooperativity is shown in the figure. This allows one to alter the binding curve for a particular target molecule, making it super- or sub-linear at low concentrations. This can be used, for example, to generate high contrast ratio measurements between low and high concentrations of the target.

FIG. 6: In this example, the synthetic antibody comprises affinity elements 1 and 2 that bind to target A. Further affinity elements 3 and 4 are spatially arranged by the linker to affinity elements 1 and 2 to provide cooperative binding of target molecule B. This is similar to FIG. 5 except that the cooperative binding (positive or negative) is between two different target molecules. This is another way of allowing B to influence the binding curve of A or the other way around. Unlike the case in FIG. 4, the interaction is not competitive, but is more like an allosteric affector in an enzyme system.

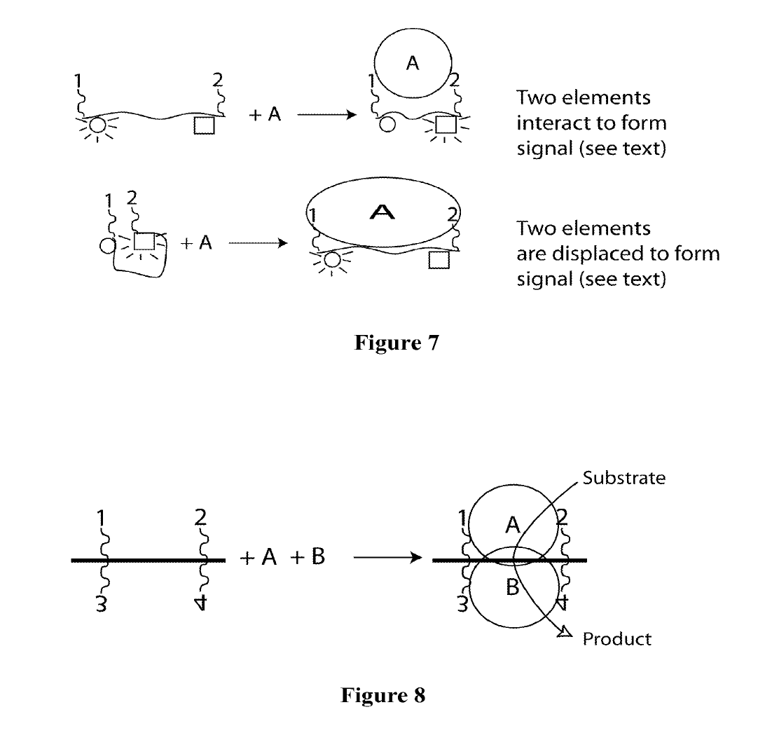

FIG. 7: The ability to design conformational or functional changes in the synbodies of the present invention upon binding and/or alter the environment of a sensor molecule upon binding is a unique capability of synbodies that cannot easily be designed into antibodies or individual ligand systems. In this example, the synthetic antibody comprises affinity elements 1 and 2 that bind to target A, and wherein binding of A to affinity elements 1 and 2 results in a spatial arrangement of two previously separated signaling elements (depicted as a circle and a square in the figure) that leads to a change in signal indicating presence of target A. The signaling elements can, for example, comprise or consist of two (or more) fluorophores that interact via fluorescence resonant energy transfer or one fluorophore and a quencher (acting either via energy transfer or electron transfer). Other interactions between a fluorophore and a second molecule or simply another part of the synbody can be designed that change the emission intensity, wavelength, spectral distribution, polarization or excited state dynamics of the fluorophores upon binding to the target. It is also possible for such conformational changes to alter the absorbance properties of the fluorophores. In other embodiments, the signaling elements can comprise or consist of one or two (or more) electrochemical sensor molecules that interact to change the observed midpoint potential or other aspects of the current voltage relationship of one or more of the molecules. Conformational changes of this kind can be directly observed via methods that measure the change in index of refraction (e.g., surface plasmon resonance) or change the surface properties of the material and thus the optical behavior at the interface (nonlinear methods such as second harmonic generation). In further embodiments, the signaling elements can comprise or consist of a series of donor and acceptor signaling molecules that are all too far apart for energy transfer to occur initially, but upon binding of multiple target molecules (can either be the same or different targets) become close enough together to form an energy (or electron) transfer network. This makes signal generation nonlinear and correlated with binding of multiple molecules (either the same or different).

FIG. 8: In this example, the synthetic antibody comprises affinity elements 1 and 2 that bind to target A. Further affinity elements 3 and 4 are spatially arranged by the linker to affinity elements 1 and 2 to self-assemble a complex of Targets A and B. This example demonstrates the ability of the synbodies of the invention to organize multiple components to direct the assembly of enzymes or other functional systems from component parts. There are many variations on this theme. In this figure, two targets are brought together to form an enzyme by binding to the synbody. Variations include, but are not limited to, bringing two subunits in close contact for some function other than catalysis, or where binding decreased enzyme activity or other functional activity. This system provides a flexible template for programming enzymatic or other functional activity in the same sense that an operon serves as a template for interactions between proteins that ultimately control gene transcription. All the same kinds of binding-based control approaches seen in transcription or other enzymatic control systems can be used here. Such systems could be used to amplify a binding signal (in the same sense as an ELISA), or to control the activity of an enzyme using in a chemical, biochemical or biomedical process.

The synthetic antibodies of the invention can be present in solution, frozen, or attached to a substrate. For example, a library of synthetic antibodies can be produced, and arrayed on a suitable substrate for use in various types of detection assays. This provides a distinct advantage over conventional antibodies, most of which do not work in array based applications. Thus, in another embodiment, one or more synthetic antibodies of the invention are bound to a surface of a substrate, either directly or indirectly. The substrate can comprise an addressable array, where the identity and location of each synthetic antibody on the array is known. Examples of such suitable substrates include, but are not limited to, microarrays, beads, columns, optical fibers, wipes, nitrocellulose, nylon, glass, quartz, mica, diazotized membranes (paper or nylon), silicones, polyformaldehyde, cellulose, cellulose acetate, paper, ceramics, metals, metalloids, semiconductive materials, quantum dots, coated beads, other chromatographic materials, magnetic particles; plastics and other organic polymers such as polyethylene, polypropylene, and polystyrene; conducting polymers such as polypyrole and polyindole; micro or nanostructured surfaces such as nucleic acid tiling arrays, nanotube, nanowire, or nanoparticulate decorated surfaces; or porous surfaces or gels such as methacrylates, acrylamides, sugar polymers, cellulose, silicates, and other fibrous or stranded polymers. In one exemplary embodiment, the substrate comprises a substrate suitable for use in a “dipstick” device, such as one or more of the substrates disclosed above.

Thus, in a further embodiment, the second aspect of the invention provides a substrate comprising:

(a) a surface; and

(b) one or more synthetic antibodies of the second aspect attached to the surface.

The substrate surface can comprise a plurality of the same synthetic antibody, or a plurality of different synthetic antibodies (where each synthetic antibody may itself also be present in multiple copies, and wherein the affinity elements in the different synthetic antibodies may be of different compounds classes (ie: some affinity elements nucleic acid-based; some polypeptide-based, etc.) When bound to a solid support, the synthetic antibodies can be directly linked to the support, or attached to the surface via known chemical means. In a further embodiment, the synthetic antibodies can be arrayed on the substrate so that each synthetic antibody (or subset of synthetic antibodies) are individually addressable on the array, as discussed herein. Thus, the substrates and/or the synthetic antibodies can be derivatized using methods known in the art to facilitate binding of the synthetic antibodies to the solid support, so long as the derivitization does not interfere with binding of the synthetic antibody to its target. The substrates may further comprise reference or control compounds or elements, as well as identifying features (RFID tags, etc.) as suitable for any given purpose.

In a third aspect, the present invention provides methods for making synthetic antibodies (according to any of the synbody embodiments disclosed herein), comprising connecting at least a first affinity element and a second affinity element for a given target via a linker;

wherein the second affinity element can bind to the target in the presence of the first affinity element bound to the target;

wherein one or both of the first affinity element and the second affinity element have a molecular weight of at least 1000 Daltons;

wherein one or both of the first affinity element and the second affinity element are not derived from the first target;

wherein the synthetic antibody has an increased binding affinity and/or specificity for the first target relative to a binding affinity and/or specificity of the first affinity element for the first target and relative to a binding affinity and/or specificity of the second affinity element for the target; and

optionally wherein the first target is not an Fv region of an antibody.

All terms and embodiments disclosed above for the first and second aspects of the invention apply to this third aspect of the invention. Connections between the affinity elements can be of any type, including but not limited to covalent binding, hydrogen bonding, ionic bonding, base pairing, electrostatic interaction, and metal coordination, depending on the type of linker and the types of affinity elements. Selection of an appropriate linker for use in the methods of making synthetic antibodies of the invention is well within level of skill in the art based on the teachings herein. In further embodiments, three, four, or more affinity elements can be physically connected by one, two, or more linkers. In each of these embodiments, the affinity elements may all be of the same compound type (nucleic acid, protein, etc.), different, or combinations thereof. In various further embodiments, the further affinity elements may bind to the same target or to one or more different targets than the target bound by the first and second affinity elements. When more than one linker is used, the linkers may all be of the same compound type (nucleic acid, protein, etc.), different, or combinations thereof.

The advantages of synthetic antibodies made by the methods disclosed herein are discussed above. In one embodiment, the methods comprise determining an appropriate spacing between the affinity elements (ie: first affinity element and second affinity element; first-second-third affinity element, etc.) in the affinity element combination. An appropriate linker distance is one that optimizes the affinity and/or specificity of the resulting synbody. Any suitable technique for determining an appropriate spacing can be used. In one non-limiting example, a predetermined set of linkers that cover increments up to 100 nm are generated, and the affinity elements are connected to each linker and the optimal distance determined using appropriate binding assays. The linker could be a derivatized PEG for example, but can be of any suitable type that can be used to determine optimal spacing, as discussed in detail above and in the examples that follow.

In another embodiment, determining optimal spacing involves systems in which in situ synthesis of linkers on a surface is used such that a series of compounds, (for example, polyalanine peptides) is made with two variably spaced lysines, differentially blocked, such that subsequent bulk attachment of the two peptides (unblocking one lysine and then the other) gives a whole range of spacings. Many other variations on this theme are possible using peptides, nucleic acids or a variety of non-natural polymers, heteropolymers, macrocycles, cavities, other scaffolds, and DNA tiling arrays.

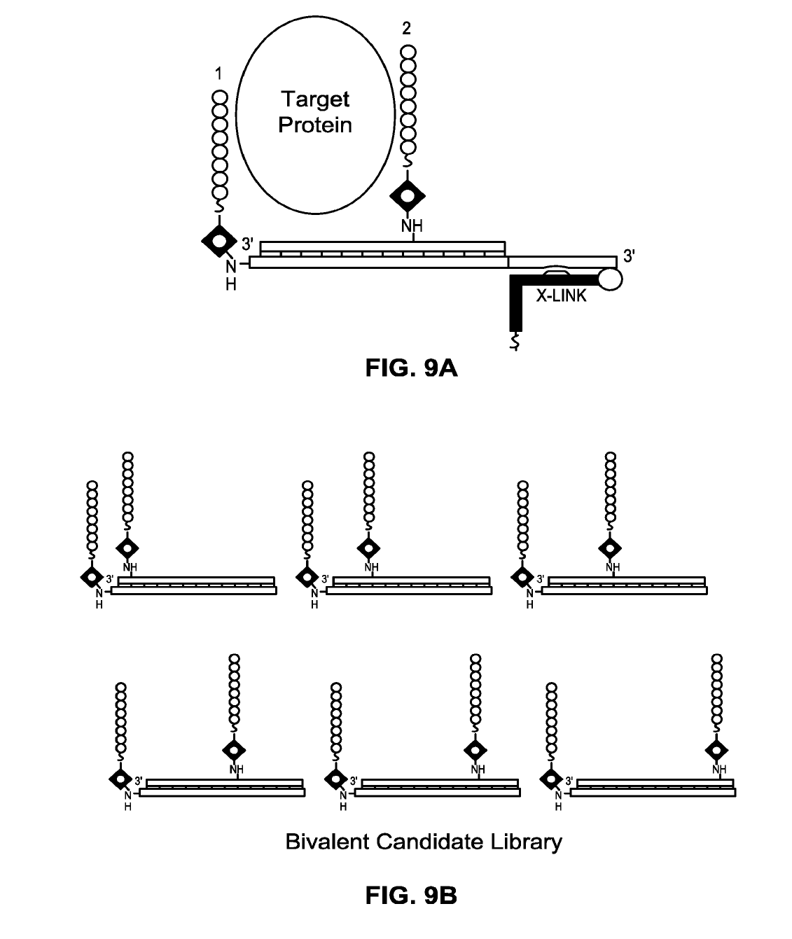

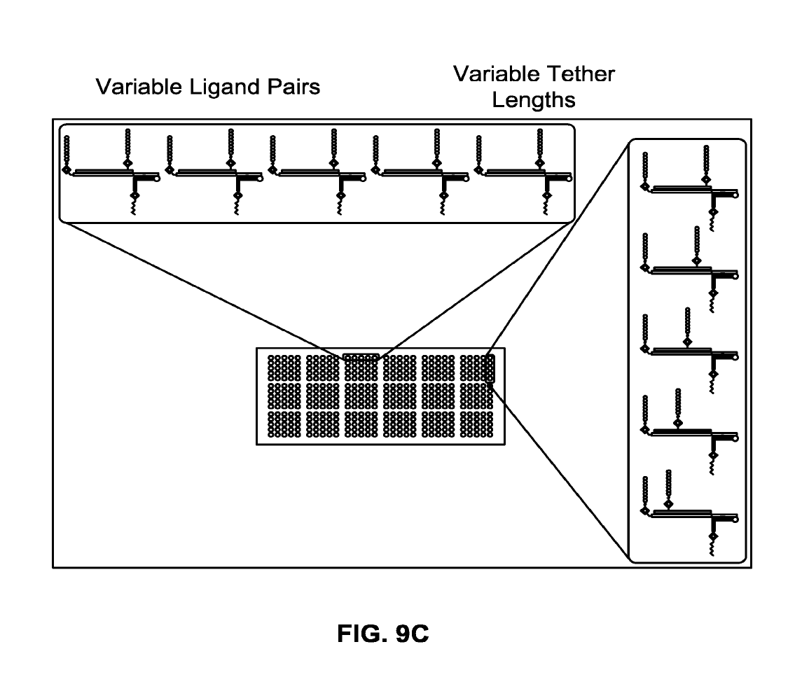

A further method involves using the flexibility of DNA to create a set of matching oligonucleotides to separate two affinity elements at set distances (FIGS. 9A and 9B). The cassette aspect of this system (as discussed in more detail below) allows ready determination of which affinity elements synergize and at what distance. Detection can be accomplished by any suitable method, including but not limited to SPR electrochemical detection, micromechanical detection (e.g., frequency shifts in resonant oscillators), electronic detection (changes in conductance or capacitance), mass spectrometry or other methods, or by spotting on a slide with florescent detection of the target. An exemplary system for SPR determination is depicted in FIG. 9C. On one slide multiple combinations of polypeptides and their distances can be tested as seen in FIG. 9C. This system is cost effective, simple, available to broad affinity element repertoire, and amenable to high throughput.

Thus, in a fourth aspect, the present invention provides a composition, comprising:

(a) a first affinity element bound to a template nucleic acid strand;

(b) a second affinity element bound to a complementary nucleic acid strand, wherein the first affinity element and the second affinity element non-competitively bind to a common target;

wherein the template nucleic acid strand and the complementary nucleic acid strand are bound to form an assembly;

wherein the first affinity element and the second affinity element are separated in the assembly; and

wherein either the template nucleic acid strand, the complementary nucleic acid strand, or both, are bound to a surface of a substrate.

In a further embodiment of this aspect, the composition further comprises the common target bound to the first affinity element and to the second affinity element.

These compositions (also referred to as a “molecular slide-rule”) can be used, for example, in the methods of the first, third, and fifth aspects of the invention for determining an optimal spatial separation of affinity elements in a synbody for a given application.

The template nucleic acid strand and the complementary nucleic acid strand are bound to form an assembly; this binding can be of any type, including but not limited to covalent binding and base pairing. One or both of the template nucleic acid strand and the complementary nucleic acid strand are also bound to the substrate surface; this binding can be of any type as discussed above, such as covalent binding, while the template and complementary nucleic acid strands are single stranded nucleic acid; preferably DNA.

Affinity elements and substrates are as disclosed above. As used in this aspect, “separated” means that the affinity elements do not bind each other, but are positioned to permit determination of optimal spacing of the affinity elements to permit binding of the first and the second affinity elements to the target simultaneously. For example, the different versions of the composition have the affinity elements separated by repetitive turns of the DNA helix (ie: the double stranded nucleic acid in the assembly formed by the template nucleic acid strand and the complementary strand base pairing).

In a further embodiment of this fourth aspect, the invention provides an array, comprising a plurality of the compositions disclosed above bound to a substrate surface, wherein the plurality of compositions comprises one or both of:

(a) a plurality of compositions wherein the first ligand and the second ligand are the same for each composition, but wherein the separation of the first ligand from the second ligand in the assembly differs; and

(b) a plurality of compositions wherein the first ligand and/or the second ligand are different for each composition.

As used in this aspect, a plurality is 2 or more; preferably 3, 4, 5, 6, 7, 8, 9, 10, or more. The compositions of option (a) are preferred for determining optimal distance between the first and second affinity elements in the synbody, while option (b) is preferred to multiplex the assay.

Binding of the compositions of the fourth aspect of the invention to the substrate can be by any suitable technique, such as those disclosed herein.

In this fourth aspect, the double stranded nucleic acid is used to template-direct the assembly of different affinity element pairs with programmed nanometer-scale spacing. DNA is an ideal material for developing synthetic architectures due to the fact that it is easy to engineer and self-assembles into highly reproducible structures of known morphology. In one non-limiting example, the template strand is conjugated to affinity element 1 and annealed to a complementary strand which is conjugated to affinity element 2. The system is designed such that affinity element 1 is separated from affinity element 2 by one additional base separations and the repetitive turns of a DNA helix (FIG. 9 b). Each base can be used to separate the two affinity agents. For each turn of the DNA helix corresponds to separation distances of roughly 4 nm, 7.5 nm, and 11 nm. Each affinity element-pair complex is spotted at independent positions on a surface and the relative or actual binding of the target to each complex is determined by any suitable technique, including but not limited to fluorescence or surface plasmon resonance (SPR).

The compositions of this fourth aspect can be attached to a surface (FIG. 9(c)) in an array format using a psoralen photocrosslinking strategy. This can be done using a psoralen-DNA ‘linker’ strand that is able to recognize a region of the template downstream of the variable strand. Once the linker strand is annealed to the template, exposure to UV light results in chemical cross linking of the linker strand to the DNA helix containing affinity element 1 and 2. Excess linker strand is then removed from the reaction mixture by affinity separation, and target binding activity and specificity is carried out. Screening can be achieved by traditional fluorescence-based assays whereby the synthetic antibody is attached to a glass slide or to a bead and then screened with fluorescently labeled target. Additionally, the synthetic antibody can be attached to a gold surface and screened with a label-free technique such as SPR, electrochemical detection, micromechanical detection (e.g., frequency shifts in resonant oscillators), electronic detection (changes in conductance or capacitance), mass spectrometry or other methods.

In a fifth aspect, the present invention provides methods for ligand identification, comprising:

(a) contacting a substrate surface comprising a target array with one or more potential ligands, wherein the contacting is done under conditions suitable for moderate to high affinity binding of the one or more ligands to suitable targets present on the substrate; and

(b) identifying targets that bind to one or more of the ligands with at least moderate affinity.

The target array can be any array of targets of interest as disclosed herein. In various embodiments, the array may comprise 50, 100, 500, 1000, 2500, 5000, 10,000; 100,000; 1,000,000; 10,000,000 or more targets. In a further embodiment, the target array is addressably arrayed (as disclosed above for compound arrays) for ease in identifying targets that have been bound. Detection of binding can be via any method known in the art, including but not limited to those disclosed elsewhere herein.

The targets may comprise any target class as described herein. In one embodiment, the targets are protein targets. In a further embodiment, the target array comprises a range of different protein targets, for protein targets not all related based on minor variations of a core sequence. In a further embodiment, the targets are not antibodies or Fv regions of antibodies. In further embodiments, the first target is not an antibody, an antibody bearing cell, or an antibody-binding cell surface receptor (or portion thereof suitable for antibody binding).

Similarly, the potential ligands can be any suitable potential ligand as disclosed herein (ie: compounds or affinity elements). In various embodiments, the potential ligand comprises a synthetic antibody according to any aspect or embodiment of the present invention. In a further embodiment, the potential ligand may be one for which a target specificity has not previously been established.

All terms and embodiments disclosed above apply equally to this aspect of the invention. In embodiments where the synthetic antibodies of the invention are used, the one or more synthetic antibodies to be screen as potential ligands comprise a first affinity element and a second affinity element, wherein one or both of the first affinity element and the second affinity element have a molecular weight of at least about 1000 Daltons; in further such embodiments, one or both of the first and second affinity elements comprise or consist of polypeptides Alternatively, the candidates could be constructed from rational design of the ligands or even from random sequences.

For artificial antibodies the starting point is almost always the protein or other target. A library of variants (single chain antibody clones, phage display of peptides, aptamer libraries, etc.) is screened against the protein target. A single clone or consensus of sequences is isolated as the specific ligand to a specific target. In all these types of examples, the starting point is a particular target for which a ligand is isolated.

In contrast, this aspect of the invention turns this standard procedure for creating ligands on its head. We first create one, a few or a library of potential ligands. For example, we create a synbody (using, for example, the methods disclosed above) consisting of two 20mer polypeptides of random (non-natural) sequence linked by a linker. In one non-limiting embodiment, the synbody has the two different polypeptides linked about 1 nM apart. The synbody is labeled and then reacted with an array with 8000 human proteins. A protein is identified that the synbody binds with high affinity and specificity. In this way a very good synthetic antibody is isolated for that particular protein. A unique aspect of this invention is that the usual process is reversed—a potential ligand is made and then a library of targets is screened for a target that is appropriately reactive.

This system is amenable to high throughput or even massively parallel screening. For example, a large number of potential ligands can be constructed by combining various binding elements, linkages, and spacing distances using, for example, the methods and synthetic antibodies disclosed above. These could be mixed (or prepared by combinatorial methods) and reacted with a large number of targets. The ligand on each target could be identified by any suitable technique, including but not limited to mass spectrometry, bar coding or mixed fluorescent tags. An advantage of this system is that it not only determines the affinity of the ligand for a particular target, but also the off-target reactivities to all the other proteins on the array.

This approach defies conventional wisdom, which would suggest that the space of possible target shapes is far too large for a screening strategy of this kind to produce synbodies having antibody-like affinities and specificities. While not being bound by a specific mechanism, the inventors believe (as described above) that there are a very limited number of distinct substructures on the surface of proteins. That is, unlike sequence space, the structural space represented on the surface of proteins is very limited. Proteins have a limited number of shapes on their surface. A second aspect of the hypothesis is that a small number of appropriately chosen ligands can represent the structural complements of all the shapes present on protein surfaces.

For example, 5,000 20-amino acid polypeptides of non-life sequence can provide most complementary shapes. A third aspect is that if two of these shape binding elements are held at a fixed distance, the resulting synbody is likely to find, in a library of reasonable size, some protein having complementary shapes at that distance, and will bind that protein in a cooperative fashion and with high specificity.

In various further embodiments of this aspect of the invention are methods for screening the antibodies and synbodies on a protein microarray in a manner that reduces the number of (very expensive) microarrays required for screening a given number of candidates. In one non-limiting example, affinity data is read using a real-time microarray reader with the protein microarray mounted in a flow chamber. Buffer containing a single antibody or synbody in very low concentration is flowed over the microarray until binding is detected on a small number of targets; these will be the highest affinity targets for that antibody or synbody. Since the antibody or synbody has very low affinity for all but the few targets for which it is specific, and since the antibody or synbody is applied at very low concentration and the flow stopped after binding is detected, nearly all targets will remain unoccupied and even the occupied targets will be far from saturation. The process can then be repeated with a second antibody or synbody, thereby obtaining maximum benefit from the protein array.

In another embodiment, the methods of this aspect of the invention can be used to identify new targets for existing antibodies, including therapeutic, diagnostic, and research antibodies. As disclosed below, the methods provide valuable information on the specificity of such antibodies in a high throughput and low cost manner, and allow identification of antibodies specific for targets for which antibodies are currently unavailable.

In a sixth aspect, the present invention provides methods for identifying a synthetic antibody profile for a test sample of interest, comprising contacting a substrate comprising a plurality of synthetic antibodies according to the present invention with a test sample and comparing synthetic antibody binding to the test sample with synthetic antibody binding to a control sample, wherein synthetic antibodies that differentially bind to targets in the test sample relative to the control sample comprise a synthetic antibody profile for the test sample.

As used in this aspect, a plurality means 2 or more; preferably 50, 100, 250, 500, 1000, 2500, 5000, or more. The test sample can be any sample of interest, including but not limited to a patient tissue sample (such as including but not limited to blood, serum, bone marrow, saliva, sputum, throat washings, tears, urine, semen, and vaginal secretions or surgical specimen such as biopsy or tumor, or tissue removed for cytological examination), research samples (including but not limited to cell extracts, tissue extracts, organ extracts, etc.), or any other sample of interest. Such a patient sample can be from any patient class of interest. The control sample can be any suitable control, such as a similar tissue sample from a known normal, or any other standard. Thus, the methods can be used, for example, as a diagnostic, prognostic, or research tool. In one embodiment, the control sample is contacted with the same substrate as the test sample; in another embodiment, the control sample is contacted with a different but similar or identical substrate as the test sample.

In this aspect, a plurality of synthetic antibody candidates (ie: 10, 20, 50, 100, 250, 500, 1000, 2500, 5000 or more) are arrayed in an addressable fashion, for example on a printed slide. The ligands in the candidates could be from pre-selected sequences, rational design or random sequence. These arrays would then be used to screen samples of interest. For example they could be serum from normal and affected subjects. Synthetic antibodies that bound components of the serum and ones that differentially bound components between the two samples could be selected. The actual target or targets bound by each synthetic antibody could be determined directly from the array by mass spectrometry or by using the synthetic antibody as and affinity agent to purify the targets.

Any one or all of the steps of the methods of the different aspects of the invention can be automated or semi-automated, using automated synthesis methods, robotic handling of substrates, microfluidics, and automated signal detection and analysis hardware (such as fluorescence detection hardware) and software.

Thus, in another aspect, the invention provides computer readable storage media comprising a set of instructions for causing a signal detection device to execute procedures for carrying out the methods of the invention. For example, the procedures comprise the signal processing, target affinity element identification steps and databasing of the second aspect of the invention, and any/all embodiments thereof. The computer readable storage medium can include, but is not limited to, magnetic disks, optical disks, organic memory, and any other volatile (e.g., Random Access Memory (“RAM”)) or non-volatile (e.g., Read-Only Memory (“ROM”)) mass storage system readable by a central processing unit (“CPU”). The computer readable storage medium includes cooperating or interconnected computer readable medium, which can exist exclusively on the processing system of the processing device or be distributed among multiple interconnected processing systems that may be local or remote to the processing device.

The invention further provides kits, comprising any one or more of the reagents disclosed herein. Such kits can be used, for example, for selecting affinity elements and making synbodies out of them, using the methods disclosed herein.

### Example 1

In one non-limiting embodiment of this second aspect of the invention, an array of 4,000 polypeptides is spotted on a slide. Each polypeptide is 20 amino acids in length, and is spotted such that its orientation is controlled to be through the C-terminus. A large amount of sequence and chemical space can be adequately sampled using only a small fraction of the possible space. For example, in the case of this array, there are 19¹⁷=5×10² possible polypeptide sequences (the first 3 amino acids are held constant, but this is not necessary and cysteine is used only at the C-terminus as attachment via a thiol), but we sampled just 4×10³ sequences and can identify polypeptides that show moderate binding affinity and specificity to a number of proteins.

The target protein is labeled with a florescent dye and incubated with the array. Polypeptides that bind the target protein are determined. Alternatively, we have incubated unlabelled affinity tagged form of the target protein and detected binding by virtue of a secondary antibody against the tag. Each sequence of the polypeptides on each spot is already known; thus, the process is a screen for elements, not a selection. Thus, the process of ligand discovery is limited only by the rate at which individual targets can be screened on pre-printed polypeptide arrays. In this sense it is distinct from aptamer, phage or other panning methods, in which recurrent selections using unknown sequences are required, and only those elements that do bind a target are determined, while those that do not bind are not known.

Whether such a small sequence space can yield effective binders depends on how the binding space is shaped. If the slope of relative binding affinity is very steep around the optimal polypeptides, it is unlikely that one of the 4,000 polypeptides will be close to one of the optimal polypeptides. If however, the slope of the binding space is gradual, one may find polypeptides that are on the “side of the mountain.” If the determination of the optimal polypeptide is by virtue of sequence similarity, it is very unlikely that in 4000 polypeptides ones with sequence similar to the optimal would be found in the 10²¹ possibilities (for 17mer polypeptides).

Most experts in this field thought this process would not work-but it does. Consistent with the logic above, most of the polypeptides that bind a particular site on a protein do not resemble each other in sequence. Therefore, while not being bound by any hypothesis, we suggest the following explanation, which represents a new insight into peptide sequence space. We propose that the 10²¹ possible 17mer polypeptides actually form a very limited number (˜4000) of structural forms. This view has several important predictions and implications. First, the space dimension would be much smaller. Therefore, around each optimal sequence would be structurally related polypeptides on the side of the mountain that would not necessarily have any sequence similarity. Second, several proteins may bind to a specific peptide but that peptide could be varied to bind better to one or the other. In other words, the same 4000 polypeptides may be all that is needed to generate synbodies to virtually an unlimited number of targets.

Once a set of affinity agents are isolated for a given target we may use these directly or use them to create an artificial antibody. For the latter we identify two or more elements that bind different sites on the targets. To do so we can, for example, block target binding with the target polypeptides or co-spot them on slides or we can put pairs onto DNA linkers to determine pairs and spacing simultaneously (FIG. 9c). The pairs of affinity elements may be valuable in themselves.

We then create a synbody using the system for measuring as described. A first affinity element is covalently attached to a DNA template strand, and separately attaching affinity element two to different nucleotide positions on a complementary strand. We anneal the two strands of DNA and immobilize the complex to 400 different sites on a surface plasmon resonance (SPR) Flexchip. We then flow the target of interest over the surface to identify different ligand pairs and ligand pair separation distances with enhanced binding. Ligand pairs and ligand pair separation distances with the greatest binding enhancement are either used directly or reconstructed with synthetic tethers based on the distance parameter determined in the SPR analysis. We have used this process to generate a synbody to Gal80 that exhibits enhanced binding as described in detail in Example 6 below. The Gal80 synbody functions with high affinity and high specificity in solution (Elisa format) and on a solid surface (see Example 8).

Synbodies developed with the techniques disclosed above in the second, third, and/or fourth aspects of the invention function when immobilized to a surface and also function as a solution phase binding agent. The highest binding synbody candidate from one experiment was used as the detection agent in an ELISA experiment and the solution phase dissociation constant (K_{d}) was determined for the synbody, each polypeptide on the synbody and the DNA backbone (see Example 8). This data demonstrates that a large increase in binding affinity can be achieved through the use of the synergistic polypeptides with the proper distance. An additional advantage to this approach is that the synbody is discovered in a single assay and then there is enough of the synbody available to immediately use as the detection agent in a functional assay. This in effect couples discovery and production into a single step, dramatically shortening the synbody development time.

### Example 2 Microarray Selection of Affinity Elements for Synbody

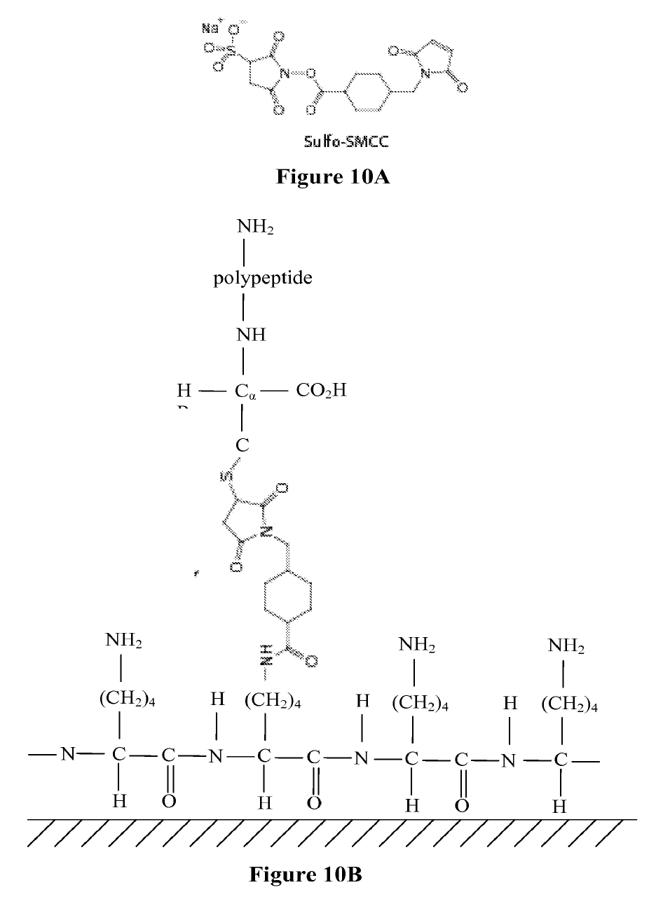

This example demonstrates the identification of affinity elements by screening a target on an array of random polypeptides. A microarray was prepared by robotically spotting about 4,000 distinct polypeptide compositions, two replicate array features per polypeptide composition, on a glass slide having a poly-lysine surface coating. Each polypeptide was 20 residues in length, with glycine-serine-cysteine as the three C-terminal residues and the remaining residues determined by a pseudorandom computational process in which each of the 20 naturally occurring amino acids except cysteine had an equal probability of being chosen at each position. Cysteine was not used except at the C-terminal position, to facilitate correct conjugation to the surface. Polypeptides were conjugated to the polylysine surface coating by thiol attachment of a C-terminal cysteine of the polypeptide to a maleimide (sulfo-SMCC, sulfosuccinimidyl 4[N-maleimidomethyl]cyclohexane-1-carboxylate, see FIG. 10A), which is covalently bonded to the E amine of a lysine monomer of the poly-lysine surface coating, as shown in FIG. 10B. The polypeptides were synthesized by Alta Biosciences, Birmingham, UK. Each polypeptide was first dissolved in dimethyl formamide overnight and master stock plates prepared by adding an equal volume of water so that the final polypeptide concentration was about 2 mg/ml. Working spotting plates were prepared by diluting equal volumes of the polypeptides from the master plates with phosphate buffered saline for a final polypeptide concentration of about 1 mg/ml. The polypeptides were spotted in duplicate using a SpotArray 72 microarray printer (Perkin Elmer, Wellesley, Mass.) and the printed slides stored under an argon atmosphere at 4° C. until used. Any other spotting/immobilization chemistry and/or method operable for immobilizing polypeptides on an array surface in a manner compatible with the intended array assay may be employed; by way of non-limiting examples, polypeptides may be conjugated directly to a polylysine surface coating via an amide bond between the C-terminal residue of the polypeptide and the c amine of a lysine, or may be conjugated to an aminosilane or other functionalized surface exposing free amines. Linkers other than or in addition to SMCC may also be employed; by way of non-limiting example, a PEG linker may be used to position the polypeptide away from the substrate. Surface functionalizations other than amine can be employed, coupled with conjugation chemistry appropriate for attachment of the affinity elements to the surface moieties provided. In some embodiments the surface immobilization may be non-covalent.

Several polypeptides were identified as candidate affinity elements for synbodies against an arbitrarily chosen protein target, transferrin, by incubating transferrin on the polypeptide microarray in the presence of E. coli lysate competitor. Transferrin was randomly direct-labeled at free amines with Alexa™ 555, and E. coli lysate was randomly direct-labeled at free amines with Alexa™ 647. Three replicate arrays were passivized by applying a mixture of BSA and mercaptohexanol for one hour. The arrays were blocked with unlabelled E. coli lysate for one hour, then washed three times with TBST (0.05% Tween) followed by three times with water. A mixture of labeled transferrin and labeled E. coli lysate was applied to the three replicate arrays and incubated for three hours. The arrays were again washed three times with TBST (0.05% Tween) followed by three times with water, and scanned at 555 nm and 647 nm using an array reader. Polypeptides were ranked as candidates for inclusion as affinity elements of synbodies by computing a score for each polypeptide equal to the mean raw 555 nm intensity over the six replicate features, squared, divided by the mean raw 647 nm intensity over the six replicate features. This simple scoring function tends to favor candidate polypeptides that bind at least moderate affinity, since otherwise the 555 nm intensity would be relatively lower, and that are relatively specific, since otherwise the 647 nm intensity would be relatively higher and contribute to a relatively lower score. Many variations of this ranking and identification process can be used, such as, by way of non-limiting examples, two-color comparisons against other competitors; comparisons with data taken in separate experiments with respect to other targets; and use of scoring functions taking into account other factors, employing other functional relationships, and/or involving statistical analysis and/or preprocessing of data and/or correcting for background fluorescence and/or other factors affecting the accuracy of the measured intensities. Ten polypeptides (Table 1) were identified for further evaluation for use as affinity elements in synbodies by choosing the polypeptides having the highest score (one polypeptide was rejected as difficult to synthesize, so the polypeptides chosen were ten of those having the eleven highest scores).

TABLE 1
Transferrin binding affinity elements

| | |
|---|---|
| TRF19 | KEDNPGYSSEQDYNKLDGSC (SEQ ID NO: 1) |
| TRF20 | GQTQFAMHRFQQWYKIKGSC (SEQ ID NO: 2) |
| TRF21 | QYHHFMNLKRQGRAQAYGSC (SEQ ID NO: 3) |
| TRF22 | HAYKGPGDMRRFNHSGMGSC (SEQ ID NO: 4) |
| TRF23 | FRGWAHIFFGPHVIYRGGSC (SEQ ID NO: 5) |
| TRF24 | SVKPWRPLITGNRWLNSGSC (SEQ ID NO: 6) |
| TRF25 | APYAPQQIHYWSTLGFKGSC (SEQ ID NO: 7) |
| TRF26 | AHKVVPQRQIRHAYNRYGSC (SEQ ID NO: 8) |
| TRF27 | LDPLFNTSIMVNWHRWMGSC (SEQ ID NO: 9) |
| TRF27 | LDPLFNTSIMVNWHRWMGSC (SEQ ID NO: 10) |
| TRF28 | RFQLTQHYAQFWGHYTWGSC (SEQ ID NO: 11) |

### Example 3 Microarray Selection of Affinity Elements for DNA Linked Synbody

This example demonstrates another embodiment of a process for identifying affinity elements for incorporation into a synbody. 15-mer polypeptide affinity elements for a DNA linked synbody specific for Gal80 were identified by obtaining and analyzing data from several polypeptide microarray experiments performed using standard 4,000 feature polypeptide microarrays each of whose features comprised a polypeptide 15 residues in length, terminating in glycine-serine-cysteine at the C-terminus, with the other 12 residues selected from 8 of the 20 naturally occurring amino acids according to a pseudorandom algorithm. Four fluorophore-labeled protein targets-gal80, gal80 complexed with gal4 binding polypeptide, transferrin, and α-antitrypsin-were supplied to LC Sciences for array analysis according to LC Sciences' proprietary protocol, and binding (fluorescence intensity) data were obtained. For screening against the random peptide array, Gal80 was labeled with Cy3 and Cy5 fluorescent dyes (GE Healthcare) according to the manufacturers protocol. The dye-to-protein ratio was determined using the Proteins and Labels settings on a Nanodrop ND-100 spectrophotometer (Nanodrop Technologies). The dye-to-protein ratio for Cy3 and Cy5 labeled Gal80 was 3.4 and 5.0 respectively. The blocking solution used to block the peptide arrays was composed of 1% bovine serum albumin (BSA), 0.5% non-fat milk, 0.05% Tween-20 in 1× phosphate buffered saline (PBS) pH 7.4. After blocking, each array was then washed 3 times with a wash buffer composed of 0.05% Tween-20 in 1 xPBS, pH 7.4. The incubation buffer was composed of 1% bovine serum albumin (BSA), 0.5% non-fat milk, in 1× phosphate buffered saline (PBS) pH 7.4. An Axon GenePix 400B Microarray Scanner (Molecular Devices, Sunnyvale, Calif.) was used to acquire images of the peptide arrays. An initial scan of the array was acquired to determine any background fluorescence from each peptide on the array. Fluorescent intensities obtained after protein incubation were subtracted from the background fluorescence and exported into Microsoft Excel for analysis.

Gal4 binding polypeptide is known to bind gal80 at a specific binding site (the gal4 binding site). 142 of the array polypeptides bound gal80 at above-threshold fluorescent intensities, 29 of the array polypeptides bound gal80 complexed to gal4 binding polypeptide at above-threshold fluorescent intensities, and 10 of the array polypeptides bound both gal80 and gal80 complexed to gal4 binding polypeptide at above-threshold fluorescent intensities. Polypeptides that bound gal80 complexed to gal4 binding polypeptide but that did not bind gal80 alone were rejected as likely to be binding to the gal4 binding polypeptide. Intensity data for polypeptides that bound gal80 alone but not gal80 complexed to gal4 binding polypeptide (implying that these polypeptides were binding to the gal4 binding site on gal80) were compared with the intensity data for the same polypeptides with respect to transferrin and α-antitrypsin; polypeptides showing significant binding to either transferrin or α-antitrypsin were excluded, and of the polypeptides remaining, the polypeptide having the highest intensity binding for gal80 was chosen as a first affinity element for incorporation in the gal80 synbody. Intensity data for polypeptides that bound both gal80 alone and gal80 complexed to gal4 binding peptide (implying that these polypeptides were binding gal80 at a site other than the gal4 binding site) were compared with intensity data for the same polypeptides with respect to transferrin and α-antitrypsin; again, polypeptides showing significant binding to either transferrin or α-antitrypsin were excluded, and of the polypeptides remaining, the polypeptide having the highest intensity binding for gal80 was chosen as the second affinity element for incorporation in the gal80 synbody. The sequences of the chosen polypeptides were as shown in Table 2.

TABLE 2
Ga180 binding affinity elements

| | |
|---|---|
| BP1 | NH₂-GTEKGTSGWLKTGSC-CO₂H (SEQ ID NO: 12) |
| BP2 | NH₂-EGEWTEGKLSLRGSC-CO₂H (SEQ ID NO: 13) |

### Example 4 SPR Verification of Binding Characteristics of Transferrin Synbody Affinity Elements

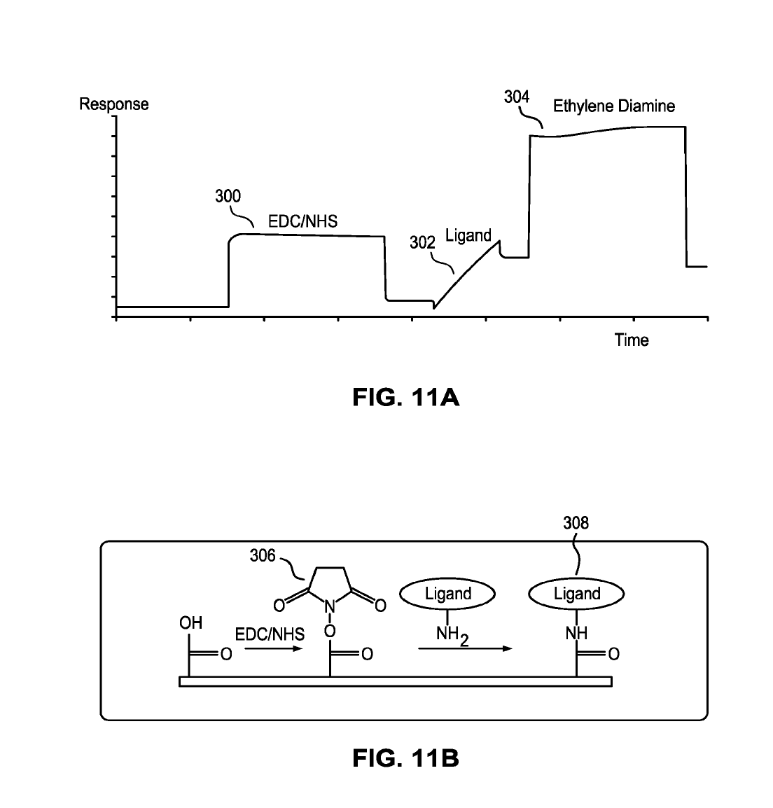

This example demonstrates SPR determination of the binding characteristics of affinity elements. Transferrin was immobilized by amine-coupling to the carboxyl-functionalized surface of a Biacore T100 CMS Dextran SPR chip as illustrated in FIGS. 11A, B. A 1:1 mixture of EDC (0.4M 1-ethyl-3-(3-dimethylaminopropyl)-carbodiimide in water) and NHS (0.1M N-hydroxysuccinimide in water) was applied 300 at a flow rate of 5 to 10 μl/min for a contact time of about 6 to 10 minutes to activate the surface by conjugating a maleimide 306 to the surface-exposed carboxyl groups. Transferrin 25 μg/ml in immobilization buffer selected for correct pH was then applied 302 at a flow rate of 5 to 10 μl/min for a contact time of about 5 to 10 minutes, allowing the amine functionality on the transferrin 308 to displace the activated NHS ester and bond to the surface via an amide bond. Finally, ethylene diamine (1-Methylene diamine-HCl at pH 8.5) was applied 304 at a flow rate of 5 to 10 μl/min for a contact time of about 6 to 7 minutes to deactivate any remaining reactive groups on the dextran chip surface. Flow rates and contact times are adjusted as necessary to provide the surface concentration of target desired for the intended application, and may vary by target. In general, for evaluating whether binding occurs, it is preferable to immobilize a relatively large quantity of target, and higher flow rates and/or longer contact times may be used. For determining kinetics, it is preferable to limit the amount of target immobilized so as to minimize rebinding and avidity effects, and lower flow rates and/or contact times may be used.

Candidate affinity elements for the transferrin synbody TRF19, TRF21, TRF23, TRF24, TRF25, and TRF26 were individually evaluated for solution phase K_{D} with respect to transferrin by SPR analysis. Because the off rates for these polypeptides were very high, K_{D} values were estimated by measuring steady-state response for at least five concentrations in a two-fold dilution series, each concentration tested in duplicate. For each experiment, response data were processed using a reference surface to correct for bulk refractive index changes and any non-specific binding. Data were also double referenced using responses from blank running buffer injections. Each experiment was conducted at 25° C. using PBST (0.01M Phosphate Buffered Saline, 0.138M NaCl, 0.0027M KCl, 0.05% surfactant Tween20, pH 7.4) as the running buffer on a Biacore T100 instrument. Analytes were injected for 60 s at a flow rate of 30 μl/min. The antigen surfaces were regenerated with 30 s consecutive pulses of NaOH/NaCl (50 mM NaOH in 1M NaCl) and Glycine (10 mM glycine-HCl, pH 2.5). Estimate K_{D} values are shown in Table 3.

TABLE 3
KD values for transferrin synbody candidate affinity elements

| | Solution Phase K_{D} | |
|---|---|---|
| TRF19 | ~150 | uM |
| TRF21 | ~60 | uM |
| TRF23 | ~50 | uM |
| TRF24 | ~50 | uM |
| TRF25 | ~60 | uM |
| TRF26 | ~100 | uM |

### Example 5 SPR Analysis of Affinity Element Binding to Distinct/Multiple Sites on Target

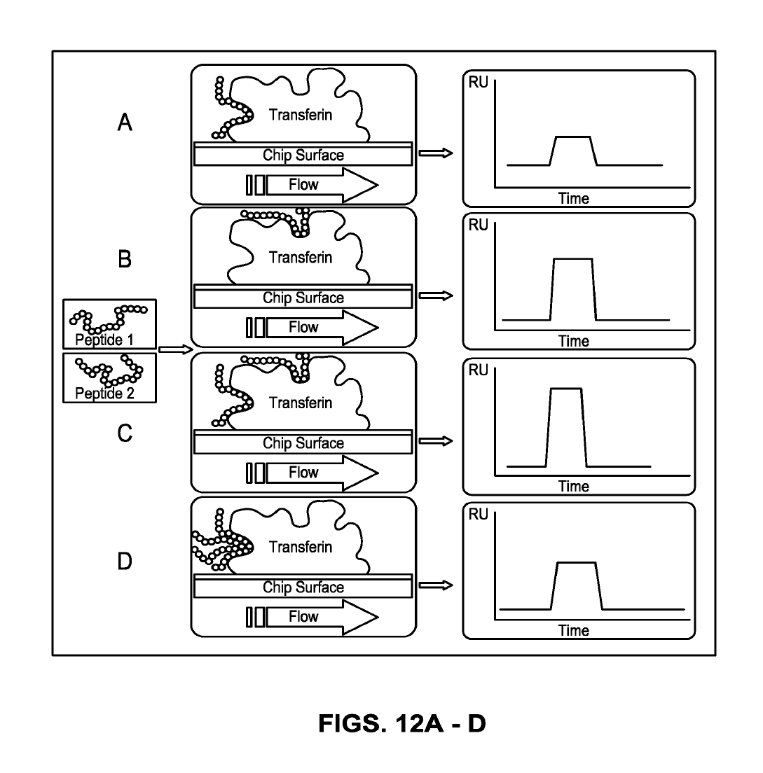

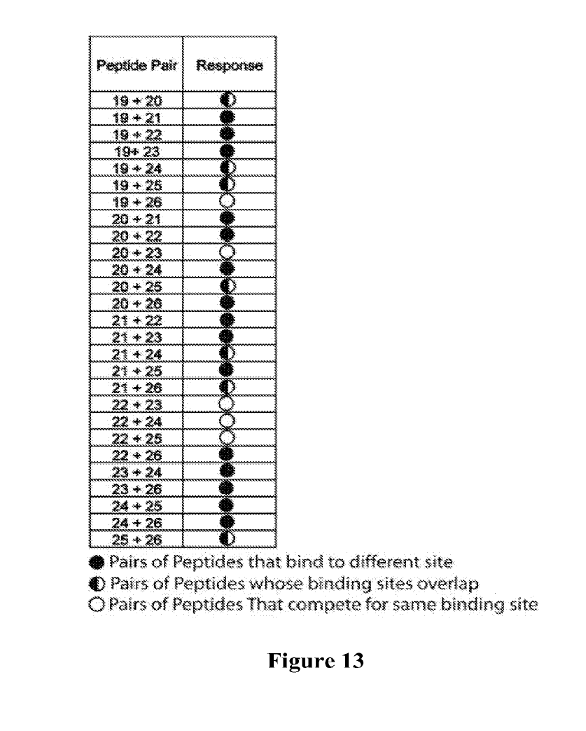

This example demonstrates an SPR-based method for identifying polypeptide affinity elements that bind distinct sites on a protein target. The transferrin target was immobilized on a Biacore T100 SPR chip, and candidate polypeptides were applied in 1:1 mixtures in pairs and response data obtained, in accordance with the methods described in Example 4 above. As illustrated in FIGS. 12A-D, upon flowing candidate polypeptides over the immobilized target, ideally one polypeptide applied alone would bind to a first binding site on the target and produce a first characteristic SPR response level (FIG. 12A), the other polypeptide would bind to a second, distinct binding site on the target, producing a second characteristic response level (FIG. 12B), and a mixture of the two polypeptides together (at the same concentrations as before) would produce a response level approximating the sum of the response levels produced by each polypeptide alone, as the polypeptides bind to distinct binding sites (FIG. 12C). However, it is also possible that the two polypeptides do not bind distinct sites on the target, but instead compete for the same binding site (FIG. 12D), in which case the expected SPR response would be intermediate between the response level produced by either polypeptide separately and the sum of the two. FIG. 13 shows the results of evaluation of a number of pairs of the polypeptides that were identified as described in Example 2 (see Table 1). Among other pairs, TRF23 and TRF26 had solution phase affinities for transferrin in a range of K_{D} of about 50 to 100 μM (see Table 3) and were found to bind distinct sites on transferrin.

Analysis to determine ability to bind distinct binding sites can be performed by any other method operable to assess whether two affinity elements do or do not mutually interfere in binding to the target. By way of non-limiting example, this may be done by comparing, by array experiment, SPR, or any other suitable method, a polypeptide's binding characteristics with respect to a target with the target pre-bound to a target-specific antibody; it may be inferred that polypeptides that bind the target with and without the antibody present are likely binding to a site other than the site that the antibody binds, and that polypeptides that bind the target without the antibody present and do not bind with the antibody present are likely binding to the site that the antibody binds.

### Example 6 Synthesis of DNA-Linker Synbody

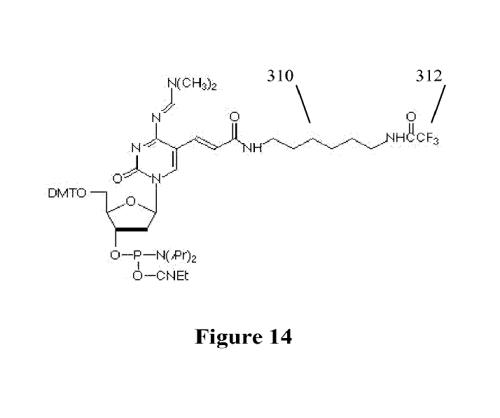

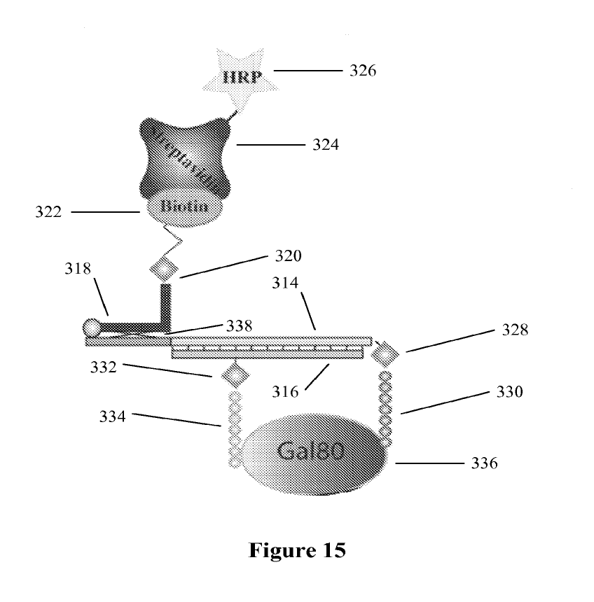

This example demonstrates the synthesis of a synbody specific for gal80, comprising two 15-mer polypeptide affinity elements identified as described in Example 3 joined by a DNA linker. The structure is illustrated schematically in FIG. 15. The DNA linker sequence was determined randomly, subject to the constraints that the sequence should not result in predicted formation of secondary structures, should not be similar or identical to any naturally occurring sequence as determined by BLAST search, and the variable strand should have cytosine residues at the locations at which attachment of the affinity elements is desired (although other attachment modalities could be used, for convenience the attachment employed involved C6 amine modification of the cytosine base). The template strand 314 was amine-modified at the 5′ terminal cytosine residue to allow attachment of the polypeptide affinity element 330 via a maleimide linker 328. The variable strand 316 was reverse complementary to the template strand and was amine-modified at an internal cytosine residue to allow attachment of the other polypeptide affinity element 334, again via a maleimide linker 332. A library of variable strands were obtained, each amine-modified at a different position, to provide a range of attachment points corresponding to a range of separation distances between the affinity elements. Determination of attachment points also took into account the angular orientation of residues along the DNA helix, so as to avoid positioning the affinity elements on opposite sides of the DNA backbone. For B-DNA in solution under physiological conditions, the double helix makes a complete rotation in about 10.4 to 10.5 base pairs and has a length of about 3.4 nm per 10 base pairs. To align the attachment points of the affinity elements at approximately the same angular position around the longitudinal axis of the helix, and keeping in mind that the affinity elements are attached to opposite strands, the bases comprising the attachment points may be chosen at a separation of approximately an even multiple of about 10.5 (one full rotation) plus about 4 (to account for the difference in angular position between the strands), plus or minus about 2 or 3 (since affinity elements do not necessarily bind optimally to the target by being perfectly aligned with each other). By screening various attachment points, various separation distances and relative orientations of the affinity elements can be tested. For the example here described, variable strands having amine-modified cytosines at positions 13, 15, 17, 24, 26, and 28 (counting from the 3′ end of the variable strand) were obtained. The amine-modified cytosines (hereafter dC C6) were incorporated in the oligonucleotides using 5′-Dimethoxytrityl-N-dimethylformamidine-5-[N-(trifluoroacetylaminohexyl)-3-acrylimido]-2′-deoxyCytidine, 3′-[(2-cyanoethyl)-(N,N-diisopropyl)]-phosphoramidite, see FIG. 14, and have a trifluoroacetylaminohexyl moiety 310 extending from the 5 carbon of the cytidine base.

The polypeptides were conjugated to synthetic DNA template 314 and variable 316 strands in accordance with methods described in detail in Williams B A R, Lund K, Liu Y, Yan H, Chaput J C: Self-Assembled Peptide Nanoarrays: An Approach to Studying Protein-Protein Interactions. Angew Chem Int Ed 2007, 46:3051-3054. The two DNA oligonucleotides, template strand 314 (5′ (dC C6)CC GAA ACA ACC GCG AGA GGC ACG CGC GTA GCC GTC ACC GGC TAT-3′ (SEQ ID NO: 14), wherein the 5′ terminal dC C6 is amine-modified cytosine as described above) and variable strand 316 (5′ GCT ACG CGC GTG CCT CTC G(dC C6)G GTT GTT TCG GG-3′ (SEQ ID NO: 15), wherein the dC C6 appearing at the position 13 counting from the 3′ terminus is amine-modified cytosine) were purchased from Keck Oligonucleotide Synthesis Facility (Yale University). These were conjugated (at the trifluoroacetyl moiety (312, FIG. 14) of the amine-modified cytosine to the bifunctional linker 4-(maleimidomethyl)-1-cyclohexane carboxylic acid N-hydroxysuccinimide ester (SMCC, Sigma Aldrich) 328, 332 by combining 200 μL of SMCC (1 mg/mL) in acetonitrile with 200 μL of DNA (20 nmol) in 0.1 M KHPO₄ buffer (pH 7.2). Following a 3 h incubation at room temperature, a second portion (20 μL) of SMCC (10 mg/ml) was added and the reaction was allowed to continue overnight at room temperature. Excess SMCC was removed from the SMCC conjugated DNA samples by size exclusion chromatography on a Nap-5 column (Amersham Bioscience). To construct the polypeptide-oligonucleotide conjugates, the Gal 80 binding polypeptide 330 (NH₂-GTEKGTSGWLKTGSC-CO₂H, (SEQ ID NO: 12) 20 nmol) was incubated with the SMCC-conjugated template strand 314 (2 nmol) in 200 μL of 0.1 M KHPO₄ buffer (pH 7.2) and the Gal 4 activation domain peptide 334 (NH2-EGEWTEGKLSLRGSC-CO₂H, (SEQ ID NO: 13) 20 nmol) was incubated with the SMCC-conjugated variable strand 316 (2 nmol) in 200 μL of 0.1 M KHPO₄ buffer (pH 7.2) for 3 h at room temperature, resulting in conjugation of the C-terminal cysteine of the polypeptides to the respective SMCC linkers 328, 332. Polypeptide-oligonucleotide conjugates were HPLC purified. The two polypeptide-oligonucleotide conjugates readily undergo hybridization by Watson-Crick base pairing.

The Gal 80-template strand conjugate 314 was cross-linked 338 to a thiol containing DNA oligonucleotide 318 (5′ (psoralen)TA GCC GGT GTG AAG TTT CTG CTA GTA ATG (thiol modifier C3) 3′) (SEQ ID NO: 16) which is partially reverse complementary to part of the 3′-terminal region of the template strand 314 and able to partially hybridize to the template strand (and was then crosslinked 338 to the template strand 314 for stability), with the 3′ end of the thiol containing oligo 318 extending single-stranded from the synbody construct and providing, via the thiol modifier 320, a conjugation site for maleimide-modified biotin 322, which in turn provides a site to which streptavidin 324 conjugated HRP 326 can be attached, enabling use of the construct in an ELISA-type assay. Inclusion of the third DNA strand 318 is optional. If the third DNA strand 318 is used, any attachment chemistry operable to attach any desired entity to the unhybridized portion of the strand may be used; by way of non-limiting example, any maleimide may be conjugated to the thiol modifier, and if maleimide-modified biotin is used, any streptavidin-linked entity may be applied to the biotin. Hybridization occurred with 40 μL of Gal 80-template conjugate (2 nmol) and 4.8 μL of the psoralen containing strand (4 nmol) in 20 μL crosslinking buffer (100 mM KCL, 1 mM spermidine, 200 mM Hepes pH 7.8, and 1 mM EDTA pH 8) at 90° C. for 5 min. then cooled on ice for 30 min. The sample was placed in one well of a 96 well flat bottom, clear NUNC plate and radiated with ultra violet light (366 nm) for 15 min. Unreacted crosslinking DNA was purified on streptavidin magnetic beads which contained the biotinylated complementary DNA strand. The flow-through was collected as the crosslinked Gal 80-template conjugate and hybridized with equal molar ratio of the Gal 4-variable strand by incubating in the presence of 1 M NaCl at 90° C. for 5 min. and then chilled on ice for 30 mM. The disulfide bond on the crosslinked DNA was reduced 30 min. before use by incubating with 10 mM TCEP (tris(2-carboxyethyl) phosphine hydrochloride) at room temperature for 30 min. The mercaptopropane was removed by using a microcon YM-10 molecular weight spin column (Millipore).

### Example 7 Synthesis of Synbody

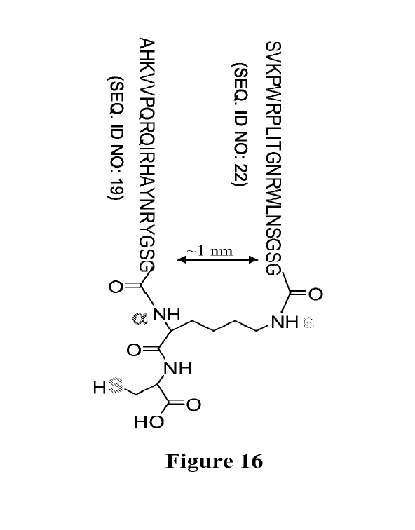

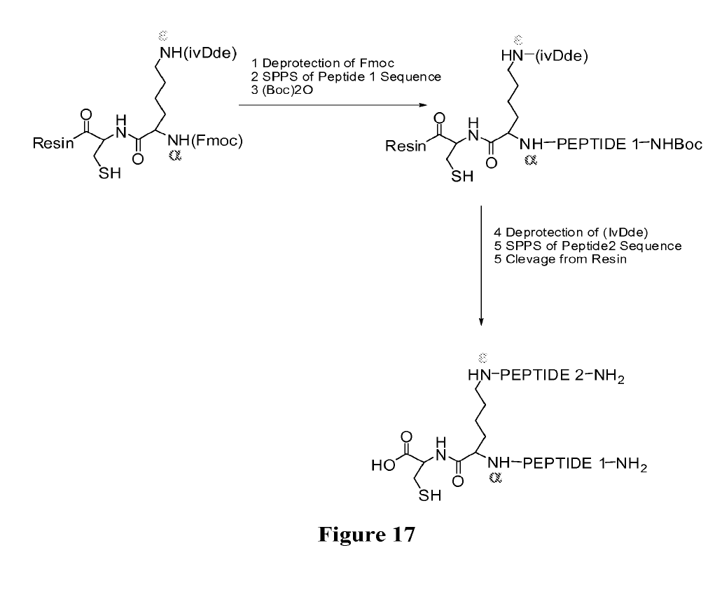

This example demonstrates the synthesis of the synbody shown in FIG. 16 using polypeptide affinity elements previously identified (sequences as shown in FIG. 16). As shown in FIG. 17, lysine, protected by an Fmoc protecting group at the a amine and by an ivDde protecting group at the c amine, was conjugated to a cysteine residue which was in turn attached to the resin support via an acid labile linkage. The Fmoc protecting group was removed, the first polypeptide affinity element was synthesized by sequential addition of residues by standard solid phase peptide synthesis techniques from the a amine of the lysine, and the terminal Fmoc protecting group was converted to Boc. The ivDde protecting group was then removed from the s amine of the lysine, and the second polypeptide affinity element was synthesized by sequential addition of residues to the exposed c amine of the lysine. The acid labile linkage of the cysteine residue to the resin was cleaved, freeing the completed synbody. The foregoing steps were performed in accordance with standard solid phase peptide synthesis techniques. See, e.g., Atherton E, Sheppard R C: Solid Phase peptide synthesis: a practical approach. Oxford, England: IRL Press; 1989, and Stewart J M, Young J D: Solid Phase Peptide Synthesis, 2d Ed. Rockford: Pierce Chemical Company; 1984, which are incorporated herein by reference. Any other technique operable for synthesizing and/or assembling the structure may be employed; by way of non-limiting example, either or both polypeptide affinity elements may be synthesized in place by sequential addition of residues using standard solid phase synthesis techniques, or by assembly of presynthesized substructures. The lysine linker provides a spacing of about 1 nm between the attachment points of the two polypeptides as shown in FIG. 16. The cysteine may be biotinylated to enable detection using fluorescently labeled streptavidin, or used for any other desired functionalization. Other C-terminal residues or structures may also be used; synbodies were also prepared having C-terminal glycine or alanine in lieu of cysteine.

The synbodies were purified on a C-18 semi-preparative column using 0.1% TFA in water and 90% CH₃CN in 0.1% TFA with gradient of 10 to 95% in 25 minutes, at flow rate of 4 ml/min and verified by MALDI-TOF.

### Example 8 SPR Analysis of DNA-Linked Synbody and Linker Distance/Orientation Optimization

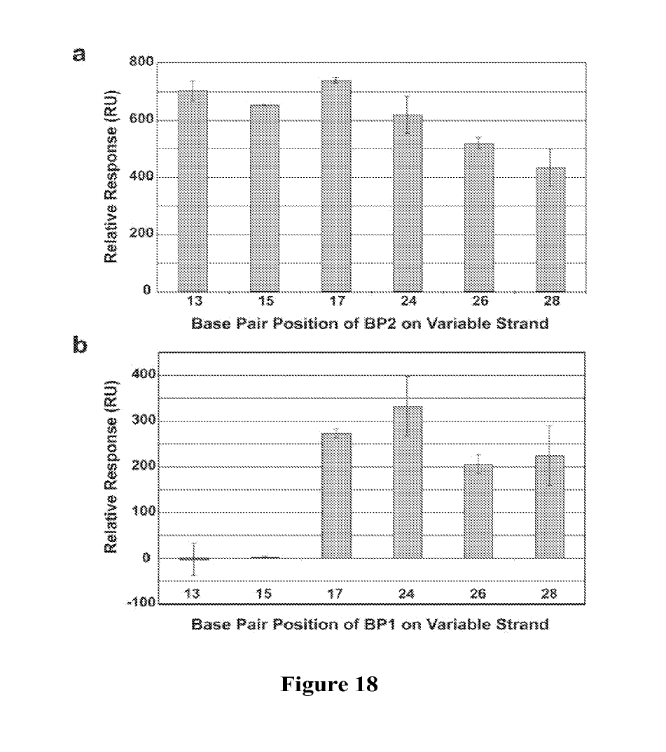

This example demonstrates the optimization of linker length for a DNA synbody, and demonstrates that the joinder of two affinity elements having moderate affinity for a target by an appropriate linker produces a synbody having affinity for the same target that is substantially improved over that of the individual affinity elements. DNA-linked synbody constructs (prepared as described in Example 6) were immobilized on a Flexchip, and gal80 in solution was flowed over the chip and response data obtained. 12 distinct synbody constructs were evaluated, each having the BP1 polypeptide as one affinity element and the BP2 polypeptide as the other affinity element. Six of the constructs had the BP1 polypeptide attached to the template strand and the BP2 polypeptide attached to the variable strand at each of six different positions (positions 13, 15, 17, 24, 26, and 28, counting from the 3′ end of the variable strand); the other six constructs were identical to the first six except that positions of the two polypeptides were reversed (i.e. the BP2 polypeptide was attached to the template strand and the BP1 polypeptide was attached to the variable strand). Relative SPR responses of these synbodies with respect to gal80 were determined and compared, with the results shown in FIG. 18. The configuration with BP1 on the template strand and BP2 on the variable strand produced a higher response than the reverse configuration, and affinity of the synbody for gal80 declined as the linker was elongated, indicating that a linker length corresponding to about 13 to 17 DNA bases, or about 5 nm, was optimal for this configuration. This corresponds well to the known dimensions of the gal80 homodimeric structure, which is approximately cylindrical, about 10 nm in length and about 5 nm in diameter.

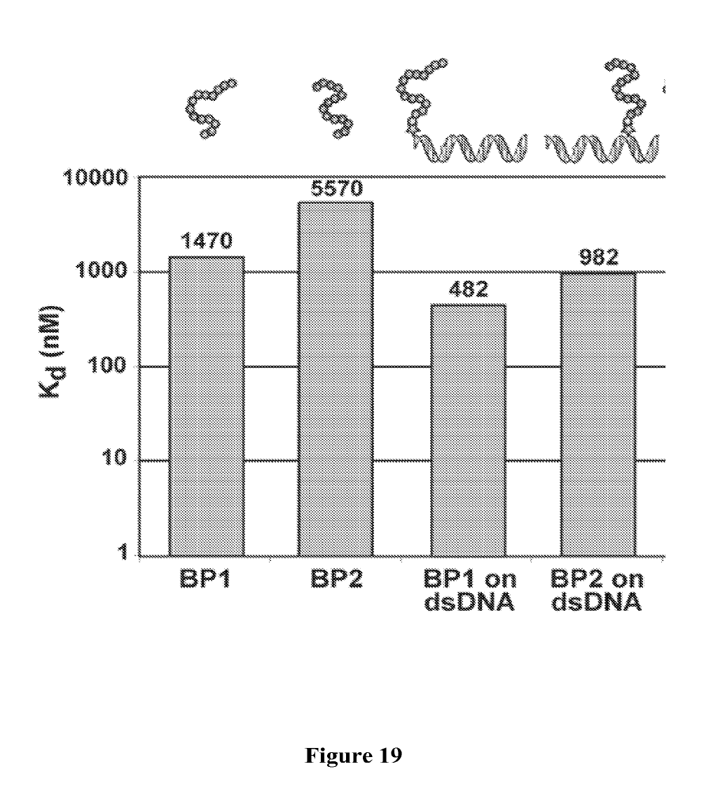

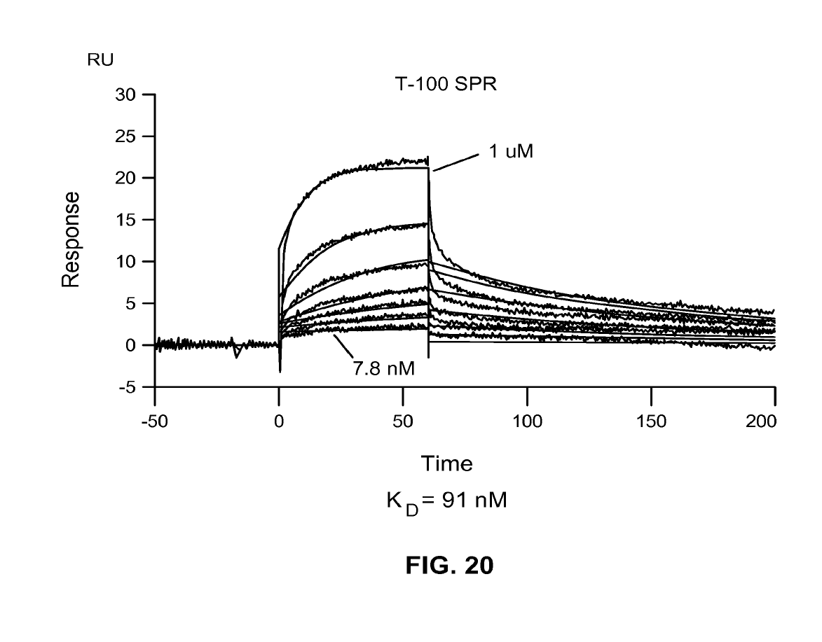

From on and off rates determined by SPR using the methods described in Example 4 with gal80 immobilized on the SPR chip, dissociation constants were obtained and compared for the linker-optimized synbody having the BP1 affinity element on the template strand and the BP2 affinity element at position 13 from the 3′ end of the variable strand, for each affinity element alone, and for each affinity element complexed by itself to the double-stranded DNA linker. As shown in FIG. 19, the affinity elements alone had affinities in a K_{d} range on the order of a few μM (K_{d}=1.5 for BP1 and Kd=5.6 for BP2). FIG. 20 shows the results of the SPR analysis of the binding of the BP1/BP2 DNA-linked synbody in solution, in a concentration series ranging from 1 μM to 7.81 nm, to surface-bound Gal80, indicating a K_{d} value of 91 nM. A gel shift assay was performed, again resulting in an estimated K_{d} value of about 100 nM.

These data were confirmed by ELISA-type analysis, where gal80 was immobilized in an ELISA well using standard methods, and the linker-optimized synbody, functionalized with streptavidin-conjugated HRP as described in Example 6, was applied in a concentration series and bound synbody detected in accordance with standard ELISA techniques. As shown in FIG. 20, the synbody was again found to have low nanomolar affinity for gal80, as compared to affinities in the K_{d} range of about 25 to 50 μM for each of the affinity elements individually with respect to gal80.

The specificity of the linker-optimized synbody was assessed by SPR determination of the affinity of the synbody for three protein targets other than gal80 (α1-antitrypsin, albumin, and transferrin). In each case the affinities were in a K_{d} range more than 1000 times greater than the K_{d} of the synbody for gal80.

### Example 9 SPR Analysis of Synbody

This example demonstrates that synbodies comprising affinity elements identified as described in Example 2 are capable of binding the target used for their identification (here, transferrin) with affinity that is significantly better than the affinity for the same target of either affinity element alone. Various synbodies comprising various pairings of affinity elements TRF-19 through TRF-26 (see Table 3) were synthesized in accordance with the methods described in Example 7 above, and their affinities for transferrin were evaluated by SPR with transferrin immobilized on the SPR chip in accordance with the methods described in Example 4 above, and with K_{d} values determined from kinetics. All of the pairings evaluated resulted in synbodies having K_{d} values less than the K_{d} values of their individual affinity elements alone (i.e., all were lower than about 50 μM). The synbody comprising TRF-26 and TRF-23 had K_{d} with respect to transferrin of 150±50 nm.

### Example 10

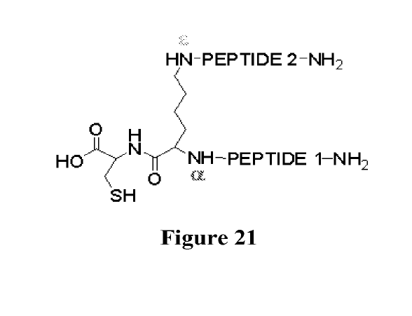

Synbodies were constructed by synthesizing two 20-mer polypeptides on the a and e amine moieties, respectively, of a lysine molecule as described in Example 7 above, thereby providing a spacing of about 1 nm as shown in FIG. 21. The thiol group of the cysteine is biotinylated to enable detection using fluorescently labeled streptavidin.

The polypeptide sequences used as binding elements in the synbodies were determined as described in Example 2. Several polypeptides corresponding to the loci at which transferrin bound were selected, synthesized (replacing the terminal cysteine with glycine to facilitate conjugation to the lysine linker for assembly of the synbody), and analyzed by SPR as described in Example 4 to identify pairs of polypeptides capable of simultaneously and non-competitively binding distinct loci on transferrin. Several such pairs were selected for incorporation into synbodies.

Two biotinylated anti-TRF synbodies (SYN23-26 and SYN 21-22) were applied to a protein microarray having 8,000 features (Invitrogen Protoarray Human Protein Microarray v. 4.0 for immune response biomarker profiling), each feature comprising a distinct human protein (GST fusion) adsorbed to a nitrocellulose coated slide. Application of the synbodies to the microarray was performed in accordance with manufacturer instructions: (see ProtoArray Human Protein Microarray, Invitrogen, Catalog no. PAH052401, Version B, 15 Dec. 2006, 25-0970, Users Manual.) After blocking the array with 1% BSA/PBS/0.1% Tween for 1 hour at 4 C with gentle shaking, 120 μl of probing buffer (1 xPBS, 5 mM mgC12, 0.5 mM DTT, 0.05% Triton X-100, 5% glycerol, 1% BSA) with synbody was applied to the array. The prescribed cover slip was placed over the array and adjusted to remove air bubbles. The array was incubated in a 50 ml conical tube, printed side up, for 1.5 hours at 4 C without shaking. The array was then removed from the conical tube inserted diagonally into the array chamber, kept on ice. 8 ml probing buffer was added to the chamber wall. The cover slip was removed and the array was incubated in probing buffer for 1 minute on ice. The probing buffer was decanted and drained. Two further washings were performed adding 8 ml probing buffer, incubating on ice for 1 minute, and decanting and draining. 5 nM fluorescently labeled streptavidin diluted in 6 ml probing buffer was incubated on the array for 30 minutes on ice in the dark, after which the solution was decanted and drained. Three wash steps were performed, each by adding 8 ml probing buffer, incubating for 1 minute on ice, decanting, and draining. The array was removed from the chamber, centrifuged at 800×g for 5 minutes at room temperature. The array was dried in the dark for 60 minutes at room temperature, after which it was scanned using a fluorescent microarray scanner and data was taken and analyzed.

The binding pattern data for SYN23-26 were compared with data obtained for a high quality anti-TRF monoclonal antibody, 1C10 (K_{d}=1.5 μm), on the same array. The sequences of the polypeptide binding elements of SYN21-22 were QYHHFMNLKRQGRAQAYGSG (SEQ ID NO: 17) and HAYKGPGDMRRFNHSGMGSG (SEQ ID NO: 18) and the sequences of SYN23-26 were FRGWAHIFFGPHVIYRGGSG (SEQ ID NO: 19) and AHKVVPQRQIRHAYNRYGSG (SEQ ID NO: 20).

Preferably SEQ ID NO: 20 is attached to the alpha nitrogen and SEQ ID NO: 19 to the epsilon nitrogen of lysine although the reverse orientation is also possible. Either SEQ ID NO: 19 or SEQ ID NO: 20 can be subject to optimization using the methods disclosed herein (e.g., linear optimization) or others. Preferably, no more than 1, 2, 3, 4, or five residue changes are made in either SEQ ID NO: 19 or SEQ ID NO: 20. A residue change can be a substitution of amino acid, deletion of amino acids, or internal addition of amino acids. Optionally, any substitutions are conservative substitutions, in which an amino acid of a given group is exchanged for another amino acid of the same group. Amino acids can be grouped as follow: Group I (hydrophobic sidechains): norleucine, met, ala, val, leu, ile; Group II (neutral hydrophilic side chains): cys, ser, thr; Group III (acidic side chains): asp, glu; Group IV (basic side chains): asn, gin, his, lys, arg; Group V (residues influencing chain orientation): gly, pro; and Group VI (aromatic side chains): trp, tyr, phe. Similarly amino acids can be derivatized and peptide bonds can be replaced with nonpeptide bonds as described in more detail above. Variants bind to human AKT1 preferably with similar or greater affinity than Syn23-26, in which SEQ ID NO: 20 is attached to the alpha nitrogen and SEQ ID NO:19 to the epsilon nitrogen of lysine. AKT1 (e.g., UniProtKBISwiss-Prot P31749 (AKT1_HUMAN)) is a well known serine-threonine kinase associated (usually by elevated expression) with many forms of cancer, including prostate, breast and ovarian (see e.g., Bellacosa et al., Adv Cancer Res. 2005; 94:29-86). Therefore, SYN23-26 and its variants are useful in detecting, prognosing, monitoring and treating cancers associated with abnormal AKT1 expression.

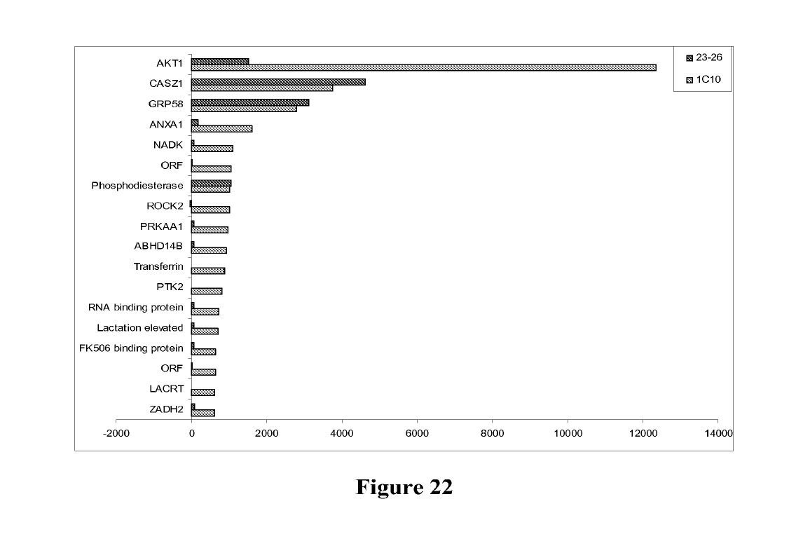

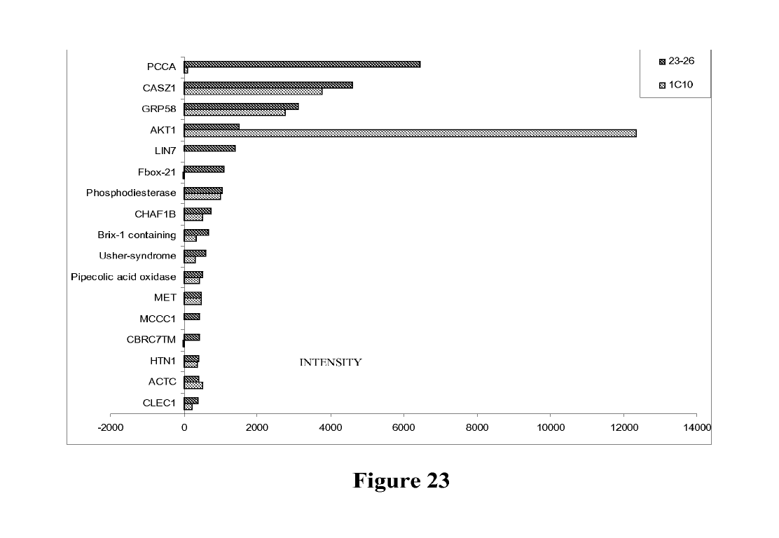

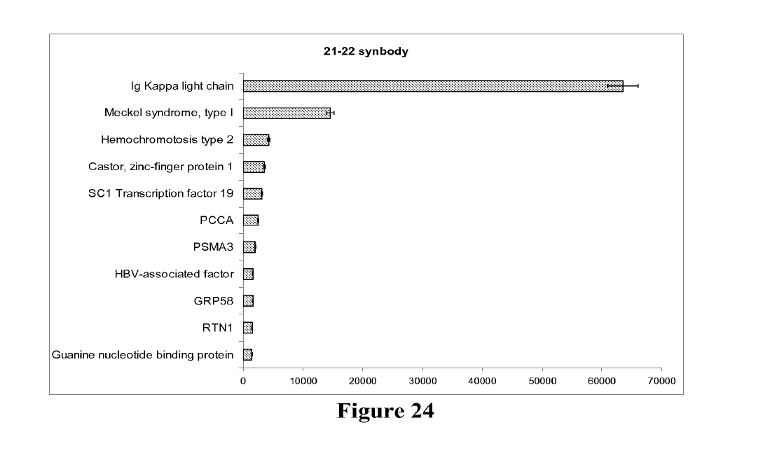

Comparisons of the measured fluorescence intensity values exceeding background (which are a measure of occupancy and, by extension, binding affinity) for SYN23-26 with those for the 1C10 antibody are shown in FIG. 22 for the 18 proteins to which 1C10 bound with highest intensity and in FIG. 23 for the 18 proteins to which SYN23-26 bound with highest intensity. Data for SYN21-22 are shown in FIG. 24. Binding of SYN23-26 to transferrin and AKT1 was evaluated by SPR, indicating estimated K_{d} values of about 1 nM with respect to AKT1 and about 141 nM with respect to transferrin.

As can be seen from the intensity plot for the highest affinity targets for the 1C10 anti-TRF antibody (FIG. 22, light bars), 1C10 bound ten other targets with intensity equal to or greater than that for TRF, and bound one target, AKT1, with more than ten-fold higher intensity. Similar results were obtained for SYN21-22 (FIG. 24).

The monoclonal antibody 1C10 and both synbody constructs exhibited high specificity, as indicated by high affinities for only a few targets, with the plot of affinities for all targets, ranked in descending order by affinity, appearing to decline rapidly and approximately exponentially. The highest affinities observed for the antibody and for both synbodies corresponded to targets other than transferrin. This data illustrates that bivalent synbodies (SYN23-26 and SYN21-22), each having binding elements chosen on the basis of their affinity for distinct sites on an arbitrarily chosen protein target (transferrin), each have, with respect to one target from a library of 8,000 (PCCA for SYN23-26 and Ig kappa light chain for SYN21-22), affinity and specificity characteristics essentially equivalent to those exhibited by the monoclonal antibody 1C10 for its highest affinity target (AKT1).

It is noteworthy that SYN23-26 bound to seven targets (FIG. 4, PCCA, CASZ1, GRP58, AKT1, LINT, Fbox-21, and Phosphodiesterase) with intensities higher than that exhibited by 1C10 for its nominal target (TRF), suggesting that SYN23-26 could be used as a synthetic antibody against any of these seven protein targets with quality equivalent to that of a high quality commercial monoclonal antibody.

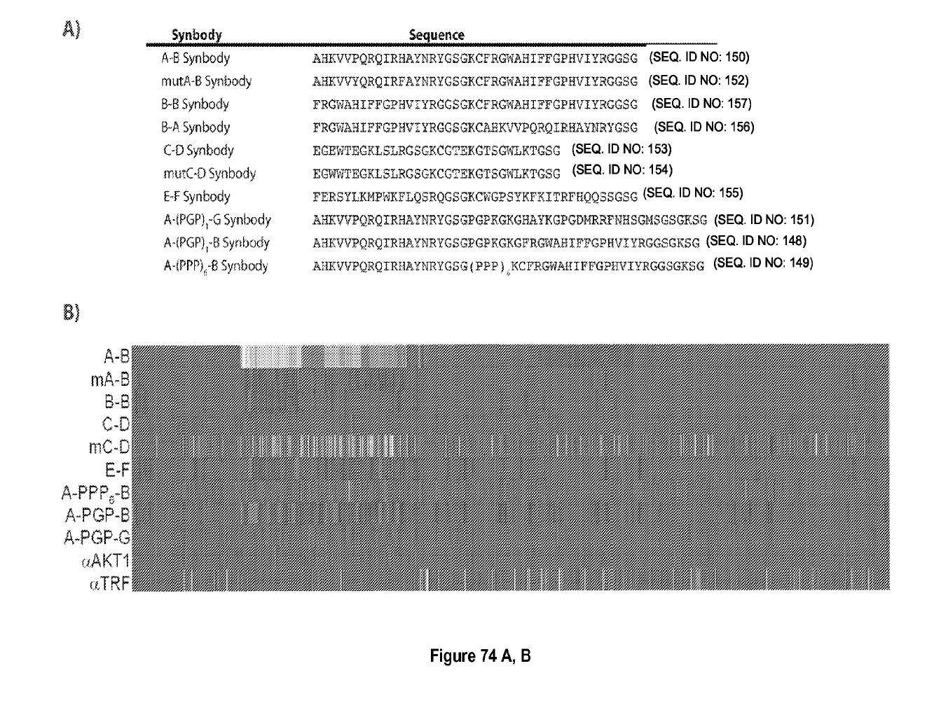

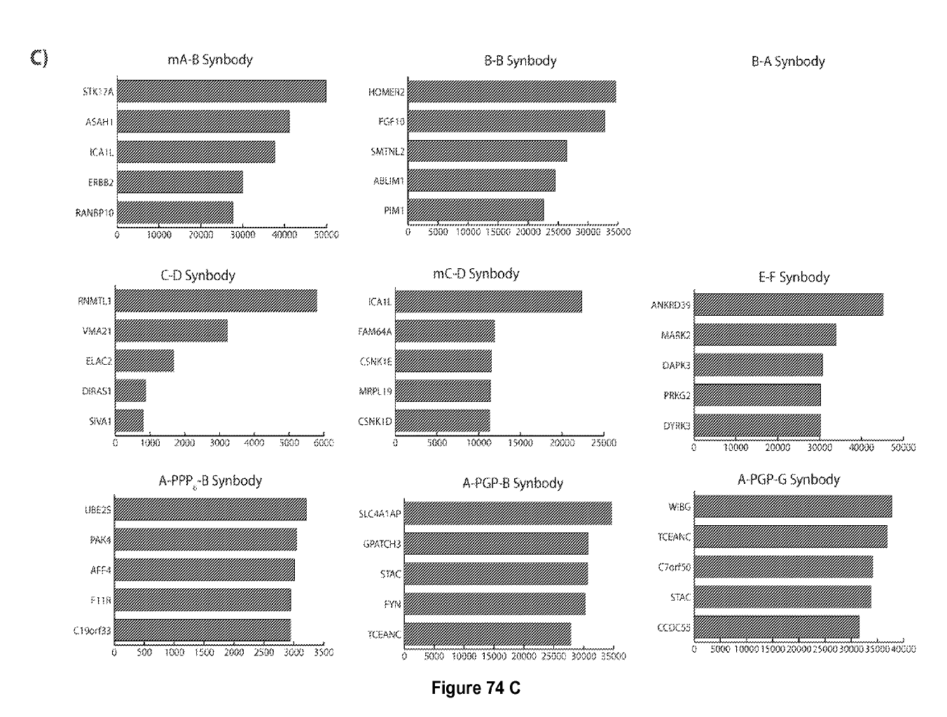

Nine additional Synbody constructs (FIG. 74A) were prepared and screened against the protein array under the same conditions as before. Each Synbody candidate produced a different binding profile (FIG. 74B). Analysis of the top five binding proteins for each Synbody showed that there was no overlap in the top binding proteins for each Synbody suggesting that each Synbody does indeed bind one or more unique proteins (FIG. 74C). The data also show that orientation of peptides and choice of linker can affect binding specificity.

### Example II

A bivalent synbody having binding elements selected for affinity for Gal80 was assembled and linked via a nucleic acid linker, providing spacing between binding elements of approximately 5 nm, as described in Example 6 above. Binding elements BP1 and BP2 were identified as described in Example 3 above.

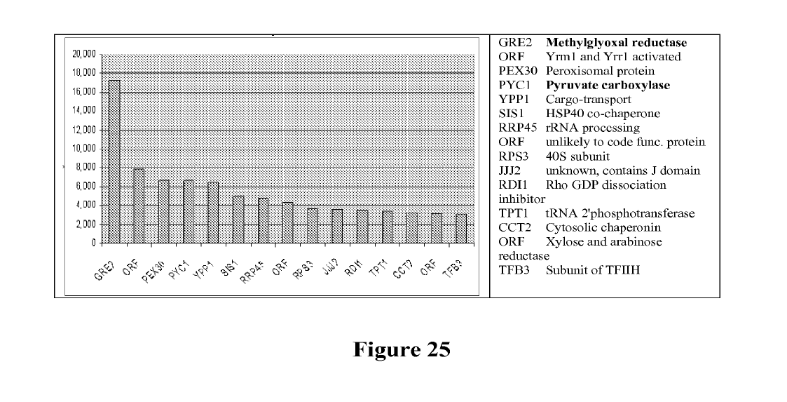

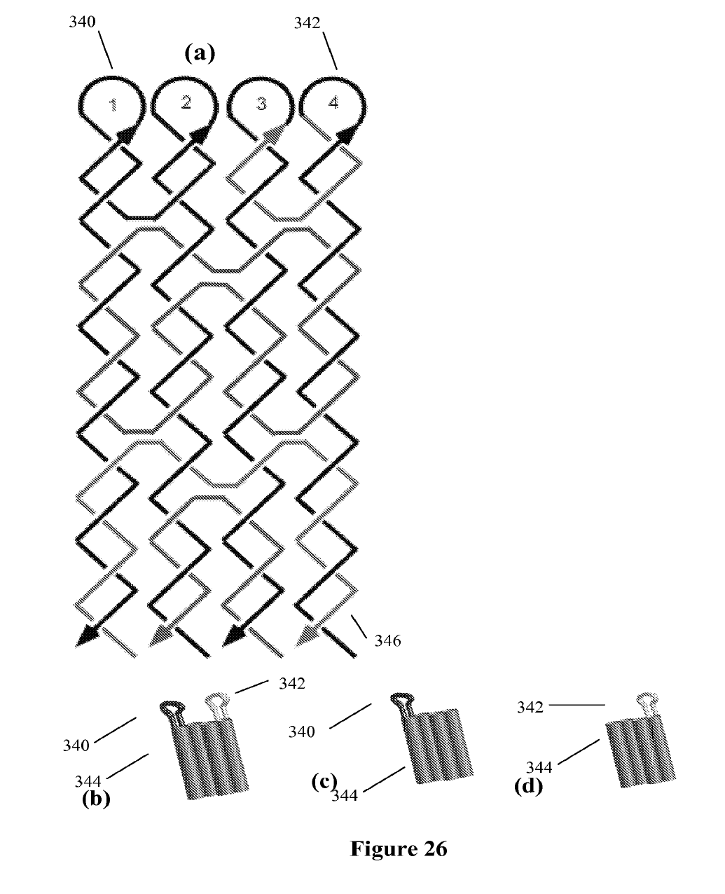

The (biotinylated) synbody was screened on an array of 4,000 yeast proteins (Invitrogen Protoarray Yeast Protein Microarray for immune response biomarker profiling), and detected using Alexa™ 555-labeled streptavidin. Fluorescence intensity data was obtained as shown in FIG. 25 (adjusted for background fluorescence). The distribution of affinities over the highest-binding protein targets was again comparable to that characteristic of a high quality monoclonal antibody, and, again, the protein targets for which the synbody exhibited the highest affinity did not include the target (Gal80) for which the binding elements were originally screened.

### Example 12 DNA Tile Synbody

This example demonstrates the assembly of a synbody having DNA aptamer affinity elements linked by a DNA tile linker, and demonstrates that the synbody so constructed has, with respect to the target used to identify the aptamer affinity elements, an affinity significantly greater than that of either of the aptamer affinity elements with respect to the same target. The 4-helix DNA tile linker was constructed from DNA oligonucleotides as shown schematically in FIG. 26 and described in detail in Ke Y G, Liu Y, Zhang J P, Yan H: A study of DNA tube formation mechanisms using 4-, 8-, and 12-helix DNA nanostructures. Journal of the American Chemical Society 2006, 128(13):4414-4421, which is incorporated by reference herein. The spacing between affinity elements is determined in part by the number of helices and the choice of loops in which to incorporate the aptamer affinity elements; the number of helices and choice of loops may be varied to achieve a desired spacing. The sequences of aptamers specific for thrombin shown in Table 4 were incorporated into the first 340 and fourth 342 single-stranded DNA loops, providing a structure in which the aptamers extend from the tile as shown schematically in FIG. 26(b), with a spacing between aptamers (for the 4-helix tile) of about 2 nm. For comparison and evaluation of binding properties of this two-aptamer synbody structure with similar structures having only a single affinity element, structures were also synthesized having only Apt1 in the first loop 340 without the presence of Apt2 (see FIG. 26(c)) and having only Apt2 in the fourth loop 342 without the presence of Apt1 (see FIG. 26(d)).

TABLE 4
Aptamer sequences used in DNA tile synbody

| | Sequence | Source |
|---|---|---|
| Apt1 | 5′-AGTCCGTGGTAGGGCAG | Tasset DM, Kubik MF, Steiner W: |
| | GTTGGGGTGACT-3 (SEQ ID NO: 21) | Oligonucleotide inhibitors of human |
| | | thrombin that bind distinct epitopes. |
| | | Journal of Molecular Biology 1997, |
| | | 272(5):688-698 |
| Apt2 | 5′-GGTTGGTGTGGTTGG-3′ (SEQ | Bock LC, Griffin LC, Latham JA, Vernnaas |
| | ID NO: 22) | EH, Toole JJ: Selection Of Single- |
| | | Stranded-DNA Molecules That Bind And |
| | | Inhibit Human Thrombin. Nature 1992, |
| | | 355(6360):564-566) |

By gel shift assay, binding of the DNA tile synbody (FIG. 26(b)) to thrombin was evaluated and compared with the binding to thrombin of each aptamer incorporated into its loop of the DNA tile without the other aptamer present (FIGS. 26(c) and (d)). Non-denaturing (8% polyacrylamide) gel electrophoresis was performed at 25° C. with constant 200V for 5 hours with 1 nM of pre-annealed Sybr-Gold stained tile/aptamer pre-incubated for 1 hr at room temperature with concentrations of human α-thrombin ranging from 0 to 100 nM. In the gel shift assay, the synbody was found to have a K_{d} with respect to thrombin of about 5 nM, the tile incorporating apt1 only or apt2 only had K_{d} values above 100 nM.

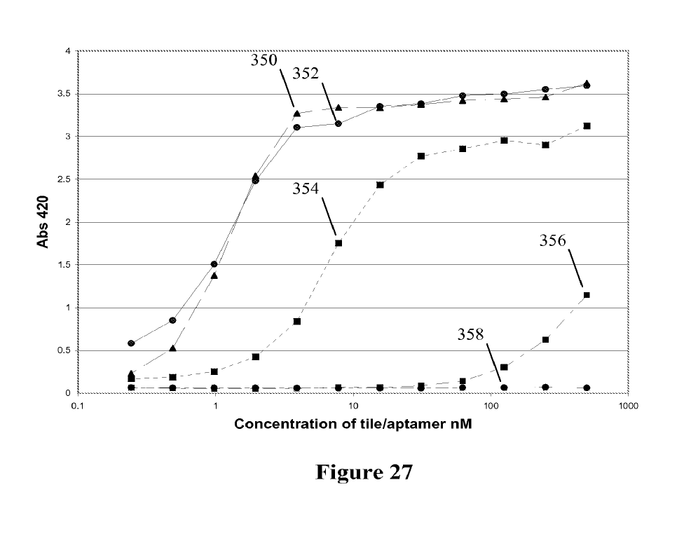

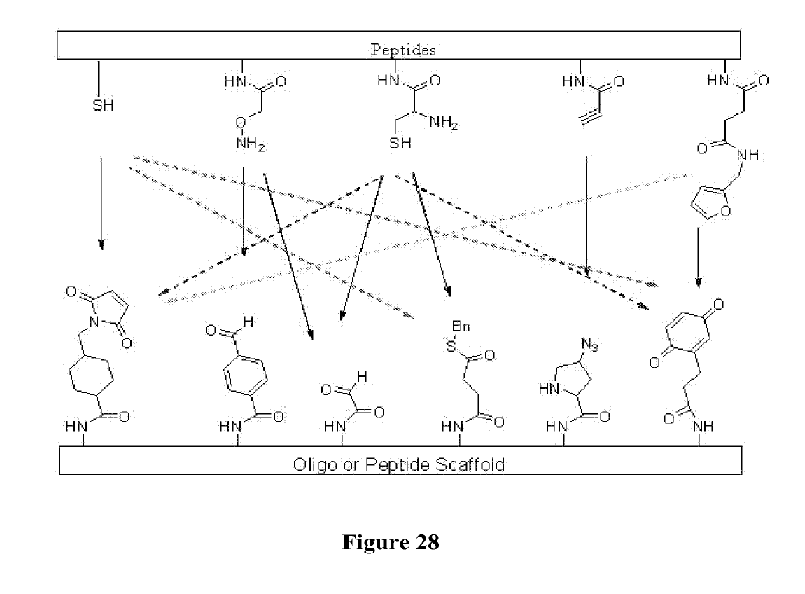

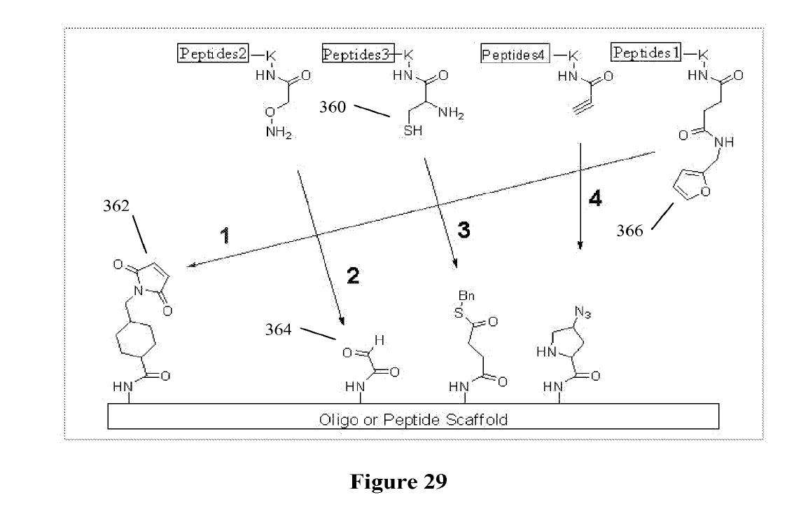

Binding to thrombin was evaluated in an ELISA-type assay. Wells of a 96 well plate were coated with 100 μL of 30 μg/mL human α-thrombin and incubated at 4C overnight. The plate was washed twice with DDI H₂O and passivated with 3% BSA in 1×PBS buffer for 1 hour. The plate was shaken out and 50 μL of varying concentrations of analyte (DNA tile synbody, DNA tile with each aptamer with the other not present, and each aptamer alone, respectively) were incubated at RT for 1 hour. DNA tiles were biotin-modified at the 5′ end of one of the distal DNA strands 346 (see FIG. 26(a)). The plate was rinsed 10 times in 1×PBS and 50 μL of 1:1000 dilution of streptavidin-HRP in 0.1% BSA in 1×PBS was pipetted and incubated for 1 hour at RT. The plate was again rinsed and 50 μL of TMB was added and incubated at RT for 15 minutes. 50 μL of 0.5M HCl was added and the plate was read immediately. Results are shown in FIG. 27 for the DNA tile synbody 350; the DNA tile with Apt1 but not Apt2 present 352; the DNA tile with Apt2 but not Apt1 present 356; Apt1 alone 354; and Apt2 alone 358. Dissociation constant values estimated from this assay were about 1 nM for the DNA tile synbody, about 10 nM for Apt1 alone, and more than 1 μM for Apt2 alone.

DNA tiles of other widths were also constructed and aptamer attachments at separation distances of about 2, 4, 6, and 8 nm were evaluated by non-denaturing gel shift assay (6% polyacrylamide). The 6 nm separation produced an approximately two-fold improvement of estimated K_{d} in comparison to the 2, 4, or 8 nm separation (K_{d} estimated about 2 nM for the 2 nm separation vs. about 1 nM for the 6 nm separation.

### Example 13 Linkers

The linker employed in the compositions and methods disclosed herein may be any structure, comprising one or more molecules, operable for associating two or more affinity elements together in a manner such that the resulting synbody has, with respect to a target of interest, affinity and/or specificity superior to that of the affinity elements when not so associated. In various embodiments, the linker may be a separate structure to which each of the two or more affinity elements is joined, and in other embodiments, the linker may be integral with one or both affinity elements. In some embodiments, it is desirable to choose linker structures that are stable and reasonably soluble in an aqueous environment, and amenable to efficient and specific chemistries for attaching affinity elements in a desired position and/or conformation.

Without limiting the generality of the foregoing, this prospective example demonstrates several linker compositions and chemistries for attaching affinity elements thereto, in addition to the DNA linkers and lysine linkers described in other examples.

Polyproline and variants thereof may be used as a linker in some embodiments. Polyproline forms a relatively rigid and stable helical structure with a three-fold symmetry, so that attachment sites spaced at three residue intervals are approximately aligned with respect to their angular relationship to the axial dimension. The distance between such attachment sites (three residues apart) is about 9.4 A for polyproline II, in which the peptide bonds are in trans conformation, and about 5.6 A for polyproline I, in which the peptide bonds are in cis conformation. Hydroxyproline may be substituted for proline in these constructs, to provide a more hydrophilic structure and improve solubility. See Schumacher M, Mizuno K, Chinger HPB: The Crystal Structure of the Collagen-like Polypeptide (Glycyl-4(R)-hydroxyprolyl-4(R)-hydroxyprolyl)9 at 1.55 Å Resolution Shows Up-puckering of the Proline Ring in the Xaa Position. Journal of Biological Chemistry 2005, 280(21):20397-20403, which is incorporated herein by reference.

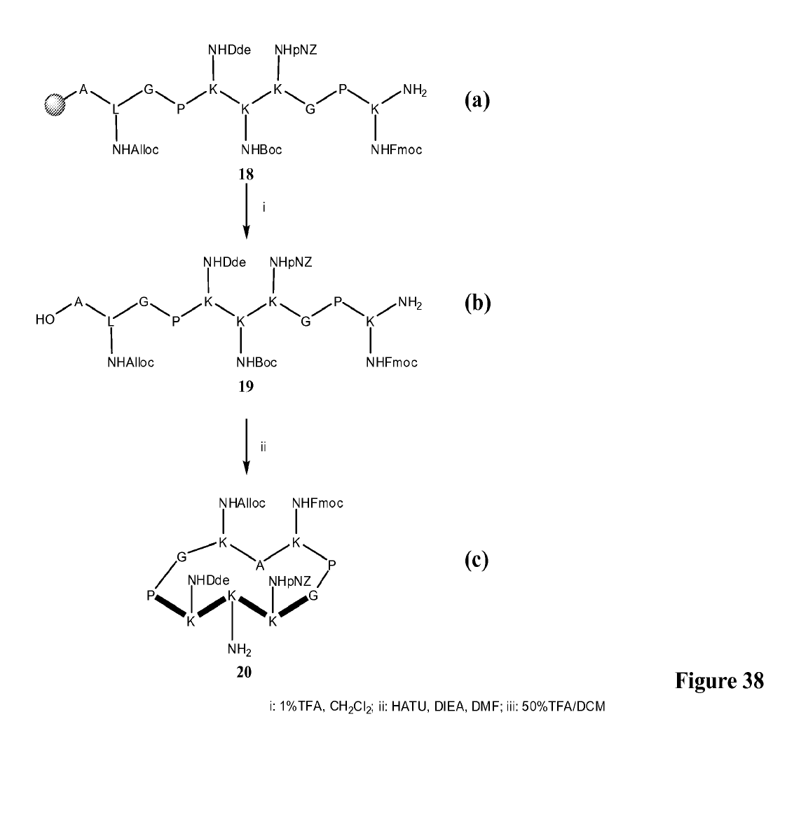

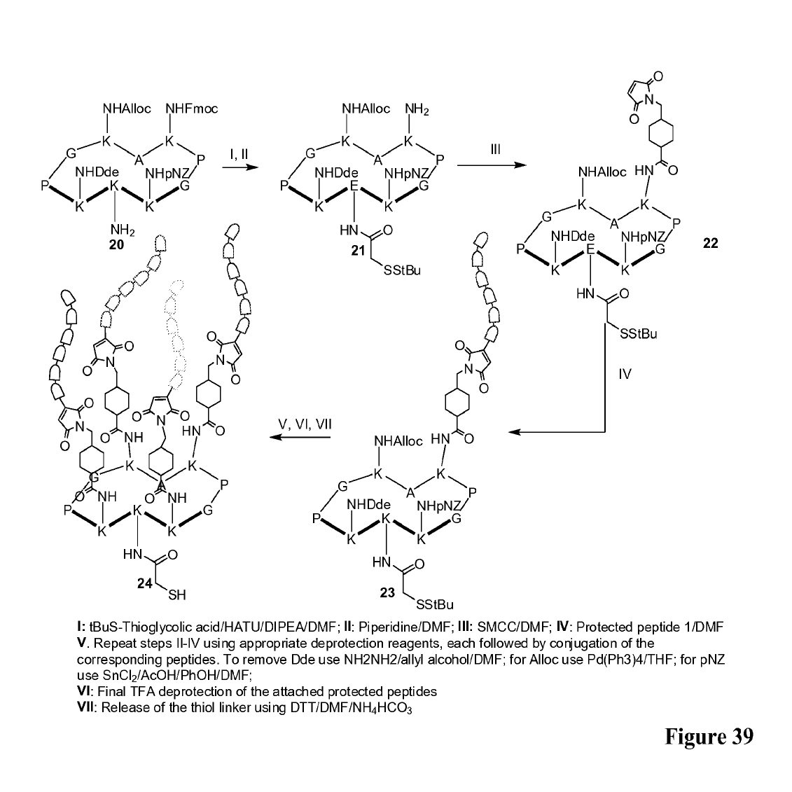

In general, synbodies comprising affinity elements and linkers that can be synthesized by standard solid phase synthesis techniques can be synthesized either by addition of amino acids or other monomers in a stepwise fashion, or by joining preassembled affinity elements and linkers or other presynthesized subunits. Techniques for stepwise synthesis of peptides and other heteropolymers are well known to persons of skill in the art. See, e.g., Atherton E, Sheppard R C: Solid Phase peptide synthesis: a practical approach. Oxford, England: IRL Press; 1989, and Stewart J M, Young J D: Solid Phase Peptide Synthesis, 2d Ed. Rockford: Pierce Chemical Company; 1984, which are incorporated herein by reference.

Where synbodies are constructed by joining presynthesized entities, it may be desirable to employ conjugation chemistries and methods that are orthogonal, so that conjugation points can be deprotected and added to without risking inadvertent deprotection or modification of other addition points, and that are rapid and high yield, so that adequate product is produced. FIG. 38 enumerates a number of conjugation pairs (pairs are denoted by the arrows in FIG. 38) each comprising a chemical moiety to be present on a peptide or other affinity element and another chemical moiety to be present on the oligonucleotide, peptide scaffold, or other linker, where the two members of the pair will react to form a covalent linkage under conditions that will be readily determinable by persons of ordinary skill in the art guided by the disclosures hereof. It will be seen that certain of the “click” moieties shown in FIG. 38 are capable of conjugating with more than one other moiety; where such moieties are employed, it may be necessary to perform the desired conjugations in an appropriate order so that the desired conjugation takes place at any moieties that are susceptible to reaction with more than one other moiety before such other moieties are applied. FIG. 39 shows an illustrative example in which four orthogonal conjugations are achieved performing four “click” reactions, which should preferably be performed in the order shown (for example, the thiol moiety 360 is intended to react with the aldehyde moiety 364, but can also react with the maleimide moiety 362; this is prevented by reacting the maleimide 362 with its intended click pair 366 first, so that when the thiol 360 is applied no maleimide 362 remains to react with it. The use of “click” chemistry to perform conjugations between biopolymers and other heteropolymers is described in detail in various references such as Kolb H C, Finn M G, Sharpless K B: Click chemistry: Diverse chemical function from a few good reactions. Angewandte Chemie-Intemational Edition 2001, 40(11):2004 and Evans R A: The rise of azide-alkyne 1,3-dipolar ‘click’ cycloaddition and its application to polymer science and surface modification. Australian Journal of Chemistry 2007, 60(6):384-395, which are incorporated herein by reference.

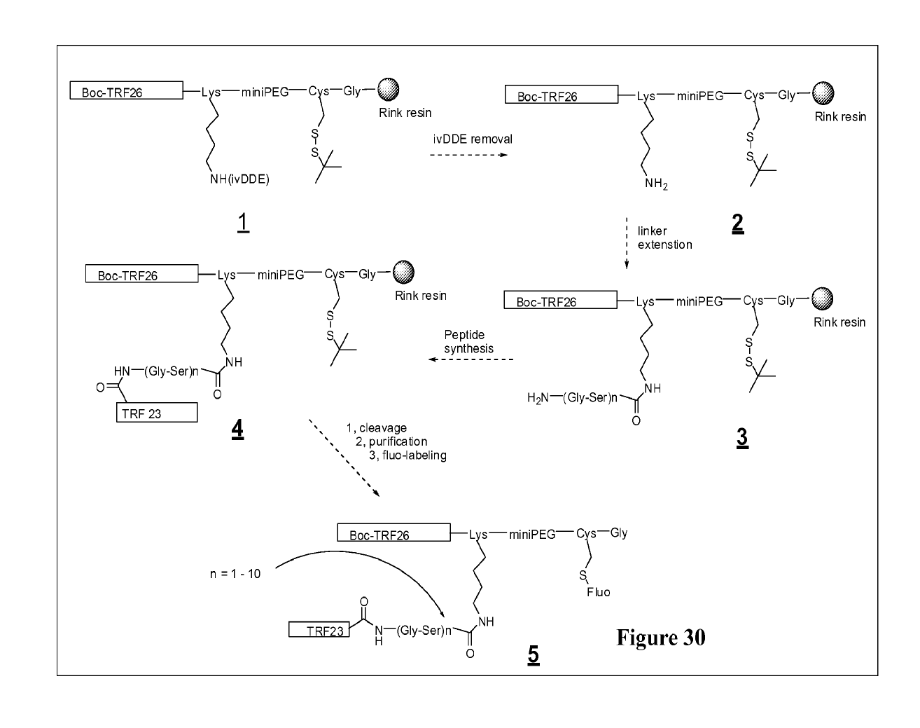

FIG. 30 shows the synthesis of a synbody comprising two peptide affinity elements (TRF26 and TRF23) joined by a poly Gly-Ser linker and further comprising a cysteine, attached via a miniPEG, for labeling with a suitable fluorescent label. The entity shown in FIG. 30(1) is first synthesized in large quantity (i.e. 0.5 to 1.0 mmole) in a microwave synthesizer by standard methods. The ivDDE protecting group is then removed and the deprotected product is split into ten aliquots. Again by microwave synthesis, to each aliquot is added a predetermined number of Gly-Ser, ranging from 1 to 10, so that each aliquot now has a linker comprising (Gly-Ser)n where n is 1 for the first aliquot, 2 for the second, and so on up to 10 (FIG. 30(3)). For each aliquot, the second peptide affinity element, TRF23, is then synthesized by stepwise addition of amino acids (FIG. 30(4)). The synbody is then cleaved from the resin. The t-butyl thiol protecting group intact on the miniPEG-linked cysteine may be removed and a fluorescent label added if desired (FIG. 30(5)).

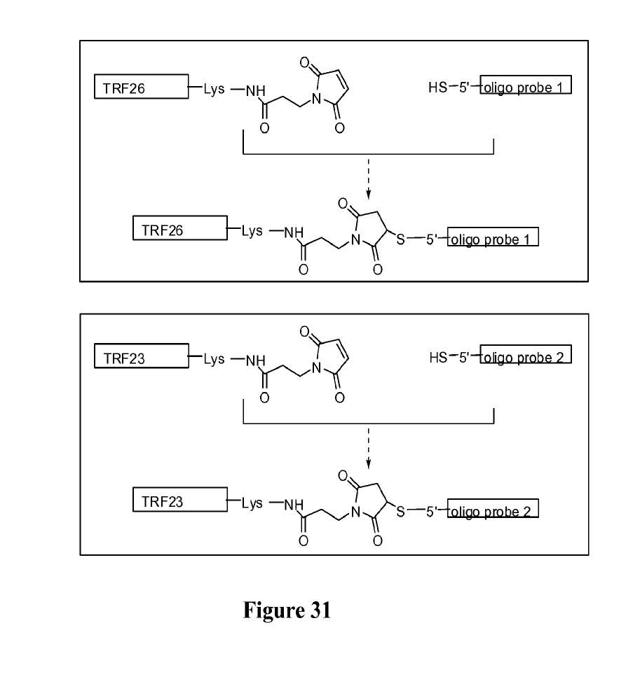

FIG. 31 shows the conjugation of a maleimide-functionalized peptide to a thiol-modified oligonucleotide, producing a peptide-oligonucleotide conjugate that may be used to enable the use of peptide affinity elements with the DNA tile linkers of Example 9 above. The oligonucleotide conjugated to the peptide is reverse complementary to an exposed DNA strand of the DNA tile and stably hybridizes thereto.

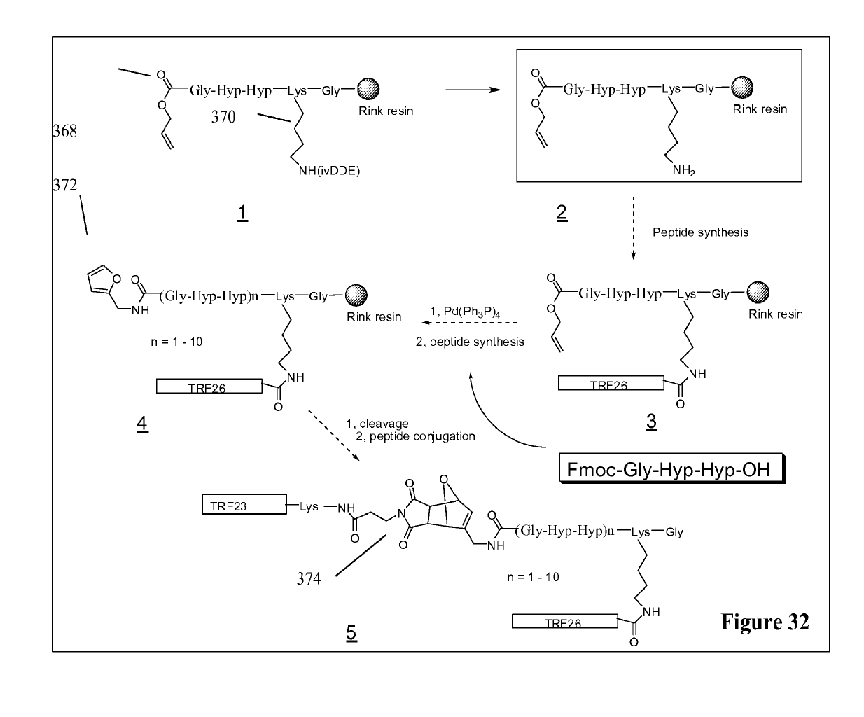

FIG. 32 shows the synthesis of a poly-(Gly-Hyp-Hyp)-linked synbody and illustrates a method for improving the ivDDE deprotection (ivDDE deprotection in the presence of a long peptide may be suboptimal due to interference by the peptides with access to an ivDDE that is close to the resin surface). The structure shown in FIG. 32(1) is first synthesized using standard solid phase synthesis techniques. The ivDDE 370 protected lysine is deprotected (FIG. 32(2)) and the first peptide affinity element TFR26 is synthesized by stepwise addition of amino acids (FIG. 32(3)). The alloc protecting group 368 is removed and Fmoc-Gly-Hyp-Hyp-OH subunits are added to the linker to the length desired (FIG. 32(4)). The structure is then cleaved from the resin, and TRF23, which has been presynthesized with a maleimide functionalization 374 of the terminal lysine, is conjugated to the furanyl moiety 372 of the poly-(Gly-Hyp-Hyp) linker (FIG. 32(5)).

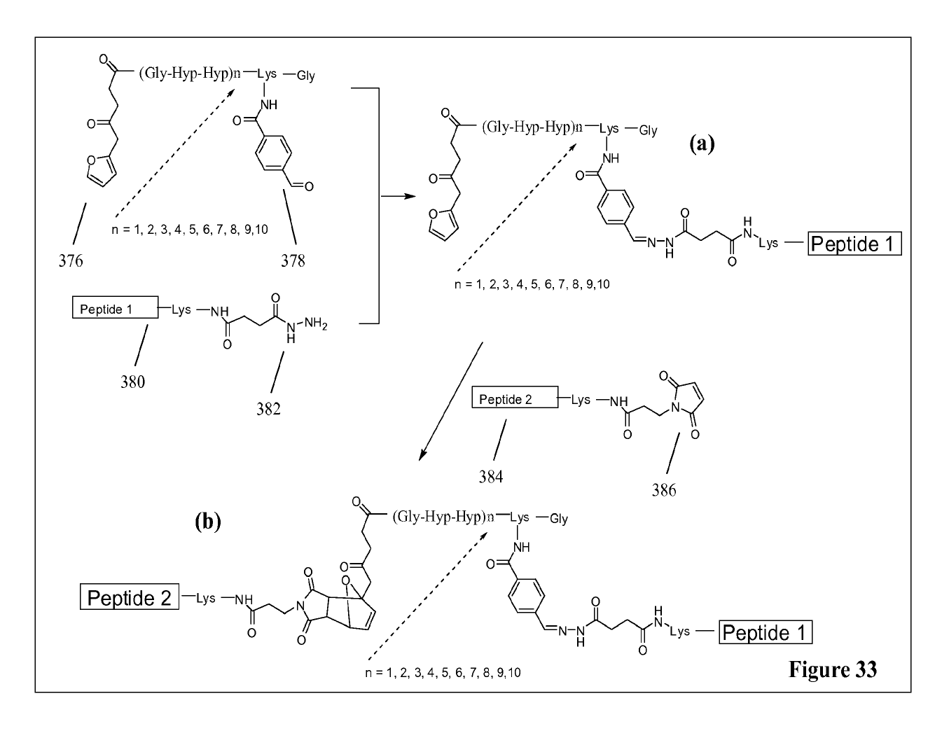

FIG. 33 shows the synthesis of synbodies using poly-(Gly-Hyp-Hyp) linkers of varying lengths by attaching both affinity elements using mutually orthogonal conjugations. (Gly-Hyp-Hyp)n linkers of varying lengths from n=1 to n=10 are presynthesized with a furanyl moiety 376 for conjugation of a first affinity element and a benzaldehyde moiety 378 for conjugation of a second affinity element. The first affinity element 380, functionalized with a hydrazide moiety, is conjugated to the benzaldehyde moiety of the poly-(Gly-Hyp-Hyp) linker (FIG. 33(a)). The second affinity element 384, functionalized with a maleimide moiety 386, is conjugated to the furanyl moiety of the linker (FIG. 33(b)). These conjugations can be performed in a reaction mixture containing multiple different linker lengths and/or multiple peptide sequences, enabling production of a combinatorial library representing multiple linker lengths and affinity element combinations, from which constructs that optimally bind the target of interest are identified using an affinity column or other suitable screening method.

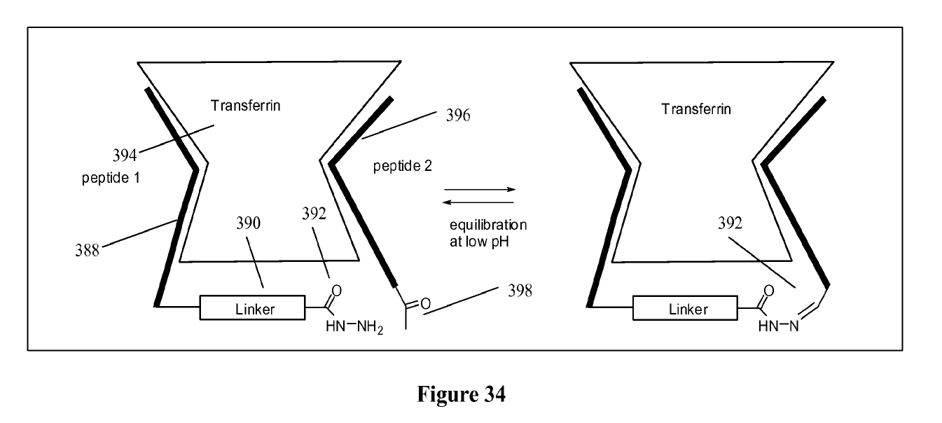

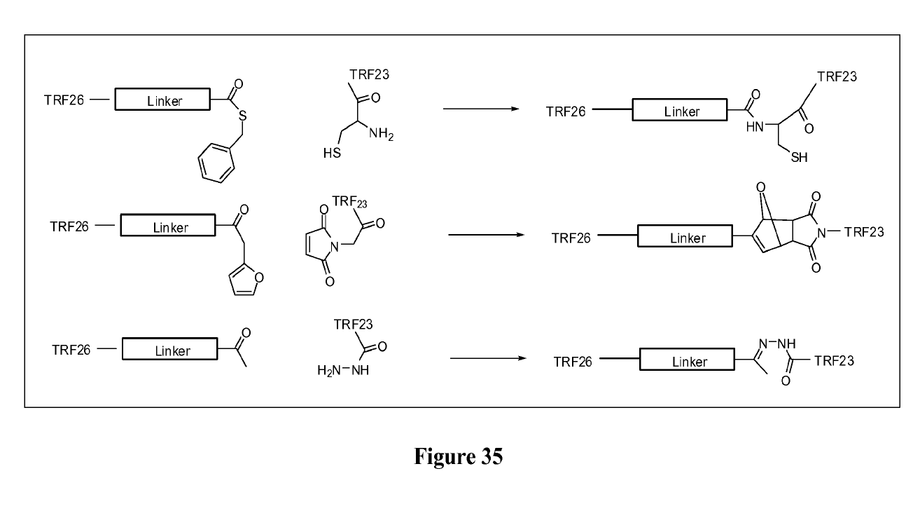

FIG. 34 illustrates schematically a method for determining suitable linker lengths and affinity element sequences by allowing the desired synbody structures to self-assemble in the presence of the target of interest 394 such as transferrin. To a solution containing transferrin 394 are added a first library combining a variety of distinct affinity elements 388 (shown as peptide 1 in FIG. 34) with linkers 390 of a variety of lengths to which the affinity elements are conjugated, each linker 390 being functionalized (at its terminus opposite the attachment point of the affinity element, or other attachment point providing a desired separation and/or orientation) with a moiety 392 suitable for conjugation of a second affinity element 396. A second library comprising a variety of distinct affinity elements 396 (peptide 2 in FIG. 34), each functionalized with a moiety 398 suitable for conjugation with the linker, is added. Affinity elements 388, 396 having affinity for loci on the target 394 will tend to associate with the target in their preferred positions and/or orientations. Where a pair comprising an affinity element 388 plus linker 390 and an affinity element 396 plus conjugation moiety 398 associate with a target molecule in such a way that the conjugation moiety 398 of the affinity element 396 and the conjugation moiety 392 of the linker are in close proximity and appropriately oriented, reaction will occur and a bond 392 will form, linking the two affinity elements into a synbody, whose position and orientation with respect to the target has been determined by the target itself. Synbodies bound to the target are then identified and characterized. The concentrations of affinity elements used should preferably be low enough to prevent significant conjugation between affinity elements and linkers that are not associated with a target molecule, but should be high enough so that affinity elements will associate with target for sufficient time to allow the desired pairs to conjugate. Also, the conjugation chemistry should be reversible so as to allow the conjugation process reach an equilibrium that favors the most suitable combinations; several conjugation chemistries that are potentially reversible under appropriate conditions are shown in FIG. 35. (Many other reversible conjugation chemistries are possible; in any, obtaining the desired reversibility will depend upon suitable reaction conditions.)

### Example 14 Cyclic Tetrapeptide Linker Synbody

This example demonstrates the synthesis of a cyclic tetrapeptide having three orthogonally protected conjugation sites for attachment of peptide or other affinity elements.

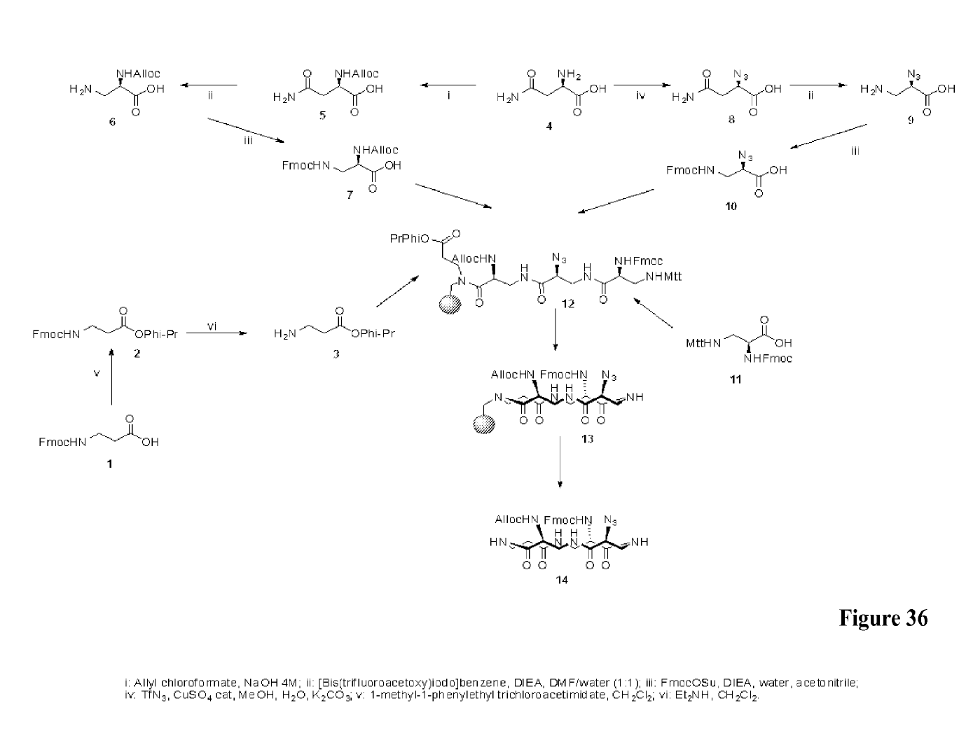

The structure shown in FIG. 36 is synthesized from three modified amino acids, and a fourth one that is commercially available, as shown. The three amino acids are first synthesized, and the resin modified; the synthesis of the tetrapeptide is then carried out, and peptides or other affinity elements are added; thus, the tetrapeptide serves as a linker for construction of a synbody.

Synthesis of the modified amino acids. 1-Methyl-1-phenylethyl 3-aminopropanoate (FIG. 36(3)) was synthesized as follows: Over a suspension of NaH (50 mg, 2.1 mmol) in diethyl ether (2 mL), a solution of 2-phenyl-2-propanol (2.5 g, 18.36 mmol) in 2 mL of diethyl ether was added dropwise. The mixture was stirred at room temperature for 20 min and then cooled at 0° C. Trichloroacetonitrile (1.9 mL) was slowly added (for 15 min) and the mixture was allowed to reach room temperature. After 1 hour of stirring, the mixture was concentrated to dryness and the resultant oil was dissolved in pentane (2 mL) and the solution was filtered. The filtrate was evaporated to dryness, to get a very dark oil that we use immediately in the next reaction. The freshly prepared 1-methyl-1,1-phenylethyl trichloroacetimidate (2.7 g, 6.424 mmol) was added over a solution of Fmoc-β-alanine, (FIG. 36(1)), (1 g, 3.212 mmol) in DCM (8 mL). After overnight stirring, the precipitated trichloroacetamide was removed by filtration, and the filtrate mixture was evaporated to dryness and purified by flash chromatography CH₂Cl₂/MeOH (0% to 1%) to yield 1.158 g (84%) of compound 2 as a colorless oil.

In a flask, (FIG. 36(2)) (1.158 g, 2.698 mmol) was dissolved in DCM (4 mL), and diethylamine (12 mL) was added. Immediately, the mixture becomes clear. The mixture was stirred for 2 hours. After adding 20 mL of toluene, the mixture was concentrated to dryness and the separation carried out by flash chromatography, using 10% of CH₂Cl₂/MeOH and 2% of Et₃N to yield 526 mg (94%) of (FIG. 36(3)) as a colorless oil.

N²-(allyloxycarbonyl)-N³-(9-fluorenylmethoxycarbonyl)-2,3-diaminopropanoic acid (7) was synthesized as follows: Over a solution of 2 g of asparagine (FIG. 36(4), 15.138 mmol) in 3.78 mL of 4M NaOH solution cooled in an ice-bath, 1.615 mL of allyl chloroformate (15.138 mmol) and 3.78 mL of 4M NaOH solution in portions were added. The reaction was kept alkaline and stirred for 15 minutes at room temperature. The mixture was extracted with ether and acidified with concentrated HCl, so the product was crystallized, filtrated, and lyophilized to afford (FIG. 36(5)) (2.816 g, 86%) as a white solid. [Bis(trifluoroacetoxy)iodo]benzene (8.402 g, 19.539 mmol) was added to a mixture of (FIG. 36(5)) (2.816 g, 13.026 mmol) and aqueous DMF (140 mL, 1:1, v/v). The mixture was stirred for 15 min, and DIEA (4.54 mL, 26.052 mmol) was added. After 8 hours the reaction, only half of the reaction went. So, the same quantities of [Bis(trifluoroacetoxy)iodo]benzene and DIEA were added, and the reaction was stirred overnight. The next day, the solution was concentrated to dryness, the residue solved in 100 mL of water and the organic side products were removed by repeated washings with diethyl ether (4×100 mL). The water phase was evaporated to dryness to yield product (FIG. 36(6)) that was used in the next reaction without further purification.

The oil previously obtained ((FIG. 36(6)) was redissolved in water (20 mL), and DIEA (2.24 mL, 13.026 mmol) and FmocOSu (4.393 g, 13.026 mmol) in acetonitrile (15 mL) were added, and the reaction was allowed to stir for 1.5 h. The mixture was acidified (to pH 2.0) by addition of HCl, and the product was extracted in DCM (5×40 mL). The organic phases were combined, dried with Na₂SO₄, and evaporated to dryness. The crude product mixture was purified by flash chromatography (10% MeOH in DCM). Hexane was added to the combined product fractions, and the precipitate formed was filtered and washed with hexane, and dried to yield a white solid (FIG. 36(7)).

2-azido-3-[(9-fluorenylmethyloxycarbonyl)amino]-propanoic acid (10) was synthesized as follows: A solution of NaN₃ (9.841 g, 151.38 mmol) in 25 mL of H₂O was cooled in an ice bath and treated with 50 mL of CH₂Cl₂. The biphasic mixture was stirred vigorously and treated with Tf₂O (8.542 g, 282.14 mmol) for over a period of 30 min. The reaction mixture was stirred at ice bath temperature for 2 h. After quenching with aqueous NaHCO₃, the layers were separated, and the aqueous layer was extracted twice with CH₂Cl₂ (2×50 mL). The organic layers were combined to afford 100 mL of TfN₃ solution that was washed once with Na₂CO₃ and used in the next reaction without further purification.

To a solution of L-asparagine (FIG. 36(4)) (2 g, 15.138 mmol) in 50 mL of H₂O and 100 mL of MeOH were added: K₂CO₃ (3.138 g, 22.707 mmol), CuSO₄ (38 mg, 0.151 mmol), and the solution of TfN₃ in CH₂Cl₂ previously prepared. The reaction was stirred at room temperature overnight. Then, solid NaHCO₃ (10 g) was added carefully, and the organic solvents evaporated. Concentrated HCl was added to the aqueous solution to obtain pH=6, and 100 mL of 0.25 M PBS was added. Then, ethyl acetate (3×150 mL) was used to do extractions. Next, more concentrated HCl was used to reach pH=2 and new extractions were carried out with ethyl acetate (5×150 mL) and the extract concentrated to dryness to afford a yellow oil (FIG. 36(8)), that was used in the next reaction without further purification.

[Bis(trifluoroacetoxy)iodo]benzene (19.529 g, 45.414 mmol) was added to a mixture of the crude (FIG. 36(8)) (15.138 mmol) and aqueous DMF (120 mL, 1:1, v/v). The mixture was stirred for 15 min, and DIEA (10.546 mL, 60.552 mmol) was added. The reaction continued overnight. The next day, the solution was concentrated to dryness, the residue dissolved in 100 mL of water and the organic products were removed by repeated washings with diethyl ether (3×100 mL). The water phase was evaporated to dryness to yield product (FIG. 36(9)) as a pale oil that was used in the next reaction without further purification.

The oil previously obtained (FIG. 36(9)) was redissolved in water (20 mL), and DIEA (2.6 mL, 15.138 mmol) and FmocOSu (5.106 g, 15.138 mmol) in acetonitrile (15 mL) were added, and the reaction was allowed to stir for 1.5 h. The mixture was acidified (to pH 2.0) by addition of HCl, and the product was extracted in DCM (5×40 mL). The organic phases were combined, dried with Na₂SO₄, and evaporated to dryness. The crude product mixture was purified by flash chromatography (10% MeOH in DCM). Hexane was added to the combined product fractions, and the precipitate formed was filtered and washed with hexane, and dried to yield a white solid (FIG. 36(10)).

Derivatization of the resin. Mixture of Boc- and Fmoc-1-alanine (2.0 eq of both, 4.0 equiv of TBTU, 8 equiv of DIEA in DMG, 1 h at 25° C.) was coupled to aminomethyl polystyrene resin (1.0 g, 0.5 mmol/g). 50% TFA in DCM was used to remove the Boc groups, and the exposed amino groups were capped with acetanhydride treatment. Thus, the loading of the resin was reduced to 0.16 mmol/g. A treatment of 20% piperidine in DMF was used to remove the Fmoc groups, and 4-(4-formyl-3,5-dimethoxyphenoxy)butyric acid was attached by HATU-promoted coupling to obtain the derivatized resin.

Synthesis of the scaffold on the resin. Previously derivatized resin (1.0 g, a loading of 0.16 mmol/g) was treated for 1 h at room temperature with a mixture of 1-methyl-1-phenylethyl 3-aminopropanoate (FIG. 36(3), 160 mg, 4 equiv) and NaCNBH₃ (48 mg, 4 equiv) in DMF, containing 1% (v/v) AcOH (16 mL). The resin was washed with DMF, DCM, and MeOH and dried on a filter.

The secondary amine was acylated with Aloc-Dpr(Fmoc)-OH 7 (5.0 equiv), using 5 equiv of PyAOP and 10 equiv of DIEA in DMF-DCM, 1:9, v/v for 2 h at 25° C. The Fmoc group was removed by treatment of piperidine-DMF, 1:4, v/v, for 20 min at 25° C. Couplings of 2-azido-3-[(9-fluorenylmethyloxycarbonyl)amino]propanoic acid (FIG. 36(10)) and Fmoc-Dpr-(Mtt)-OH (11) were carried out in each case, by treatment with 5 equiv of the amino acid, 5 equiv of HATU and 10 equiv of collidine in DMF for 1 h at 25° C. to afford product (FIG. 36(12)). The removal of Mtt and PhiPr protections was carried out by treatment with a solution of TFA in DCM (1:99, v/v, for 6 min at 25° C.), followed by immediate neutralization by washings with a mixture of Py in DCM (1:5, v/v).

Cyclization of the peptide (FIG. 36(13)) was then performed using PyAOP as an activator (5 equiv of PyAOP, 5 equiv of DIEA in DMF for 2 h at 25° C.). After each coupling (including the cyclization step), potentially remaining free amino groups were capped by an acetic anhydride treatment.

Then, the resin was treated with TFA in DCM (1:1, v/v, 30 min at 25° C.) to release the final product (FIG. 36(14)).

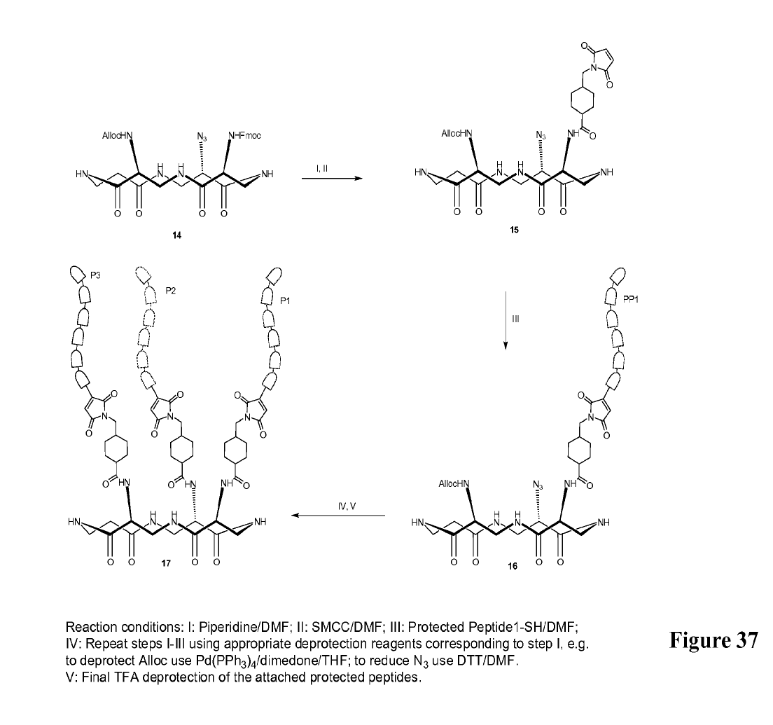

Sequential addition of peptides to the scaffold. The three amino acid residues can be sequentially deprotected, reacted with sulfosuccinimidyl-4-(N-maleimidomethyl)cyclohexane-1-carboxylate (Sulfo-SMCC) or other heterobifunctional linker, and the corresponding peptide added. Thus, this scaffold allows incorporation of up to three same or different peptides as shown in FIG. 37. Peptides are chosen based on screening of target on a random peptide microarray as described in preceding examples.

### Example 15 Cyclic Decapeptide Linker Synbody

This example demonstrates the synthesis of a cyclic decapeptide scaffold from commercial Fmoc amino acids by solid phase synthesis, using Trt-Lys(Fmoc)OH as the N-terminal amino acid, and SASRIN resin as shown in FIG. 38. The cyclization of the decapeptide is carried out in high dilution. This decapeptide structure provides orthogonally protected conjugation sites enabling attachment of up to four distinct peptides or other affinity elements, and thus serves as a linker for the synbody.

Synthesis of the Decapeptide

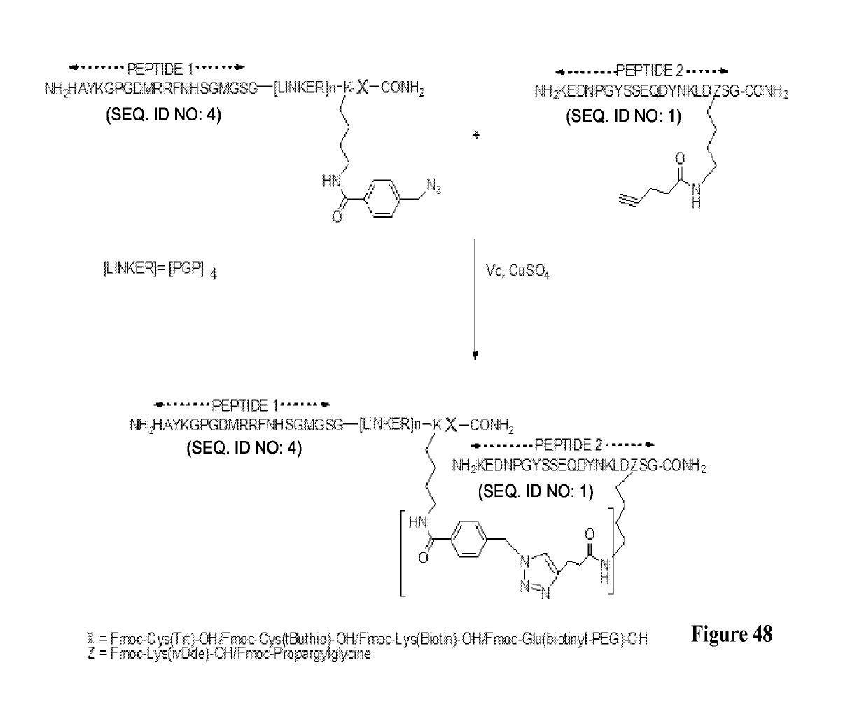

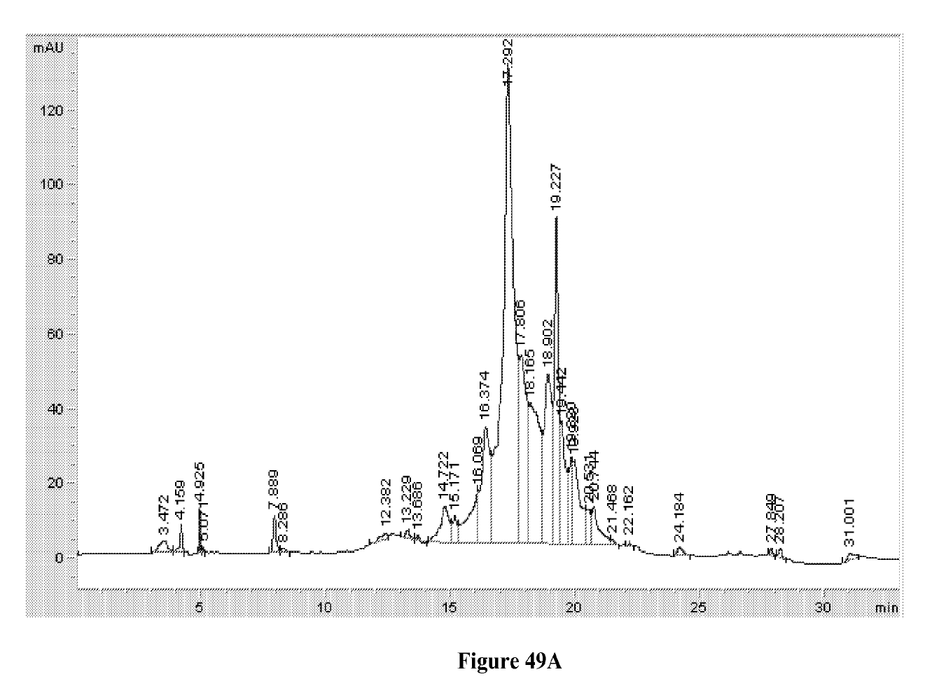

H₂NLys(Fmoc)ProGlyLys(pNz)Lys(Boc)ProGly-Lys(Aloc)AlaOH (FIG. 48(b)). Assembly of the protected peptide was carried out manually. Fmoc-Ala-SASRIN (0.5 g, 0.75 equiv/g) was washed and swollen with CH₂Cl₂ (2×10 mL×15 min) and DMF (2×50 mL×15 min). Coupling reactions were performed using, relative to the resin loading, 4 equiv of N-α-Fmoc-protected amino acid activated in situ with 4 equiv of PyBOP and 8 equiv of DIEA in 8 mL of DMF for 30 min. The completeness of each coupling was confirmed by Kaiser tests. N-α-Fmoc protecting groups were removed by treatment with piperidine:DMF 1:4 (10 mL×4×10 min), the completeness of each deprotection being verified by the UV absorption of the piperidine washings at 299 nm.

Peptide resin was treated repeatedly with TFA:CH₂Cl₂ 1:99 until the resin beads became dark purple (10×10 mL×3 min). Each washing solution was neutralized with pyridine:MeOH 1:4 (5 mL). The combined washings were concentrated under reduced pressure, and white solid was obtained by precipitation from EtOAc/petroleum ether. This solid was dissolved in EtOAc, and pyridinium salts were extracted with water. The organic layer was dried over Na₂SO₄, filtered, and concentrated to dryness. Precipitation from CH₂Cl₂/Et₂O afford white solid which was further desalted by solid-phase extraction and lyophilized to afford the linear peptide. This material was used in the next step without further purification.

Cyclization in solution (FIG. 38(c)). The above linear peptide was dissolved in DMF (100 mL), and the pH was adjusted to 8-9 by addition of DIEA. HATU (1.1 equiv) was added, and the solution was stirred at room temperature for 3 h. Solvent was removed in vacuo; the residue was dissolved in TFA:CH₂Cl₂ 1:1 (15 mL) and allowed to stand for 45 min at room temperature. The solution was then concentrated under reduced pressure and the residue was triturated with Et₂O and filtered to yield the crude product shown in FIG. 38(c). The scaffold can be functionalized in order to attach it to different surfaces, or to add a dye that will help in the studies.

Addition of linker. The scaffold can be functionalized in order to attach it to different surfaces, or to add a dye that will help in the studies. Thus, the linker in can be engineered to have a thiol (SH) group at a terminal position. This thiol can be oxidized to yield a dimer of the scaffold with attached affinity elements. Also, the thiol can be used to attach the structure to various other scaffolds and surfaces. The functionalization takes place at the free NH₂ group as shown in FIG. 39. As an example, this amino group can be acylated using tert-butylthio protected thioglycolic acid. At this point, the scaffold is ready for sequential addition of peptides of interest.

Sequential addition of peptides to the scaffold. The four lysine residues can be orthogonally (without affecting each other) deprotected, reacted with sulfosuccinimidyl-4-(N-maleimidomethyl)cyclohexane-1-carboxylate (Sulfo-SMCC) or other similar heterobifunctional linker, and the corresponding NH₂-protected peptide added. Thus, this scaffold allows incorporation of up to four different peptides as shown in FIG. 39.

The linker shown in FIG. 39 can be engineered to have a thiol (SH) group at a terminal position. This thiol can be oxidized to yield a dimer of the scaffold with attached affinity elements. Also, the thiol can be used to attach the structure to various other scaffolds and surfaces.

### Example 16 PGP Linker Synbody

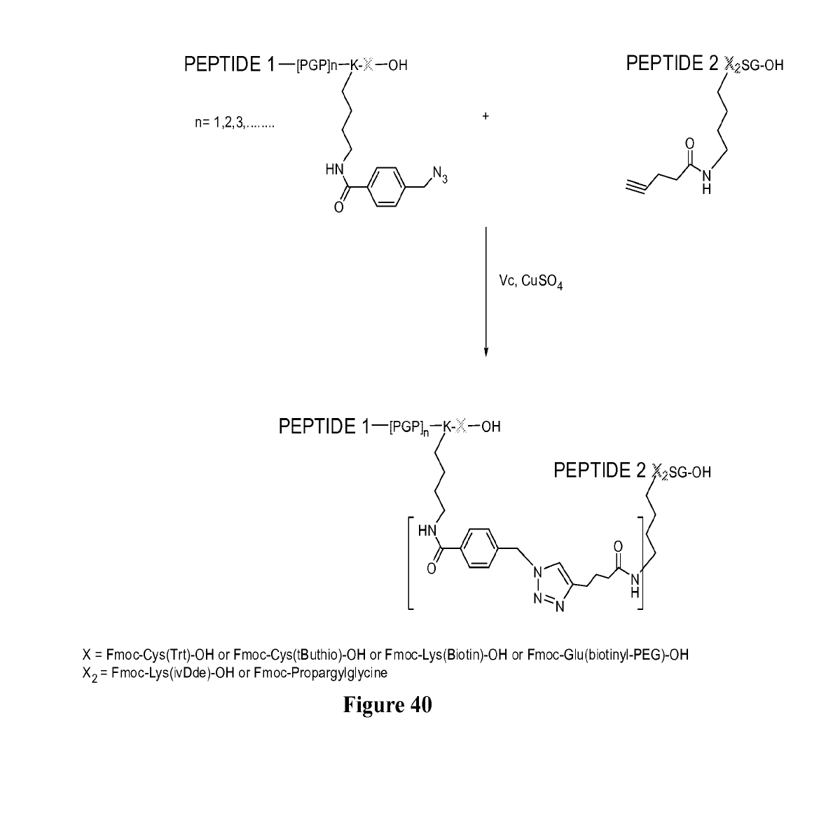

This example demonstrates the synthesis of a synbody having polypeptide affinity elements joined by a poly-(Pro-Gly-Pro) linker, whose length can be determined by inserting the desired number of (Pro-Gly-Pro) subunits, and its assembly by click conjugation. Standard solid phase peptide synthesis methods were used to synthesize, on a Symphony peptide synthesizer, the structure shown in FIG. 40, comprising a polypeptide affinity element 400, a poly-(Pro-Gly-Pro) linker 410, and an azide moiety attached to lysine 402 as shown. A second structure, comprising a second polypeptide affinity element 406, and having an alkyne moiety 404 as shown, was separately synthesized. The two structures were reacted in solution in the presence of vitamin C and CuSO₄ to produce the linked synbody structure 408. Synthesis of the correct synbody structure was verified by MALDI.

In this method, any linker can be used that can be incorporated in the affinity element/linker/azide structure during solid phase synthesis; thus, this method provides a way of testing a variety of linker compositions.

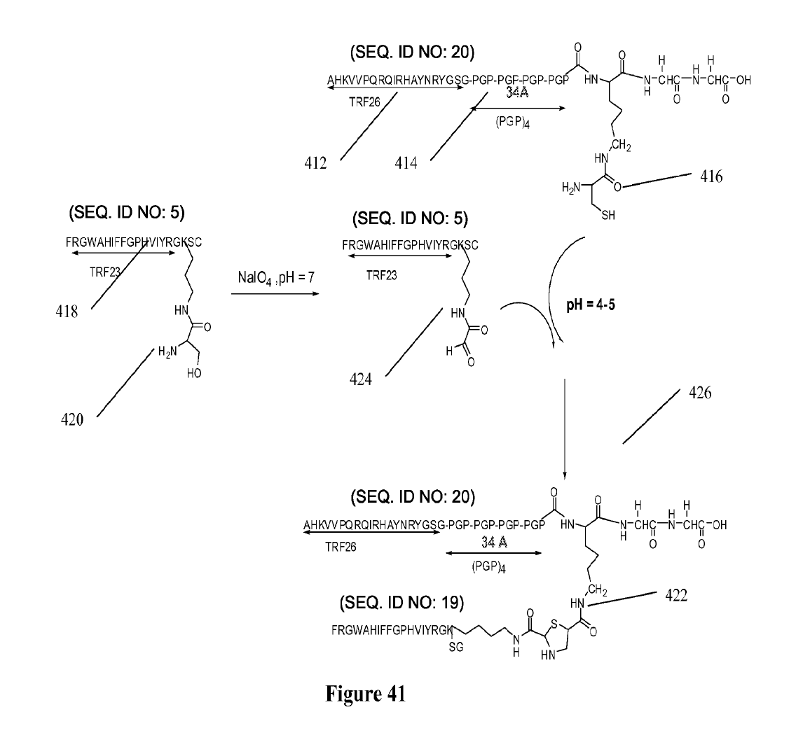

A poly-(Pro-Gly-Pro) linked synbody was also constructed by the thiazolidine formation process shown in FIG. 41. In this synthesis, a polypeptide affinity element TRF 26 (SEQ ID NO. 8) 412 was synthesized together with its poly-(Pro-Gly-Pro) linker 414 by standard solid phase peptide synthesis methods, having a cysteine residue 416 at or near the opposite end of the linker from the polypeptide affinity element 412 as shown. A second polypeptide affinity element TRF 23 (SEQ ID NO. 5) 418 was synthesized having a serine residue 420 near its C terminus, which was modified as shown 424. The two entities were reacted in solution at pH 4.5 to produce the thiazolidine ring linkage 422 shown. Synthesis of the correct synbody structure 426 was verified by MALDI.

### Example 17 Synthesis of Synbody

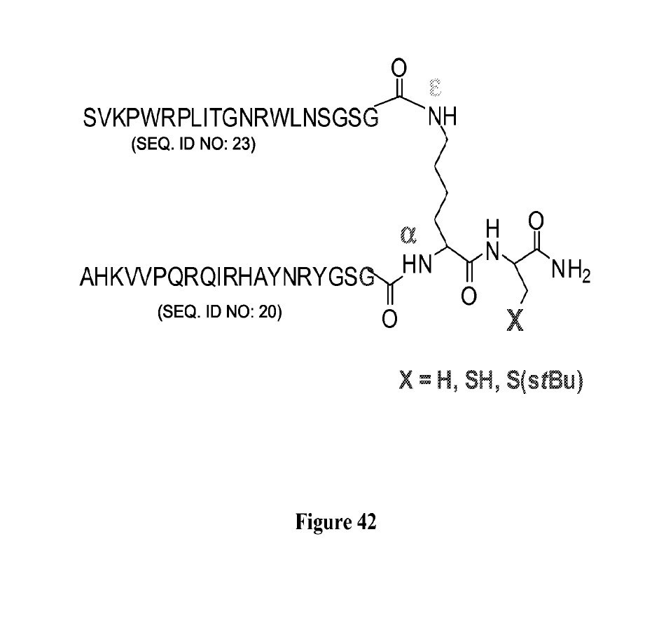

This example demonstrates the synthesis of a synbody having two peptide affinity elements, linked by conjugating them to the a ands amine moieties of a lysine monomer as shown in FIG. 42.

All reagents and solvents were analytical, HPLC or peptide synthesis grade. Commercial reagents and solvents were obtained from Aldrich and Fisher respectively and used without further purification unless otherwise noted. All amino acids and resins were purchased from Novabiochem, Chem Impex International Inc. as well as from Advanced Chem Tech and used without further purification. Fmoc-L-Propargylglycine was purchased from Peptech. All peptides were synthesized via standard Fmoc stepwise solid phase peptide synthesis (SPPS) on Symphony Multiple Peptide Synthesizer at 25 umole scale. Matrix-assisted laser desorption/ionization time-of-flight mass spectrometry (MALDI-TOF/MS) was carried out on Bruker Daltonic multiplex instrument. UV measurements were carried out on a ND-1000 spectrophotometer instrument. All reversed-phase HPLC analysis and purifications were conducted on an Agilent 1200. Phenomenex Luna 5u analytical (4.6×250 mm) and semi-preparative (10×250 mm) C-18 columns were used for the analysis and purification. As used in these examples, “DMSO” refers to Dimethylsulphoxide; “DMF” refers to N,N-Dimethylformamide (DMF); “AcCN refers to Acetonitrile; “MeOH” refers to methyl alcohol; “DCM” refers to Dichloromethane; “HOBt” refers to 1-Hydroxybenzotriazole; “HBTU” refers to 2-(1-H-benzotroazole-1-yl)-1,3,3-tetramethyluronium Hexafluorophosphate; “NMM” refers to N-methylmorpholine; “TFA” refers to Trifluoroacetic acid; “DIPEA” refers to N,N-Diisopropylethylamine; “TIPS” refers to Triisopropylsilane; “DoDt” refers to 3,6-Dioxa-1,8-octane-dithiol; “ivDDe” refers to 1-(4,4-Dimethyl-2,6-dioxo-cyclohexylidene)-3-methyl-butyl; “Fmoc” refers to Fluorenylmethoxycarbonyl; “Kaiser reagents” refers to (1) Ninhydrine solution, 6% in ethanol, (2) Potassium cyanide in pyridine, and (3) Phenol in 80% ethanol.

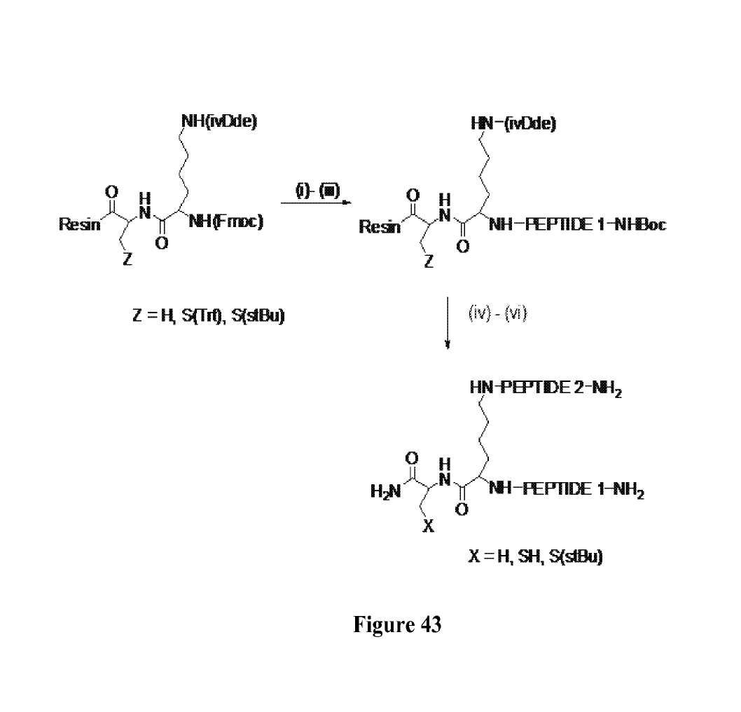

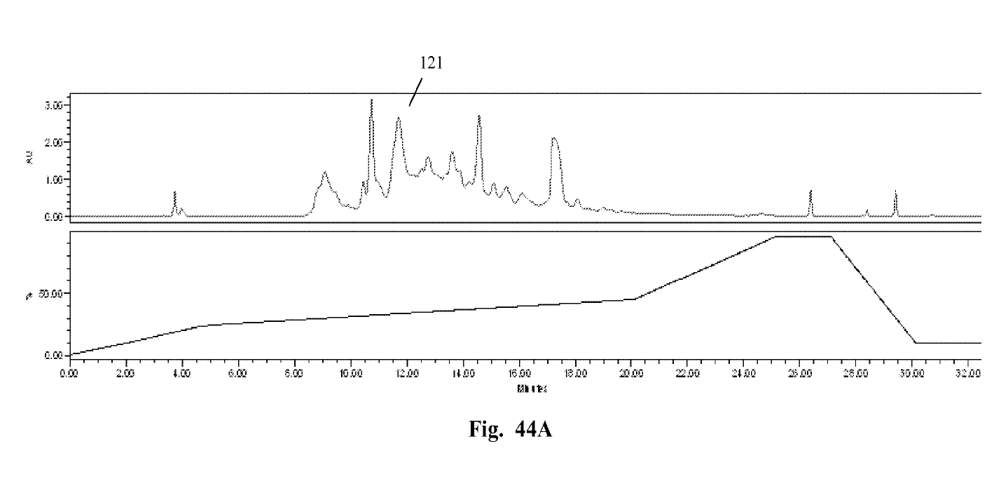

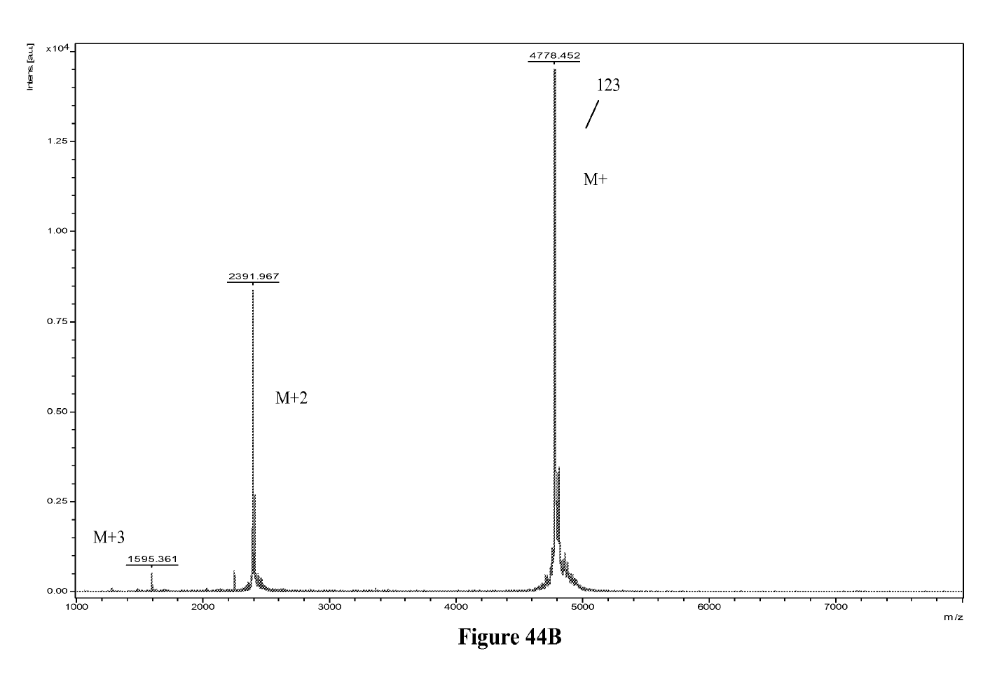

Synbodies were synthesized via standard Fmoc divergent solid phase peptide synthesis using orthogonal protecting groups on branched lysine. Two orthogonal groups were introduced using Fmoc-Lys(ivDde)-OH at the very C-terminus. The synthesis was carried out at 25 umole scale on Rink amide resin (0.7 mmole/g) and PEGA resin functionalized with Rink amide linker (0.35 mmole/g). As illustrated in FIG. 43, the general strategy followed for the synthesis of the synbodies to which this example pertains is: (i) Rink Resin/PEGA Rink Amide Resin, 20% Piperidine in DMF (5+15 mins); (ii) Stepwise coupling of amino acids (SPPS) for Peptide sequence 1; (iii) 20% Piperidine in DMF (5+15 mins); 5×(Boc)₂O 10×DIPEA; (iv) 5% Hydrazine in DMF (2 hrs); (v) Stepwise coupling of amino acids (SPPS) for Peptide sequence 2; (vi) TFA Cleavage.

Following removal of Fmoc-protecting group by 20% piperidine in DMF for 5+15 mins, peptide sequence 1 was synthesized on α-amino group of Lysine through stepwise addition of Fmoc amino acids. N-terminus Fmoc group was substituted with Boc group manually by treating with 5 fold excess of (Boc)2O (125 umol, 0.027 g) in presence of 10×DIPEA (250 umol, 2.6 mL). The resin was agitated at room temperature for 1 hr followed by standard washings with DMF (3×, 1 min each), MeOH (2×, 1 min each), DCM (2×, 1 min each), DMF (3×, 1 min each). An aliquot of resin was taken after MeOH wash for qualitative Kaiser test. At this point, NE-(ivDde) protecting group was deprotected manually using 5% hydrazine monohydrate in DMF followed by standard washings. Removal of (ivDde) was monitored spectrophotometrically by absorption of the resulting 3,6,6-trimethyl-4-oxo-4,5,6,7-tetrahydra-1H-indazole at 300 nm and was completed in 2 hrs. Deprotection was also was verified by standard qualitative Kaiser Test.

The stepwise assembly of the peptide sequence 2 was then accomplished at NE-lysine position again on Peptide synthesizer. A five fold molar excess of Fmoc-amino acids, HOBt and NMM was used throughout the synthesis in a stepwise manner. The final protected di-epitopic MAP was treated with cleavage cocktail (TFA:phenol:DoDt:H₂O:TIPS::85:3:5:5:2) for 2 hrs at room temperature and precipitated in cold diethyl ether. (DoDt was not used in the cleavage cocktail when stBu was used as a protection group at the very C-terminal cysteine.) The precipitated construct was cooled for 15 mins in −80° C. refrigerator to ensure complete precipitation. The solid was separated from the diethyl ether by centrifugation and the top phase was decanted off and pellet re-suspended with another addition of dry diethyl ether. The cooling and centrifugation process was done in triplicate. Upon completion, the construct was dried and dissolved in water for HPLC purification and MALDI characterization (see Example 18).

### Example 18 HPLC/MALDI Purification and Verification of Synbody

This example demonstrates the isolation and verification of synthesis of a synbody synthesized according to the methods described in Example 17. The peptide affinity elements of the synbody had the sequences H₂N-FRGWAHIFFGPHVIYRGGSG and H₂N-AHKVVPQRQIRHAYNRYGSG, extending from the E and a amine moieties, respectively, of the lysine linker. After synthesis according to the method described in Example 17, the construct was purified on reverse-phase HPLC on Phenomenex Luna 5u semi-preparative (10×250 mm) C-18 column using solvent system A: 0.1% TFA in H2O; solvent B: 90% CH3

[symbol]

CN in 0.1% TFA with a linear gradient method, 0 min, 10% B; 2 min, 10% B; 20 min, 45% B; 25 min, 95% B; 27 min, 95% B; 30 min, 100% B; 33 min, 10% B) with flow rate of 4 mL/min at a wavelength of 280 nm. See the chromatogram shown in FIG. 44A. The fractions were pooled off and analyzed by MALDI-TOF mass spectrometry. FIG. 44B shows the MALDI spectrum of the fraction 121 corresponding to the correct product (computed mass 4780.3, MALDI peak 123 mass 4778.452); this fraction was then lyophilized. Example 19 Construction of Synbody Library

This example demonstrates the construction of a library of synbodies for further screening, with the synbodies synthesized according to the methods described in Examples 17 and 18. The synbodies shown in Table 5 were synthesized. Synbody compositions are shown in Table 1 in the form Peptide 1-Peptide 2-linker. In all cases, affinity elements peptide 1 and peptide 2 were conjugated at their C termini to the E and a mine moieties, respectively, of the lysine monomer of the linker. The sequences of peptide 1 and peptide 2 in these constructs are given in Table 6. The suffixes “KC”, “KA”, and “KC(StBu)” in Table 5 indicate the choice of group X (see FIG. 43) as H, SH, or S(StBu), respectively.

TABLE 5
Library of lysine-linked synbodies

| Peptide Sequence | Mol wt |
|---|---|
| TRF21-TRF19-KC | 4766.1 |
| TRF21-TRF21-KC | 4912.5 |
| TRF21-TRF22-KC | 4725.3 |
| TRF24-TRF19-KC | 4642 |
| TRF24-TRF20-KA | 4805.4 |
| TRF24-TRF21-KC | 4788.4 |
| TRF24-TRF22-KA | 4569 |
| TRF24-TRF24- KA | 4632.2 |
| TRF24-TRF25-KA | 4615.1 |
| TRF23-TRF19 - KC | 4678 |
| TRF23-TRF23-KC(stBu) | 4825 |
| TRF23-TRF23-KA | 4704.3 |
| TRF21-TRF23-KA | 4927 |
| TRF26-TRF19 - KC | 4754.1 |
| TRF26-TRF20-KA | 4917.5 |
| TRF26-TRF21- KA | 4868.4 |
| TRF26-TRF22-KG | 4667.1 |
| TRF26-TRF23- KA | 4780.3 |
| TRF26-TRF23-KC | 4812.4 |
| TRF26-TRF23-KCC | 4915.5 |
| Scramble-TRF26-TRF23-KC | 4812.4 |
| m-TRF26-TRF23- KC | 4803.4 |
| TRF26-TRF24 - KA | 4744.3 |
| TRF26-TRF26-KA | 4856.4 |
| TNFa1-TNFa 4- KC(stBu) | 4526.4 |
| TN Fa 2-TNFa 3-KC(stBu) | 4477.1 |
| TN Fa 1-TN Fa 3-KC(stBu) | 4496.2 |
| TNFa 2-TNFa5-KC(stBu) | 4731.4 |
| TNFa1-TNFa10-KC(stBu) | 4630.3 |
| BP1-BP1- KA | 3250 |
| BP1-BP1-KC(stBu) | 3372.5 |
| Bx3-Bx7-KC | 4747.4 |
| Bx3-Bx9-KC | 4585.2 |
| 6′SL-6′SL-KC | 4443.9 |

TABLE 6
Peptide affinity element sequences

| | |
|---|---|
| TRF19 | KEDNPGYSSEQDYNKLDGSG |
| TRF20 | GQTQFAMHRFQQWYKIKGSG |
| TRF21 | QYHHFMNLKRQGRAQAYGSG |
| TRF22 | HAYKGPGDMRRFNHSGMGSG |
| TRF23 | FRGWAHIFFGPHVIYRGGSG |
| TRF24 | SVKPWRPL1TGNRWLNSGSG |
| TRF25 | APYAPQQIHYWSTLGFKGSG |
| TRF26 | AHKVVPQRQIRHAYNRYGSG |
| TRF27 | LDPLFNTSIMVNWHRWMGSG |
| BP1 | GTEKGTSGWLKTGSG |
| BP2 | EGEWTEGKLSLRGSG |
| TNFa 1 | MKSIIPMSVAQHQGPIKGSG |
| TNFa 2 | RTTEMPFVFALGSVHPGGSG |
| TNFa 3 | SMKMVQPGHLLISYGHQGSG |
| TNFa 4 | FMNYPIKVPILVVPIGRGSG |
| TNFa 5 | VMLYNWHIMQHRNNKPVGSG |
| TNFa 10 | FRGWAHIFFGPHVIYRGGSG |
| Bx3 | AKGMFKAPYYKTPDRNRGSG |
| Bx7 | LSIMQSERLPHSWKGYRGSG |
| Bx9 | GTQPMVAWKDVYGIVVYGSG |
| 6′SL | AQYSFVVGVKGFIHAQYGSG |

### Example 20 Synthesis of Peptide with Azido-Modified PGP Linker

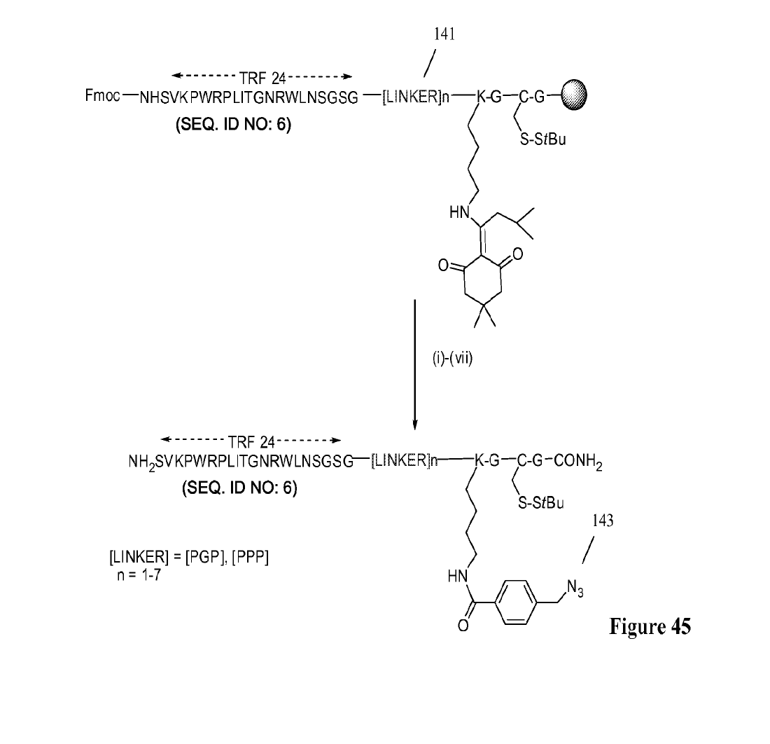

This example demonstrates the synthesis of a peptide affinity element conjugated, as shown in FIG. 45, to a poly-proline or poly-[proline-glycine-proline] linker 141, with the distal portion of the linker azido-modified 143 to facilitate conjugation of a second peptide affinity element thereto via azide-alkyne “click” conjugation. The general strategy, as illustrated in FIG. 45, is: (i) Rink Resin/PEGA Rink Amide Resin, 20% Piperidine in DMF (5+15 mins); (ii) Stepwise coupling of amino acids (SPPS) for Peptide Sequence; (iii) 20% Piperidine in DMF (5+15 mins); (iv) 5×(Boc)₂O, 10×DIPEA; (v) 5% Hydrazine in DMF (2 hrs); (vi) Coupling with 4-(azidomethyl)benzoic acid; (vii) TFA Cleavage.

More specifically, peptides with varying lengths of poly-[proline-glycine-proline] and poly-proline linkers were synthesized at 25 umole scale using Rink amide resin (0.7 mmol)/PEGA Rink amide resin (0.35 mmol/g) on a Symphony Multiple Peptide Synthesizer. In the example shown in FIG. 45, a linker 141, which may be either poly-proline or poly-[proline-glycine-proline], followed by peptide TRF-24 (see Table 6 above) was assembled through stepwise addition of Fmoc amino acids using HOBt/HBTU/NMM as activating agents. All Arginines and Valines were double coupled. The peptide assembly was terminated by N-capping with di-t-butyl dicarbonate manually by treating resin with 5 fold excess of (Boc)₂O (125 umol, 0.027 g), in presence of 10×DIPEA (250 umol, 2.6 mL) for 1 hr. Reaction mixture was then removed by suction followed by standard washings with DMF (3×, 1 min each), MeOH (2×, 1 min each), DCM (2×, 1 min each), DMF (3×, 1 min each). An aliquot of resin was taken after MeOH for qualitative Kaiser Test. The Ns-(ivDde) protecting group introduced via Fmoc-Lys(ivDde)-OH at the very C-terminus was then deprotected manually through treatment of 5% hydrazine monohydrate in DMF followed by standard washings. Removal of (ivDde) was monitored spectrophotometrically by absorption of the resulting indazole at 300 nm and was completed in 2 hrs. Deprotection was again verified by standard qualitative Kaiser Test. 4-(Azidomethyl)benzoic acid (125 umol, 0.2 g) was incorporated at 6-amino group through HOBt:HBTU:DIPEA (1:1:2) (50 uL of 0.5M solution of each HOBt and HBTU in DMF; 2.6 mL of DIPEA). The resin was agitated for 1.5 hr at r.t. Azido modified peptide with linker is then, dried in vacuo before cleavage. The peptides were cleaved from resin by treatment of TFA in the presence of phenol, TIPS and water as scavengers. The resin was agitated with cleavage cocktail (TFA:phenol:H₂O:TIPS::85:3:5:2) at r.t. for 2 hrs and precipitated in cold diethyl ether. The precipitated construct was cooled for 15 mins in a −80° C. refrigerator to ensure complete precipitation. The solid was separated from the diethyl ether by centrifugation and the top phase was decanted off and pellet re-suspended with another addition of dry diethyl ether. The cooling and centrifugation process was done in triplicate. Upon completion, the construct was dried and dissolved in water for HPLC purification, and fractions collected and verified by MALDI-TOF mass spectrometry, and the correct fraction was lyophilized, all according to the methods described in Example 18 above.

For use in the foregoing synthesis, 4-(Azidomethyl)benzoic acid was synthesized as follows: 4-(Chloromethyl)benzoic acid (30 mmol, 5.12 g) was added in one portion to a solution of sodium azide (59.9 mmol, 3.9 g), crown-ether (2.9 mmole, 0.8 g) in DMSO (30 mL). The reaction mixture was stirred over night at r. t. The solvent was removed in vacuum and diluted with ethyl acetate, followed by washing with 0.1N HCl (10 mL×2), brine and dried over sodium sulfate. Product was concentrated by removing excess solvent in vacuum and crystallized with ethyl acetate/hexane. 4.37 g of solid white powder was obtained. The product was characterized by 1H NMR and ESI mass spectrometry. (1-H NMR (CDCl3, 400 MHz) 4.45 (s, 2H), 7.46 (d, J=8.1, 2H), 8.12 (d, J=8.1, 2H); (m/z, calcd for C8H7N3O2: 177.16; found 177 (M+), 200 (M++Na)).

### Example 21 Synthesis of Alkyne-Modified Peptide

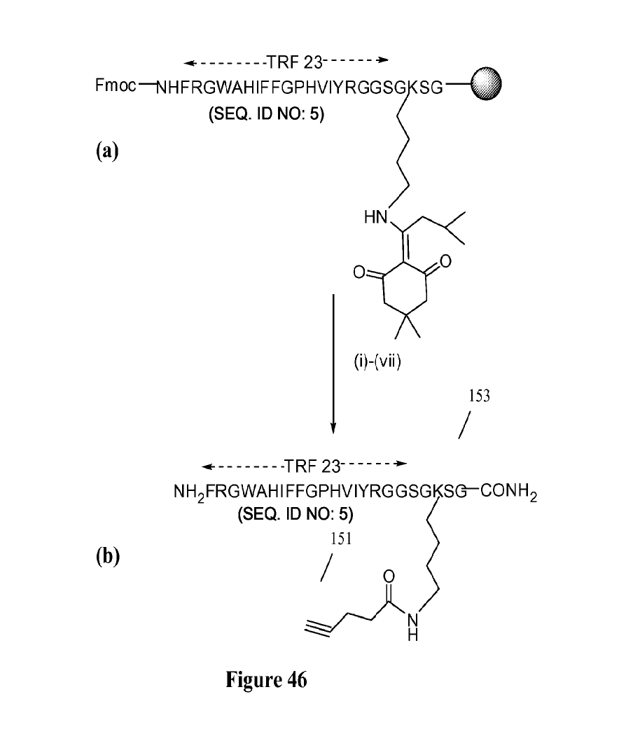

This example demonstrates the synthesis of an alkyne-modified peptide affinity element for assembly by azide-alkyne “click” conjugation with an azido-modified peptide-linker construct (see Example 20), so as to produce a bivalent synbody (see Example 22). Synthesis and alkyne modification was performed as follows (see FIG. 46 upper): (i) Rink amide (0.7 mmol/g)/PEGA Rink Amide (0.35 mmol/g) Resin, 20% Piperidine in DMF (5+15 mins); (ii) Stepwise coupling of amino acids (SPPS) for Peptide Sequence; (iii) 20% Piperidine in DMF (5+15 mins); (iv) 5×(Boc)20, 10×DIPEA; (v) 5% Hydrazine in DMF (2 hrs); (vi) Coupling with 4-pentynoic acid; (vii) TFA Cleavage. Peptides were synthesized without linker and functionalized with 4-pentynoic acid (125 umol, 0.1 g) in presence of HOBt:HBTU:DIPEA (1:1:2) (50 uL of 0.5M solution of each HOBt and HBTU in DMF; 2.6 mL of DIPEA) for 1.5 hr, resulting in the structure diagrammed in FIG. 46 lower, with the alkyne functionalization 151 on the side chain of the lysine residue 153 two residues inward from the C terminus of peptide TRF-23 (see Table 6) as shown. Cleavage and purification was performed according to the methods described in Examples 17 and 18 above. (In the alternative, an alkyne moiety may be introduced in a peptide sequence by coupling with the unnatural amino acid Fmoc-L-Propargylglycine during SPPS.)

### Example 22 Assembly of a Synbody by Coupling of Peptide with Azido-Modified PGP Linker with Alkyne Modified Peptide

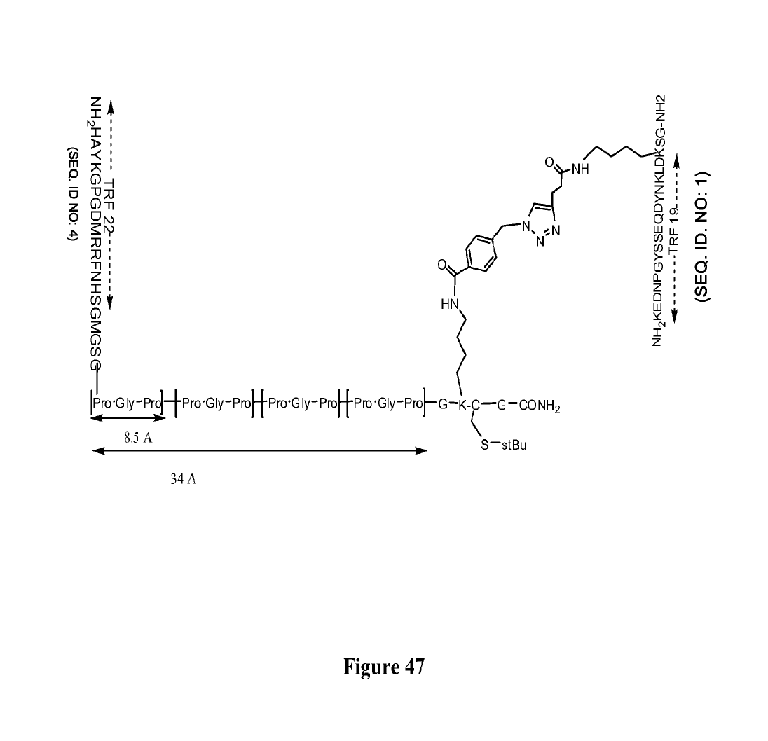

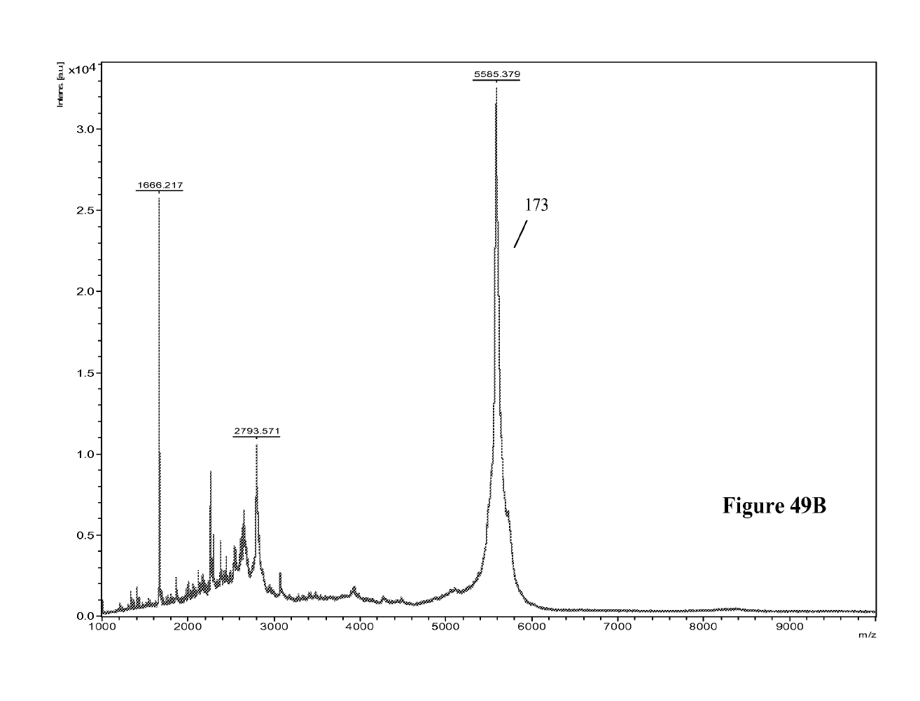

This example demonstrates the Cu(I) catalyzed [3+2] cycloaddition conjugation of a first peptide affinity element, alkyne-modified according to the methods described in Example 21, with the azido-modified linker of a peptide-linker construct, synthesized according to the methods described in Example 20, to produce a bivalent synbody. FIG. 47 diagrams the method as applied to alkyne modified peptide TRF19 (see Table 6 for sequence) and peptide-linker construct where the peptide is sequence TRF22 (see Table 2) and the linker is [proline-glycine-proline]4 as shown. Using this method, libraries of synbodies having poly-[proline-glycine-proline] and poly-proline linkers were synthesized and purified, having the compositions shown in Tables 7 and 8, respectively. In Tables 7 and 8, “(PGP)N” or “(PPP)N” indicate poly-(proline-glycine-proline) or poly-(proline-proline-proline) linkers, respectively, with the indicated tripeptide repeated N times. The plus sign denotes azide-alkyne click conjugation of the two indicated constructs according to the methods described in this example. The conjugations were performed, as diagrammed in FIG. 48, via Cu(I) catalyzed Huisgen reaction. The azido-modified peptide with linker (0.1 umol) and alkyne functionalized peptide (0.2 umol) were dissolved in water. To this was added sodium ascorbate (Vc) (1 umole, freshly prepared in water) followed by copper(II) sulfate solution (1 umol, freshly prepared in water). The reaction mixture was stirred at room temperature for 12 hrs. The reaction mixture was purified on reverse-phase HPLC on Phenomenex semi-preparative (10×250 mm, Luna 5u) C-18 column using solvent system A: 0.1% TFA in H₂O; solvent B: 90% CH₃CN in 0.1% TFA with a linear gradient method, 0 min, 10% B; 2 min, 10% B; 20 min, 45% B; 25 min, 95% B; 27 min, 95% B; 30 min, 100% B; 33 min, 10% B) with flow rate of 4 mL/min at a wavelength of 280 nm. The fractions were pooled off and the fraction containing the desired product identified by MALDI-TOF mass spectrometry. The identified fraction was then lyophilized. FIG. 49 shows an example of the HPLC separation and MALDI-TOF mass spectrographic verification of a synbody from one of the libraries described in this example (TRF26GSG-(PPP)1-K(Azido)G+TRF23GSGK(4-pentynoic acid)SG). The synbody has a computed mass of 5,587.1 D; as shown in the chromatograph (FIG. 49A) and MALDI spectrum of the selected fraction (FIG. 49B), the selected HPLC fraction 161 produced a MALDI peak 163 of 5,585.379.

TABLE 7
Poly-PGP synbodies

| [PGP]ₙ-SYNBODIES (TRIAZOLE) | MW |
|---|---|
| TRF26GSG (PGP)1 - K(Azido)GK(Biotin)G + TRF23GSGK(4-pentynoic acid)SG | 5956 |
| TRF26GSG (PGP)1 - K(Azido)GK(Biotin)G + TRF28GSGK(4-pentynoic acid)SG | 5964 |
| TRF26GSG - (PGP)1 - K(Azido)GK(Biotin)G + TRF22PropargylglycineSG | 5543 |
| TRF26GSG (PGP)4 - K(Azido)GC(StBu) + TRF23GSGK(4-pentynoic acid)SG | 6491 |
| TRF26GSG (PGP)4 - K(Azido)GC(StBu) + TRF22PropargylglycineSG | 6078 |
| TRF26GSG - (PGP)4 - K(Azido)GA + TRF23GSGK(4-pentynoic acid)SG | 6371 |
| TRF26GSG - (PGP)1 - K(Azido)GC(StBu)G + TRF23GSGK(4-pentynoic)SG | 5795.2 |
| TRF26GSG - (PGP)2 - K(Azido)GC(StBu)G + TRF23GSGK(4-pentynoic)SG | 6046.5 |
| TRF26GSG (PGP)3 - K(Azido)GC(StBu)G + TRF23GSGK(4-pentynoic)SG | 6297.8 |
| TRF26GSG - (PGP)4 - K(Azido)GC(StBu)G + TRF23GSGK(4-pentynoic)SG | 6549 |
| TRF26GSG - (PGP)5 - K(Azido)GC(StBu)G + TRF23GSGK(4-pentynoic)SG | 6800.3 |
| TRF26GSG - (PGP)6 - K(Azido)GC(StBu)G + TRF23GSGK(4-pentynoic)SG | 7051.6 |
| m- TRF26GS G - (PGP)4 - K(Azido)GC(StBu)G + TRF23GSGK(4-pentynoic)SG | 6540.9 |
| TRF26GSG - (PGP)1 - K(Azido)GC(StBu)G + TRF20K(4-pentynoic)SG | 5731.1 |
| TRF26GSG - (PGP)2 - K(Azido)GC(StBu)G + TRF20K(4-pentynoic)SG | 5982.4 |
| TRF26GSG - (PGP)3 - K(Azido)GC(StBu)G + TRF20K(4-pentynoic)SG | 6233.7 |
| TRF26GSG - (PGP)4 - K(Azido)GC(StBu)G + TRF20K(4-pentynoic)SG | 6484.9 |
| TRF26GSG - (PGP)5 - K(Azido)GC(StBu)G + TRF20K(4-pentynoic)SG | 6736.2 |
| TRF26GSG - (PGP)6 - K(Azido)GC(StBu)G + TRF20K(4-pentynoic)SG | 6986.8 |
| TRF26GSG - (PGP)1 - K(Azido)GC(StBu)G + TRF24K(4-pentynoic)SG | 5731.1 |
| TRF26GSG - (PGP)2 - K(Azido)GC(StBu)G + TRF24K(4-pentynoic)SG | 5809.2 |
| TRF26GSG - (PGP)3 - K(Azido)GC(StBu)G + TRF24K(4-pentynoic)SG | 6060.5 |
| TRF26GSG - (PGP)4 - K(Azido)GC(StBu)G + TRF24K(4-pentynoic)SG | 6311.7 |
| TRF26GSG - (PGP)5 - K(Azido)GC(StBu)G + TRF24K(4-pentynoic)SG | 6563 |
| TRF26GSG - (PGP)6 - K(Azido)GC(StBu)G + TRF24K(4-pentynoic)SG | 6814.3 |
| TRF26GSG - (PGP)1 - K(Azido)GC(StBu)G + TRF22(Propargylglycine)SG | 5380.76 |
| TRF26GSG - (PGP)2 - K(Azido)GC(StBu)G + TRF22(Propargylglycine)SG | 5632.06 |
| TRF26GSG - (PGP)3 - K(Azido)GC(StBu)G + TRF22(Propargylglycine)SG | 5888.36 |
| TRF26GSG - (PGP)4 - K(Azido)GC(StBu)G + TRF22(Propargylglycine)SG | 6134.56 |
| TRF26GSG - (PGP)5 - K(Azido)GC(StBu)G + TRF22(Propargylglycine)SG | 6385.86 |
| TRF26GSG - (PGP)6 - K(Azido)GC(StBu)G + TRF22(Propargylglycine)SG | 6637.16 |
| TRF24GSG - (PGP)4 - K(Azido)G + TRF23GSGK(4-pentynoic)SG | 6188.8 |
| TRF24GSG - (PGP)4 - K(Azido)G + TRF22(Propargylglycine)SG | 5774.8 |
| BP1GSG - (PGP)4 - K(Az)G + BP2K(4-pentynoic acid)SG | 4568.8 |

TABLE 8
Poly-Proline Synbodies

| [PPP]ₙ-SYNBODIES (TRIAZOLE) | MW |
|---|---|
| TRF26GSG - (PPP)2 - K(Azido)GA + TRF23K(4-pentynoic acid)SG | 5747 |
| TRF26GSG - (PPP)3 - K(Azido)GA + TRF23K(4-pentynoic acid)SG | 6038.9 |
| TRF26GSG - (PPP)4 - K(Azido)GA + TRF23KK(4-pentynoic acid)SGG | 6515 |
| TRF26GSG - (PPP)5 - K(Azido)GA + TRF23KK(4-pentynoic acid)SGG | 6804 |
| TRF26GSG - (PPP)1 - K(Azido)G + TRF23GSGK(4-pentynoic acid)SG | 5587.1 |
| TRF26GSG - (PPP)1 - K(Azido)GC(StBu)G + TRF23GSGK(4-pentynoic)SG | 5835.3 |
| TRF26GSG - (PPP)2 - K(Azido)GC(StBu)G + TRF23GSGK(4-pentynoic)SG | 6127.6 |
| TRF26GSG - (PPP)3 - K(Azido)GC(StBu)G + TRF23GSGK(4-pentynoic)SG | 6418 |
| TRF26GSG - (PPP)4 - K(Azido)GC(StBu)G + TRF23GSGK(4-pentynoic)SG | 6709.3 |
| TRF26GSG - (PPP)5 - K(Azido)GC(StBu)G + TRF23GSGK(4-pentynoic)SG | 7000.7 |
| TRF26GSG - (PPP)6 - K(Azido)GC(StBu)G + TRF23GSGK(4-pentynoic)SG | 7292 |
| TRF26GSG - (PPP)7 - K(Azido)GC(StBu)G + TRF23GSGK(4-pentynoic)SG | 7583.4 |
| m-TRF26GSG - (PPP)2 - K(Azido)GC(StBu)G + TRF23GSGK(4-pentynoic)SG | 6117.9 |
| TRF24GSG - (PPP)3 - K(Az)G + TRF22(Propargylglycine)SG | 5643.3 |

### Example 23 Synthesis of Double-“Click” PPP-Linked Synbodies

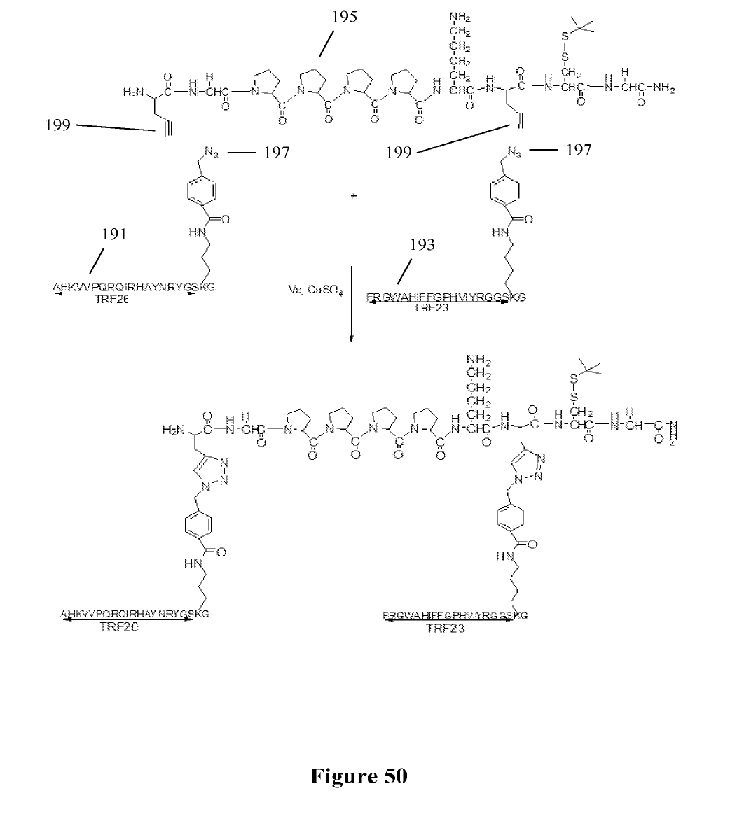

This example demonstrates the assembly of a synbody having two peptide affinity elements 191, 193 (sequences TRF26 and TRF 23, see Table 6) conjugated to opposite ends of a poly-proline linker 195. The C-terminal sequences of the peptides are GSKG, and the peptides are azido-modified at the s amine of the lysine residue 197 adjacent to the C-terminal glycine, as shown in FIG. 50. The poly-proline linker is alkyne-modified 199, and the peptide affinity elements are click-conjugated to the alkyne moieties of the poly-proline linker to form a bivalent synbody. The reaction produces four distinct synbody products (of which, for brevity, only one is shown in FIG. 50), since each peptide sequence can conjugate to either end of the linker; however, if a single product is desired, this can be readily accomplished by employing orthogonal click conjugation chemistries at the two ends of the linker. The azido-modification of the peptide affinity elements and the alkyne modification of the linker were accomplished generally according to the methods described in Examples 20 and 21 above. The click conjugation reaction was performed as follows: Alkyne modified linker (0.05 umol, 49 uL of 1.09 mM solution in water) and azido functionalized peptides (TRF26Az: 0.1 umol, 55 uL of 1.8 mM solution in water; TRF23Az: 0.1 umol, 200 uL of 0.5 mM solution in water) were added to a vial containing magnetic stir bar. To this added, sodium ascorbate (Vc) (0.2 umole, freshly prepared in water) followed by copper(II) sulfate solution (0.2 umol, freshly prepared in water). The reaction mixture was stirred at room temperature for 12 hrs. The reaction mixture was purified on reverse-phase HPLC on Phenomenex semi-preparative (10×250 mm, Luna 5u) C-18 column using solvent system A: 0.1% TFA in H₂O; solvent B: 90% CH₃

[symbol]

CN in 0.1% TFA with a linear gradient method, 0 min, 10% B; 2 min, 10% B; 20 min, 45% B; 25 min, 95% B; 27 min, 95% B; 30 min, 100% B; 33 min, 10% B) with flow rate of 4 mU/min at a wavelength of 280 nm. The fractions were pooled off and analyzed by MALDI-TOF mass spectrometry. The correct fraction was then lyophilized.

The synbodies shown in Table 9 were synthesized according to the method described. “GP4”, “GP5”, and “GP6” refer to the linker molecule 195 depicted in FIG. 50, with 4, 5, or 6 proline monomers, respectively.

TABLE 9
Double-“click” synbodies

| Synbody | MW |
|---|---|
| GP4 -TRF26 - TRF26-StBu (From rxn GP4 with TRF26-23) | 6335.6 |
| GP5 -TRF26 - TRF26-StBu (From rxn GP5 with TRF26-23) | 6432.6 |
| GP5 -TRF26 - TRF23-StBu (From rxn GP5 with TRF26-23) | 6356.5 |
| GP5 -TRF26 - TRF26-StBu (From rxn GP5 with TRF26-26) | 6432.6 |
| GP6 -TRF23 - TRF23-StBu (From rxn GP6 with TRF26-23) | 6529.6 |
| GP6 -TRF26 - TRF26-StBu (From rxn GP6 with TRF26-23) | 6377.4 |
| GP6 -TRF23 - TRF23-StBu (From rxn GP6 with TRF23-23) | 6529.4 |

### Example 24 Construction of Linker Libraries

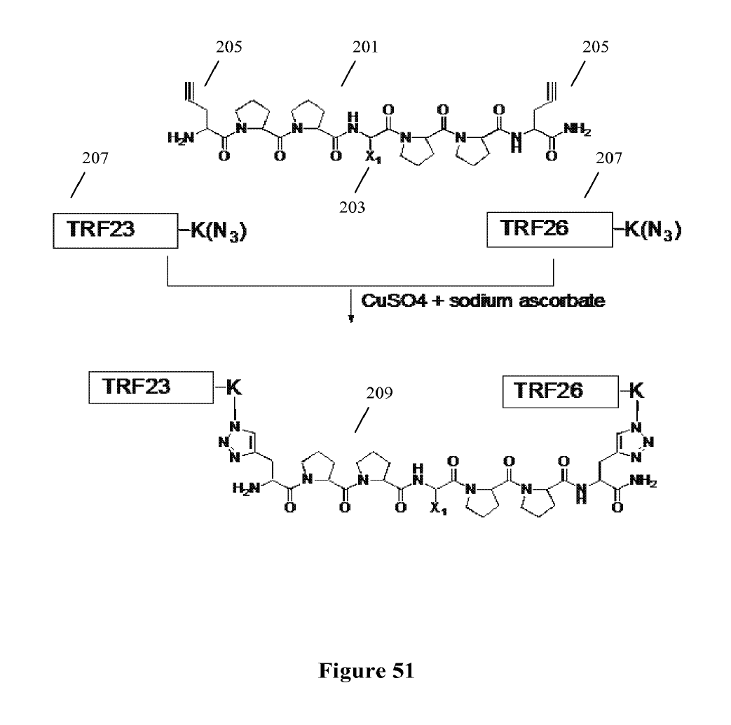

This example demonstrates the construction of a library of linkers in which the length and composition of the linker is varied among the members of the library. This was accomplished by preparing a combinatorial library wherein each linker was a peptide having a length and sequence based on one of the templates PGP1, PGP2, PGP3 or PGP4 shown in Table 10. The linkers according to templates PGP1, PGP2, PGP3 and PGP4 have, respectively, one, two, three, or four variable positions, with each variable position occupied by a residue corresponding to one of the six residues shown for the variable position in question under the “Amino Acids” column in Table 10. FIG. 51 depicts a PGP linker 201 having a single variable position 203. The linkers have propargyl glycine residues 205 at the N and C termini, which provide the alkyne moieties for click conjugation to the peptide affinity elements. As indicated in the sequence templates in Table 10 (but not shown in FIG. 51), in all libraries described in this example, a lysine residue was added to the N terminus to improve ionizability so as to facilitate mass spectrographic characterization. The linker libraries are then click-conjugated to azido-modified peptide affinity elements 207 to produce a library of bivalent synbodies 209 having a diversity of linker lengths and/or variable position residues.

TABLE 10
Linker sequence templates

| | | Random | | | | | | |
|---|---|---|---|---|---|---|---|---|
| Name | Sequence Template | Residue | Amino Acids | | | | | |
| PGP I | K-Pra-PP-X1-PP-Pra | X1 | Lys | Ser | Asp | Asn | Gly | Trp |
| PGP2 | K-Pra-PP-X2-PP-X1-PP-Pra | X1 | Lys | Ser | Asp | Asn | Gly | Trp |
| | | X2 | Arg | Thr | Glu | Gln | Gly | Phe |
| PGP3 | K-Pra-PP-X3-PP-X2-PP-X1-PP-Pra | X1 | Lys | Ser | Asp | Asn | Gly | Trp |
| | | X2 | Arg | Thr | Glu | Gln | Gly | Phe |
| | | X3 | His | Tyr | Ala | Met | Gly | Leu |
| PGP4 | K-Pra-PP-X4-P-X3-PP-X2-PP-X1-PP-Pra | X1 | Lys | Ser | Asp | Asn | Gly | Trp |
| | | X2 | Arg | Thr | Glu | Gln | Gly | Phe |
| | | X3 | His | Tyr | Ala | Met | Gly | Leu |
| | | X4 | Lys | Ser | Asp | Asn | Gly | Trp |

Fmoc solid phase peptide synthesis methods were used to assemble the peptide linker library starting at the C-terminus as usual. A split-mix methodology was applied to the first two positions of diversity (at position X1 and X2), resulting in a sub-library of PGP1 (a mixture of six linkers) and a sub-library of PGP2 (a mixture of 36 linkers). After X2, the synthesis continues by a split-only method, resulting in six sub-libraries of PGP3 and thirty-six sub-libraries of PGP4; each of these sub-libraries contains 36 linkers. The PGP3 sub-libraries are denoted herein by the (known) X3 amino acid, and PGP4 sub-libraries are denoted by the known X3 and X4 residues. Thus, for example, HXX refers to a sub-library of PGP3 that has His at X3 position, while KAXX refers to a sub-library of PGP4 that has Lys at X4 and Ala at X3 position. Table 11 shows the sequences and molecular weights, with and without protonation, of the linkers making up the PGP2 sub-library.

For the Fmoc peptide synthesis, 8 grams of Rink Amide ChemMatrix resin (0.56 mmole/gram, Matrix Innovation, Montreal, Canada) were used in the synthesis of a total of 1554 peptide linkers (2.8 pmole/linker). Organic solvents and other peptide synthesis reagents were obtained from current commercial sources and used without further purification. During the synthetic process, a four-step reaction cycle was followed for the addition of amino acids using Fmoc-Pra (Advanced ChemTech, Louisville, Ky.), other Fmoc-protected amino acids (Novabiochem, San Diego, Calif.) to the growing peptide chain: (1) Fmoc deprotection: the resin was treated twice with a volume of 20% piperidine in DMF (10 mU/gram), once for 5 min and again for 20 min. (2) Resin wash: the resin was washed by filtration with DMF (3×), MeOH (2×), DCM (2×), and DMF (3×); a volume of 10 mU/gram was used at each washing step. (3) Amino acid coupling: to the resin is added a volume of amino acid coupling solution in dry DMF (10 mU/gram): Fmoc-amino acid (0.2 M), HBTU (0.2 M), HOBt (0.2 M) and NMM (0.4 M). Normally, the coupling reaction is complete in one hour. The completeness can also be monitored by Kaiser test. (4) Resin wash: same as step 2.

The synthetic process employed may be described in four stages:

Stage 1: Synthesis of 8-mer peptide chain and PGP1. The resin was first swelled in 100 mL of DMF in a 225-mL polyethylene bottle. By following the 4-steps reaction cycle described above, the first three amino acids (Pra, Pro, Pro) were added to the resin in the same plastic bottle. The resin was then split into six aliquots and each aliquot was placed into a 50-mL polyethylene syringe with a frit at its bottom. Washing solvents and reaction solutions (e.g., deprotection and coupling, 10 mL/gram) can be added to the resin through a syringe needle by pulling the syringe plunger and can be removed from the resin either by pushing the syringe plunger or by connecting it to a solvent-vacuum line. By following the 4-steps reaction cycle described above, each of the amino acids in group X1 (see Table 11) was added to one of the syringes for coupling. The resins were then combined in a 225-mL plastic bottle for the next two cycles of amino acid (Pro) addition.

Before the resin was split again for the addition of the X2 amino acids, a portion of the resin (^{˜}30 mg) was removed from the bottle and capped with Propargylglycine (Pra) and Lysine (Lys), resulting in a sub-library that contained six PGP1 linkers.

Stage 2: Synthesis of 11-mer peptide chain and PGP2. The remaining resin was split for the addition of X2 amino acids in a same manner described above for the X1 amino acid addition. Afterward, the resin was combined again in a 225-mL bottle for two cycles of Proline addition. A portion of the resin (^{˜}180 mg) was removed from the bottle and capped with Propargylglycine (Pra) and Lysine (Lys), resulting in a sub-library that contained thirty-six PGP2 linkers.

Stage 3: Synthesis of 14-mer peptide chain and PGP3. The remaining resin was split again for the addition of X3 amino acids as described above for the X1 and X2 amino acid addition. Each syringe was labeled with an amino acid from the group-X3. For example, a syringe was labeled with “H”, indicating histidine was to be added to resin in that syringe. After the addition of the group X3 amino acids, the resins remained divided and the next two cycles of Proline addition were performed in the same syringes. Resin in each syringe was further divided into 7 aliquots and each was placed in a 5-mL syringe with a frit at its bottom for retaining the resin beads; one of every seven aliquots was be capped with Propargylglycine (Pra) and Lysine (Lys), resulting in six sub-libraries of PGP3 linkers, each containing 36 distinct linker species. Each of the remaining 5-mL syringes was labeled with a four-letter code indicating the group X4 residue to be added and the group X3 residue already present.

Stage 4: Synthesis of 17-mer peptide chain and PGP4. Using the same 4-step reaction cycle described above, each of the amino acids in group X4 was added to the corresponding syringe, followed by two more cycles of proline addition. Resins in all the PGP4 syringes were capped with Propargylglycine (Pra and Lysine (Lys), resulting in thirty-six sub-libraries, each containing thirty-six PGP4 linkers.

Both TFA-gas phase cleavage and Solution phase cleavage methodologies were used in cleaving the peptides from resins. In the gas cleavage technique, 5 mg of resin was removed from each of the 44 sub-libraries and each placed in a specific well in a 96-well plate. The plate was placed in a desicator connected, through a two-way valve, to a vacuum pump and a flask containing trifluoroacetic acid (TFA). The desicator was first subjected to high vacuum for ten minutes before being switched to the TFA-containing flask; TFA evaporated under reduced pressure and filled the desicator. After exposure to TFA gas overnight (20-24 hours), the plate was removed from the desicator. To the resin-containing well was then added 20 μL of Acetonitrile (ACN) to elute the peptide from the resin beads. 2 μL of the eluted peptide was used for analysis by MALDI-MS.

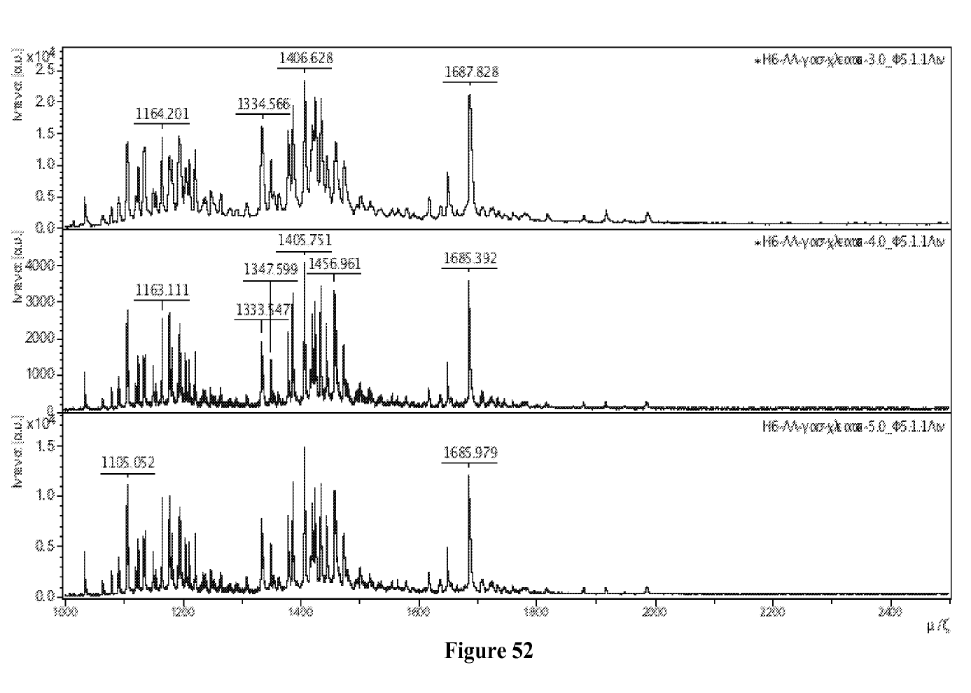

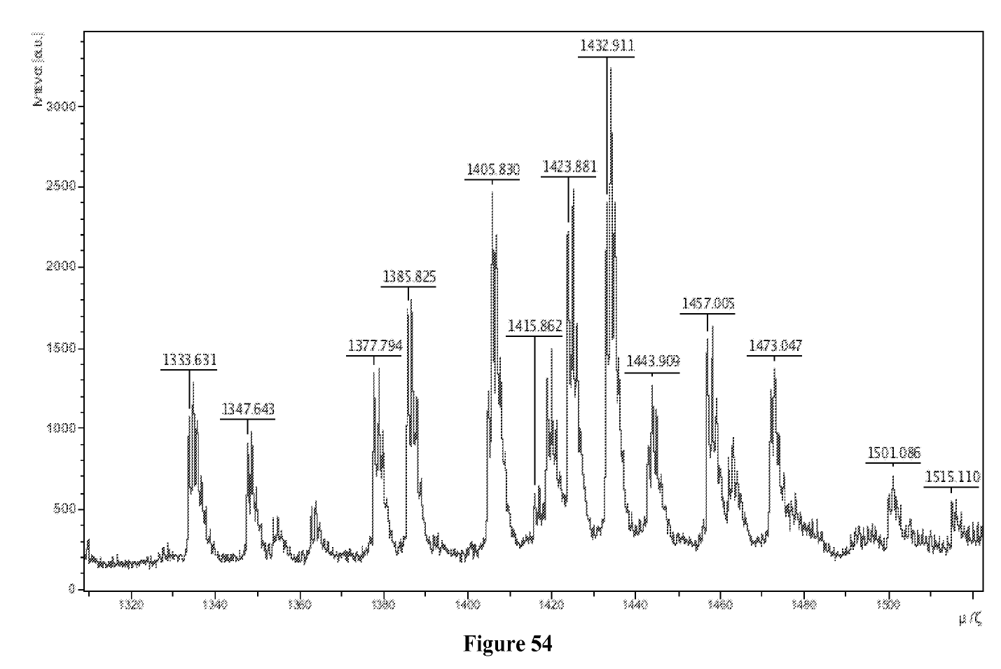

FIGS. 52, 53, and 54 show MALDI-mass spectra of the gas phase cleaved sample of the PGP2 sub-library shown in Table 11, at increasing levels of detail. By comparing to the calculated molecular weights of the linkers as shown in Table 11 (“Molecular weight” column is without protonation, “MH+” column is with protonation), it will be seen that the molecular ions in the region of 1000-1300 correspond to the expected molecular weights of the linkers. Approximately 80% of the linkers in Table 11 can be identified from FIGS. 52, 53, and 54, which is quite good considering that many of the ions have expected molecular weights within one atomic unit (au) of each other. FIG. 54 shows an expanded view of section 1310-1520 from FIG. 52. Most of the molecular ions in this section appear to correspond to the expected molecular weights of linkers that still bear either one or two protection groups (pbf and Trt, MW 253 and 243, respectively). For example, the molecular ion corresponding to the peak at 1432.911 likely corresponds to the linker K-Pra-PP-Arg-PP-Asn-PP-Pra in table 5 that has a trityl (Trt) on the Asn residue. This result indicates that, after over night exposure, the cleavage did not completely remove the side chain protection groups on some of the linkers.

TABLE 11
PGP2 Sub-library

| | Molecular | |
|---|---|---|
| Sequences | Weight | MH+ |
| K-Pra-PP-Gly-PP-Gly-PP-Pra | 1032.539 | 1033.547 |
| K-Pra-PP-Gly-PP-Ser-PP-Pra | 1062.549 | 1063.557 |
| K-Pra-PP-Thr-PP-Gly-PP-Pra | 1076.565 | 1077.573 |
| K-Pra-PP-Gly-PP-Asn-PP-Pra | 1089.56 | 1090.568 |
| K-Pra-PP-Gly-PP-Asp-PP-Pra | 1090.544 | 1091.552 |
| K-Pra-PP-Gln-PP-Gly-PP-Pra | 1103.576 | 1104.584 |
| K-Pra-PP-Gly-PP-Lys-PP-Pra | 1103.612 | 1104.62 |
| K-Pra-PP-Glu-PP-Gly-PP-Pra | 1104.56 | 1105.568 |
| K-Pra-PP-Thr-PP-Ser-PP-Pra | 1106.576 | 1107.584 |
| K-Pra-PP-Phe-PP-Gly-PP-Pra | 1122.586 | 1123.594 |
| K-Pra-PP-Arg-PP-Gly-PP-Pra | 1131.624 | 1132.632 |
| K-Pra-PP-Thr-PP-Asn-PP-Pra | 1133.586 | 1134.594 |
| K-Pra-PP-Gln-PP-Ser-PP-Pra | 1133.586 | 1134.594 |
| K-Pra-PP-Thr-PP-Asp-PP-Pra | 1134.57 | 1135.578 |
| K-Pra-PP-Glu-PP-Ser-PP-Pra | 1134.571 | 1135.579 |
| K-Pra-PP-Thr-PP-Lys-PP-Pra | 1147.638 | 1148.646 |
| K-Pra-PP-Phe-PP-Ser-PP-Pra | 1152.596 | 1153.604 |
| K-Pra-PP-Gln-PP-Asn-PP-Pra | 1160.597 | 1161.605 |
| K-Pra-PP-Glu-PP-Asn-PP-Pra | 1161.581 | 1162.589 |
| K-Pra-PP-Gln-PP-Asp-PP-Pra | 1161.581 | 1162.589 |
| K-Pra-PP-Gly-PP-Trp-PP-Pra | 1161.597 | 1162.605 |
| K-Pra-PP-Arg-PP-Ser-PP-Pra | 1161.634 | 1162.642 |
| K-Pra-PP-Glu-PP-Asp-PP-Pra | 1162.565 | 1163.573 |
| K-Pra-PP-Gln-PP-Lys-PP-Pra | 1174.649 | 1175.657 |
| K-Pra-PP-Glu-PP-Lys-PP-Pra | 1175.633 | 1176.641 |
| K-Pra-PP-Phe-PP-Asn-PP-Pr | 1179.607 | 1180.615 |
| K-Pra-PP-Phe-PP-Asp-PP-Pra | 1180.591 | 1181.599 |
| K-Pra-PP-Arg-PP-Asn-PP-Pra | 1188.645 | 1189.653 |
| K-Pra-PP-Arg-PP-Asp-PP-Pra | 1189.629 | 1190.637 |
| K-Pra-PP-Phe-PP-Lys-PP-Pra | 1193.659 | 1194.667 |
| K-Pra-PP-Arg-PP-Lys-PP-Pra | 1202.697 | 1203.705 |
| K-Pra-PP-Thr-PP-Trp-PP-Pra | 1205.623 | 1206.631 |
| K-Pra-PP-Gln-PP-Trp-PP-Pra | 1232.634 | 1233.642 |
| K-Pra-PP-Glu-PP-Trp-PP-Pra | 1233.618 | 1234.626 |
| K-Pra-PP-Phe-PP-Trp-PP-Pra | 1251.644 | 1252.652 |
| K-Pra-PP-Arg-PP-Trp-PP-Pra | 1260.682 | 1261.69 |

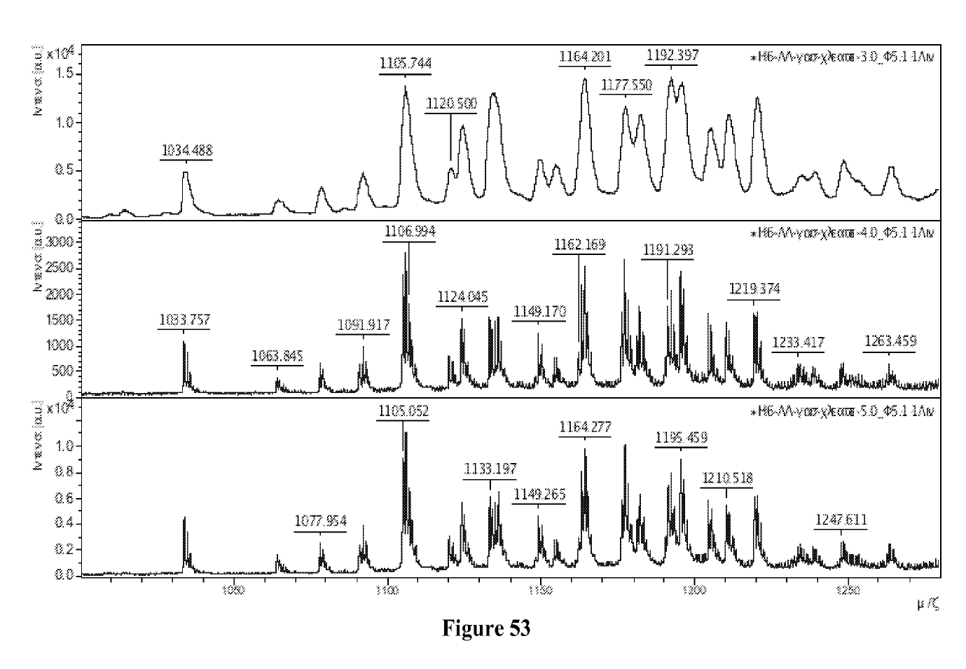

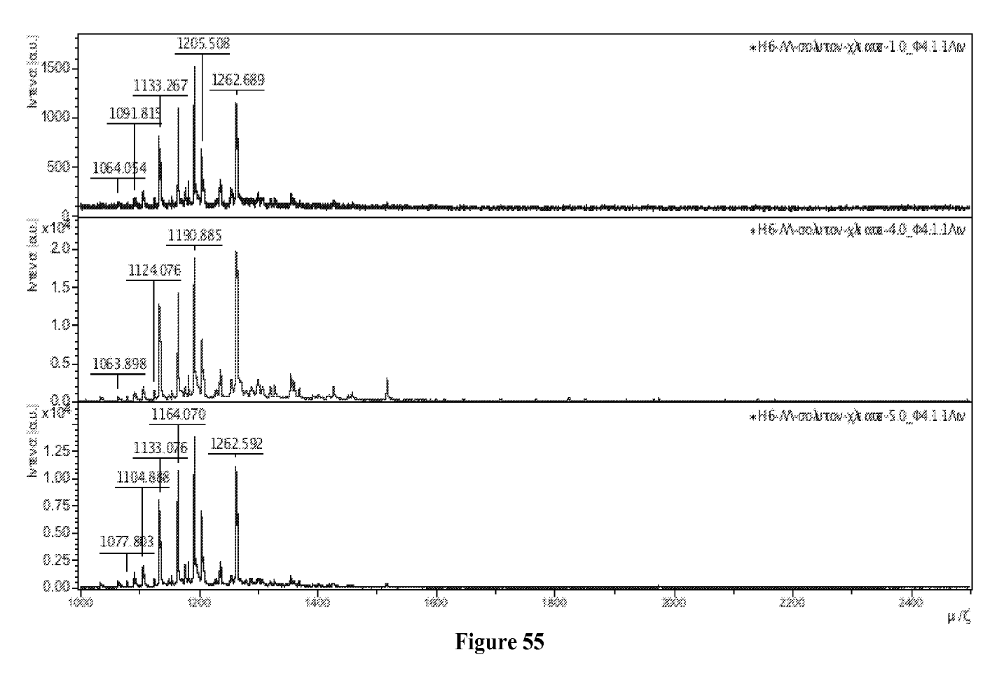

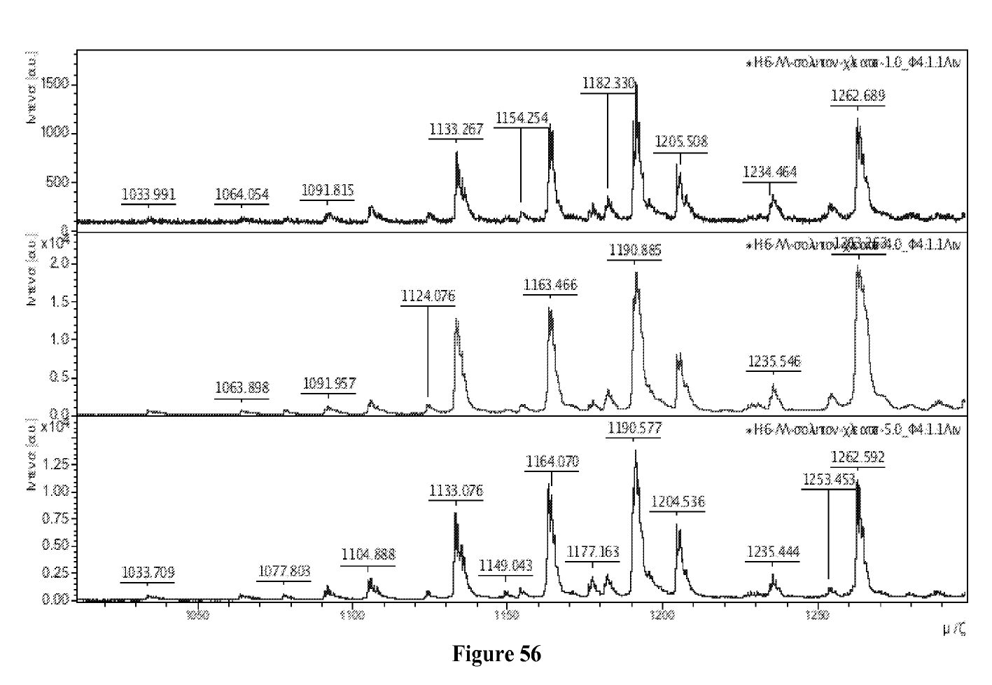

Solution phase cleavage of sublibraries was also performed and the results compared with those for gas phase cleavage. Each sub-library (^{˜}180 mg resin) was treated with 5 mL of cleavage solution (TFA 90%, Phenol 2.5%, TIPS 2.5%, water, 5%) for 2-3 hours. The cleavage solution was then removed from the resins and dropwise added to 45 mL cold ether; after centrifugation, the precipitated peptide linkers were washed with cold ether (3×). Each linker sub-library was dissolved in 5 mL water/acetonitrile (2/1) and lyophilized. A small sample was prepared from each sub-library and analyzed by MALDI-MS. By way of example, the MALDI mass spectra acquired for the solution phase cleavage sample of the PGP2 linker sub-library (Table 7) are shown in FIGS. 55 and 56. Comparing to the mass spectra acquired from the gas phase cleavage sample (FIGS. 52, 53, and 54), it is clear that all the side chain protection groups were completely removed from the peptide linkers. Also, as shown in the expanded view (FIG. 56), almost all the molecular ions listed in Table 11 are recognizable from the mass spectra; however, many molecular ions are much weaker as compared to the intensities of the same molecular ion observed from the gas phase cleavage sample (FIGS. 53 and 54).

### Example 25 Construction of Synbodies Using Linker Libraries

This example demonstrates the construction of bivalent synbodies having azido-modified peptide affinity elements conjugated to the linker libraries described in Example 24 by Cu(I)-catalyzed Huisgen azido-alkyl 1,3-cycloaddion reaction (Click chemistry). Synthesis of synbody TRF23-PGP1-TRF26, whose structure is shown in FIG. 51, is described in this example; synbodies incorporating other peptide affinity elements and/or other linker sublibraries were synthesized in the same manner. Since the click conjugation chemistry used was the same at both linker attachment points, conjugation of two distinct peptide affinity elements results in four distinct synbodies corresponding to each linker species. For example, conjugation of TRF23 and TRF26 to PGP1 results in four different synbodies, TRF23-PGP1-TRF23, TRF26-PGP1-TRF26, TRF23-PGP1-TRF26, and TRF26-PGP1-TRF23, for each PGP1 species present.

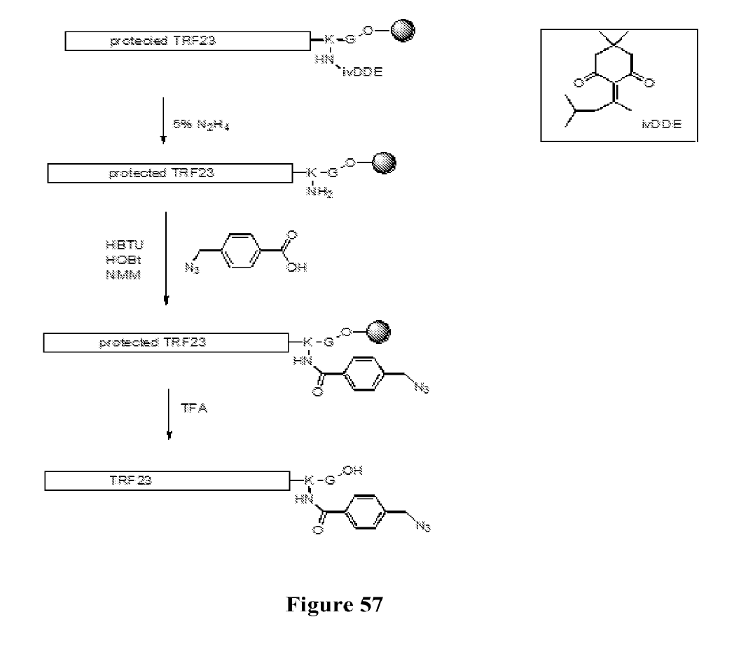

Synthesis of the bivalent synbodies was carried out as follows (see FIG. 57): Materials. All the Fmoc-amino acids were purchased from Novabiochem (San Diego, Calif.). Other synthetic reagents and organic solvents used in peptide synthesis were obtained from current commercial sources and used without further purification. Peptides were synthesized on a liberty microwave peptide synthesizer (CEM Corporation, NC).

Synthesis of azido-modified peptides. Peptides that were selected for conjugation to the linkers were synthesized on a microwave peptide synthesizer with Lys(ivDDE) at their C-terminus and modified with an azido-bearing group as shown in FIG. 57.

Specifically, fully protected peptide obtained from the microwave synthesizer was treated with a solution of 5% hydrazine in DMF (10 mL/gram resin) for 20 hours at room temperature. The resin was washed with DMF, MeOH, DCM and DMF before it was treated again with a coupling solution of azidomethylbenzoic acid (0.2 M) in the presence of HBTU (0.2 M), HOBt (0.2 M), NMM (0.4 M) in DMF (10 mL/gram resin). This coupling step takes at least 24 hours, the completeness of coupling needing to be monitored by Kaiser test. The resin was treated with a TFA cleavage solution (TFA 90%, Phenol 2.5%, TIPS 2.5%, and water 5%). After 3 hours of reaction, the cleavage solution was separated from the resin and dropwise added to cold ether to obtain the precipitate of the peptide. The peptide was purified by HPLC and the product verified by MALDI-MS. (TRF23-K—N3, MALDI-MS: 2546.28 (calculated), 2546.18 (measured)).

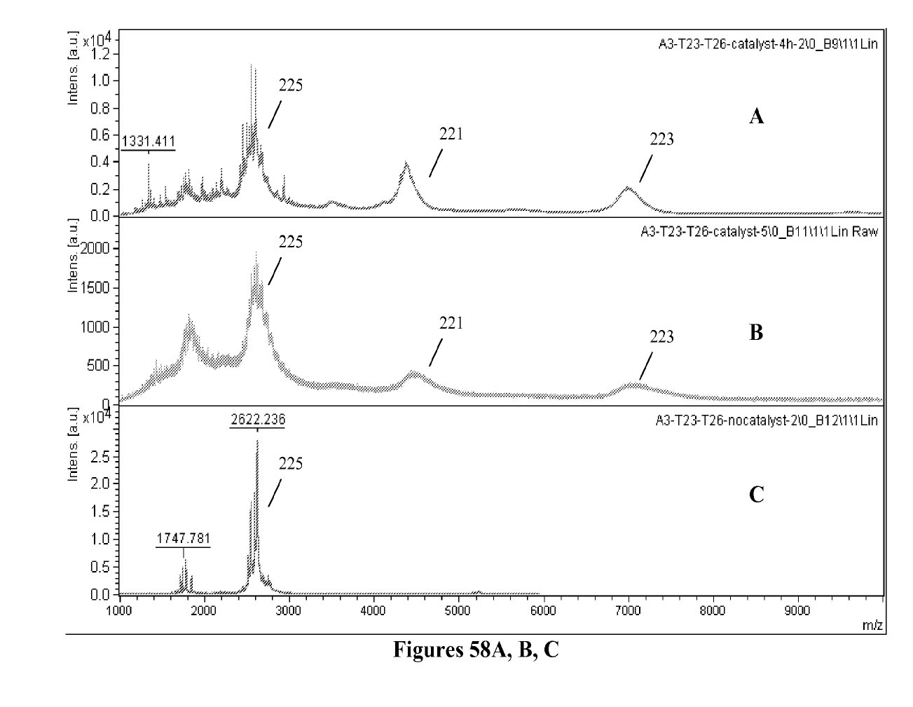

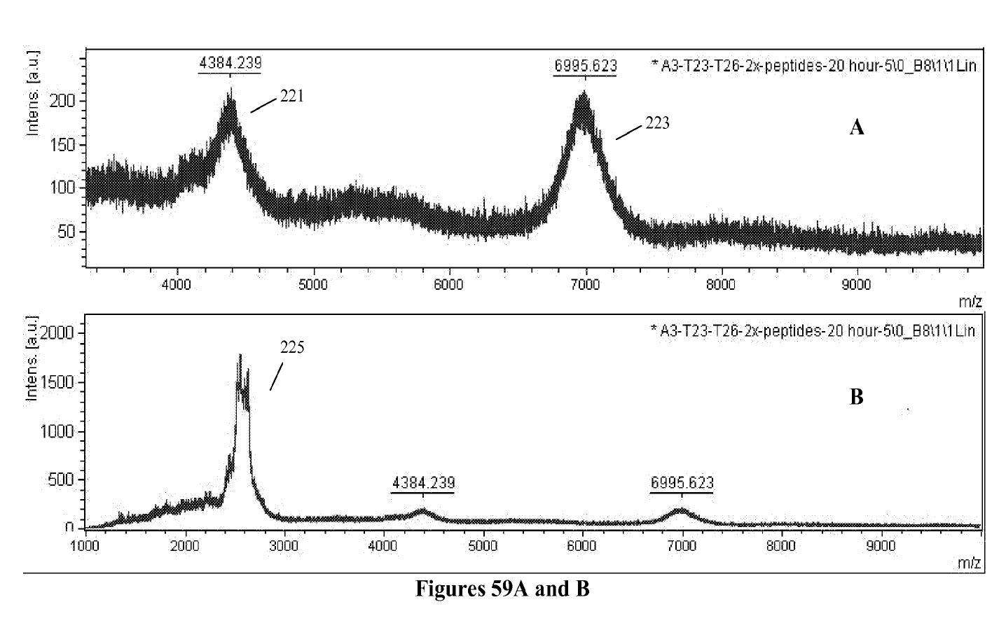

Synthesis of Synbodies. Following the process depicted in FIG. 51, two azido-modified transferrin-binding Peptides, TRF23-K—N3 and TRF26-K—N3 were conjugated to the linker libraries described Example 24. In this example, conjugation of these two peptides to the KAXX sub-library is described (KAXX refers to a sub-library of PGP4 that has Lys at X4 and Ala at X3 position; see Table 10). The following solutions were made before the conjugation: 10 mM KAXX in MeOH/H₂O (1/1) (solution A), 10 mM TRF26-K—N3 in MeOH/H₂O (solution B), 5 mM TRF23-K—N3 in MeOH/H₂O (solution C), 20 mM CuSO₄ in H₂O (solution D), and 20 mM Vitamin C in H₂O (sodium ascorbate, solution E). The reaction was carried out in a 1.5-mL polypropylene centrifuge tube at 45° C. Reagents were added in the following sequence: 10 μL of solution A, 10 μL solution B, 20 μL solution C, 20 μL solution D and 40 μL solution E. The solution becomes turbid immediately after the addition of solution E. 50 μL H₂O and 50 μL MeOH are then added to make the solution clear. The reaction was monitored by MALDI-MS. FIG. 58 shows the MS analysis before addition of catalyst (Cu and vitamin C) (FIG. 58C), immediately after the addition of catalyst (FIG. 58B), and 4 hours after the addition of catalyst and reaction at 45° C. (FIG. 58A). The MALDI-MS results show that the catalytic reaction proceeded reasonably fast. However, the group of molecular ions 221 observed around 4200 indicated significant presence of mono-conjugates, in comparison to the peak 223 corresponding to the bis-conjugated products and the peak 225 corresponding to the unconjugated peptides. To facilitate further conjugation to achieve a higher yield of the desired bis-conjugation product, 10 μL of solution B and 20 L of solution C were added to the reactor and the reaction was allowed to proceed for additional 15 hours at 45° C. The MALDI-MS result following this additional step (FIG. 59A, full spectrum, FIG. 59B, expanded view of 3500-9800 MW range) showed that linkers were completely consumed, the mono-conjugates peak 221 was substantially reduced, and the desired bis-conjugates peak 223 was increased correspondingly. Unreacted peptide 225 remained in the solution.

### Example 26 High Throughput Screening of Peptides Using SPR

This example demonstrates the high throughput screening of peptide affinity element candidates in solution phase by SPR assay, and demonstrates that peptide affinity elements having moderate affinity (K_{D}^{˜}10-200 μM) for a predetermined protein target can be identified within a relatively small library (on the order of 10⁴) of random sequence peptides. A library of peptides, 20 amino acids in length, was synthesized by Alta Biosciences (Birmingham, UK) in 96 well plates and used without further purification. The sequences of the first 17 positions of the peptides from and including the N terminus were determined computationally by a pseudorandom process with each of the 19 naturally occurring amino acid types except cysteine weighted equally, and the last three C-terminal residues were glycine-serine-cysteine. Peptides were re-suspended by adding 500 μL of DMF and shaking overnight at 4° C. Five hundred microliters of 100 mM phosphate buffered saline (PBS) was then added to each well. A Beckman FX robotic liquid handling system was used to transfer 50 μL per well from 4 96-well plates into a 384 well plate that contained 50 μL of PBS per well, thus creating a stock plate of peptides. Peptide concentration per well was approximately 1-2 mg/mL and the purity of each peptide was ^{˜}50 to 70%.

Peptide affinity element candidates were screened against target proteins immobilized on the SPR surface. Each target protein was modified with biotin using the following procedure: NHS-LC-LC-Biotin (Pierce Biotechnology) was re-suspended in DMSO at a concentration of 7.13 mM. Each protein was prepared in 100 mM PBS pH 7.5 at a concentration of ^{˜}50 μM. NHS-LC-LC-Biotin was then added to the protein solution at a 3:1 or 5:1 molar ratio. The reaction was performed for 2 hours at room temperature and the protein sample was analyzed by MALDI mass spectrometry to determine the number of biotin molecules added per molecule of protein. Excess NHS-LC-LC-Biotin was removed using a 3 kDa spin filter. The target proteins for which data is shown in this example were pooled human transferrin (Sigma) and purified bovine ubiquitin.

A Biacore A-100 Surface Plasmon Resonance (SPR) system was used to measure the binding response of each peptide to several different target proteins immobilized on a gold surface. The A-100 has four different flow cells and within each flow cell are five addressable spots. Therefore four different proteins and a negative control reference can be used per flow cell. Depending on the purpose of the assay, up to 16 different target proteins can be immobilized on a single SPR chip. The instrument is equipped to evaluate up to 10 384-well plates unattended and can process approximately four 384-well plates per day. Sensorgrams are collected from each immobilized protein, so a binding profile for each analyte versus each of the protein targets is generated for each injection. Target proteins were immobilized using a biotin capture approach in which a CM5 chip was activated using standard amine coupling chemistry and Neutravidin was covalently coupled to the chip. Each biotinylated protein was injected over a single spot and the amount of protein captured was measured. In this manner four proteins were immobilized per flow cell. In this example, the same four proteins were captured in all four flow cells for this experiment.

A 384-well plate of peptides was prepared by adding 5 μL of each peptide to 45 μL of SPR running buffer. A second dilution was performed by adding 10 pt of the new peptide solution to 90 μL of SPR running buffer in a second 384-well plate. This reduces the peptide concentrations to a range from ^{˜}100 to 10 M.

A binding assay was performed in which each peptide was injected across the surface for 60 seconds, to monitor the association phase, and then buffer was flowed across the surface for 60 seconds to measure the dissociation phase. Each sensorgram contains information on the maximum binding of the peptide to each protein and can also contain information about the association and dissociation rates for each peptide-protein complex. The surface was periodically washed with 0.1 M glycine at pH 2.5 to remove any peptide that did not dissociate.

Data analysis was performed using the A-100 Evaluation software package that analyzes and filters the data using a variety of measures of quality control for each sensorgram. The filtered data was then reference subtracted and adjusted for the molecular weight differences between peptides to normalize the response across the run. Plots were generated that compare the binding response from each peptide to each protein. In this manner a relative measure of the specificity of binding for each peptide was determined.

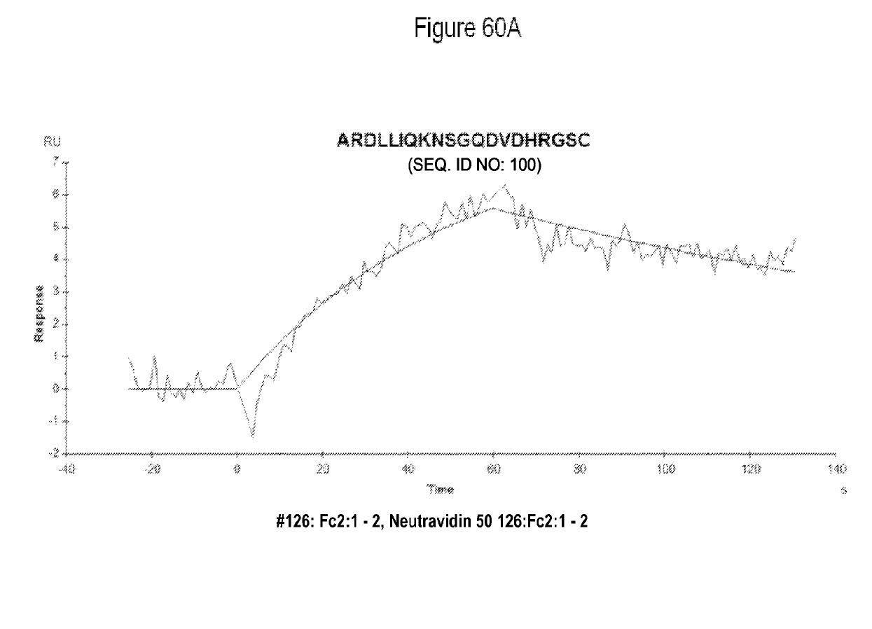

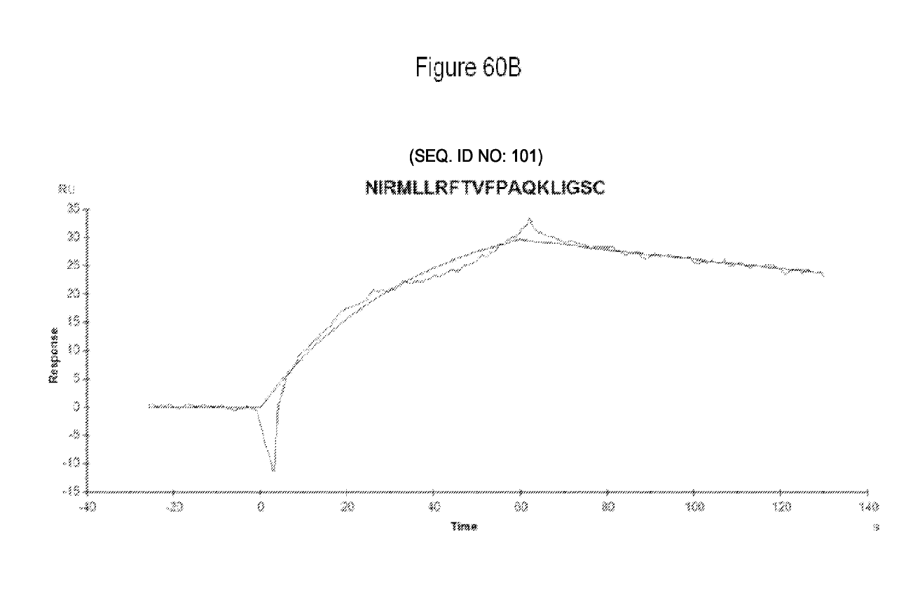

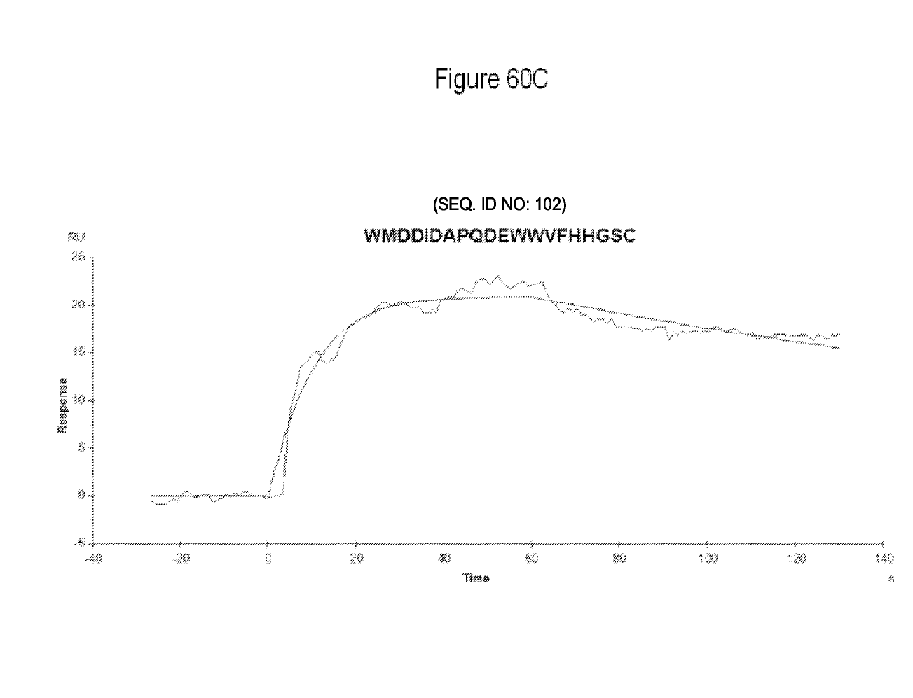

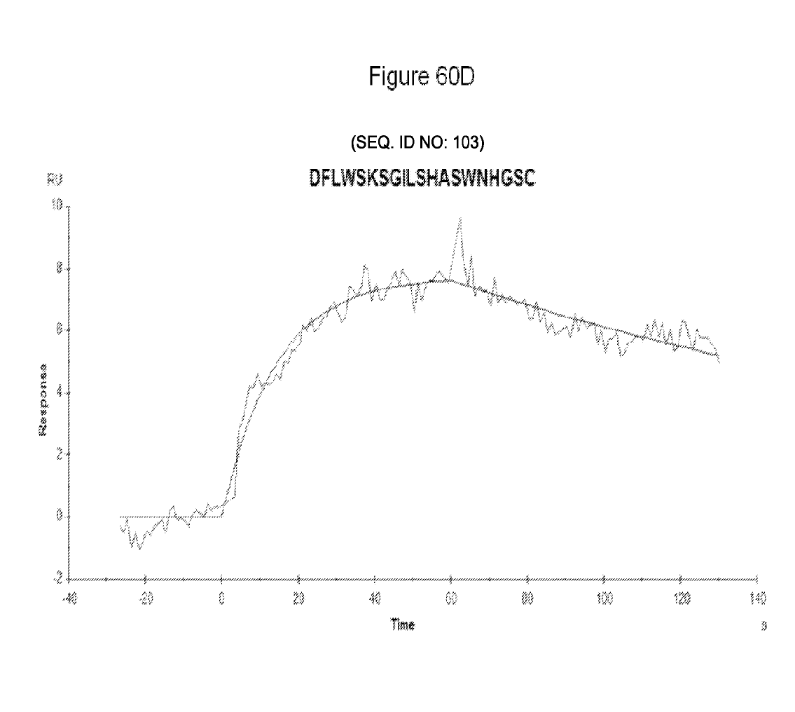

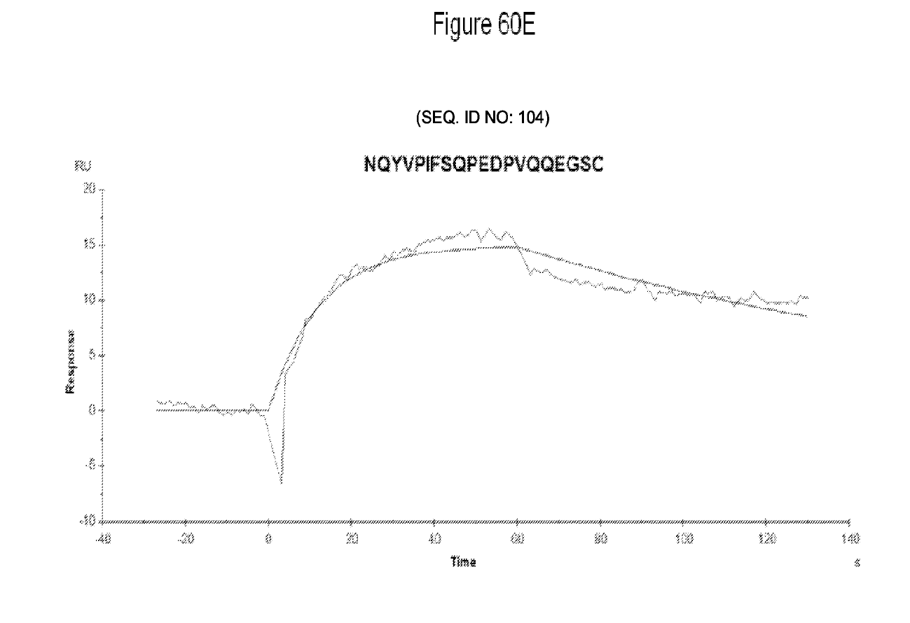

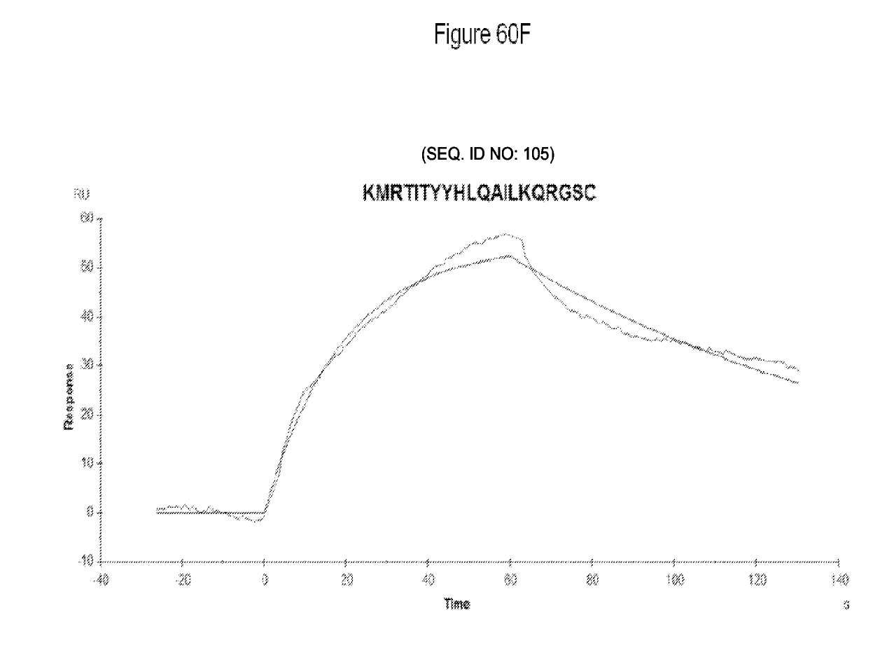

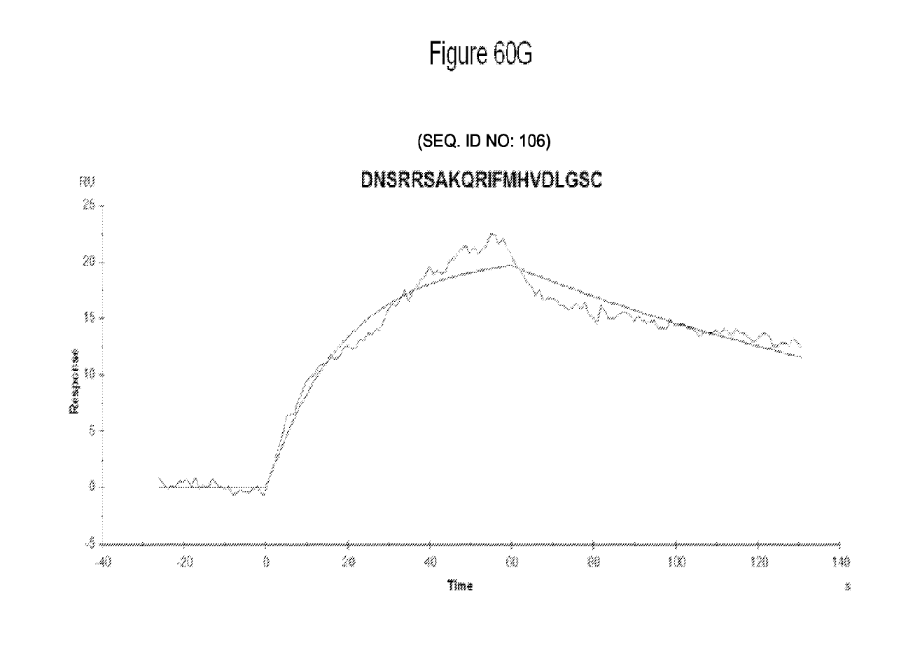

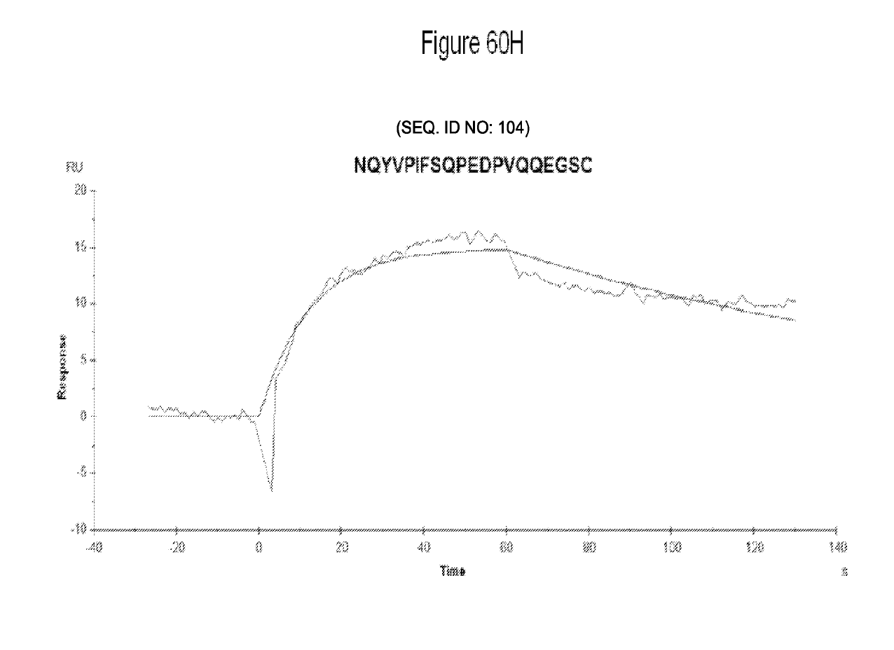

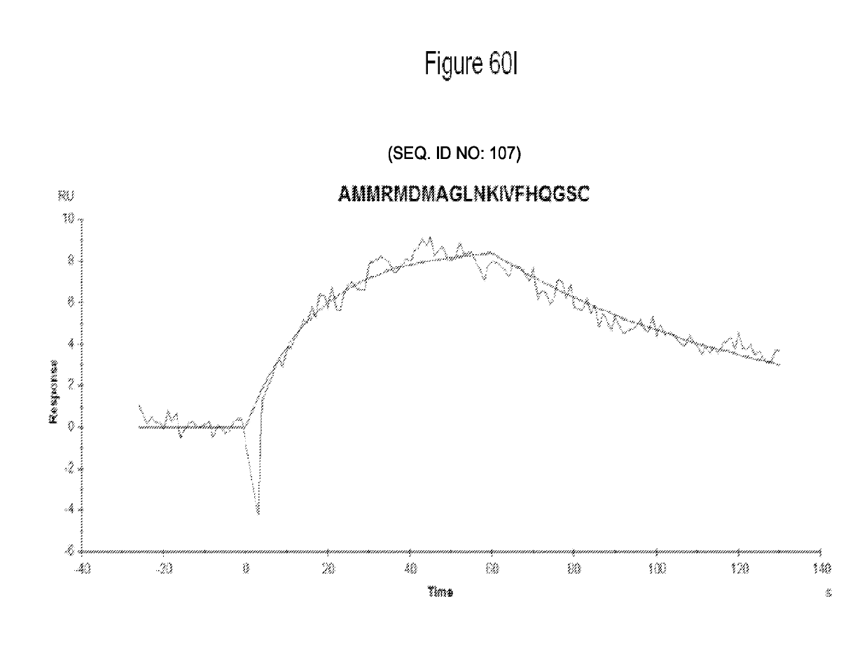

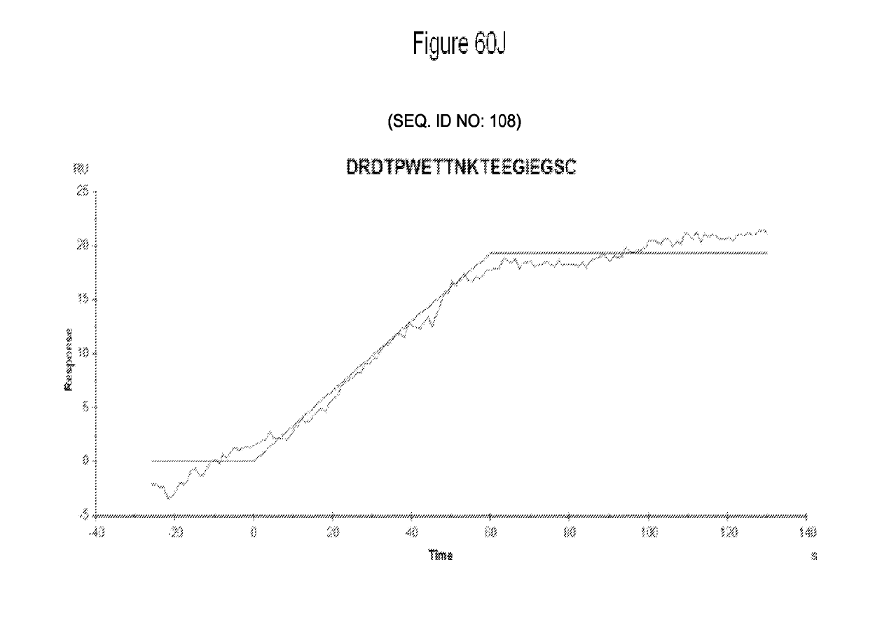

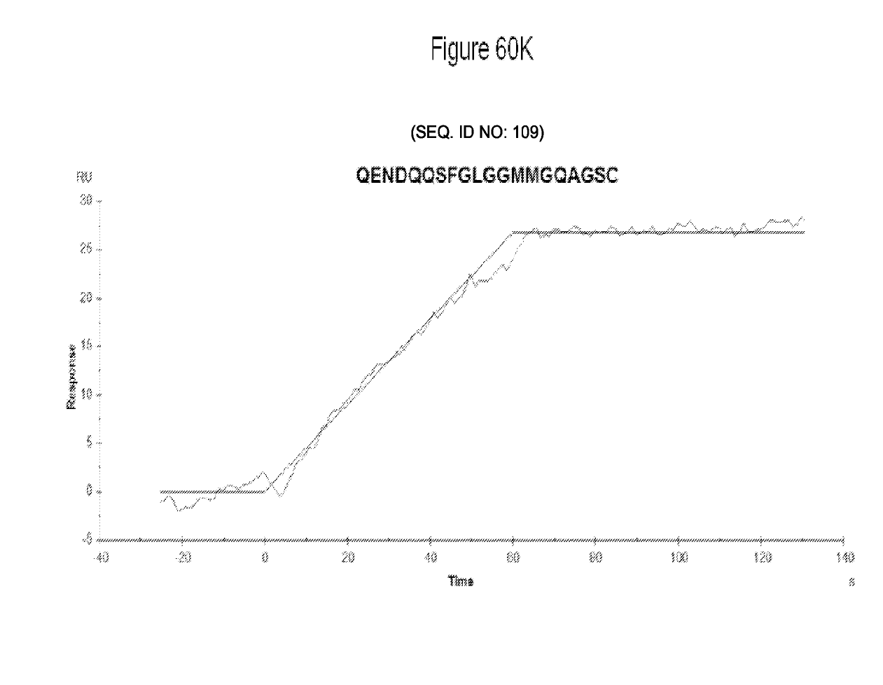

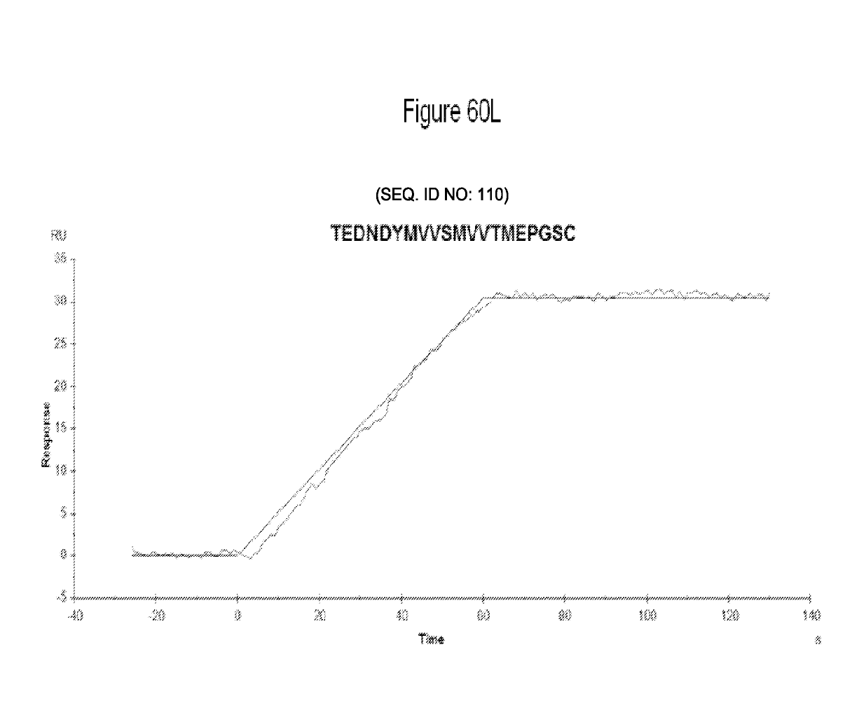

FIG. 60 shows sensorgrams for the binding of 12 selected peptides to transferrin, indicating dissociation rates in the range of 10⁻² to 10⁻³ sec⁻¹. Sequences of the peptides corresponding to each sensorgram are shown in Table 12.

TABLE 12
Sequences of transferrin binding peptides

| | Figure | Sequence |
|---|---|---|
| TRF101 | 60A | ARDLLIQKNSGQDVDHRGSC |
| | 60B | NIRMLLRFTVFPAQKLIGSC |
| | 60C | WMDDIDAPQDEWWVFHHGSC |
| | 60D | DFLWSKSGILSHASWNHGSC |
| TRF102 | 60E | NQYVPIFSQPEDPVQQEGSC |
| | 60F | KMRTITYYHLQAILKQRGSC |
| | 60G | DNSRRSAKQRIFMHVDLGSC |
| | 60H | NQYVPIFSQPEDPVQQEGSC |
| | 601 | AMMRMDMAGLNKIVFHQGSC |
| | 60J | DRDTPWETTNKTEEGIEGSC |
| | 60K | QENDQQSFGLGGMMGQAGSC |
| | 60L | TEDNDYMVVSMVVTMEPGSC |

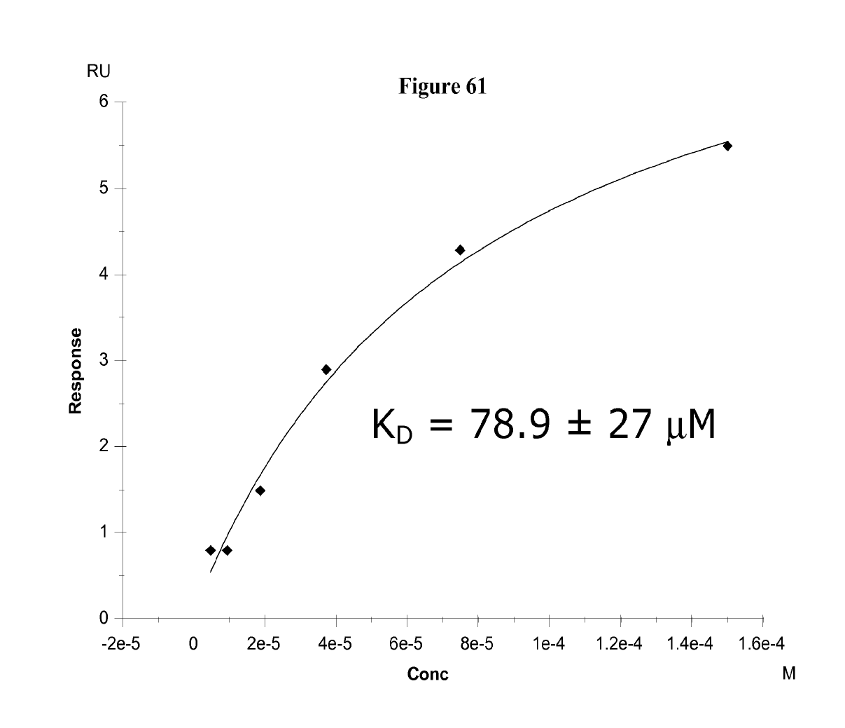

Two of the peptides (TRF101 and TRF102, see Table 12) that showed preferential binding for transferrin and exhibited dissociation rates in the range of 10⁻² to 10⁻³ sec⁻¹ as shown in FIGS. 60A-L, and four peptides (data not shown) similarly identified from the SPR assays for binding to ubiquitin, were selected for further study and verification of results. These peptides were on a Symphony Peptide Synthesizer (Protein Technologies, Tucson, Ariz.) and then purified using an Agilent 1100 HPLC system. Each purified peptide was then checked by MALDI mass spectrometry to verify the correct molecular weight, and lyophilized to dryness. The purified candidate peptides were then re-screened against transferring and ubiquitin on the A-100 at several different concentrations to measure equilibrium dissociation constants (K_{D}) for each peptide. One of the six peptide sequences (TRF101, see Table 12) was found to exhibit kinetic properties similar to those observed in the original (unpurified peptide) data, and showed a K_{D}=78.9±27 μM with respect to transferrin as shown in FIG. 61. The other five sequences, when evaluated in purified form, failed to exhibit the previously observed binding characteristics. MALDI-MS characterization of the (unpurified) peptide samples originally screened showed that the TRF101 sample was relatively free of impurities, while the other five unpurified samples showed a number of off-target peaks.

### Example 27 Specificity Screening by SPR Assay

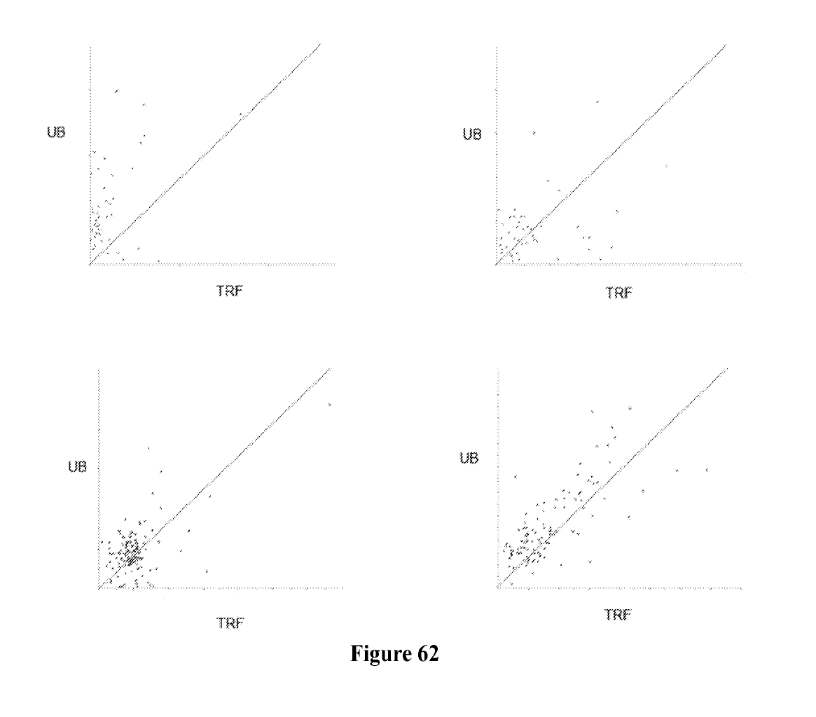

This example demonstrates the use of the high throughput SPR assay described in Example 25 to evaluate the specificity of peptides by comparing their binding properties with respect to a target of interest with their binding properties with respect to one or more other targets. Two 384 well plates of peptides were prepared and screened by A-100 SPR assay against transferrin and ubiquitin as described in Example 25. The binding response of each peptide against each target was determined; plots of these values are shown in FIG. 62. (192 peptides were screened on each of the four flow cells; each plot shows results from one flow cell.) Peptides that lie along the diagonal have poor specificity, while those close to either axis show preferential binding for one protein or the other, and can be selected for further evaluation.

### Example 28 Chromatographic Affinity Screening of Candidate Synbodies

This example demonstrates the identification of synbody or other ligand species in a library that are capable of preferentially binding a target of interest, by using the target of interest to retain the preferentially binding species in a chromatographic assay and identifying the bound species by mass spectrographic evaluation.

The target proteins, Transferrin (TRF) and Tumor Necrosis Factor-alpha (TNF-α), were each covalently attached to pipette tips (one protein per pipette tip) containing carboxymethyl dextran matrix (Intrinsic Bioprobes, Tempe, Ariz.) using standard amine coupling chemistry. The unmodified tips were first washed with 0.5 M HCl followed by acetone. Each tip was activated using a 50 mg/mL solution of 1,1-carbonyldiimide (CDl) in N-methyl-pyrolidone (NMP). Each tip was washed with NMP to remove excess CDl. Each protein was prepared as a 50 μg/mL solution in 100 mM sodium acetate pH 5.0 and cycled through a CDl activated tip for 30 minutes. Un-reacted CDl in the tip was then quenched with the addition of 1.5 M ethanolamine pH 8.5 and then washed extensively with HBS—N buffer. The protein-coupled tips were then stored in HBS—N buffer at 4° C. Negative control tips were prepared in the same manner except that no protein was added to the sodium acetate solution during the protein coupling step.

A library of 14 candidate synbodies (Table 9) was prepared by making 12 μM stock solutions in 1× phosphate buffered saline (PBS) of each HPLC purified synbody and 50 μL of each stock solution was added to 600 μL of E. Coli Lysate that had been treated with a protease inhibitor. Thus the final concentration of each synbody was 500 nM. (The structures and peptide affinity element sequences of the synbodies shown in Table 13 are as described in Example 19 and shown in Tables 9 and 10.)

TABLE 13
Synbody library for chromatographic screening

| No. | Synbody | MW (avg) |
|---|---|---|
| 1 | TRF24-TRF 19-KC | 4642.1 |
| 2 | TRF26-TRF19-KC | 4754.1 |
| 3 | TRF21-TRF22-KC | 4725.3 |
| 4 | TRF26-TRF24-KA | 4774.2 |
| 5 | TRF24-TRF25-KA | 4615.2 |
| 6 | TNF2-TNF3-KC-stBu | 4477.18 |
| 7 | TNF1-TNF4- KC-stBu | 4526.48 |
| 8 | TNF2-TNF5- KC-stBu | 4731.48 |
| 9 | TNF1-TNF10- KC-stBu | 4630.38 |
| 10 | TRF23-TRF23- KC-stBu | 4736.4 |
| 11 | mTNF26-TRF23-KC | 4803.5 |
| 12 | TRF26-TRF23-KC | 4812.5 |
| 13 | TRF26-TRF21-KA | 4868.4 |
| 14 | TRF24-TRF20-KA | 4805.5 |

A negative control pipette tip (blank tip), a TRF tip, and a TNF-α tip, were washed with 0.1% sodium dodecyl sulfate (SDS) to remove any non-covalently bound protein and then washed with HBS buffer. The tips were then incubated for 15 minutes in 150 μL of the synbody library. Each tip was then washed 5 times in 150 μL of HBS—N. This step was then repeated and each tip was washed 5 times in 150 μL of 0.25 M NaCl. Each tip was then washed 5 times in 150 μL of Milli-Q water and this step was repeated. The tips were then eluted with 150 μL of a saturated solution of α-cyano-4-hydroxycinnamic acid prepared in 33% acetonitrile and 0.7% trifluoroacetic acid (TFA).

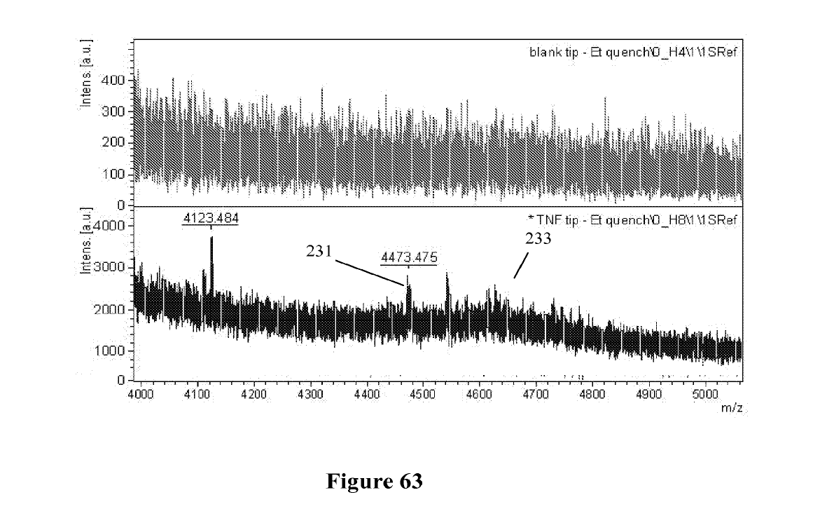

Each elution sample was spotted onto a MALDI plate and analyzed in reflectron mode on a Bruker Daltonics UltraFlex III TOF/TOF MALDI Mass Spectrometer. FIG. 63 shows MALDI spectra for the elutions from each of the three tips. The spectrum from the TNF-α tip elution showed a peak 231 at 4473.475 and a peak 233 at 4630.4, corresponding to synbodies TNF2-TNF3-KC-stBu and TNF1-TNF10-KC-stBu (see Table 13).

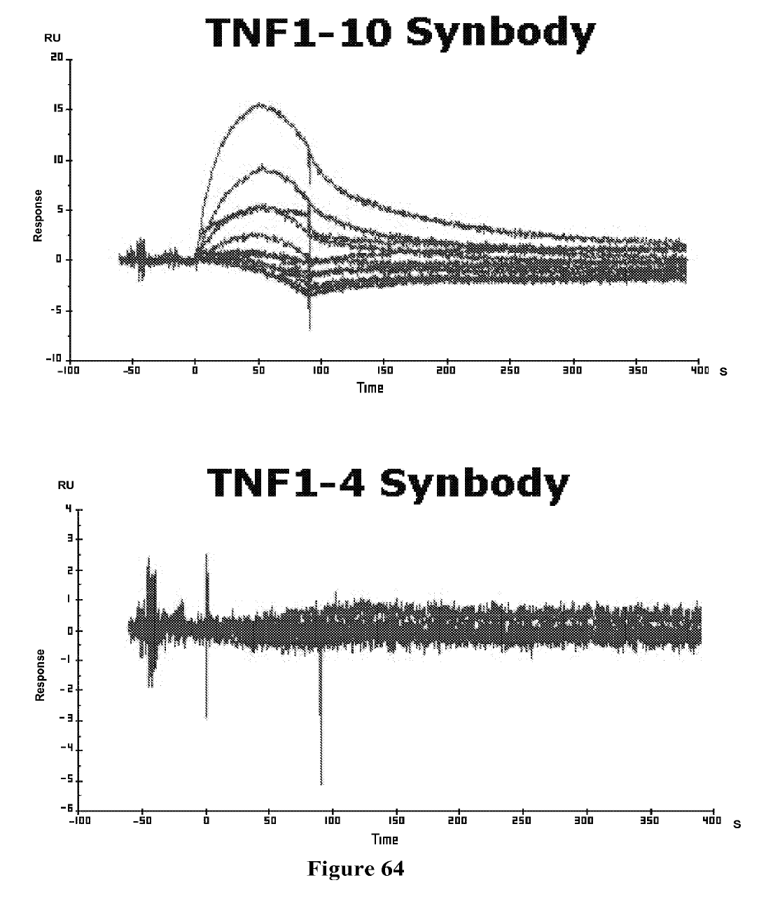

Candidate TNF-α binding synbodies were screened by surface plasmon resonance (SPR) on a Biacore T-100 SPR instrument to verify binding for TNF-α. A CM5 chip was activated using standard amine coupling chemistry and TNF-α was immobilized. Each synbody was prepared in HBS—N buffer with excess carboxymethyl dextran added to the running buffer to minimize non-specific binding to the chip surface. A concentration series of each synbody was prepared where the concentrations ranged from 1.25 μM to 9.8 nM. FIG. 64 shows a comparison of synbody TNF1-TNF10-KC-stBu to synbody TNF1-TNF4-KC-stBu (for which no peak was observed in the MALDI spectrum from the TNF-α tip elution). The sensorgrams (FIG. 64) show relatively strong binding kinetics for synbody TNF1-TNF10-KC-stBu and no binding for synbody TNF1-TNF4-KC-stBu.

### Example 28A Linear Optimization

Initial screening of a peptide library of 10,000 peptides against TNF-α identified 171 sequences as potential leads with affinity for TNF-α. The significant number of potential lead sequences allowed for the application of more stringent lead criteria. First, the 171 potential anti-TNF-α lead peptides were screened for acceptable sample purity using MALDI-MS, peptide leads with a sample purity less than 70% were removed from the list of potential leads. Next, the remaining potential lead peptides were further filtered by comparing TNF-α SPR response to the response from four unrelated proteins ((AKT1, Neutravidin, Transferrin, and Ubiquitin) on the SPR chip as well. Peptides that showed significant response with proteins other than TNF-α were removed from the list of potential leads. Finally, the remaining 10 potential anti-TNF-α leads were subject to further validation with a second SPR affinity assay across a series of peptide concentrations. From this, the lead peptide sequence FERDPLMMPWSFLQSRQGSC (referred to as TNF1) was chosen based on its dissociation constant (K_{d}) of 160±19 μM for TNF-α; the minimal binding observed to other protein targets; and its relative solubility as suggested by a GRAVY (Kyte, Journal of Molecular Biology 157(1):105-132, 1982) score of −0.52. Although TNF1 did not have the highest TNF-α SPR binding response out of all 10⁴ peptides in the initial library, the combination of favorable properties made it a solid lead candidate for input into the AMPLI algorithm.

Scanning Mutagenesis of the TNF1 Lead Peptide. After lead identification, the next step in the AMPLI algorithm is characterization of point mutations in the lead heteropolymer. Using short peptides makes it chemically feasible to synthesize a significant fraction of the point-mutant space, which can then be screened for enhanced point mutations. For example, all possible point mutations in the 17 randomized positions using all 20 natural amino acids could be synthesized and screened within a single 384-well plate (323 total point-mutants). However, libraries containing all 20 natural amino acids are not required for affinity optimization of protein-protein interactions. A library of TNF1 point-mutants containing all substitutions of the amino acid set {Y, A, D, S, K, N, V, W} in each of the 17 randomized positions (132 unique point-mutants) was synthesized. Tyrosine (Y), alanine (A), aspartic acid (D) and serine (S) were selected because of their effectiveness in producing high affinity interactions when substituted into the complementary-determining regions (CDRs) of synthetic antibodies (Felouse, Proceedings of the National Academy of Sciences 101(34):12467, 2004), lysine (K) was selected to balance the charge in the substitution set, asparagine (N), valine (V) and tryptophan (W) were selected to span the hydropathicity range (Kyte J & Doolittle R F, Journal of Molecular Biology 157(1):105-132, 1982). This set of 132 point-mutants was synthesized and screened for relative TNF-α binding response using SPR at 50 μM peptide concentration, which is approximately 3-fold below the K_{d} of TNF1. This concentration was used to increase the high-end dynamic range for quantifying enhancing point mutations at the expense of low-end dynamic range for quantifying detrimental point mutations.

Point-mutant libraries were prepared in 96-well stock plates From the stock plate, peptides were diluted to 50 μM concentration in Biacore HBS-EP buffer (GE Healthcare, Piscataway, N.J.) containing 1 mg/ml carboxymethyl-dextran (Sigma-Aldrich, St. Louis, Mo.) to reduce non-specific binding to the CM-5 SPR chip surface. TNF-αt was captured on a CM-5 chip surface at different capture levels on spots 1, 2, 4, and 5 across all four flow cells corresponding to a 40-200 RU range of predicted Rₘₐₓ binding responses. Spot 3 contained only immobilized neutravidin and served as a reference spot.

Using the prepared 96-well plates and Biacore A100 SPR instrument, four peptides were flowed separately, in parallel, through the four flow cells over all 4 TNF-α spots and the neutravidin reference spot, with a 60 second association phase and 300 second dissociation phase. SPR sensorgrams were recorded for each peptide response with all 4 TNF-α spots and the neutravidin reference spot across the four flow cells on the SPR chip. Surface regeneration was performed after every 12 injections in each flow cell with Biacore Glycine 2.5 regeneration solution (GE Healthcare, Piscataway, N.J.). Point-mutant reference subtracted, peptide molecular weight adjusted, responses at the late binding region of the sensorgram (a few seconds before dissociation) were compared to the response of the TNF1 lead

Several enhanced point-mutants from the point-mutant screen were synthesized and purified using standard solid-phase FMOC synthesis and HPLC purification. Purified point-mutant affinities were measured on the Biacore A100 using SPR equilibrium binding response across a series of peptide concentrations on an SPR chip with TNF-α captured as described above.

Enhanced point mutations were combined into several multiple mutant sequences. These sequences were synthesized and purified using standard solid-phase FMOC synthesis and HPLC purification. Purified multiple-mutant affinities were measured on the Biacore A100 using SPR equilibrium binding response across a series of peptide concentrations on an SPR chip with TNF-α captured as described above at four different capture levels giving a predicted binding max (Rₘₐₓ) range of ^{˜}40-120 RU. Responses were normalized to the predicted maximum binding response so results from different TNF-α capture levels can be directly compared.

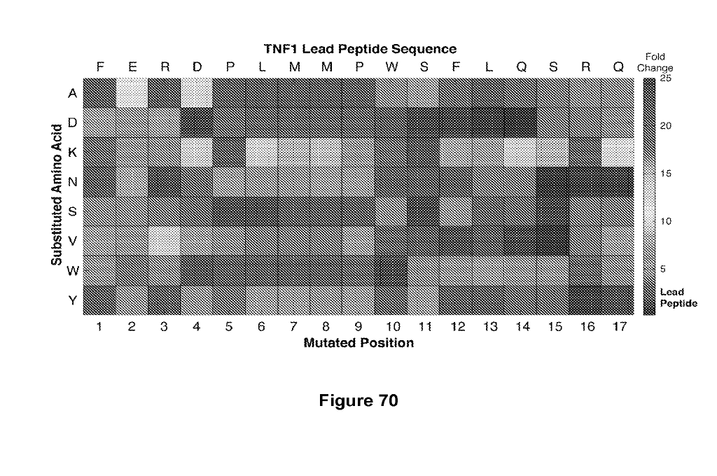

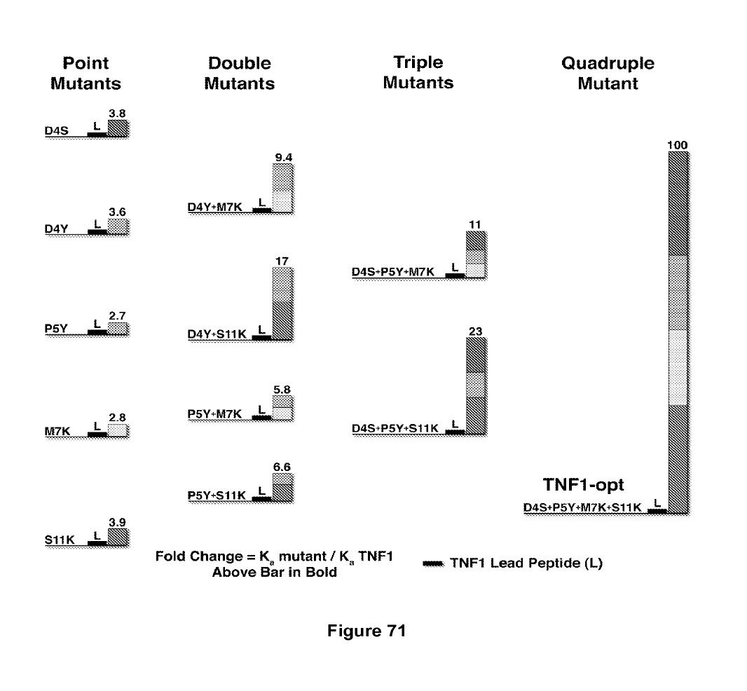

The effect of different point mutations can be displayed as a heat matrix (FIG. 70), in which columns represent different positions in the peptide and rows different substitutions, and the squares in the matrix are occupied by different colors from a color scale correlated with effect of the mutation on peptide relative to the amino acid in the same position of the lead peptide. Both positive and negative fold reductions can be displayed on a heat chart. The heat chart provides a simple visualization of the positions and types of substitution having the greatest influence on binding affinity of the lead peptide. Several point mutations at 9 unique positions in the sequence conferred better than 10-fold SPR binding response relative to TNF1, with all peptides at 50 μM concentration. Negative charge in the lead peptide may be an inhibitory factor for TNF-α binding because almost any mutation in position 2 (E) or 4 (D) enhances affinity, including alanine, which is usually considered to be a neutral mutation in alanine scanning mutagenesis (Cunningham B C & Wells J A, Science 244(4908):1081-1085, 1989). Further support for an inhibitory effect from negative charge comes from the fact that substituting lysine in several positions enhances affinity, suggesting that the optimized peptide should have a higher pl than TNF1. In addition to an inhibitory effect by negative charge, the heat map indicates that tyrosine is a particularly favorable substitution in the N-terminal half of the peptide. Tyrosine is the most favorable uncharged substitution in the point-mutant library, with 7 out of the 17 mutated positions substituted with tyrosine producing better than 5-fold enhancement in SPR response at 50 μM peptide concentration.

Several mutant sequences (D4S, D4Y, P5Y, M7K, S11K point-mutants) were selected for further characterization. Specifically, these point-mutants were selected because they showed a ≥15-fold enhancement in SPR binding response relative to TNF1 as well as low non-specific binding to the neutravidin coated reference flow-cell on the SPR chip when screened at 50 μM concentration. TNF-α affinities (K_{d}) for the D4S, D4Y, P5Y, M7K and S11K point-mutant sequences were determined by SPR (Table 13A).

Affinity Prediction of an Optimized Mutant. Component binding energy contribution of a point mutation can be calculated by subtracting the binding energy of a point-mutant sequence from the binding energy of the lead sequence. Using this formula, component binding energy contributions for the D4S, D4Y, P5Y, M7K and S11K mutations were determined and are given in Table 13A. From these individual contributions and the assumption of energetic additivity, predictions can be made on the binding energies of mutant sequences containing multiple substitutions.

The goal of this study was to produce a peptide approaching a TNF-α affinity (K_{d}) of 1 μM, an approximate 100-fold improvement over the TNF1 lead peptide. Based on the predictions from energetic additivity, a combination of 4 point mutations would be required to reach a K_{d}˜1 μM starting from a lead peptide K_{d}=160 μM. As a result of these predictions, the D4S+P5Y+M7K+S11K quadruple mutant, referred to as TNF1-opt, was selected as the optimized sequence. The D4S substitution was selected over the D4Y substitution because a tyrosine substitution in position 5 (P5Y) also showed significant improvement, which suggests a proximity effect for a tyrosine substitution in this region of the peptide. In other words, tyrosine can produce an affinity enhancement in either position 4 or 5 but potentially not both positions. Therefore, the serine substitution was used in position 4 (D4S) and the tyrosine substitution in position 5 (P5Y). In addition to the TNF1-opt quadruple mutant, several intermediate mutants (double, triple mutants) were characterized to compare predicted affinities to observed TNF-α affinities.

Affinity Characterization of Double, Triple and Quadruple Mutants. Four double (D4Y+M7K, D4Y+S11K, P5Y+M7K, P5Y+S11K), two triple (D4S+P5Y+M7K, D4S+P5Y+S11K) and one quadruple (D4S+P5Y+M7K+S11K) mutant sequence were synthesized and characterized with SPR. In all cases, an improvement in TNF-α affinity was observed when an additional enhancing substitution was added to the sequence. Double mutants were better than the corresponding single mutants, triple mutants were better than the corresponding single/double mutants and the quadruple mutant was better than the corresponding single/double/triple mutants (FIG. 71, Table 13B). The optimized quadruple mutant sequence (TNF1-opt) has a K_{d}=1.6±0.3 μM determined by SPR. Further validation of TNF1-opt affinity was done using fluorescence anisotropy, which gave a K_{d}=1.1±0.2 μM, in agreement with the affinity determined by SPR.

Kinetic fits of the TNF1 and TNF1-opt sensorgrams indicate that TNF1-opt has approximately an order of magnitude or better improvement in both on-rate (kₒₙ), and off-rate (k_{off}), when compared to TNF1. The significantly slower off-rate for TNF1-opt (TNF1 k_{off}=1.6±0.5 s⁻¹, TNF1−opt kₒₙ=0.2±0.02 s⁻¹) is visually apparent. In addition, a K_{d}=0.7±0.02 μM determined from kinetic fits of several TNF1-opt sensorgrams, is comparable to the affinities determined from a concentration series of TNF1-opt equilibrium SPR binding responses and fluorescence anisotropy.

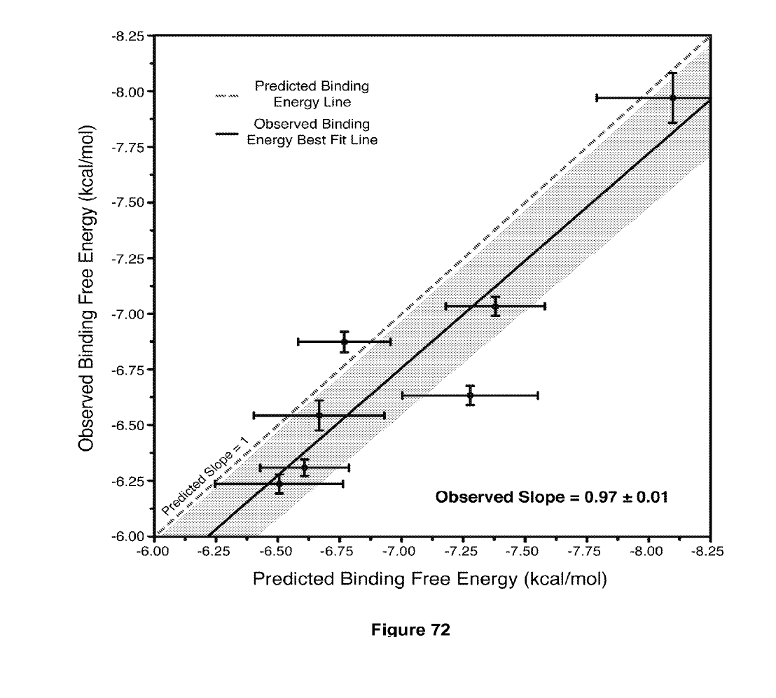

Comparison of Observed Affinities to Predicted Affinities. The observed TNF1-opt affinity (Observed K_{d}=1.6±0.3 μM) is within the affinity range predicted from energetic additivity of component mutations (Predicted K_{d}=0.7−1.9 μM) (Table 13B). This suggests that the affinity enhancements contributed by each of the four point mutations in the optimized peptide are acting nearly independently of each other (Wells, Biochemistry 29(37):8509-8517 (1990)). If the combinations of point mutations are acting additively, then a plot of the observed vs. predicted affinity should produce a slope of 1 (FIG. 72). The slope of the best-fit line for the mutants tested is 0.97±0.01, indicating that the binding energy contributions of point mutations are significantly additive when these individual mutations are combined in a multiple mutant sequence. The mutant sequence that deviates most from the predicted value is the D4S+P5Y+M7K triple mutant, which is possibly caused by the accumulation of three mutations in a proximal region of the peptide sequence that produce nearest neighbor interactions (Pál, J Biol Chem 281(31):22378-22385, 2006). Combining the S11K mutation, a three-residue separation from the nearest mutation, with these three proximal mutations appears to contribute purely additively, further supporting a potential nearest neighbor interaction between mutations in close proximity, those separated by one residue or less.

Further evidence for mutational additivity is apparent when binding energies of double mutants are compared to triple mutants and double/triple mutants are compared to the quadruple mutant. The difference in observed binding energy between the P5Y+S11K and D4S+P5Y+S11K mutants is −0.72±0.06 kcal/mol, in agreement with the calculated D4S component contribution of −0.77±0.08 kcal/mol. Furthermore, the observed binding energy differences between the P5Y+M7K, P5Y+S11K, D4S+P5Y+S11K mutants and the D4S+P5Y+M7K+S11K quadruple mutant are −1.73±0.12, −1.66±0.12, and −0.94±0.12 kcal/mol respectively, in agreement with the predicted differences calculated from the component contributions.

Molecular Dynamics Simulation of TNF1 and TNF1-opt Peptide Structure. One precondition of mutational energetic additivity is that mutated residues do not structurally overlap (Wells J. Biochemistry 29(37):8509-8517, 1990). Molecular dynamics (MD) simulations were performed to elucidate potential structure or structural tendencies in TNF1 and the effect of mutations on possible conformations.

For each sequence, 100 molecular dynamics trajectories, each of 10 ns in length, were generated using AMBER v. 9 ((University of California, San Francisco, 2006). Each trajectory was begun from a conformation generated by assigning random values to all rotatable bonds, then randomly rotating bonds to eliminate any steric collisions, then minimizing. Trajectories were run using a 2 fs time step, with bonds to hydrogens constrained with SHAKE (Ryckaert, Journal of Computational Physics, 1997). AmberParm96 force field parameters, and the GB/SA implicit solvent model, with parameter settings SALTCON=0.15, SURFTEN=0.003, and EXTDIEL=75 to simulate the salt, surfactant, and organic content of the SPR running buffer used for affinity measurements. Temperature for all runs was maintained at 300K via the Andersen thermostat (Andrea, The Journal of Chemical Physics, 1983) applied at 4 ps intervals. Conformations were sampled at 200 ps intervals after discarding the first 5 ns of each trajectory, yielding a total of 2600 samples for each sequence. A 2600×2600 pairwise distance matrix was computed reflecting average RMS distances following structural alignment of the backbone atoms of residues 4 through 11, as computed for each pair of conformations using Pymol's (DeLano, DeLano Scientific, Palo Alto, Calif., USA, 2008) “fit” function. Clustering was performed by repeatedly identifying the largest subset of samples having RMS distances within a 1 Å threshold, and removing the cluster so identified from the distance matrix. The graphical representations were produced using Pymol.

In these simulations, 2600 sampled conformations were generated from a total of 1 μs of MD trajectories, each for TNF1 and for TNF1-opt. Based on an analysis of the distribution of conformations, both peptides are loosely structured, with three main characteristics: 1) Both peptides have a tendency to form a loose and fluid hairpin, with the exact locus of the turn shifting among various positions in the region of residues 9-14, consistent with a negative band at 234 nm in their circular dichroism (CD) spectra (Fasman, Circular Dichroism and the Conformational Analysis of Biomolecules (Plenum Press, New York, 1996); Rana, Chem Commun (Camb) (2):207-209 (2005); Roy, Biopolymers 80(6):787-799 (2005)). The mutated region of TNF1-opt, residues 4 through 11, substantially favored an extended conformation (though by no means rigid) in both TNF1 and TNF1-opt (FIG. 73). Otherwise, the structures of both peptides were quite flexible and variable overall.

Dominant conformations for both TNF1 and TNF1-opt were defined in each case by the largest cluster of backbone structural alignments within 1□ pair-wise root-mean-square deviation (RMSD) of each other. This analysis shows that in the mutated region (residues 4-11), the dominant conformation comprised about 15% of the total resulting conformations of TNF1 but only about 3% of the total resulting conformations of TNF1-opt (FIG. 73). The broader distribution of conformations observed in TNF1-opt may increase the probability of a productive binding event, such as in a conformational selection binding model

(Lange, Science 320(5882):1471-1475, 2008), where the dominant conformation of TNF1 is not the conformation that binds TNF-α.

Although MD simulations suggest less rigidity in the TNF1-opt mutated region, these simulations along with CD spectroscopy suggest that any tendency towards forming a hairpin present in TNF1 is retained in TNF1-opt. Similar structural tendencies in TNF1 and TNF1-opt imply that the four mutations in TNF1-opt are not significantly structurally connected and therefore do not dramatically alter any structure or structural tendencies present in the lead, which supports the general hypothesis that relatively unstructured heteropolymers serve as good scaffolds for affinity optimization by additive mutagenesis.

TNF1-opt has one of the highest affinity anti-TNF-α peptides reported to date (Chirinos, J Immunol 161(10):5621-5626, (1998); Takasaki, Nat Biotechnol 15(12):1266-1270, (1997)) and has comparable or even slightly better affinity than a recently reported TNF-α small-molecule ligand (He., Science 310(5750):1022-1025, 2005). The AMPLI algorithm produced a peptide in only two rounds of limited chemical synthesis with better affinity than a peptide selected after three rounds of phage selection (Zhang., Biochemical and Biophysical Research Communications, 2003), even though the phage selection was done from a library of ^{˜}10⁸ peptides. Unlike a selection strategy, the AMPLI algorithm allows prediction of the potential affinities that can be achieved from the lead heteropolymer and the point-mutants that are screened.

One distinct advantage of a chemical approach to optimization is that, with judicious combination of point mutations, specific desirable properties of the final affinity reagent can be maintained or improved throughout the optimization process. This is a powerful feature of the AMPLI algorithm that is difficult or impossible to do with alternative selection strategies and adds to the utility of this algorithm if the final heteropolymer is to be used as a therapeutic or diagnostic reagent.

Another advantage of the purely chemical approach employed by the AMPLI algorithm is that it is amenable to high-throughput and automation. Because this is a predictive algorithm, it can be implemented by software implementation that has the capability not only to combine the appropriate point mutations to reach a desired affinity range, but also the ability to control robotics for library synthesis and screening. As a result, this automated system can take a lead sequence as ‘input’ and ‘output’ an optimized sequence with predictable affinity.

TABLE 13A
TNF1 lead and point-mutant binding energies and dissociation
constants (K_{d}).

| | TNF1 | | | | | |
|---|---|---|---|---|---|---|
| Peptide | Lead | | | | | |
| Mutation | Peptide | D4S | D4Y | P5Y | M7K | S11K |
| Binding ΔG | −5.21 ± 0.07 | −5.98 ± 0.04 | −5.95 ± 0.06 | −5.79 ± 0.04 | −5.93 ± −.20 | −6.03 ± −.10 |
| (kcal/mol) | | | | | | |
| K_{d} (μM) | 160 ± 19 | 42 ± −2.4 | 44 ± 4.8 | 58 ± 3.4 | 57 ± 20 | 40 ± 7.2 |
| K_{d} Fold- | — | 3.8 ± 0.5 | 3.6 ± 0.6 | 2.7 ± 0.4 | 2.8 ± 1.0 | 3.9 ± 0.9 |
| Change | | | | | | |
| Relative to | | | | | | |
| Lead | | | | | | |
| Component | — | −0.77 ± 0.08 | −0.74 ± 0.10 | −0.58 ± 0.08 | −0.72 ± 0.22 | −0.82 ± 0.13 |
| Binding ΔG | | | | | | |
| Contribution | | | | | | |
| (kcal/mol) | | | | | | |

TABLE 13B
Observed and predicted dissociation constants and binding free
energies for double, triple and quadruple mutants.

| | Peptide | | | | | D4S + | D4S + P5Y + | D4S + P5Y + |
|---|---|---|---|---|---|---|---|---|
| | Mutations | D4Y + M7K | D4Y + S11K | P5Y + M7K | P5Y + S11K | P5Y + M7K | S1IK | M7K + S11K |
| Observed | Binding | −6.54 ± 0.07 | −6.67 ± 0.05 | −6.24 ± 0.04 | −6.63 ± 0.04 | −6.63 ± 0.04 | −7.03 + 0.04 | −7.97 ± −0.11 |
| | ΔG | | | | | | | |
| | (kcal/mol) | | | | | | | |
| | K_{d} (μM) | 17 ± 1.9 | 93 ± 0.7 | 27 ± 1.8 | 24 ± 1.4 | 14 ± 1.0 | 7.0 ± 0.5 | 1.6 ± 0.3 |
| | K_{d} Fold- | 9.4 + 1.6 | 17 + 2.5 | 5.8 + 0.8 | 6.6 + 0.9 | 11 + 1.6 | 231⁷⁻3.2 | 100 + 22 |
| | Change | | | | | | | |
| | Relative to | | | | | | | |
| | Lead | | | | | | | |
| Predicted | Binding | −6.66 ± 0.25 | −6.67 ± 0.18 | −6.51 ± 0.24 | −6.61 ± 0.17 | −7.28 ± 0.26 | −7.38 ± 0.19 | −81.0 ± 0.29 |
| | ΔG | | | | | | | |
| | (kcal/mol) | | | | | | | |
| | K_{d} (μM) | 20-8.5 | 15-8.0 | 25-11 | 19-11 | 7.0-3.0 | 53-2.8 | 1.9-0.7 |

### Example 29 Peptide Affinity Element Optimization by Evaluation of Synthetically Mutated Sequences

This example demonstrates that peptide binding elements with significantly improved target binding characteristics can be identified by screening a small number (<1000) of point-mutant variants of a lead peptide, selected according to any of the methods described in the preceding examples and having moderate or low affinity/specificity for a selected target, for optimized target affinity/specificity as compared to that of the lead peptide.

In general, variant peptide sequences may be designed so that the variant peptide differed in one or more amino acid positions when compared to the lead peptide. In each mutated position any chemically compatible residue can be substituted, including but not limited to natural and unnatural amino acids. Also, instead of a substitution at a particular position, variant peptides may be designed to incorporate point-deletions and point-insertions as compared to the lead peptide. These deletion/insertion variants may be particularly useful when structural models of the peptide-target complex are available and the structure suggests removal/addition of a particular residue would be more optimal. Once the point-mutant variants are screened for target affinity, an affinity/specificity profile can be generated that compares the effect of a particular point mutation to the original amino acid in the lead peptide. From this profile, specific point mutations can be combined into additional variants that differ in multiple positions (multinomial variants) relative to the lead peptide. The individual effects of the point mutations should have an additive effect in some (if not all) of the multinomial variants thereby producing peptide(s) with further improved affinity/specificity.

In this example, a small library of ^{˜}300 variant peptides was synthesized in 96-well format. Each variant had a single point mutation relative to the lead peptide sequence. The lead peptide (TRF26, see Table 6) was selected as a moderate-affinity binder of the target protein transferrin. The library of variant peptides contained all possible point mutations of the lead peptide using the following set of amino acids {M,A,V,P,L,I,G,W,Y,F,S,T,N,Q,K,R,H,D,E}.

Relative affinities/specificities of the lead peptide and point-mutants were characterized using SPR as follows:

Peptide sample preparation. Lyophilized peptides were individually diluted in 96-well plates to approximately equal concentration (1 mg/ml) in 1×PBST buffer pH 7.4. Peptide sample purity was determined by MALDI-MS analysis of the diluted peptide samples.

SPR gold substrate preparation. Gold substrates used for SPR analysis were first modified with a monolayer of cysteamine by immersing the substrate in a 10 mM cysteamine/EtOH solution for 1 hour, thereby exposing a layer of primary amines just above the gold surface. After addition of the monolayer, the gold substrates were rinsed extensively with EtOH then further modified by immersing in a solution of 2 mM Sulfo-SMCC/PBS pH 7.4 for 1 hour, thereby exposing a surface-bound maleimide which can be used to covalently couple peptides to the gold substrate via the C-terminal cysteine.

Peptide spotting on gold substrate. Diluted peptide samples were spotted on the modified gold substrate using a commercial robotic spotter in an array format. The array contained ^{˜}440 peptide spots (including replicates and blank reference spots), each spot having ˜200 um diameter. Spotted substrates were kept in a humidity chamber overnight to ensure complete reaction between the surface exposed maleimide and the C-terminal cysteine in the peptides. After ˜12-hours, the substrates were washed with PBST buffer to remove excess peptide not bound to the gold substrate. Finally, unreacted maleimide groups were quenched using a 2 mM β-mercaptoethanol/PBST solution thereby presenting a hydrophilic surface in regions not containing peptide.

Determination of target affinity using SPR. Gold substrates containing arrays of peptide variants and the lead peptide were loaded into a FlexChip SPR (Biacore) instrument. To ensure binding specificity, three injections of 0.2% BSA sample were flowed across the array using the FlexChip fluidics. The array was then washed with a continuous 1 mL/min flow of PBST buffer until the sensorgram reached a stable baseline. After reaching a baseline, the array was washed 2 additional minutes using PBST, then a 10 μM Transferrin/PBST sample was injected and continuously recycled over the array surface for 8 minutes. After the recycle the array was washed for 12 minutes with continuous 1 mL/min PBST flow for 10 minutes. Sensorgrams were continuously recorded during the 2 minute prewash (to ensure baseline stability), 8 minute Transferrin sample recycle and post sample recycle wash.

Quantification of relative target affinities. Sensorgram values were taken from the stability region, that is the region ˜10 seconds into the post sample recycle wash. Sensorgram values at this point should allow identification of peptides that have both high levels of target binding and off-rates slower than the lead peptide. The blank reference values were subtracted from the value obtained at the peptide spots and this data was processed using custom data processing software. Data processing included identification of the mutated position at a particular SPR array spot as well as signal normalization relative to the lead peptide (lead peptide=1), enhanced binders have positive values and reduced binders have negative values.

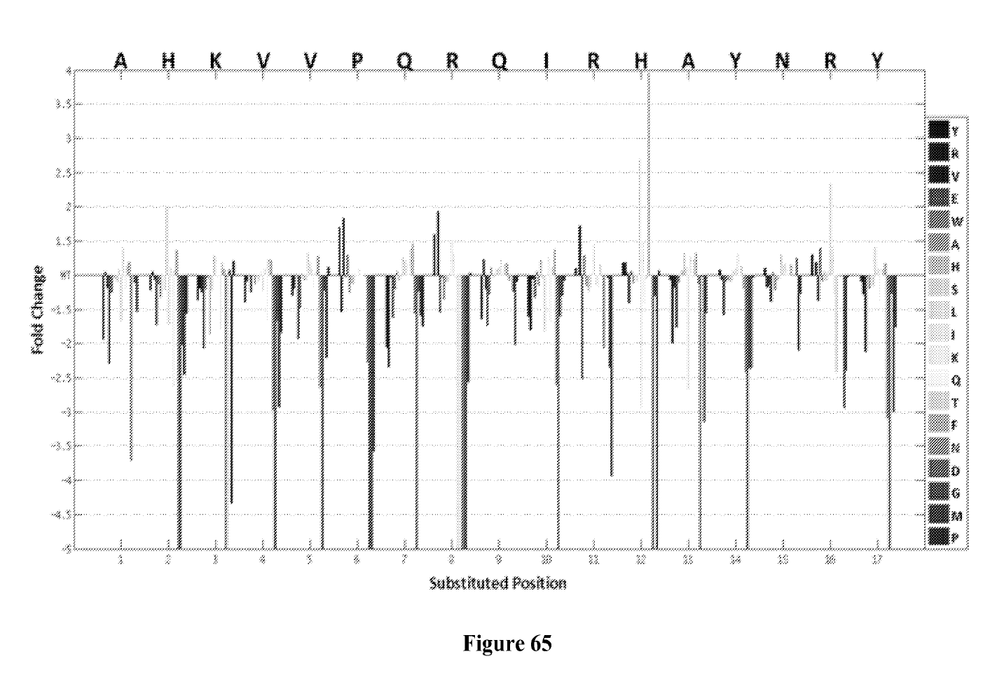

Graphical representation of the affinity profile for all variants is shown in FIG. 65. Several variants having improved affinity were identified; for example, several substitutions for the His residue at position 12 produced as much as 4 fold improvement in affinity.

Two TRF26 point-mutants (P6Y, H₁₂F) were selected for further affinity characterization. The P6Y and H₁₂F point-mutants have dissociation constants of 8.6±1.6 μM and 9.8±1.6 μM respectively. A substitution set of 19 amino acids in the TRF26 point-mutant screen did not produce proportionally more enhanced point mutations than the 8 amino acid TNF1 point-mutant screen, which suggests that a large amino acid substitution set is not required in a point-mutant screen to identify affinity enhancing point mutations. A TRF26 double mutant sequence containing the P6Y+H₁₂F mutations was synthesized and characterized. Assuming energetic additivity of point mutations, the P6Y+H₁₂F mutant should have a K_{d} in the range of 0.7-1.3 μM. The observed P6Y+H₁₂F mutant K_{d}=0.5±0.1 μM is in agreement with the affinity range predicted from energetic additivity of mutations.

TABLE 13C
Observed binding energies and dissociation constants
for the TRF26 lead peptide and point mutants selected
from the point mutant library screen.

Peptide Mutation

| | TRF26 Lead | | |
|---|---|---|---|
| | Peptide | P6Y | H12F |
| Binding ΔG | −5.56 ± 0.10 | −6.93 ± 0.11 | −6.85 ± 0.10 |
| (kcal/mol) | | | |
| K_{d} (μM) | 85 ± 14 | 8.6 ± 1.6 | 9.7 ± 1.6 |
| K_{d} Fold-Change | — | 10 ± 2.5 | 8.8 ± 2.0 |
| Relative to Lead | | | |
| Component | — | −1.37 ± 0.15 | −1.29 ± 0.14 |
| Binding ΔG | | | |
| Contribution | | | |
| (kcal/mol) | | | |

TABLE 13D
Observed and predicted binding energies and dissociation
constants for the TRF26 P6Y + H12F double mutant peptide

| | TRF26 Mutations | P6Y + 1112F |
|---|---|---|
| Observed | Binding ΔG | −8.68 ± 0.15 |
| | (kcal/mol) | |
| | K_{d} (μM) | 0.5 ± 0.1 |
| | K_{d} Fold-Change | 190 ± 57 |
| | Relative to Lead | |
| Predicted | Binding ΔG | −8.22 ± 0.18 |
| | (kcal/mol) | |
| | K_{d} Range (μM) | 1.3-0.7 |

### Example 30 Peptide Affinity Element Optimization by Evaluation of Multinomial Variants Generated by Light Directed Array Synthesis

This example demonstrates the identification of variants of a lead peptide, where the variants have improved binding properties with respect to a target of interest, by generating multinomial variants designed to contain substitutions in more than one position relative to the lead peptide and screening them for optimized target affinity/specificity. Because the number of multinomial variants increases exponentially with the size of the substitution set and number of varied positions (Xⁿ: X=size of substitution set, n=number of variable position), large libraries of variants are required to sample the sequence space encompassed by the defined set of amino acids and variable positions. Photolithographic patterning is one method that can be used to pattern a large number of variants in a small surface area that can be imaged by commercial fluorescence imagers. Once a patterned library is synthesized, the multinomial variants can be screen for target specificity/affinity. One advantage of this approach is that both additive and non-additive substitutions within a variant peptide can be captured in the screen.

Photolithographic patterning of variant arrays. Glass slides coated with a thin, optically transparent amine functionalized polymer were used as the sold-phase array substrate for all arrays. Variant peptides in the array were designed to contain both invariable and variable positions. Invariable positions were coupled using standard Fmoc solid-phase synthesis protocols. Briefly, the Fmoc protecting group was removed with 20% piperidine in DMF for 20 minutes. After deprotection, the next Fmoc amino acid was coupled to the N-terminus of the peptide chain (0.1 M Fmoc amino acid, 0.1 M HATU, 0.4 M DIPEA in DMF). Amino acid coupling times were typically 60 minutes. Variable positions in the peptide were coupled using light-directed chemistry. First, the N-terminal Fmoc group was removed from all peptides using 20% piperidine in DMF and the photolabile protecting group MeNPOC—Cl was coupled to the liberated N-terminal amines for 30 minutes. The array was then immersed in photolysis solution containing 30% β-mercaptoethanol, 7% DIPEA in acetonitrile. A photolithographic mask was projected on the substrate using a Digital Mirror Device, to selectively remove the MeNPOC protecting group in the illuminated regions. The substituted FMOC amino acid was added and allowed to couple to the selectively deprotected regions. After coupling, photodeprotection was repeated for different regions on the array and the next amino acid was coupled. This photodeprotection/coupling cycle was repeated for all substituted amino acids at a particular position in the peptide. After all peptides on the array are grown to the desired length a final side-chain deprotection is done using 95% TFA, 2.5% TIPS, 2.5% H₂O for 1 hour.

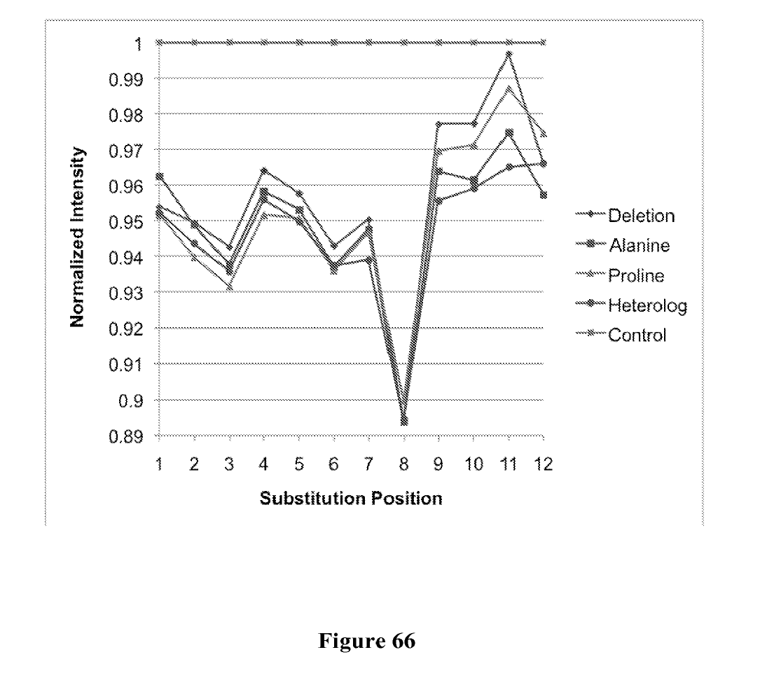

Multinomial mutant library synthesized for GAL80. The lead peptide EGEWTEGKLSLRGSC (BP2, Table 6) was selected for its moderate GAL80 affinity/specificity. Residues in the lead peptide most important for GAL80 binding were determined by alanine scanning mutagenesis. An array of all alanine point-mutants of the lead peptide was synthesized using photolithographic synthesis described above. After synthesis, the array was preblocked with 2% BSA in PBS for 2 hours, washed, then fluorescently labeled GAL80 (250 μM) in 1 mg/ml E. Coli lysate competitor was incubated with the array for 1 hour. Fluorescence images were obtained and analyzed and affinity relative to the lead peptide was plotted as shown in FIG. 66 (lead peptide=1).

Variable positions 4, 9, 11, and 12 were selected as those neighboring the positions identified as most important in the alanine scan (positions neighboring those which showed the greatest drop in intensity with an alanine substitution). The chemically diverse set of 10 amino acids {I,D,W,L,E,G,T,S,K,R} were selected as the amino acids to substitute into the four variable positions for a total of 10,000 unique variant peptides. Three replicates were included in the array to produce a total of 30,000 array features. The variant array (including the lead peptide) was synthesized using light-directed synthesis described above. After synthesis the array was preblocked with 2% BSA in PBS for 2 hours, then the array was incubated with 25 μM fluorescently labeled GAL80 in the presence of 1 mg/mL E. Coli lysate competitor for 1 hour. The resulting array was imaged using a commercial fluorescence scanner. The 25 variants showing the highest affinity for the Gal80 target had affinities on the order of 10 fold higher than the original template sequence (BP2); these are shown in Table 14.

TABLE 14
Variants with most improved affinity

| Fold | | Replicate Std. |
|---|---|---|
| Enhancement | Sequence | Error (%) |
| 11.3 | EGEITEGKKSKIGSC | 1.83 |
| 11.1 | EGEITEGKKSKLGSC | 5.94 |
| 11.1 | EGEWTEGKKSKGGSC | 4.83 |
| 11.0 | EGEWTEGKKSKRGSC | 6.12 |
| 10.9 | EGEITEGKKSKEGSC | 6.60 |
| 10.8 | EGEDTEGKKSKGGSC | 4.27 |
| 10.8 | EGEITEGKKSKGGSC | 5.13 |
| 10.7 | EGEWTEGKKSKLGSC | 8.91 |
| 10.7 | EGEWTEGKKSKEGSC | 6.70 |
| 10.6 | EGEITEGKKSKTGSC | 4.05 |
| 10.5 | EGEWTEGKKSKTGSC | 4.47 |
| 10.5 | EGEITEGKKSKRGSC | 6.21 |
| 10.4 | EGEDTEGKKSKLGSC | 6.71 |
| 10.4 | EGEDTEGKKSKIGSC | 2.03 |
| 10.4 | EGEWTEGKKSKIGSC | 3.80 |
| 10.4 | EGEDTEGKKSKRGSC | 6.97 |
| 10.2 | EGEDTEGKKSKTGSC | 3.75 |
| 10.1 | EGEDTEGKKSKEGSC | 6.09 |
| 9.91 | EGEITEGKKSKSGSC | 6.05 |
| 9.87 | EGEITEGKGSKKGSC | 6.04 |
| 9.81 | EGEKTEGKKSKLGSC | 8.56 |
| 9.72 | EGEITEGKLSKKGSC | 3.42 |
| 9.72 | EGEITEGKLSKKGSC | 3.42 |
| 9.70 | EGEKTEGKKSKEGSC | 4.25 |
| 9.24 | EGEKTEGKKSKGGSC | 7.89 |
| 8.50 | EGEKTEGKKSKTGSC | 3.77 |
| Template | EGEWTEGKLSLRGSC | 9.38 |
| Sequence | | |

### Example 31 Peptide Affinity Element Optimization by mRNA Display

This example demonstrates an mRNA display-based method for searching the sequence space surrounding a lead peptide so as to identify variants that have improved binding characteristics as compared to the lead peptide.

An oligonucletide library (5′-TTC TAA TAC GAC TCA CTA TAG GGA CAA TTA CTA TTT ACA ATT ACA ATG 126 246 445 135 135 226 245 216 245 436 216 246 126 346 446 216 346 ATG GGA ATG TCT GGA TC-3′, 1=97% G+1% C+1% T+1% A, 2=97% C+1% G+1% T+1% A, 3=97% T+1% G+1% C+1% A, 4=97% A+1% G+1% C+1% T, 5=98% G+2% C, 6=98% C+2% G) was purchased from Keck Oligonucleotide Synthesis Facility (Yale University). The library design was based on the sequence of peptide TRF26 (see Table 6) doped with a 4% mutation rate on each nucleic acid, so as to produce a library of peptides closely related to the original peptide TRF 26. The double stranded DNA library was attained using Klenow (New England BioLabs) and PCR was used to amplify the DNA for the mRNA display selection. The DNA primer (synthesized in house) (5′-ATAGCCGGTGCTACCGCTCAGGGCCTGATAAGATCCAGACATTCCCAT) was used to add the TMV and T7 promoter sites.

The mRNA selection was carried out according to a standard mRNA Display protocol (see Current Protocols in Molecular Biology (Wiley 2007), Unit 24.5, Anthony D.

Keefe, Protein Selection Using mRNA Display). The transferring target protein was immobilized on carboxyl derivatized MagnaBind™ beads (Pierce) using the manufacturer's suggested protocol (http://www.technochemical.com/instruction/0726 as4.pdf). Primers 5′-TTCTAATACGACTCACTATAGGGACAATTACTATTTACAATTACA and 5′-ATAGCCGGTGCTACCGCTCAGGGCCTG were used for the PCR amplification step of each round. Three rounds of selection were carried out with increasing selection stringency. The concentration of selection target, transferrin, decreased from 1.074 mg/100 μl beads at round one, to 0.1074 mg/10 μl beads at round two, then 0.0537 mg/5 μl beads at round three. The binding reaction took place at 4 C, shaking at 1,000 rpm for 1 hour. After three rounds, the sequences were cloned into E. coli Top 10 using TOPO TA kit, then miniprepared and sequenced in the DNA sequencing lab at Arizona State University.

Five clones (see Table 15) were selected, synthesized and purified by HPLC for characterization by surface plasmon resonance (SPR) (T100 instrument from Biacore). Transferrin was immobilized using standard NHS/EDC immobilization chemistry according to the methods described in Frostell-Karlsson, A., Remaeus, A., Andersson, K., Borg, P., Hamalainen, M., and Karlsson, R. (2000) J. Med. Chem. 43, resulting in 9758 RU of immobilized protein. HPLC purified peptides were injected over the surface and sensograms were recorded at multiple concentrations (32, 16, 8, 4, 2, 1, 0.5, 0.25, 0.125, and 0.0625 μM). Affinity plots were generated for each peptide and fit using a steady state affinity model. The affinities are shown in Table 15. The affinity of TBPMO23 is more than 10 fold improved in comparison to the original peptide TRF26.

TABLE 15
Sequences selected by mRNA display

| Clone | Sequence | MW(g/mol) | KD uM |
|---|---|---|---|
| TBPL005 | GHKVVPQRQIRHAYNRYGSC | 2370 | 150 |
| TBPL025 | GHKVVPQRQMRHAYNRNGSC | 2339 | 150 |
| TBPM023 | AHKVVPQRQMRHAYSRYGSC | 2375 | 11.6 |
| TBPM021 | ATRWCPSARPATPTTATGSC | 2035 | >300 |
| TBPM003 | PTGWCPAPDPPRLHPLHGSC | 2138 | >300 |

### Example 32 Microarray Screening of Peptides with Controlled Spacing

This example demonstrates an alternative peptide microarray screening methodology in which the spacing of peptide probes on the microarray is controlled, thereby affecting the extent to which an applied target can interact with multiple probes simultaneously.

Peptide microarrays were prepared by robotically spotting approximately 10,000 distinct polypeptide compositions, two replicate array features per polypeptide sequence. Each polypeptide was 20 residues in length, with glycine-serine-cysteine as the three C-terminal residues and the remaining residues determined computationally by a pseudorandom process in which each of the 20 naturally occurring amino acids except cysteine had an equal probability of being chosen at each position. Peptides were synthesized by Alta Biosciences, Birmingham, UK. Each polypeptide was first dissolved in dimethyl formamide overnight and master stock plates prepared by adding an equal volume of water so that the final polypeptide concentration was about 2 mg/ml. Working spotting plates were prepared by diluting equal volumes of the polypeptides from the master plates with phosphate buffered saline for a final polypeptide concentration of about 1 mg/ml. The polypeptides were spotted in duplicate using a SpotArray 72 microarray printer (Perkin Elmer, Wellesley, Mass.) and the printed slides stored under an argon atmosphere at 4° C. until used.

Spacing-controlled NSB arrays were prepared by robotically spotting the peptides on NSB amine slides (Nano Surface Biosciences Postech) according to the manufacturer's recommended protocol (http://www.nsbpostech.com/products/User %20Manual.pdf), conjugating the peptides to the amine functionalized surface via a maleimide linker (SMCC) to the C-terminal cysteine of the peptides. NSB slides employ a dendrimer cone surface with the cone tips functionalized for conjugation of probes, and the cones having a predetermined spacing of 3-4 nm for NSB-9 slides and 6-7 nm for NSB-27 slides. Both NSB-9 and NSB-27 slides were evaluated; the NSB-27 slides did not spot adequately so NSB-9 slides were used.

Anti-P53 (Lab Vision, clone PAB-240) was applied to the array according to the following protocol and binding was detected by applying biotinylated secondary antibody with fluorescent labeled (Alexa555) streptavidin and scanning with an array reader:
1. Prepare blocking buffer (5 mL of 30% BSA, 6.9 uL of Mercaptohexanol, 25 uL of Tween20, plus 1×PBS to 50 mL)
2. Block the surface of the slide for 1 hour using 350 uL of blocking buffer. Spread the buffer out evenly, and incubate at 37° C. in a humidity chamber.
3. Wash the slide 1× with TBST.
4. Wash 2× with water, making sure there is no tween left (no bubbles).
5. Dry the blocked slide in a 50 mL conical tube by spinning for 5 minutes at 1500 rpm in a swinging bucket rotor.
6. Place an AbGene gene frame on the surface of the slide.
7. Prepare primary at desired concentration (100 nM for sera, a 1:500 dilution), diluted in blocking buffer (same formula as above, but without mercaptohexanol).
8. Add the appropriate volume to the slide and seal using the provided cover slips.
9. Incubate for 1 hour at 37° C. in the dark.
10. Remove the slide cover but not the gene frame, and wash the slide 3 times with 1 xTBST, for 5 minutes each wash.
11. Wash with water 3 times, 5 minutes each.
12. Do not dry the slides.
13. Rinse the slide covers with water and dry them off.
14. Prepare the labeled secondary antibody at desired concentration (0.1-5 nM), again diluted in blocking buffer without mercaptohexanol.
15. Add to slide and seal.
16. Incubate 1 hour at 37° C. in the dark.
17. Wash as before, and dry by spinning for 5 minutes at 1500 rpm in a conical tube.
18. Scan the slides at the appropriate wavelength with 70% PMT and 100% laser power.

For comparison, binding of anti-P53 was evaluated on peptide arrays having the same peptides as the NSB arrays spotted in the same pattern on a glass surface in accordance with the protocol previously described, which does not attempt to control probe spacing (see Example 2) Both array types were evaluated both with and without the organic prewash procedure described in Example 17 below.

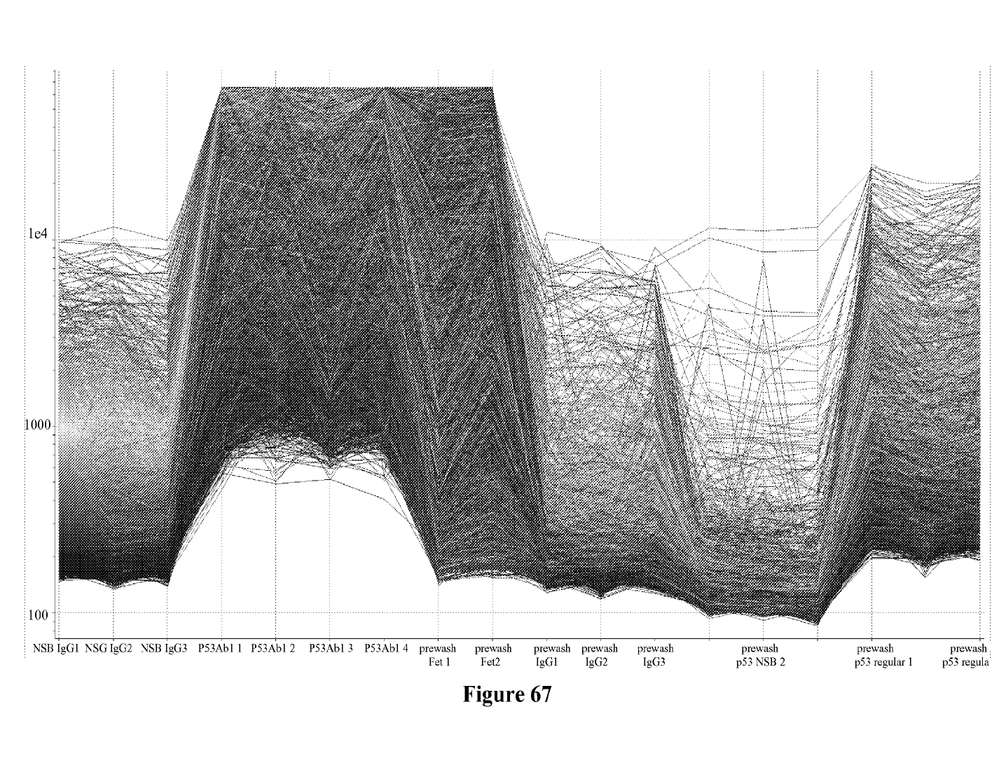

The arrays included, as positive controls, peptides corresponding to the known anti-P53 epitope; however, no significant binding of the anti-P53 to the corresponding spots was observed for either type of array. FIG. 67 shows a plot of the intensities corresponding to the spotted peptides for various experiments as follows (“prewash” refers to the organic prewash procedure described in Example 33 below): from left, the first three columns 251 show three replicates of the non-prewashed NSB array with only biotinylated secondary antibody and Alexa 555-labeled streptavidin applied as a negative control; the next four columns 252 show four replicates of non-prewashed NSB arrays with anti-P53 applied; the next two columns 253 show two replicates of prewashed NSB arrays with fetuin (a standard positive control) applied; the next three columns 254 show three replicates of prewashed NSB arrays with only biotinylated secondary antibody and Alexa 555-labeled streptavidin applied as a negative control; the next three columns 255 show three replicates of prewashed NSB arrays with P53 applied; and the rightmost three columns 256 show three replicates of prewashed non-NSB slides (i.e. ordinary glass slides without controlled spacing of probes) with P53 applied. Without organic prewash, the anti-P53 bound many more species of peptides on the non-spacing-controlled arrays than on the spacing-controlled slides. As described in Example 33 below, organic prewash reduces the number of peptide species bound on the ordinary non-spacing-controlled arrays 254 considerably, and, as FIG. 67 shows use of the spacing-controlled arrays 255 reduced the number of peptide species bound still further as compared to the prewashed non-spacing controlled arrays 256. In general, peptide species that strongly bound on the spacing-controlled arrays also tended to bind preferentially to the non-spacing-controlled arrays, both with and without organic prewash.

### Example 33 Organic Prewash

This example demonstrates a method for improving the screening power of peptide microarray affinity assays by washing the arrays with an organic solvent after spotting and prior to applying the protein target, so as to remove any peptides that may be aggregated with other peptides on the array but not covalently attached to the array surface. After preparation of the array in accordance with the methods previously described in Example 2, the array was washed one time for five minutes in 7.33% acetonitrile, 37% isopropanol, 0.55% trifluoroacetic acid, and 55% water. Alexa 555 labeled target protein transferrin was applied, together with Alexa 647 labeled E. coli lysate competitor, to the prewashed array and to an identical array without organic prewash. Table 15 shows the relative ranks of the transferrin-binding peptides whose sequences are shown in Table 6, ranked according to the ranking formula previously described in Example 2. As Table 5 shows, peptide TRF-19, previously determined by SPR analysis to be a poor binder of transferrin, ranked no. 5,010 on the array without organic prewash, but ranked no. 9601 on the prewashed array. Conversely, peptide TRF-21, shown by SPR analysis to be a relatively strong binder of transferrin, rose in rank from 84 on the non-prewashed array to rank no. 5 on the prewashed array. Peptides TRF-23 and TRF-26, both relatively strong binders, also improved in rank. The number of peptides scoring above a predetermined threshold was considerably reduced for the prewashed arrays as compared to non-prewashed arrays. These results illustrate that the organic prewash procedure is helpful for reducing false positives and focusing the screen in favor of stronger binders.

TABLE 16
Relative ranks of transferrin-binding peptides

| Peptide | Rank - non-prewash | Rank -- prewash |
|---|---|---|
| TRF19 | 5010 | 9601 |
| TRF20 | 18 | 289 |
| TRF21 | 84 | 5 |
| TRF22 | 711 | 61 |
| TRF23 | 2722 | 2091 |
| TRF24 | 71 | 958 |
| TRF25 | 736 | 603 |
| TRF26 | 596 | 436 |
| TRF27 | 1289 | 3325 |
| TRF28 | 601 | 712 |

### Example 34 Selection Criteria

This prospective example describes the selection of peptides as candidates for further evaluation as potential synbody binding elements, based on the results of SPR testing as described in Example 26. For each peptide, after data analysis and filtering for quality control, and after reference subtraction, as described in Example 26, the magnitude of the peak response is compared to the computed theoretical maximum (“Rmax”). Peptides having peak responses greater than 110 percent of Rmax are tentatively screened out as likely reflecting aggregation effects or other artifacts and not indicative of true specific binding levels. Peptides having peak responses less than 90 percent of Rmax are tentatively screened out as having insufficient affinity for the protein target. Recognizing that for most applications a long half-life of association is useful, those of the remaining peptides having less than five percent decline in response over one minute after termination of injection of peptide are selected for further evaluation by MALDI-MS. Of the peptides selected for evaluation by MALDI-MS, those producing spectra whose major peak corresponds to the correct peptide sequence (rather than a truncation product or impurity) are reevaluated by SPR using a longer injection time so as to facilitate obtaining a more accurate measurement of off rate. Those peptides displaying the longest half lives in this reevaluation are selected for conjugation to linkers for screening as synbodies. The various thresholds for peak response, decline in response, and MALDI evaluation may be adjusted as necessary to produce a desired quantity of candidates after screening.

### Example 35 Comparison of Peptide Screening Methods

The preceding examples have described several methods for screening peptides as candidates for use as binding elements for synbodies, including peptide affinity microarray evaluation without organic prewash (see Example 2), peptide affinity microarray evaluation with organic prewash (Example 33), peptide affinity microarray evaluation using controlled-spacing arrays (Example 32), SPR evaluation of peak response, off-rate, and/or affinity (Examples 26, 27 and 34), and chromatographic screening (Example 28). These and any other screening modalities may be compared and/or their results combined or otherwise taken into account for purposes of selection of peptides as candidates for further evaluation. One screening modality may preferentially detect behavior that another modality may be less well suited to detect; for example, in the array modality, the protein target is applied in solution phase and the peptide is surface bound, while in the SPR method, the protein is surface-affixed and the peptide is applied in solution phase. FIG. 68 compares fluorescence intensity measured by peptide array experiment with SPR response for several of the transferrin-binding peptides shown in Table 6.

### Example 36 Analysis of Peptide Conformations and Energies in Complexes of Known Structure

This example demonstrates that many peptides when complexed in protein/peptide complexes of known structure adopt bound conformations wherein their end to end length in Angstroms lies in the range between 3.8*Sqrt[N] and 0.66*(3.8 N). Approximately 45,000 structure files from the Protein Data Bank (all available structures at the time of downloading) were obtained and screened to identify all structures containing any chain having a length from 8 to 30 residues, inclusive (2731 structure files). These were further screened to eliminate non-peptide structures, backbone-only structures, and other structure files not analyzable under the analysis methods to be applied, and from the remaining structures were extracted 9,163 separate interface structure files, each relating to a single peptide/protein interface and containing the full peptide sequence together with a continuous protein chain containing all residues containing any atom within 5 Angstroms of any atom of any residue of the peptide chain, but truncated to remove the non-interacting regions at either end of the protein chain, and with any non-interacting protein chains removed. Through an exception handling strategy during the analysis, structures having anomalies such as missing atoms were filtered out, leaving 5,998 interface structure files that were analyzable without generating exceptions. Hydrogen bonds, salt bridges, and pi-cation interactions were identified by the geometric relationships between atoms, and energies were estimated for each interaction so identified. The contribution of hydrophobic contributions of each residue to binding free energy were estimated by computing the accessible surface area of each atom for each chain of the interface absent the other chain, and for the complex, weighting each by a solvation parameter corresponding to the atom type, summing these for each residue to obtain an energy of solvation, and taking the difference for each residue between the solvation energy when bound and when unbound, generally in accordance with the method of Femandez-Recio, et al. Proteins: Structure Function and Bioinformatics 58: 134-143 (2005).

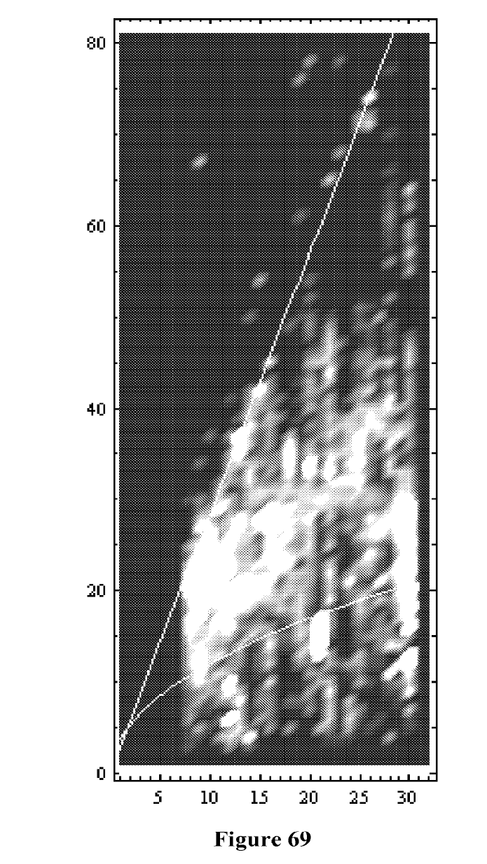

The end to end length of each peptide in the 9,163 interfaces was computed from the residue coordinates by determining the distance between the opposite-terminal alpha carbon atoms. FIG. 69 shows a density plot comparing the end to end length of each peptide as so determined with the theoretical random flight length for the same peptide (3.8*Sqrt[N], where N is the number of residues). The lower line corresponds to an end to end length equal to the theoretical random flight length. The upper line corresponds to an end to end length equal to 0.75 times the theoretical maximum length (3.8*N Angstroms). FIG. 69 shows most of the density (white areas correspond to high counts of peptides, black to zero) lies between the two lines.

An evaluation was also made of the distribution of peptide residues contributing at least −1.5 kcal/mole to the free energy of binding, as compared with those contributing less than −0.5 kcal/mole (the latter group including residues tending to detract from binding, due typically to burial of hydrophilic residues on binding). For the 5,998 analyzable interfaces, on average the size of the largest contiguous (in sequence) group of residues each contributing at least −1.5 kcal/mole to AG of binding was 1.7 residues (sigma=1.17), and the average number of residues (in the sequence) separating the two outermost residues each contributing at least −1.5 kcal/mole was 6.21 residues (sigma=7.25, reflecting the relatively large range of peptide lengths).

Although the foregoing invention has been described in some detail for purposes of clarity and understanding, it will be clear to one skilled in the art from a reading of this disclosure that various changes in form and detail can be made without departing from the true scope of the invention. The above examples are provided to illustrate the invention, but not to limit its scope; other variants of the invention will be readily apparent to those of ordinary skill in the art are encompassed by the claims of the invention. The scope of the invention should, therefore, be determined not with reference to the above description, but instead should be determined with reference to the appended claims along with their full scope of equivalents. All publications, references, GenBank citations, Swiss-Prot citation and the like, and patent documents cited in this application are incorporated by reference in their entirety for all purposes to the same extent as if each individual publication or patent document were so individually denoted. If more than one form of a sequence is associated with an accession number at different times, the form associated with the accession number as of the filing date of this application or priority document if the sequence is disclosed in the priority document is meant. Unless otherwise apparent from the context, any step, feature, element embodiment, aspect or the like can be used in combination with any other.

### REFERENCES

1. Tang, D. C., Nature 356, 1524 (1992).
2. Chambers, Nat Biotechnol 21, 1088-92 (2003).
3. Barry, Biotechniques 16, 616-8, 620 (1994).
4. Hust, Methods Mol Biol 295, 71-96 (2005).
5. Ellington, Nature 346, 818-22 (1990).
6. Binz, Nat Biotechnol 23, 1257-68 (2005).
7. Peng, Nat Chem Biol 2, 381-9 (2006).
8. MasipComb Chem High Throughput Screen 8, 235-9 (2005).
9. Roque, Biotechnol. Prog. 20, 639-654 (2004).
10. Silverman, Nat. Biotechnol. 23, 1556-1561 (2005).
11. Bes, C., et. al., Biochem. Biophys. Res. Comm. 343, 334-344 (2006).

We claim:

1. A multimeric peptide comprising a first affinity element conjugated to a second affinity element, wherein the first affinity element comprises a first peptide conjugated to a first DNA strand, the second affinity element comprises a second peptide conjugated to a second DNA strand, the first peptide and second peptide comprise a random combination of amino acids selected from the group of G, T, Q, K, S, W, L, and R; and the first affinity element is conjugated to the second affinity element by hybridization of the first DNA strand and the second DNA strand.

2. The multimeric peptide of claim 1, further comprising a first template DNA strand and a second template DNA strand wherein the at least one template DNA strand conjugates the first peptide with the first DNA strand and the at least one template DNA strand conjugates the second peptide with the second DNA strand.

3. The multimeric peptide of claim 2, wherein the first template DNA strand is conjugated to the first peptide at the C-terminus of the first peptide and the second template DNA strand is conjugated to the second peptide at the C-terminus of the second peptide.

4. The multimeric peptide of claim 3, wherein the first template DNA strand is conjugated to the first peptide using standard amine coupling chemistry and the second template DNA strand is conjugated to the second peptide using standard amine coupling chemistry.

5. The multimeric peptide of claim 2, wherein the first DNA strand is conjugated to the first peptide by conjugating with the first template strand and the second DNA strand is conjugated to the second peptide by conjugating with second template strand.

6. The multimeric peptide of claim 5, wherein the first DNA strand is conjugated to the first template strand by UV cross-linking and the second DNA strand is conjugated to the second template by UV cross-linking.

7. The multimeric peptide of claim 1, wherein the first peptide and the second peptide each comprise 8 to 35 amino acids.

8. The multimeric peptide of claim 1, wherein the first peptide and the second peptide each comprise 8 to 20 amino acids.

9. The multimeric peptide of claim 1, wherein the first DNA strand and the second DNA strand are synthetic DNA.

10. The multimeric peptide of claim 1, wherein the total distance between the first peptide and the second peptide is 0.5 nm to 30 nm.

11. The multimeric peptide of claim 1, wherein the total distance between the first is peptide and the second peptide is 0.5 nm to 10 nm.

12. The multimeric peptide of claim 1, wherein the total distance between the first peptide and the second peptide is 4.3 nm.

13. The multimeric peptide of claim 1, wherein the total distance between the first peptide and the second peptide is 2 nm.
